SEQUENCE LISTING (6 sequences)

SEQ ID NO: 1 - DNA, 41 bp, Artificial Sequence
  based on Escherichia coli
gccttgccagcccgctcagtcagagtttgatcctggctcag

SEQ ID NO: 2 - DNA, 52 bp, Artificial Sequence
  based on Escherichia coli
gcctccctcgcgccatcagnnnnnnnnnnnncatgctgcctcccgtaggagt

SEQ ID NO: 3 - DNA, 21 bp, Artificial Sequence
  based on Escherichia coli
gacggtatcgataagcttgat

SEQ ID NO: 4 - DNA, 15 bp, Artificial Sequence
  based on Escherichia coli
gacctcgaggggggg

SEQ ID NO: 5 - DNA, 24 bp, Artificial Sequence
  based on Escherichia coli
gaattcattaaagaggagaaaggt

SEQ ID NO: 6 - DNA, 24 bp, Artificial Sequence
  based on Escherichia coli
tttcgttttatttgatgcctctag

(19) United States
(12) Patent Application Publication
(10) Pub. No.: US 2014/0045744 A1
(43) Pub. Date: Feb. 13, 2014

(54) CULTURED COLLECTION OF GUT MICROBIAL COMMUNITY

(71) Applicant: THE WASHINGTON UNIVERSITY, St. Louis, MO (US)

(72) Inventors: Jeffrey I. Gordon, St. Louis, MO (US); Jeremiah J. Faith, St. Louis, MO (US); Nathan P. McNulty, St. Louis, MO (US); Federico E. Rey, St. Louis, MO (US); Andrew Goodman, St. Louis, MO (US); George Kallstrom, St. Louis, MO (US); Vanessa K. Ridaura, St. Louis, MO (US)

(21) Appl. No.: 14/022,000

(22) Filed: Sep. 9, 2013

Related U.S. Application Data
(63) Continuation-in-part of application No. PCT/US2012/028600, filed on Mar. 9, 2012.
(60) Provisional application No. 61/450,741, filed on Mar. 9, 2011.
(60) Provisional application No. 61/485,887, filed on May 13, 2011.
(60) Provisional application No. 61/497,663, filed on Jun. 16, 2011.

(51) Int. Cl. C12Q 1/02

(52) U.S. Cl. CPC C12Q 1/025
USPC 514/5.7; 435/252.4; 435/29; 506/14; 506/10

(57) ABSTRACT

The present invention encompasses cultured collections of a gut microbial community, models comprising such cultures, and methods of use thereof.

### CROSS REFERENCE TO RELATED APPLICATIONS

This application claims priority to PCT application PCT/US2012/028600, filed Mar. 9, 2012, which claims priority to U.S. provisional applications 61/450,741, filed Mar. 9, 2011, 61/485,887, filed May 13, 2011, and 61/497,663, filed Jun. 16, 2011, each of which is hereby incorporated by reference in its entirety.

### GOVERNMENTAL RIGHTS

This invention was made with government support under DK70977 and DK30292 awarded by the National Institutes of Health. The government has certain rights in the invention.

### FIELD OF THE INVENTION

The present invention encompasses cultured collections of a gut microbial community, models comprising such cultures, and methods of use thereof.

### BACKGROUND OF THE INVENTION

The largest microbial community in the human body resides in the gut and comprises somewhere between 300 and 1000 different microbial species. The human body, consisting of about 100 trillion cells, carries about ten times as many microorganisms in the intestines. The gut microbiome contains at least two orders of magnitude more genes than are found in the Homo sapiens genome. However, efforts to dissect the functional interactions between microbial communities and their environmental or animal habitats are complicated by the long-standing observation that, for many of these communities, the great majority of organisms have not been cultured in the laboratory, and some may not have been previously identified. Furthermore, experiments to determine the effect of a perturbation on a gut microbial community are hampered because teasing out the effects of a particular perturbation on each of the hundreds or thousands of different species in a gut microbial community using current techniques is difficult at best and may well be impossible. A need exists, therefore, for methods of culturing and dissecting the gut microbial community populations both in vitro and in vivo.

### SUMMARY OF THE INVENTION

One aspect of the invention encompasses a composition. The composition comprises (i) an in vitro cultured collection of a gut microbial community or (ii) a clonally arrayed culture collection of a gut microbial community. In certain aspects, the gut microbial community is from a human or a germfree mouse colonized with a gut microbial community or an arrayed culture collection of a gut microbial community.

Another aspect of the invention encompasses a composition. The composition comprises (i) an in vitro cultured collection of a gut microbial community or (ii) a clonally arrayed culture collection of a gut microbial community. The cultured gut microbial community has (i) at least 60%, at least 70%, at least 80% or at least 90% of the order-level phylotopic composition of the original gut microbial community; or (ii) at least 60%, at least 70%, at least 80% or at least 90% of the metagenome, transcriptome, or proteome composition of the original gut microbial community; or (iii) at least 60%, at least 70%, at least 80% or at least 90% of the order-level phylotopic composition of the original gut microbial community and at least 60%, at least 70%, at least 80% or at least 90% of the metagenome, transcriptome, or proteome composition of the original gut microbial community. In certain aspects, the clonally arrayed culture collection was prepared (i) without colony picking and/or (ii) using a most probable number (MPN) technique.

Another aspect of the invention encompasses a method of determining the effect of a perturbation on a gut microbial community. The method comprises applying the perturbation to a cultured collection of a gut microbial community and determining the difference in the community before and after the application of the perturbation, wherein the difference in the cultured collection represents the effect of the perturbation on the original gut microbial community.

Another aspect of the invention encompasses a composition. The composition comprises (i) an in vitro cultured collection of a gut microbial community or (ii) a clonally arrayed culture collection of a gut microbial community. The cultured gut microbial community has (i) at least 98% of the order-level phylotopic composition of the original gut microbial community; or (ii) at least 98% of the metagenome, transcriptome, or proteome composition of the original gut microbial community; or (iii) at least 98% of the order-level phylotopic composition of the original gut microbial community and at least 98% of the metagenome, transcriptome, or proteome composition of the original gut microbial community. In certain aspects, the clonally arrayed culture collection was prepared (i) without colony picking and/or (ii) using a most probable number (MPN) technique.

Another aspect of the invention encompasses a method of specifically manipulating the abundance of a member of a gut microbiome of a host to a target level by changing the diet of the host. The method comprises (a) determining the linear coefficient for a particular gut microbiome member in relation to protein, fat, polysaccharide, and simple sugar; (b) determining the amount of protein, fat, polysaccharide and sugar in a diet necessary to achieve the target level of the gut microbiome member based on the linear coefficients from (a); and (c) feeding a diet to the host that contains the amount of protein, fat, polysaccharide and sugar determined in (b). In certain aspects, the abundance of a member of a gut microbiome may be calculated with the equation

yᵢ=β₀+βₚᵣₒₜₑᵢₙXₚᵣₒₜₑᵢₙ+β_{Polysaccharide}X_{polysaccharide}+β_{sucrose}X_{sucrose}+β_{fat}X_{fat},

where yᵢ is the abundance of the member of the gut microbiome, β₀ is the calculated parameter for the intercept, X is the amount in g/(kg of total diet) of the diet ingredient, and βₚᵣₒₜₑᵢₙ, β_{polysaccharide}, β_{sucrose}, and β_{fat} are the linear coefficients for a particular gut microbiome member for each of the diet components.

Other aspects of the invention are described more thoroughly below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

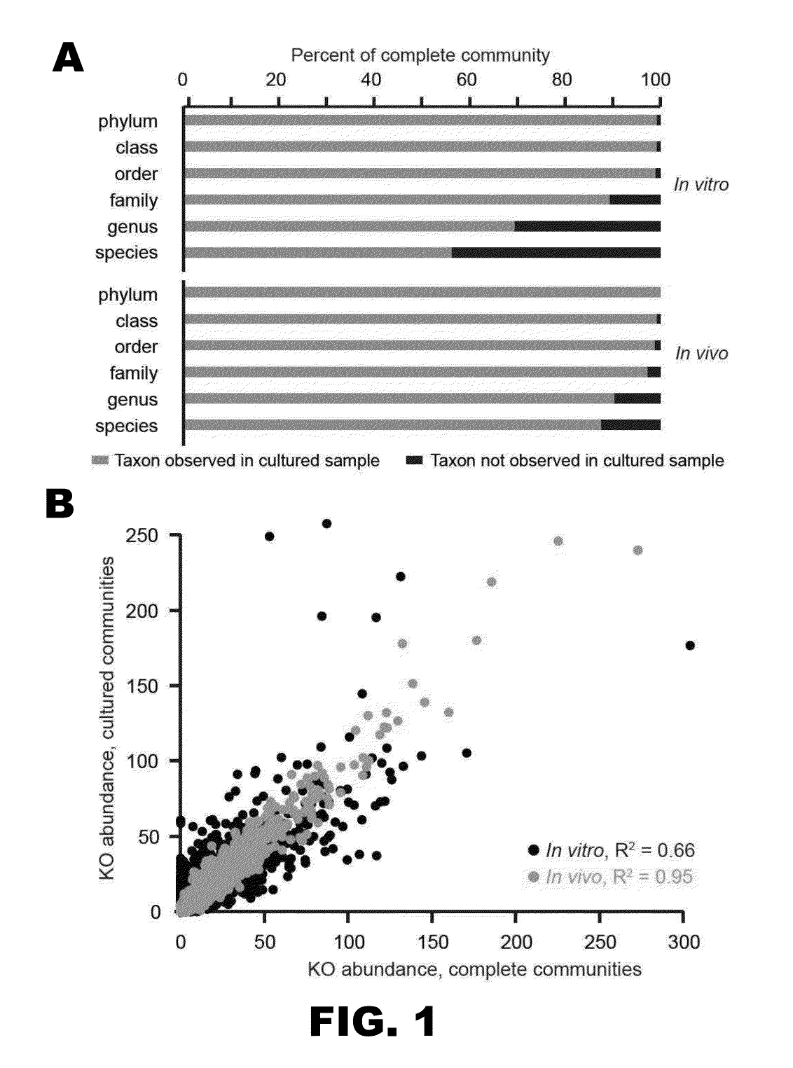

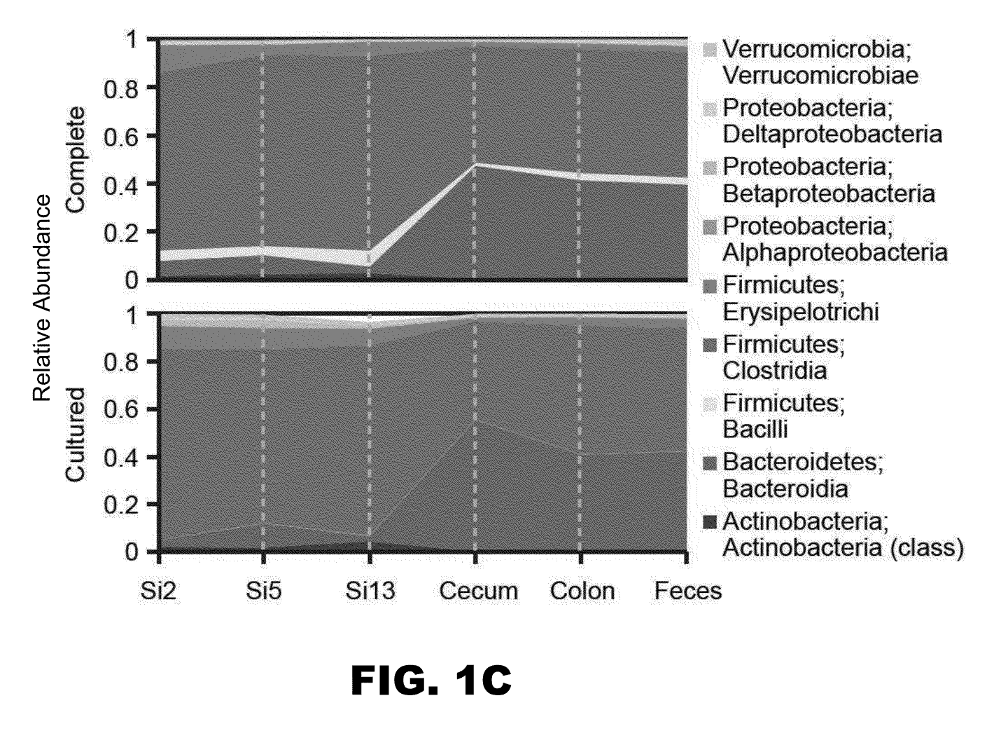

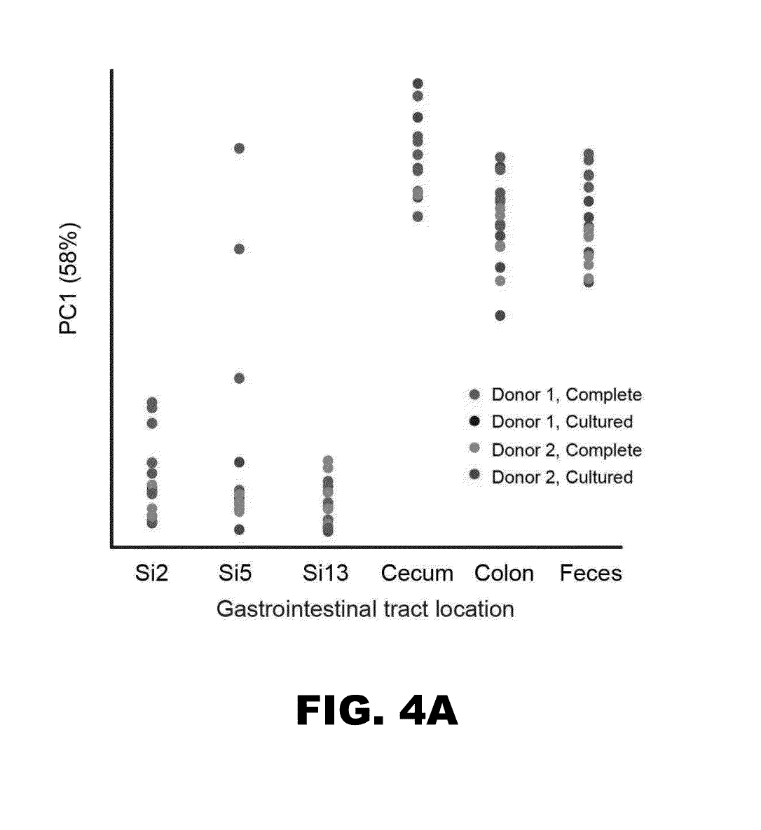

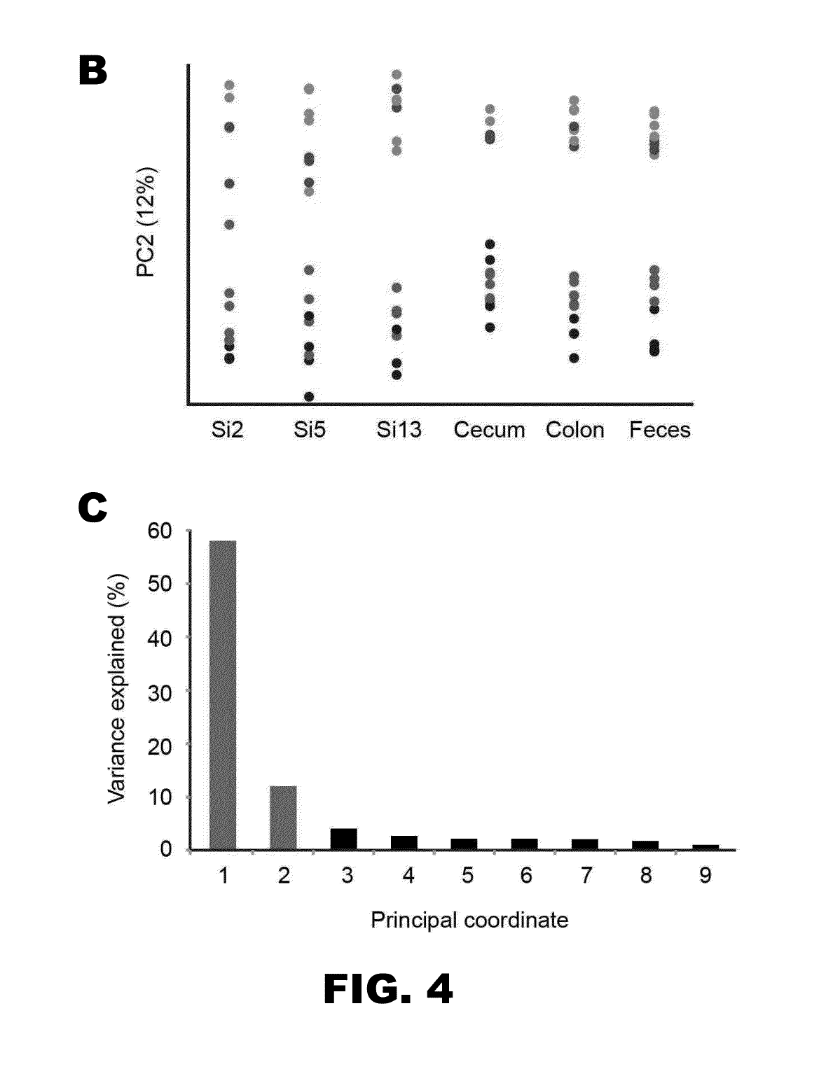

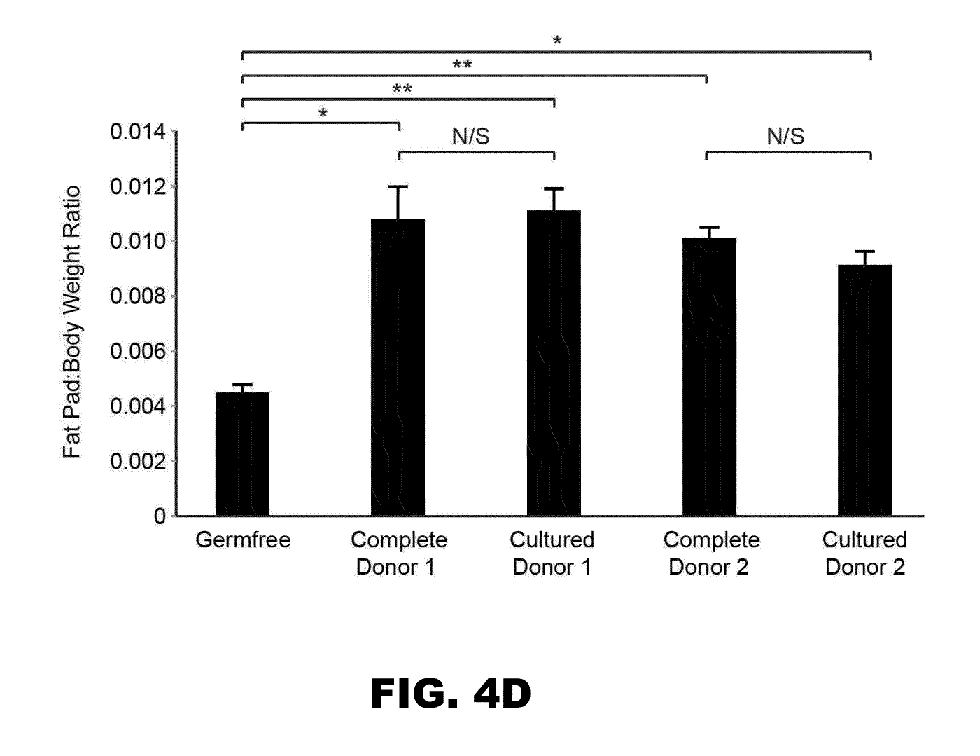

FIG. 1 Comparison of the taxonomic representation of bacterial species and gene content in complete versus cultured human fecal microbial communities before and after their introduction into gnotobiotic mice. (A) 16S rRNA sequences from complete microbiota were compared with those identified from microbial communities cultured from the same human donors. At each taxonomic level, the proportion of reads in the complete community belonging to a taxonomic group observed in the cultured sample is shown in blue; the proportion of reads belonging to a taxonomic group not observed in the cultured sample (or lacking taxonomic assignment) is shown in black. (Data shown are the average of two unrelated human donors.) In vitro samples refer to comparisons between human fecal samples and plated material. In vivo samples refer to comparisons between gnotobiotic mice colonized with a complete human fecal microbiota and mice colonized with the readily cultured microbes from the same human fecal sample. (B) Annotated functions identified in the microbiomes of complete and cultured human gut communities. Each point represents a KO designation plotted by relative abundance (average across two donors, per 100,000 sequencing reads). Black points represent KO comparisons between the in vitro samples; orange points represent comparisons between in vivo samples. (C) The distribution of taxa and their relative abundance along the length of the intestine are similar in the two groups of animals. Relative abundances of class-level taxa at six locations are shown; data represent the average of mice colonized from two unrelated donors. SI, small intestine divided into 16 equal-size segments and sampled at SI-2 (proximal), SI-5 (middle), and SI-13 (distal). PCoA suggests that gut biogeography, rather than donor or culturing, explains the majority (58%) of variance between samples (FIG. 4 A-C).

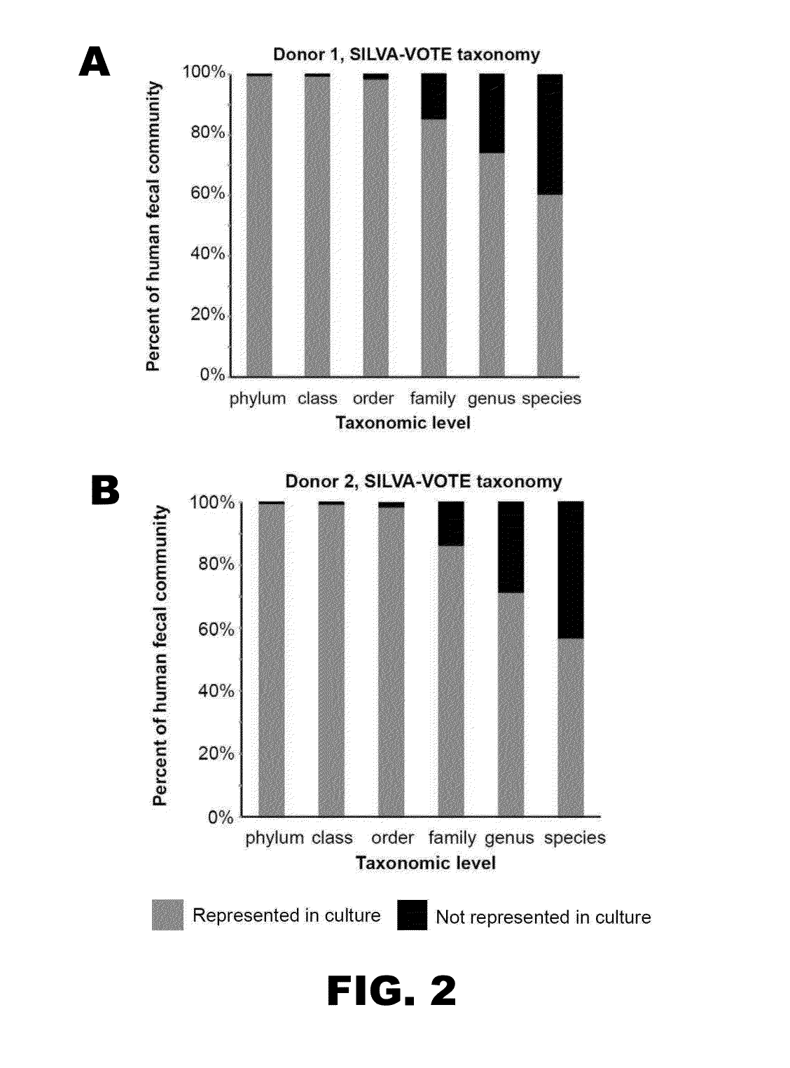

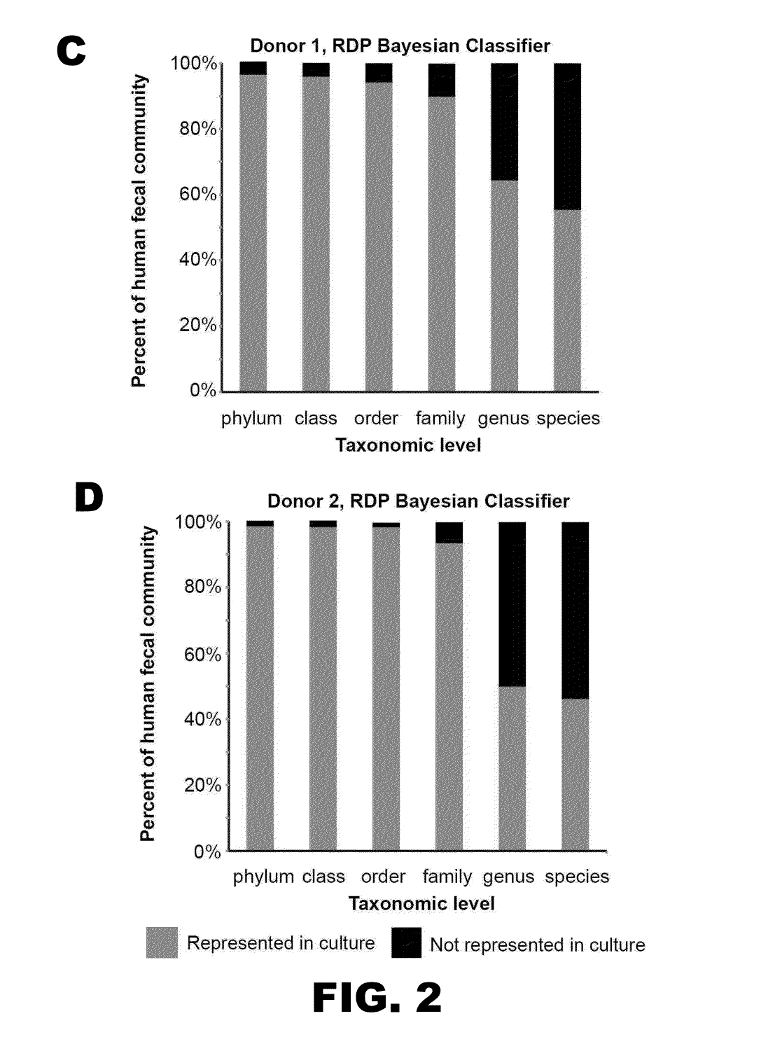

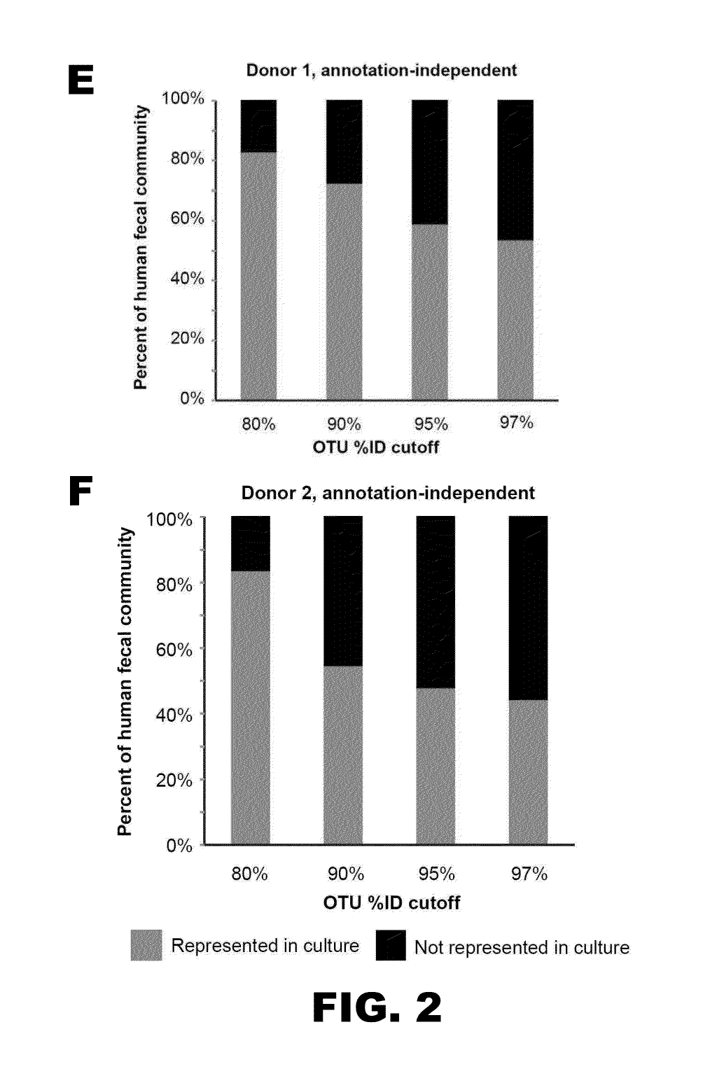

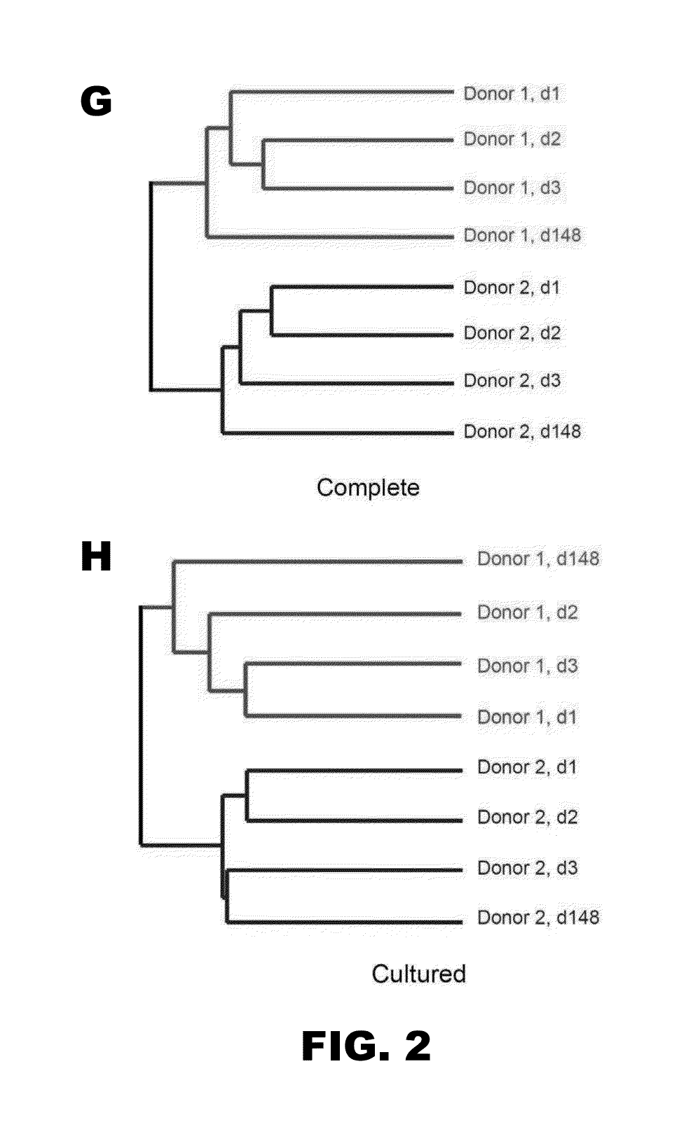

FIG. 2 Abundance of readily cultured taxa in fecal samples from two unrelated human donors (Donor 1: A, C, E; Donor 2: B, D, F), as determined by SILVA-VOTE (A, B), Ribosomal Database Project (RDP)-based 16S rRNA annotation (C, D), and annotation-independent (OTU % ID cutoff) methods (E-F) Analyses were performed as described in FIG. 1. Unsupervised hierarchical clustering of 16S rRNA datasets generated from either complete uncultured (G) or readily cultured (H) human gut microbial communities separates all samples from Donor 1 (red) from Donor 2 (blue). Unweighted pair group method with arithmetic mean (UPGMA) clustering of unweighted UniFrac distances between samples (rarefied to 1,000 de-noised, chimera-checked sequences each) is shown; nonphylogenetic distance metrics (Jaccard, Hellinger, Bray-Curtis) produce similar results (data not shown). In both G and H, bootstrap support separating Donor 1 samples from Donor 2 samples is 100% [100 iterations; 500 sequences subsampled from each complete (nonrarefied) dataset].

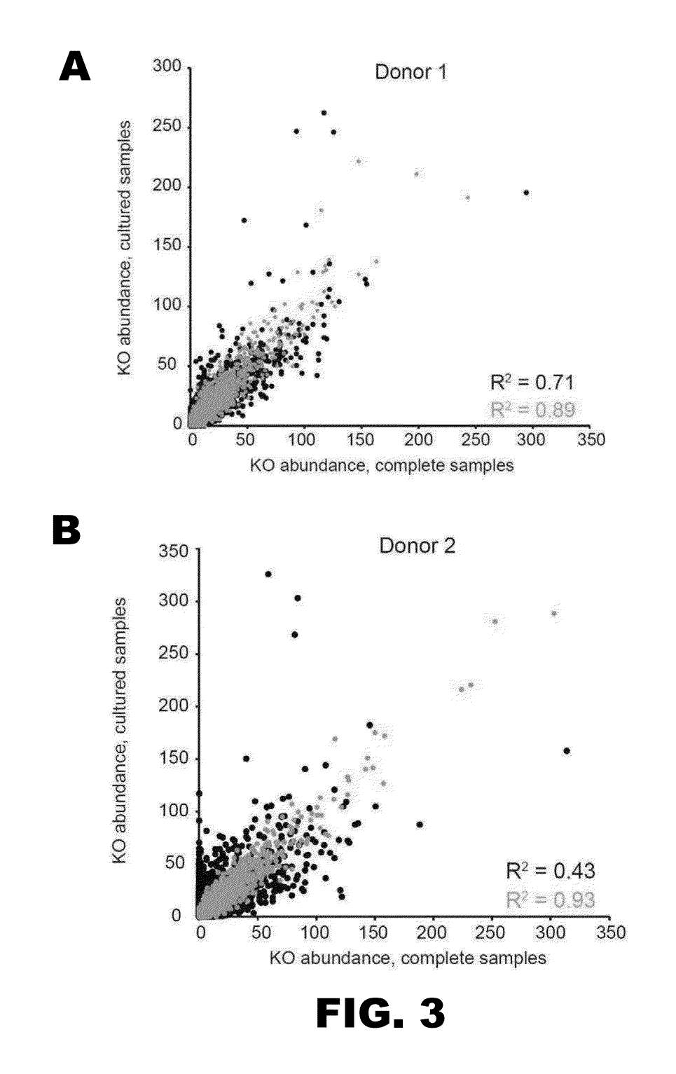

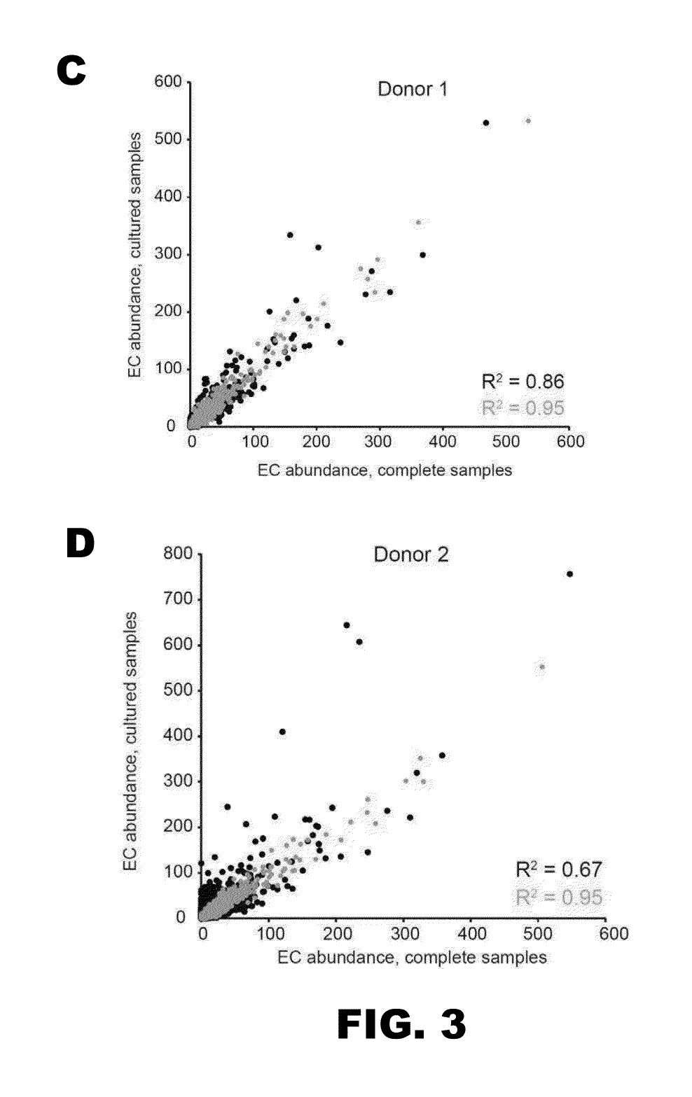

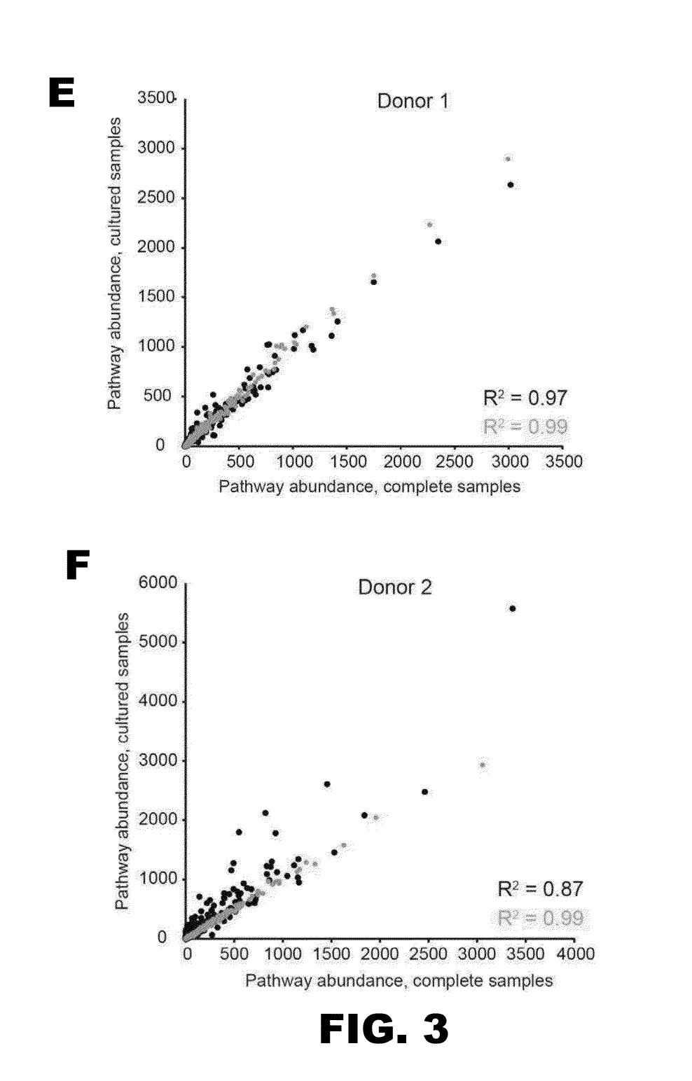

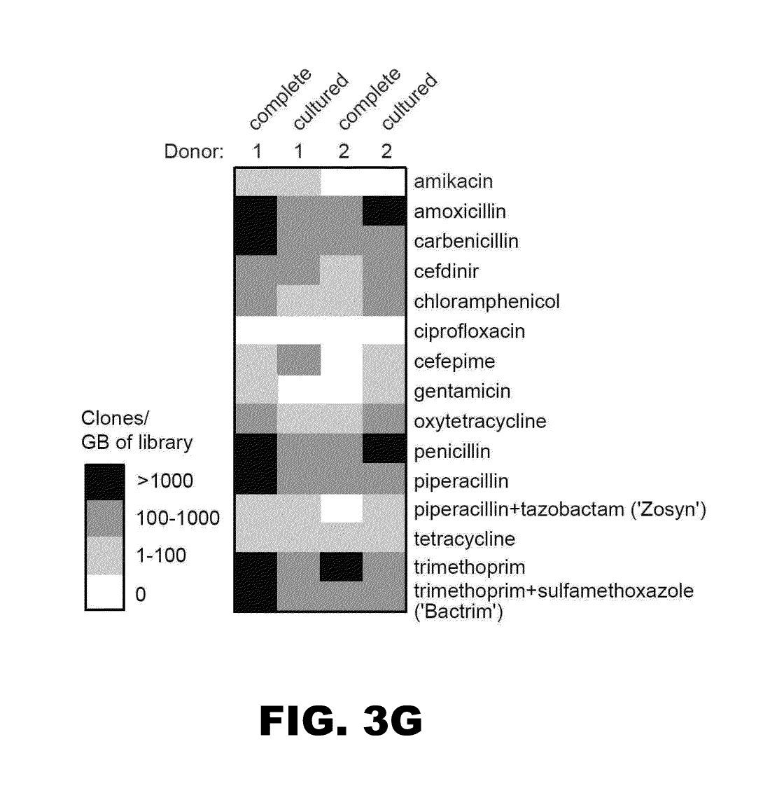

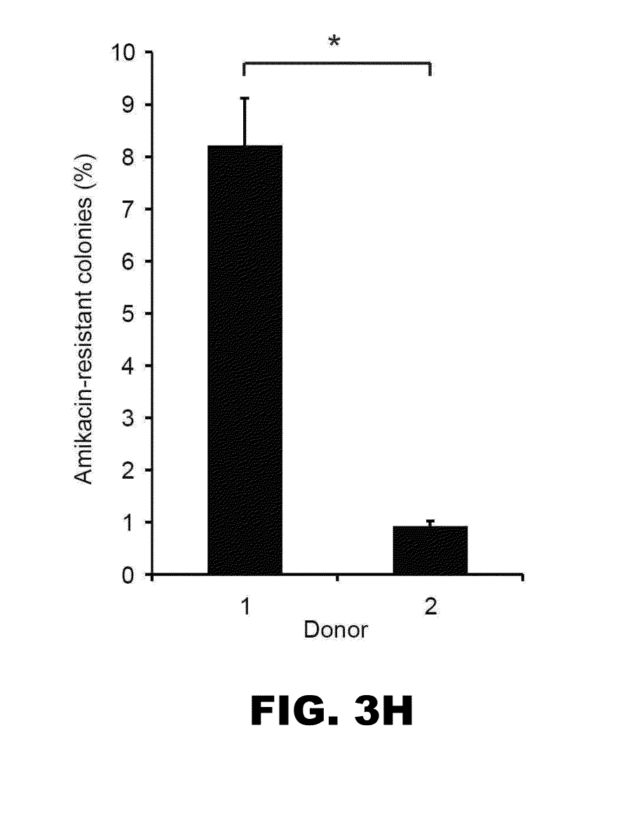

FIG. 3 Relative abundance of functional annotations in the uncultured (complete) and readily cultured fecal communities of two unrelated donors. From each donor, complete and cultured fecal samples also were introduced into germfree mice. After a 4-wk acclimatization period on a standard LF/PP diet, fecal microbiomes were characterized by shotgun pyrosequencing. Reads were mapped to KO (A and B), EC (C and D), and level 2 KEGG pathways (E and F). In these graphs, each point represents a functional annotation, and the axes represent the relative abundance (per 100,000 shotgun pyrosequencer reads) of these predicted functions in comparisons of complete versus cultured microbiomes (black points) and in comparisons of complete versus cultured microbiomes after each had been introduced into germfree mice (orange points). In each comparison, the goodness of fit (R2) values increase in communities that share the same environment (mouse gut) regardless of the donor. (G) Annotation-independent comparison of functions encoded in the microbiomes of uncultured complete or cultured fecal communities: capture of antibiotic resistance genes. Shades represent number of E. coli clones (per GB of subcloned DNA from complete or readily cultured microbial communities from each of two human donors), resulting from each of 15 antibiotic selections. (H) Proof-of-principle study connecting captured genes with their associated bacterial sources. Fecal DNA fragments cloned into E. coli from Donor 1, but not from Donor 2, conferred resistance to the aminoglycoside amikacin. Replating these fecal samples directly on high levels of amikacin (4,100 μg/mL) reveals that this functional difference is mirrored in the source communities. Mean values±SEM of triplicate samples (separate frozen aliquots of the original fecal material) are plotted. *P<0.005 based on student's t test (unpaired, two-tailed, assuming equal variance; P<0.02 by heteroscedastic test).

FIG. 4 Biogeography of complete and readily cultured human gut microbial communities in gnotobiotic mice and the impact of colonization on host adiposity. (A-C) Principal coordinate analysis (PCoA) of weighted UniFrac distances between samples collected along the length of the gut indicates that mice colonized with readily cultured microbial communities have microbiota similar to those colonized from an uncultured source. (A) Principal coordinate 1 (PC1) separates samples by location. (B) Principal coordinate 2 (PC2) separates samples by donor. (C) No other coordinate explains ≧5% of the total variance between samples. (D) Epididymal fat pad:body weight ratios in germfree mice and those colonized with complete or readily cultured microbial communities from the two human donors. Ratios represent the average±SEM from n=5 mice per group (except the Donor 2 cultured community; n=3). Asterisks indicate statistically significant differences based on an unpaired, two-tailed student's t test. *P<0.005; **P<0.001; N/S, not significant.

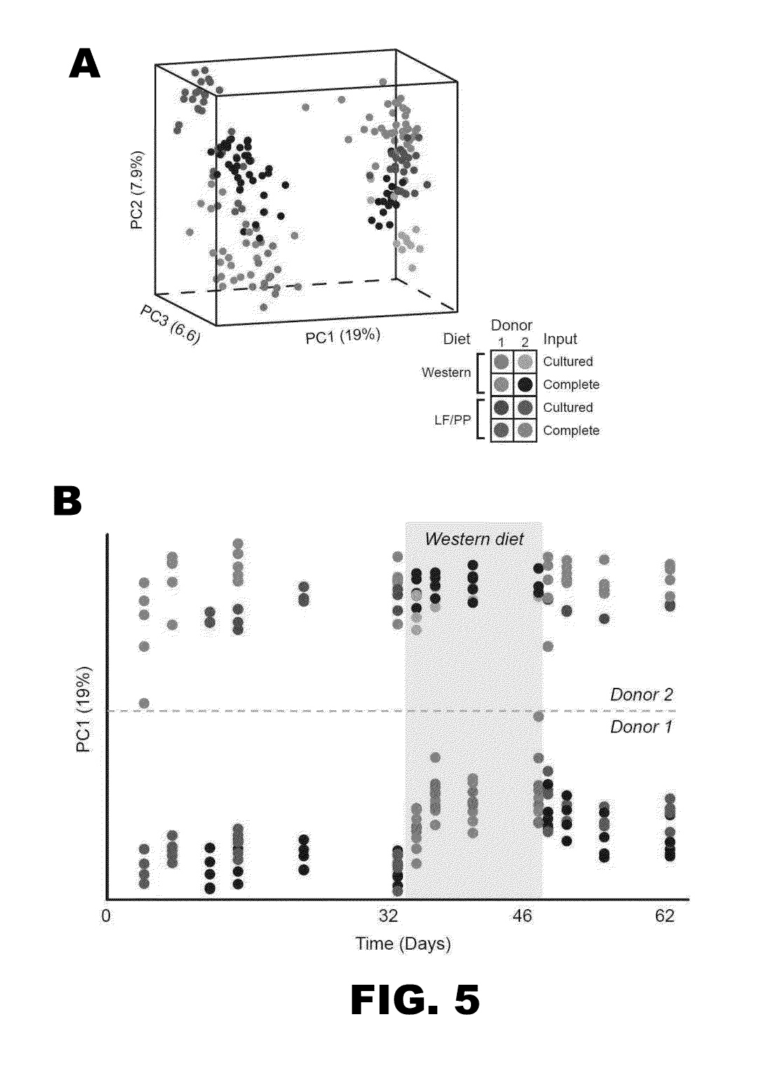

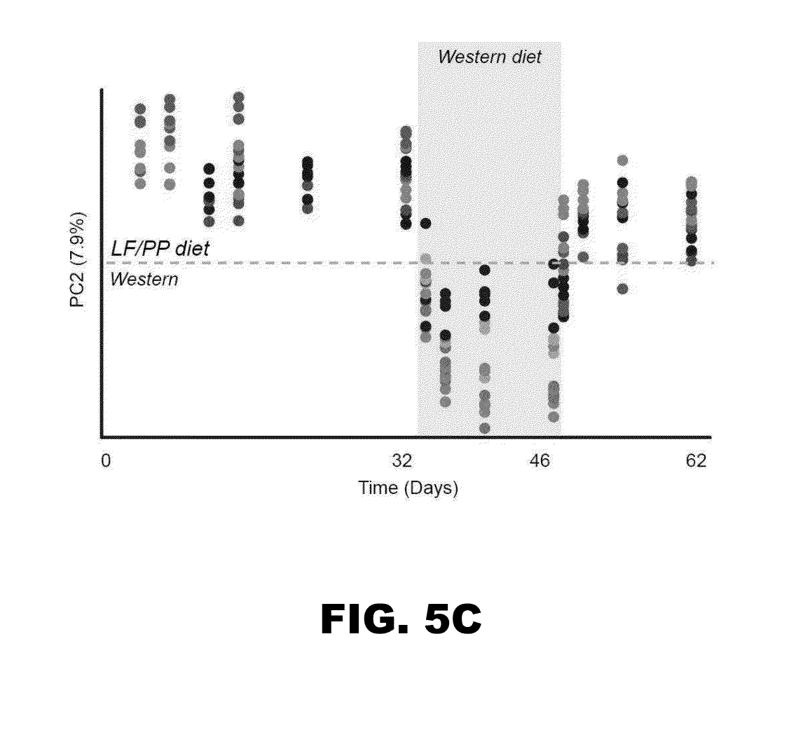

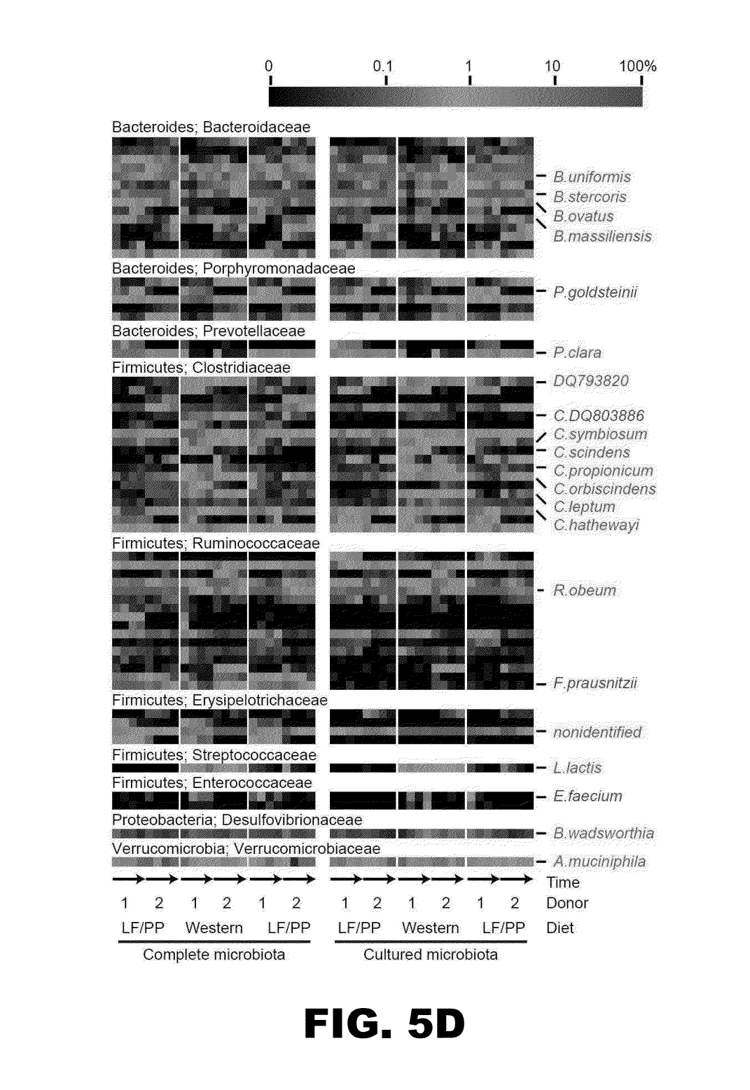

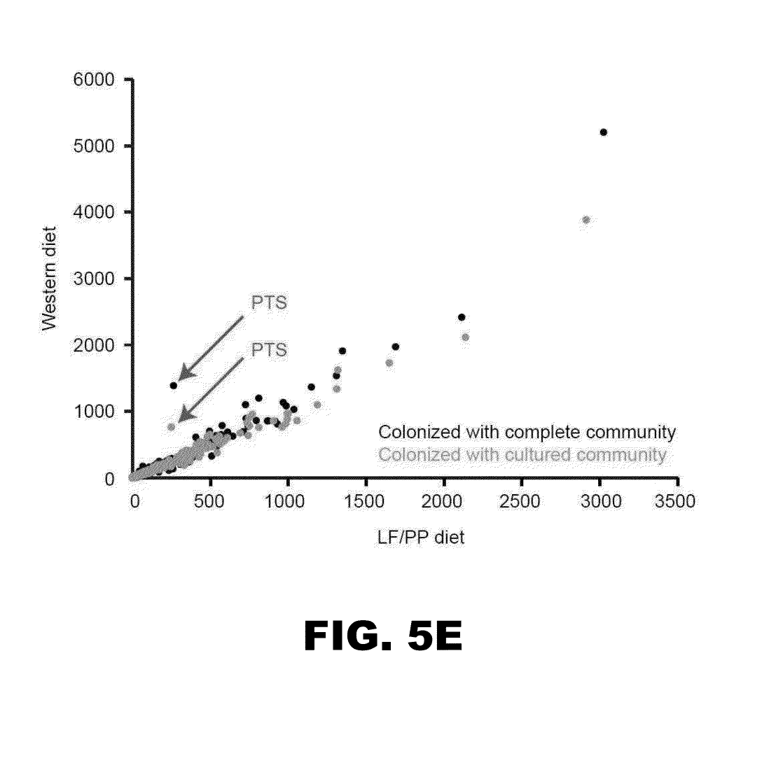

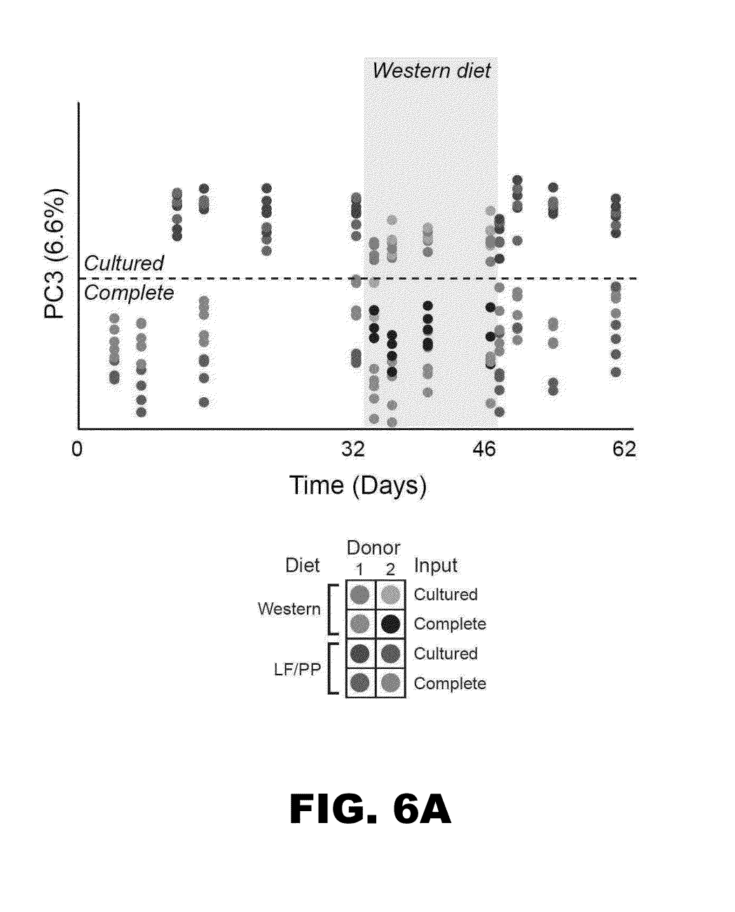

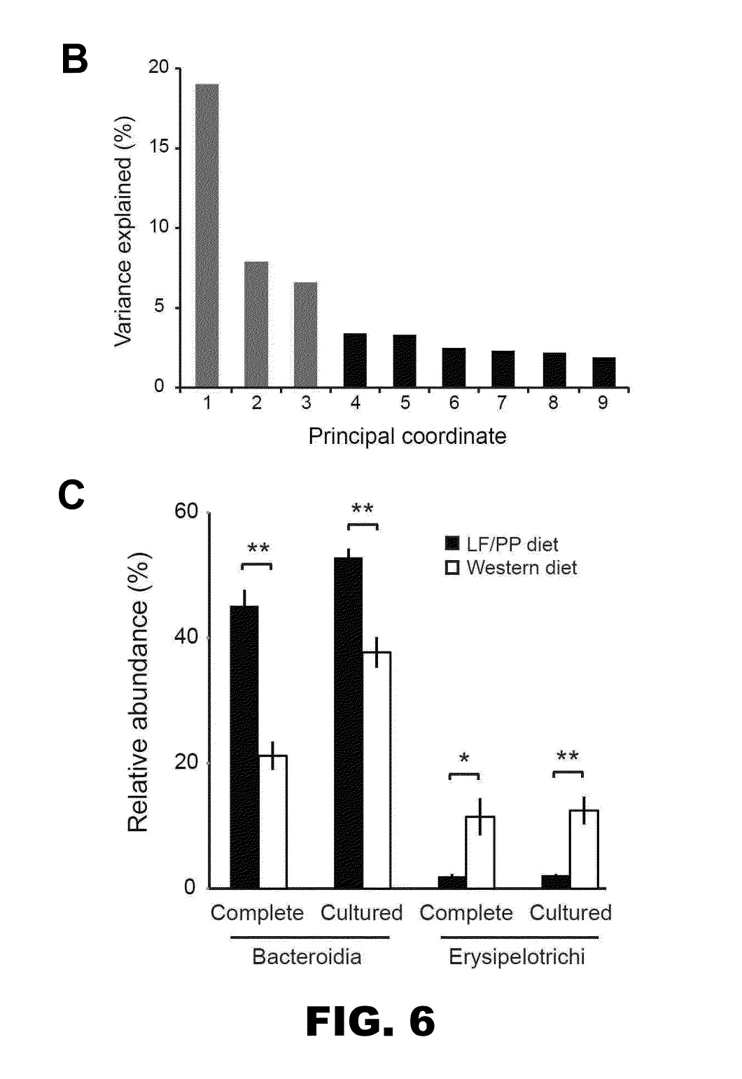

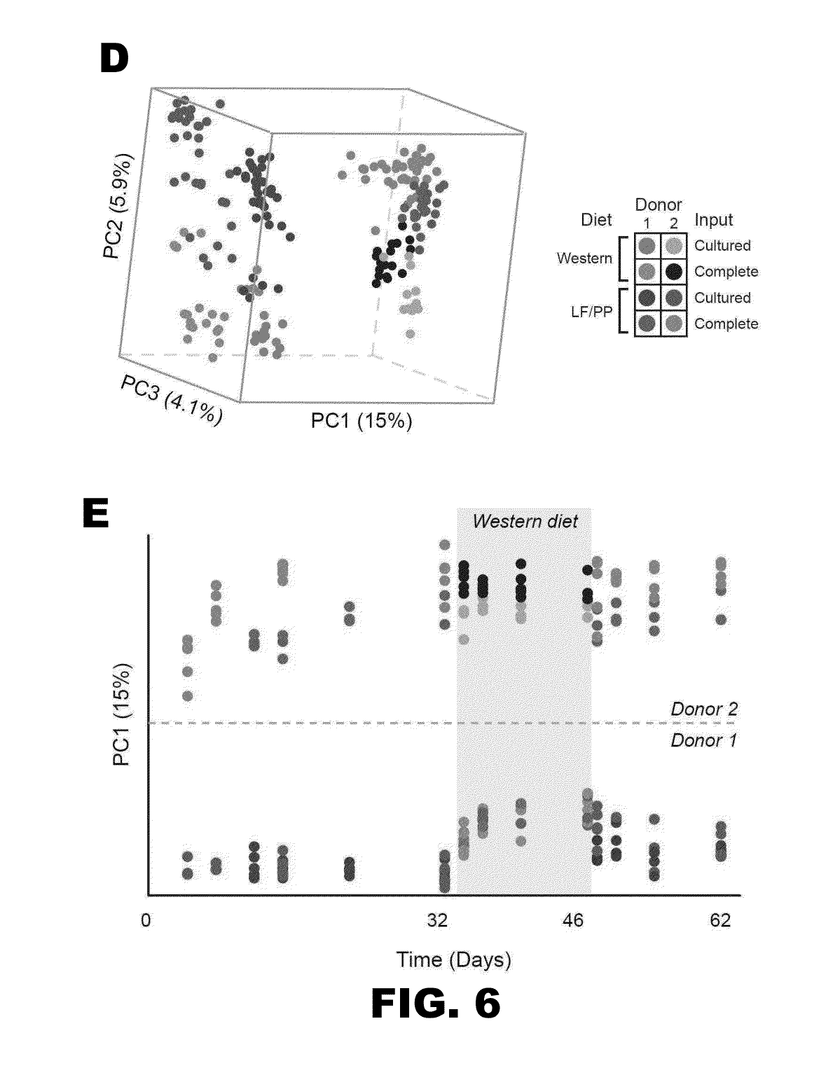

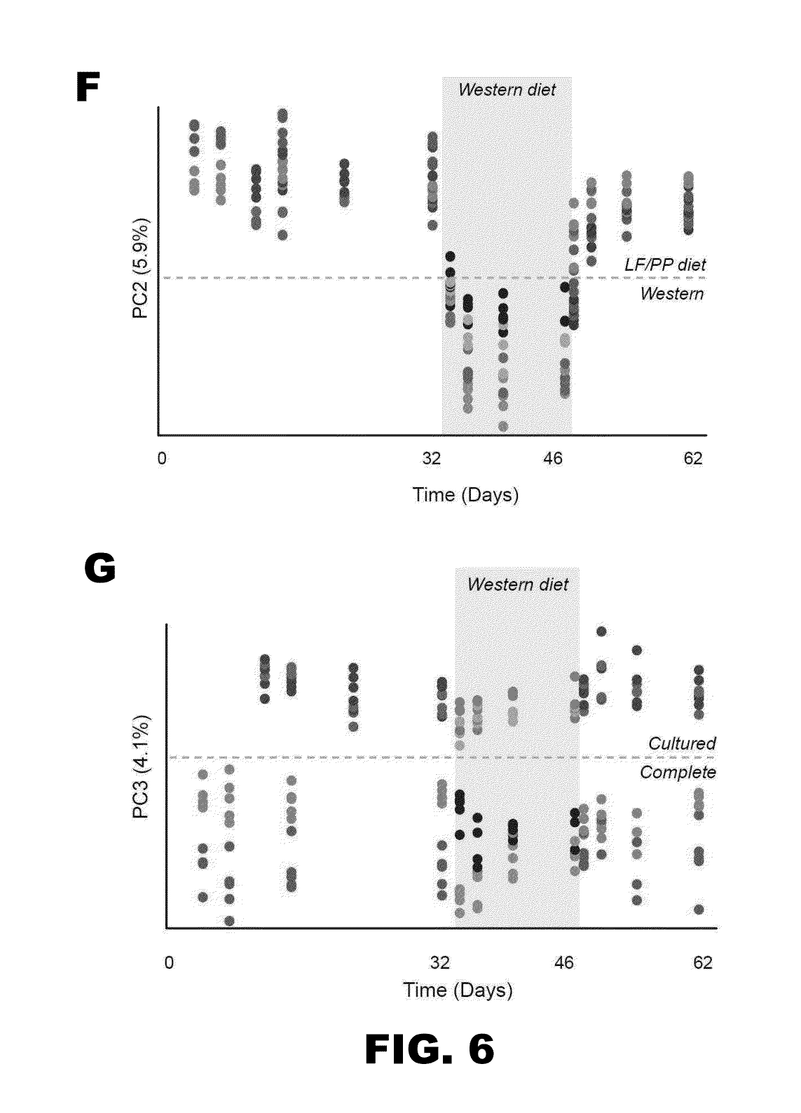

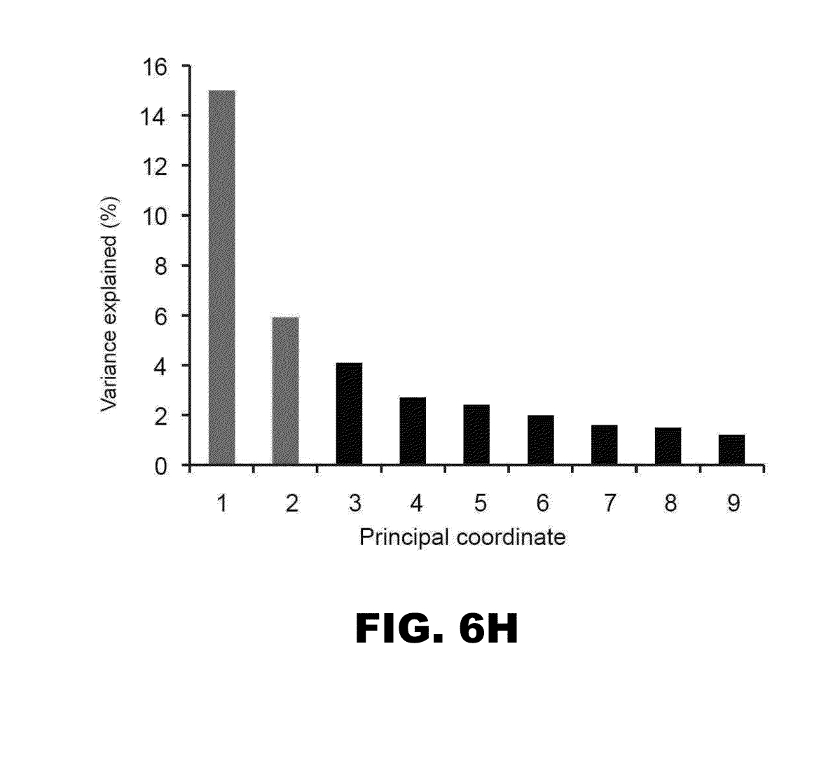

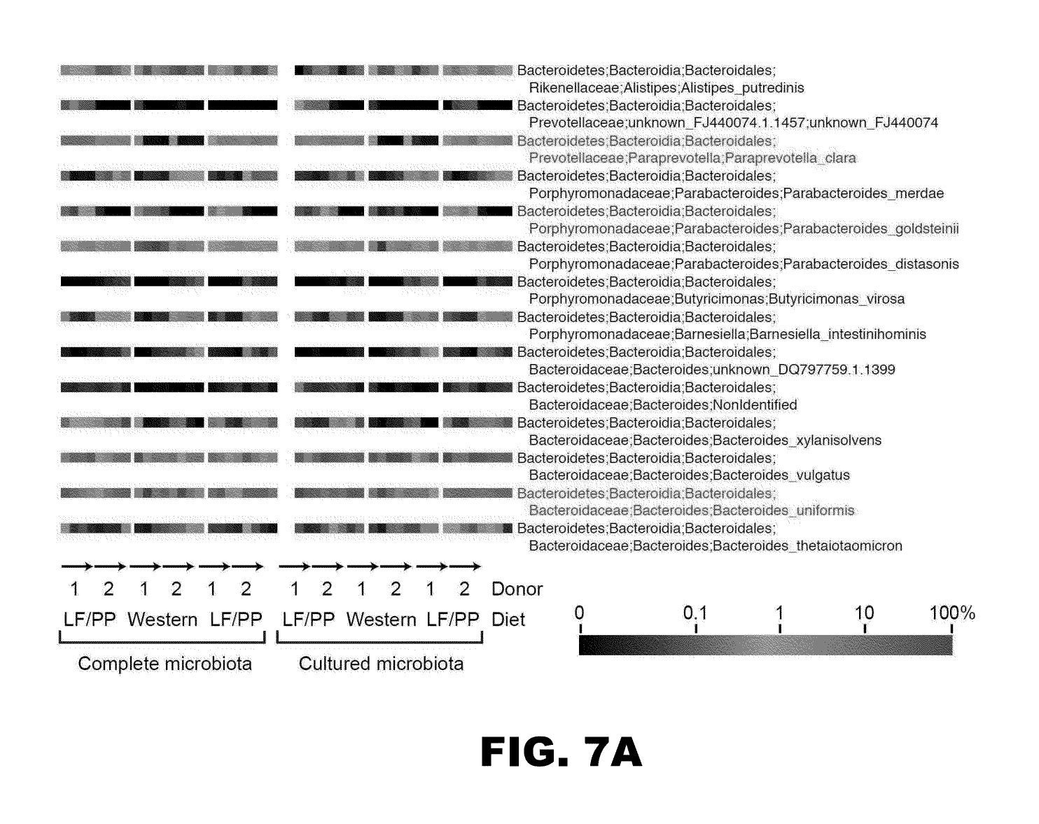

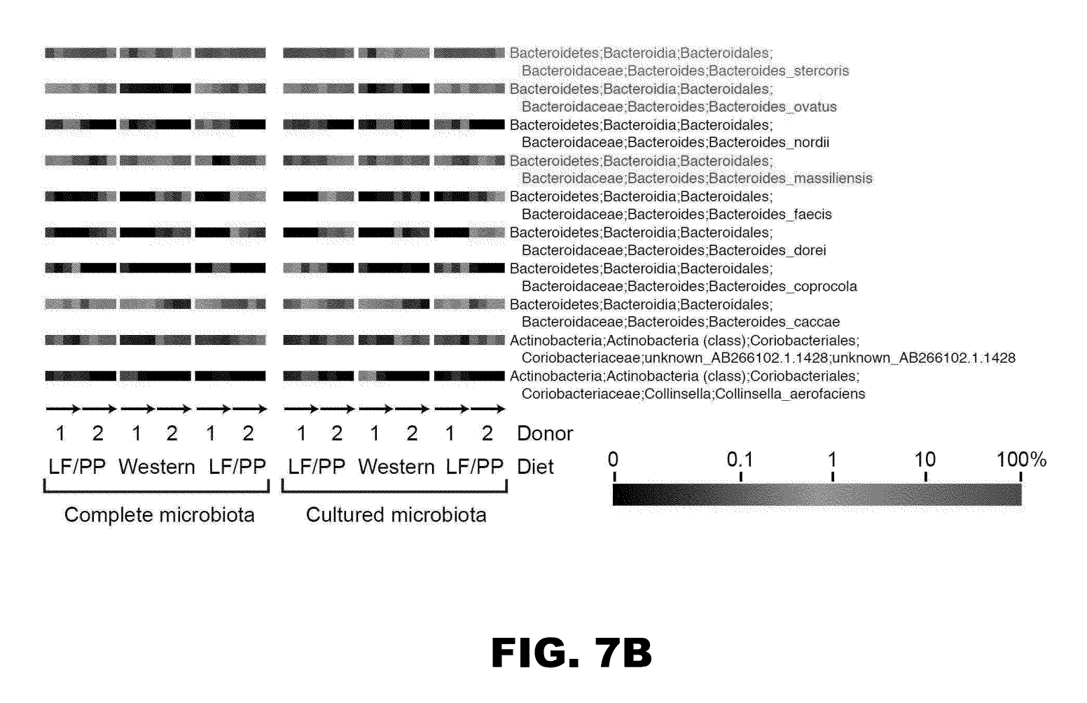

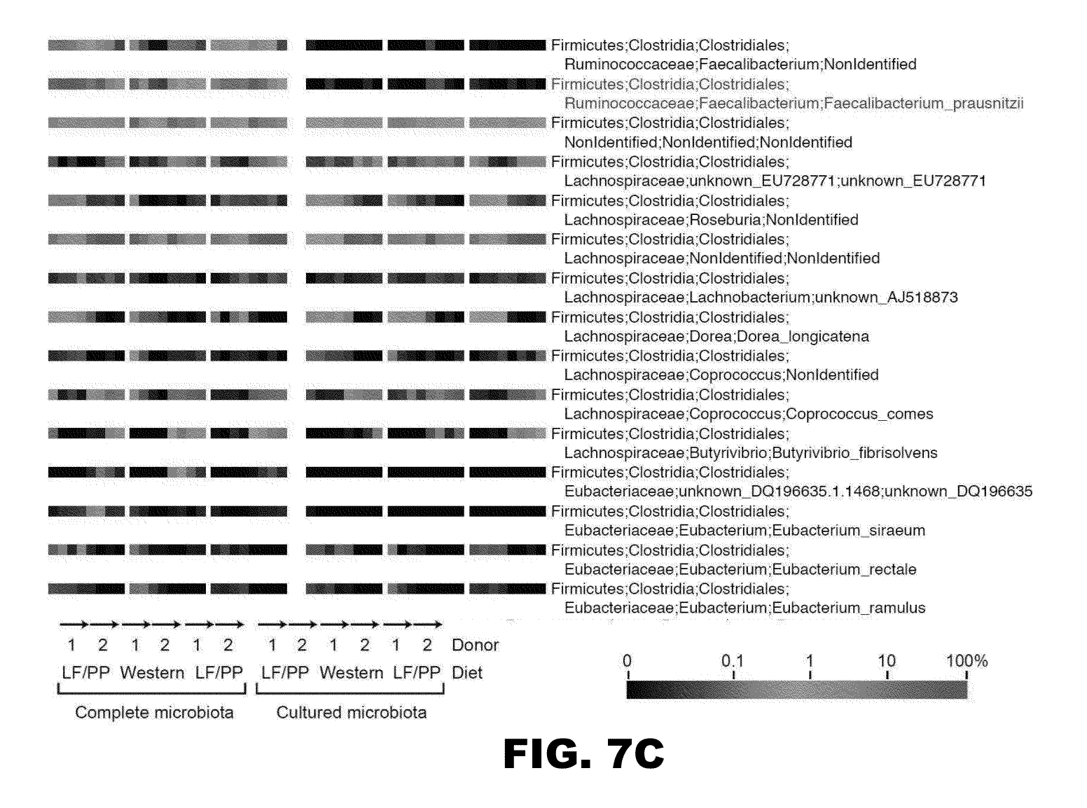

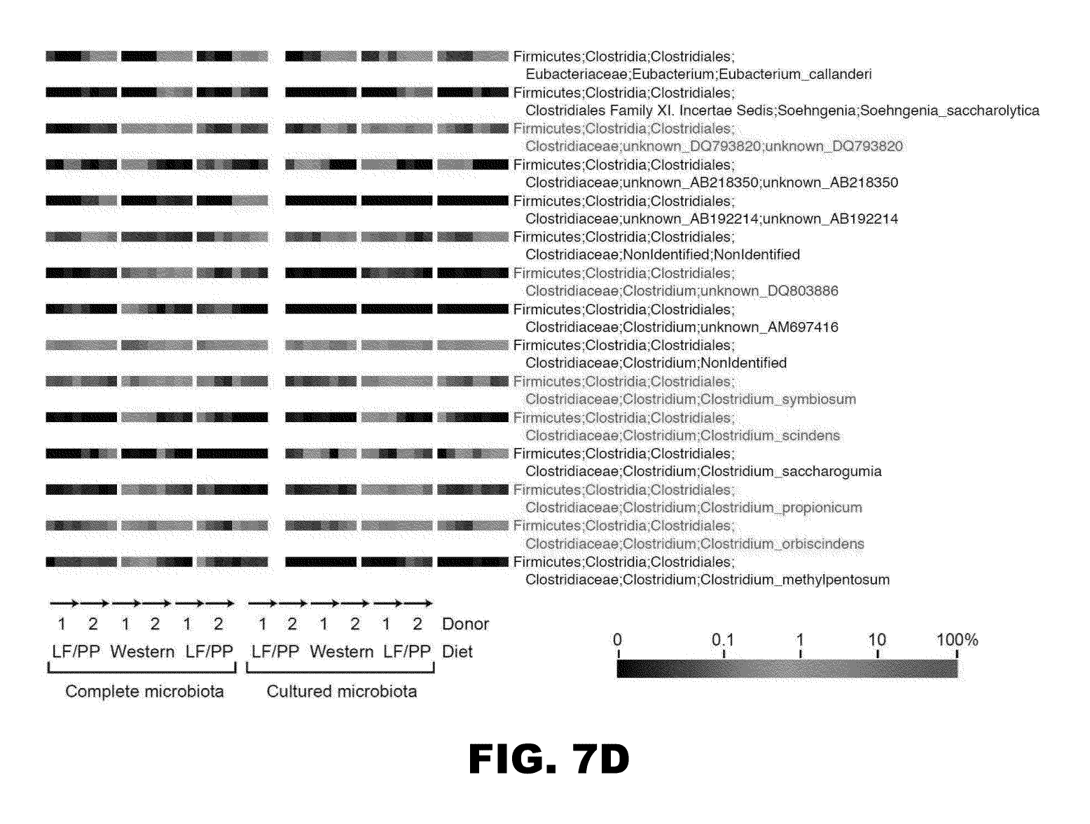

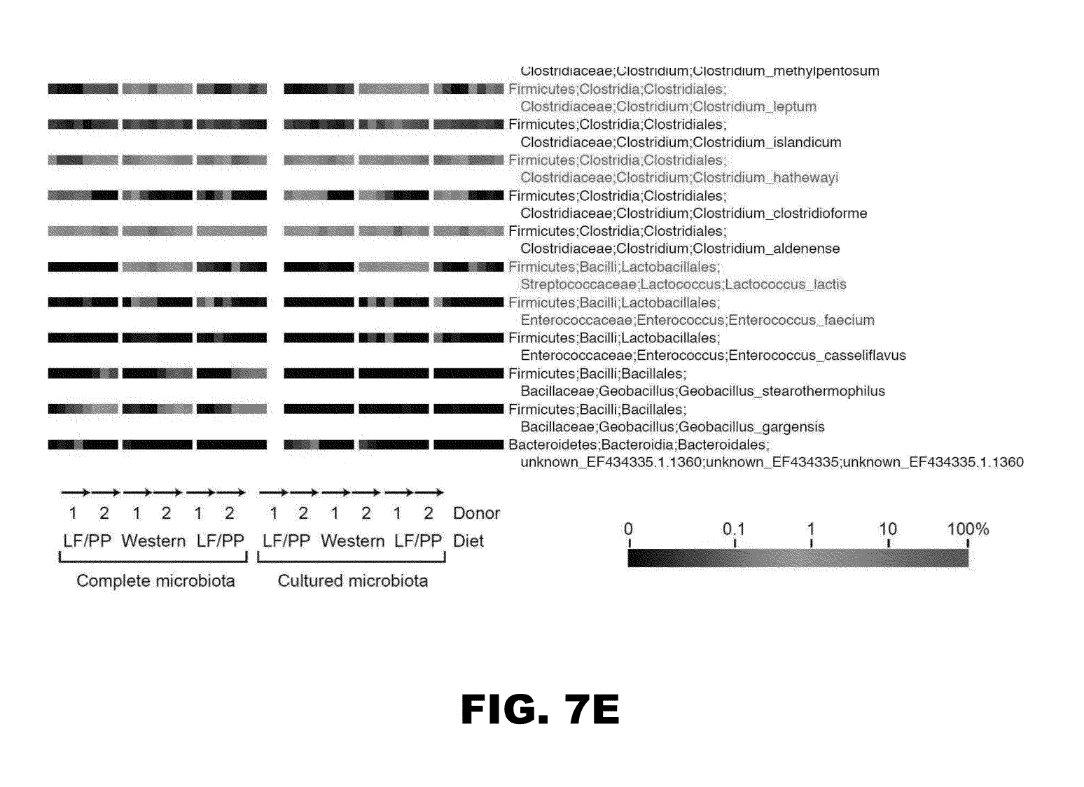

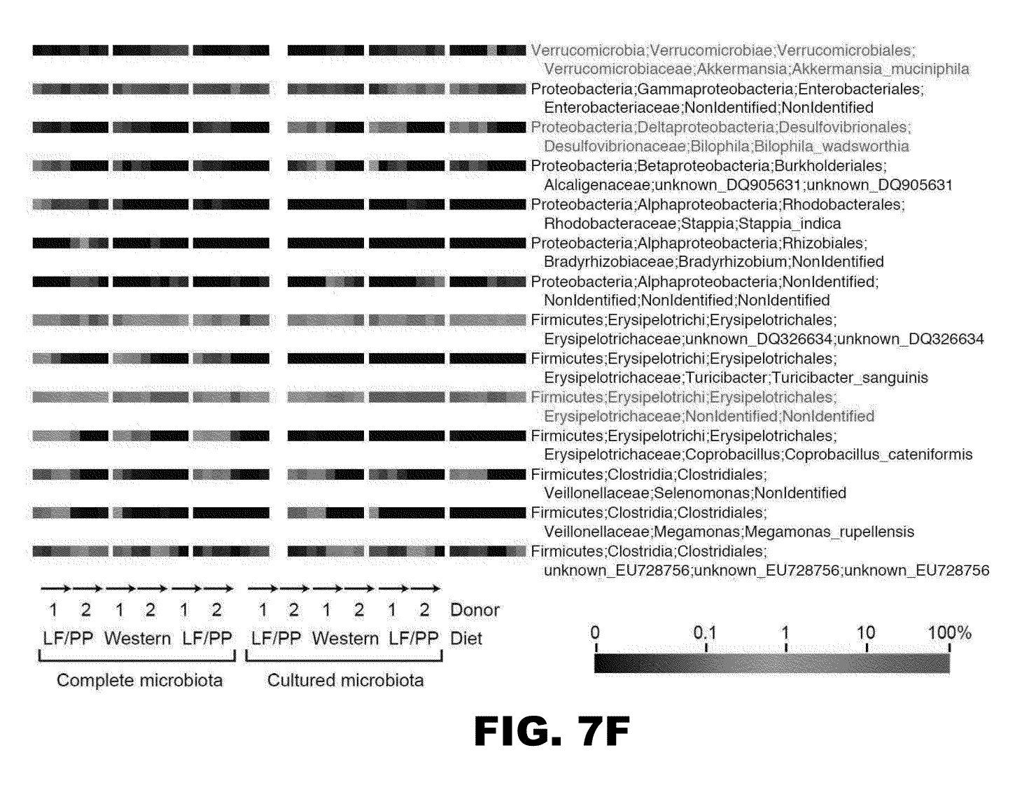

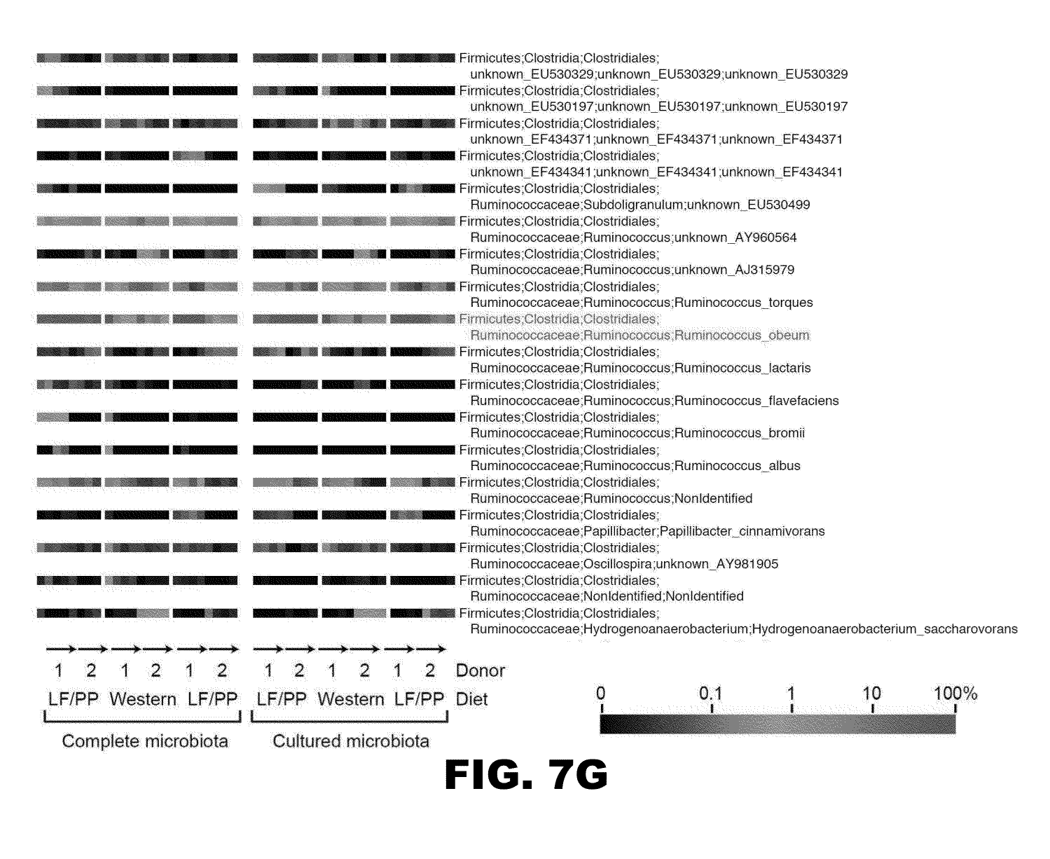

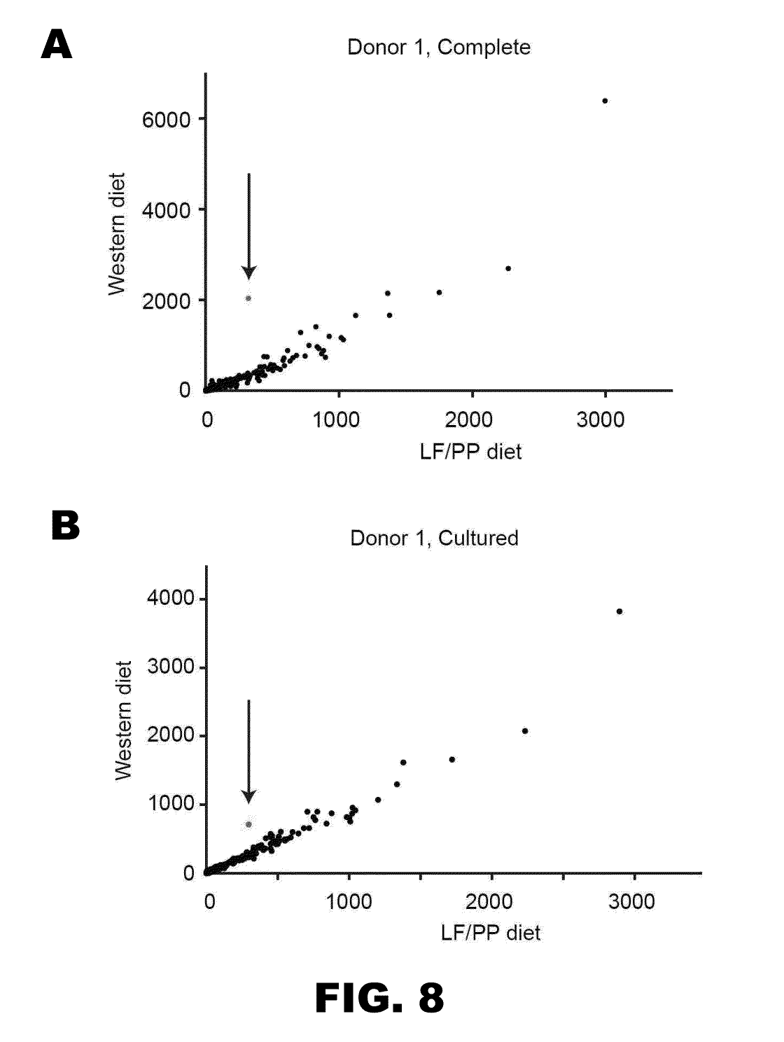

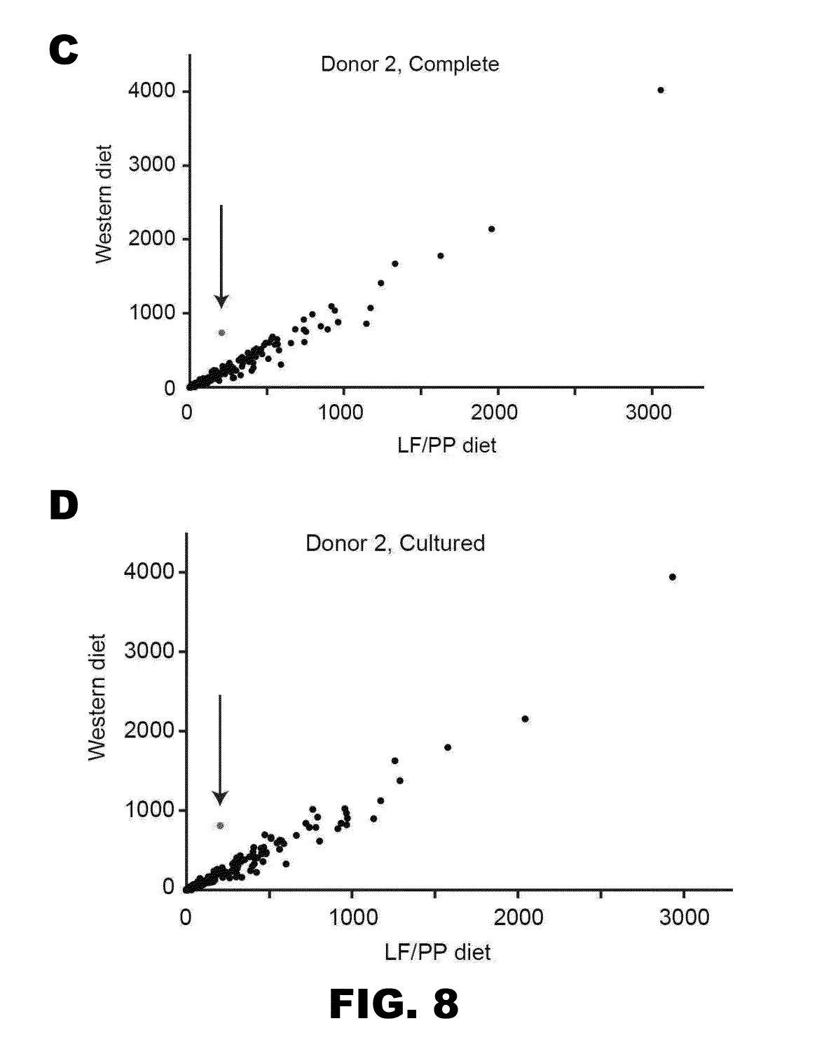

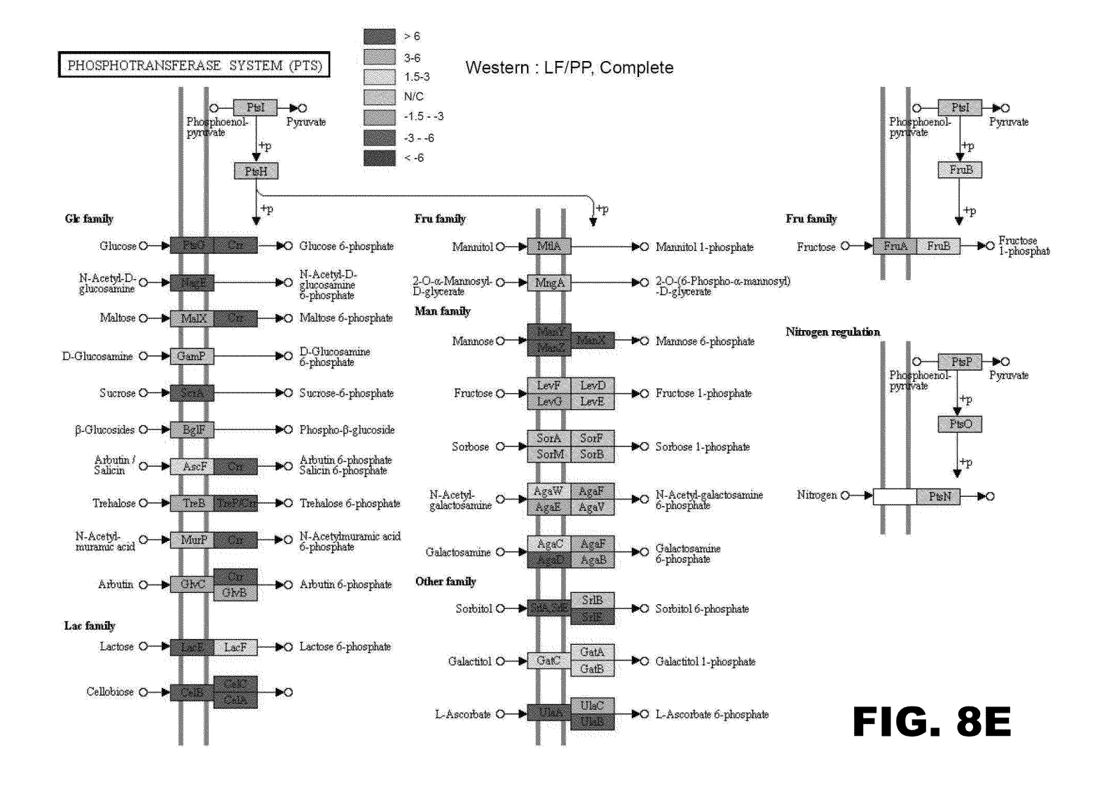

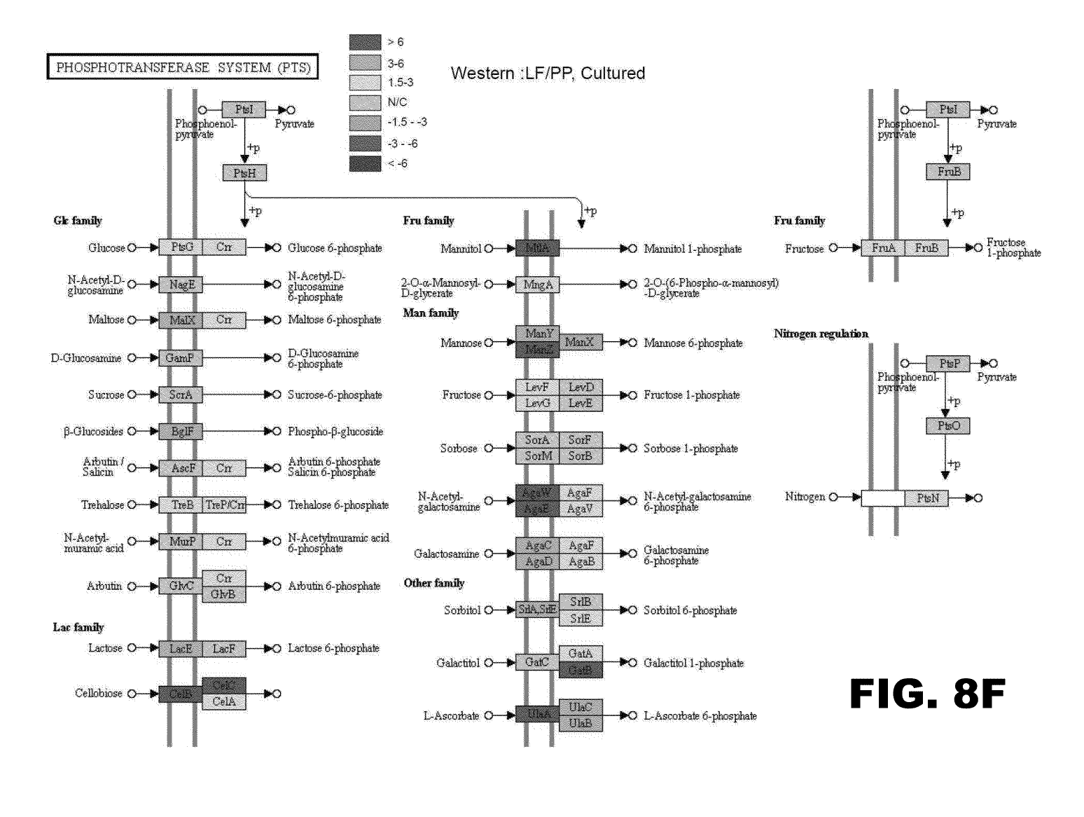

FIG. 5 Human gut microbial communities composed only of cultured members exhibit in vivo dynamics similar to those in their complete counterparts. (A) PCoA of UniFrac distances between 16S rRNA datasets generated from fecal samples from gnotobiotic mice, colonized with complete or cultured human fecal microbial communities from two unrelated donors and sampled over time. From day 33-46, mice were switched from their standard LF/PP chow to a high-fat, high-sugar Western diet. Time series analysis of community structure as viewed along the first two principal coordinates from A shows that interpersonal (donor) differences separated communities on PC1 (B), and host diet separated communities on PC2 (C). Principal coordinate 3 (PC3) separated samples from mice colonized with complete communities from those colonized with cultured populations (FIG. 6A). Nonphylogenetic distance metrics produced similar results (FIG. 6 D-H). (D) Evidence that the community response to diet is driven by readily cultured bacteria and that members of the same taxonomic group manifest distinct responses to diet perturbations. Species-level taxa significantly influenced by diet (student's t test P≦0.01 after Bonferroni correction; n=97 taxa tested) in either the complete communities (blue names), the cultured communities (green names), or both (red names) are plotted over time (arrows). Each column represents the average relative abundance in fecal samples harvested from three to five individually caged mice that were sampled at various times: (i) during the initial LF/PP diet phase; (ii) during the subsequent shift to the Western diet; and (iii) upon return to LF/PP chow. Members of family-level groups with at least one diet-responsive species are shown (excluding rare species with average abundance <0.1% across each time point). The names of all taxa are shown in FIG. 7. (E) The functional gene repertoire in the fecal microbiomes of humanized gnotobiotic mice. Each point represents a KEGG level 2 pathway; the number of hits to each pathway per 100,000 shotgun pyrosequencing reads is plotted for mice consuming LF/PP (x axis) or Western (y axis) diets. Data represent the averages of mice colonized with microbial communities from two unrelated donors. The results show that the fecal microbiome associated with the Western diet is enriched for genes in pathways associated with PTS (red arrows) both in mice colonized with complete uncultured human gut communities (black points) and mice colonized with communities of readily cultured members (orange points). Donor-specific data and results from alternate annotation schemes are shown in FIG. 8.

FIG. 6 Diet shapes complete and readily cultured human gut microbial communities in a similar manner. (A) PCoA of unweighted UniFrac distances between fecal samples obtained from mice colonized with complete or cultured microbial communities from two unrelated human donors. On day 33 after gavage, mice were switched from an LF/PP chow to a high-fat, high-sugar Western diet (gray rectangle). On day 47 they were returned to the original LF/PP diet. Variance along principal coordinate 3 (PC3) is plotted against time. (B) Scree plot from PCoA analysis. Only PC1-PC3 (red) account for ≧5% of the variance between samples. (C) In gnotobiotic mice, communities composed of readily cultured human gut microbes and communities containing a complete human gut microbiota undergo similar diet-dependent changes in abundance of Bacteroidia and Erysipelotrichi upon changes in host diet. Each mouse in each treatment group was caged individually, and each group that received a given community was housed in a separate gnotobiotic isolator. Mean values±SEM and P values (*P<0.05; **P<0.01 based on a paired, two-tailed student's t test) are indicated when mice were consuming a LF/PP diet (black bars) and then switched to the Western diet (white bars). (D-H) Diet-dependent community-wide shifts in bacterial species representation as measured by PCoA analysis based on a nonphylogenetic (binary Jaccard) distance measurement. (D) PCoA plot of binary Jaccard distances between all samples. (E-G) Separate PCoA values plotted against time. (H) Scree plot of variance explained by PCoA axes.

FIG. 7 Relative abundances of species-level taxa in fecal samples obtained from gnotobiotic mice over time. (A-G) All identified taxa present at an abundance of ≧0.1% in at least a single time point are shown. Species significantly influenced by diet in either the complete community (blue names), the readily cultured community (green names), or both (red names) are plotted over time (arrows) during the initial LF/PP, subsequent Western, and final LF/PP phases of the diet oscillation experiment. Significance (P≦0.01 after Bonferroni correction) was determined by unpaired, two-tailed student's t test, assuming equal variances; n=97 taxa tested. The assumption of equal variances was tested by F test (P<0.02).

FIG. 8 KEGG level 2 pathway-based analysis of fecal microbiomes obtained from LF/PP- and Western diet-fed mice colonized with a complete or cultured human gut microbiota from two human donors. (A-D) Phosphotransferase system (PTS) pathways are marked in red and highlighted with arrows. (E and F) Multiple predicted PTS pathway components are enriched in the fecal microbiomes of mice colonized with complete (E) or cultured (F) human gut microbial communities and maintained on a high-fat, high-sugar Western diet. KO level-predicted functional annotations are colored by average fold-difference in their representation in microbiomes obtained from mice on the different diets (Western versus LF/PP). Data represent averages from mice colonized with the complete or cultured fecal communities from two unrelated human donors.

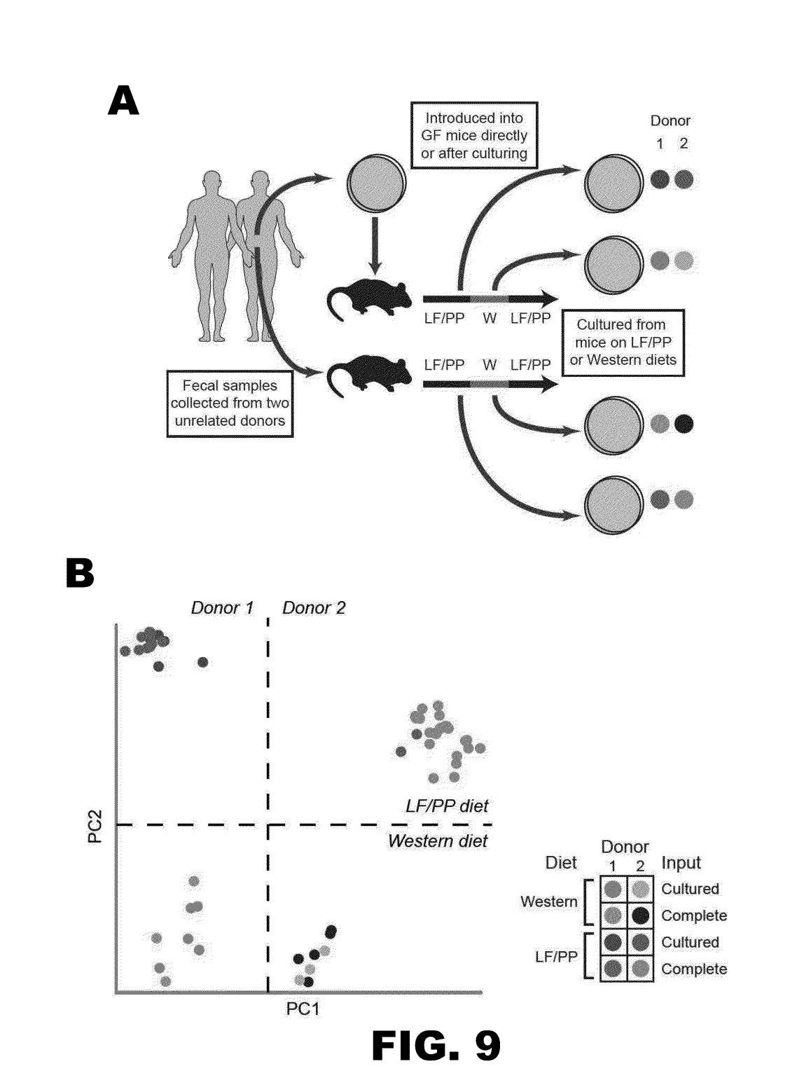

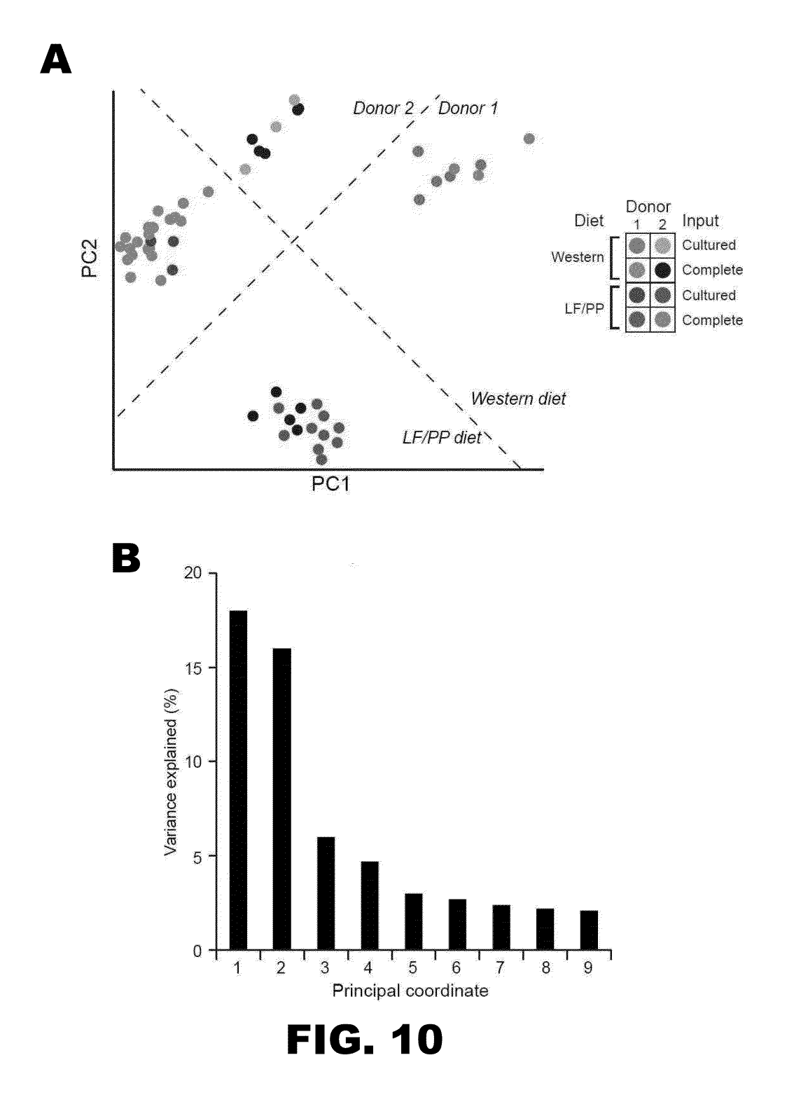

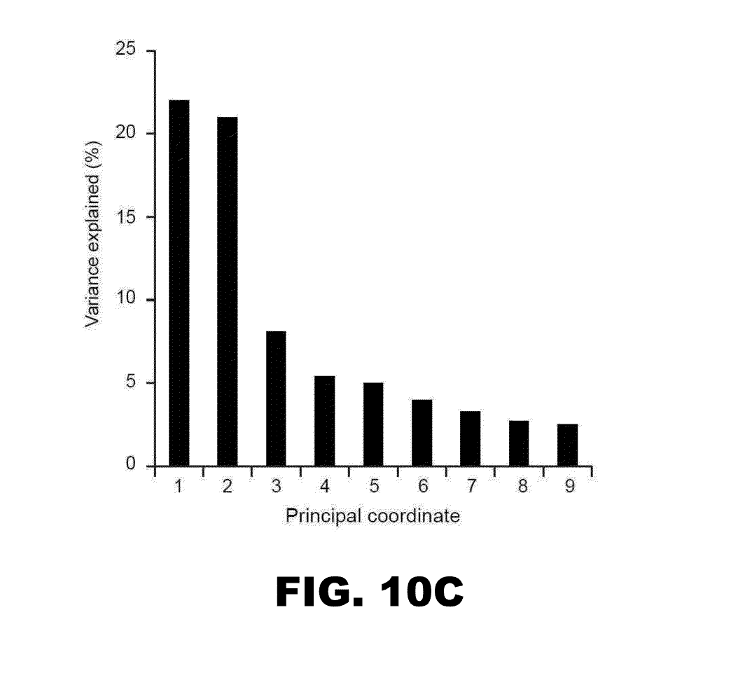

FIG. 9 The community composition of microbes cultured from humanized gnotobiotic mice can be reshaped by altering host diet. (A) Culture collections were generated from fecal samples obtained from gnotobiotic mice colonized with complete or cultured human gut microbial communities and maintained on LF/PP or Western diets. (B) PCoA of nonphylogenetic (binary Jaccard) distances between cultured samples indicates that manipulation of host diet can be used to shape the composition of communities recovered in culture from these animals. Analysis of phylogenetic (UniFrac) distances between samples produced similar clustering by donor and host diet (FIG. 10).

FIG. 10 Plated communities of human gut microbes can be reshaped through diet selection in gnotobiotic mice. (A) PCoA analysis of unweighted UniFrac distances between communities collected from mice before and after a diet switch and plated on GMM. Scree plots display variance explained by PCoA analysis of binary Jaccard (B) or unweighted UniFrac (C) distances between samples.

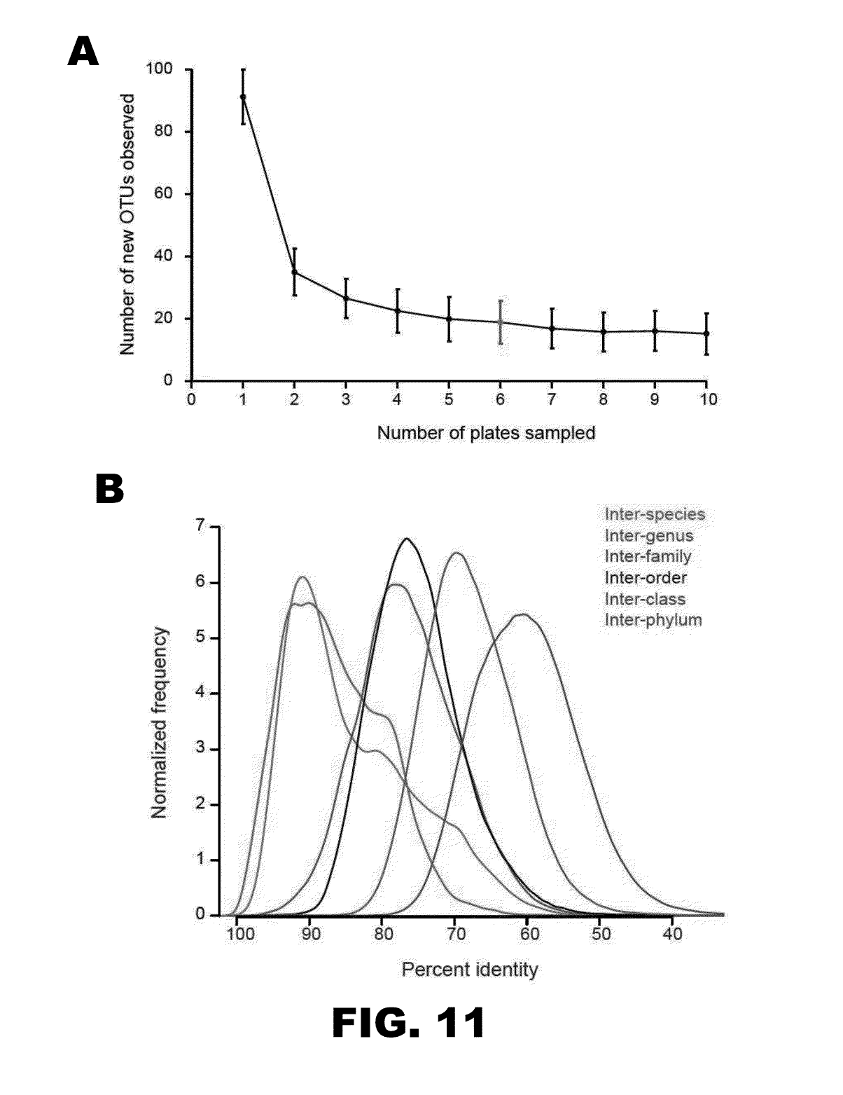

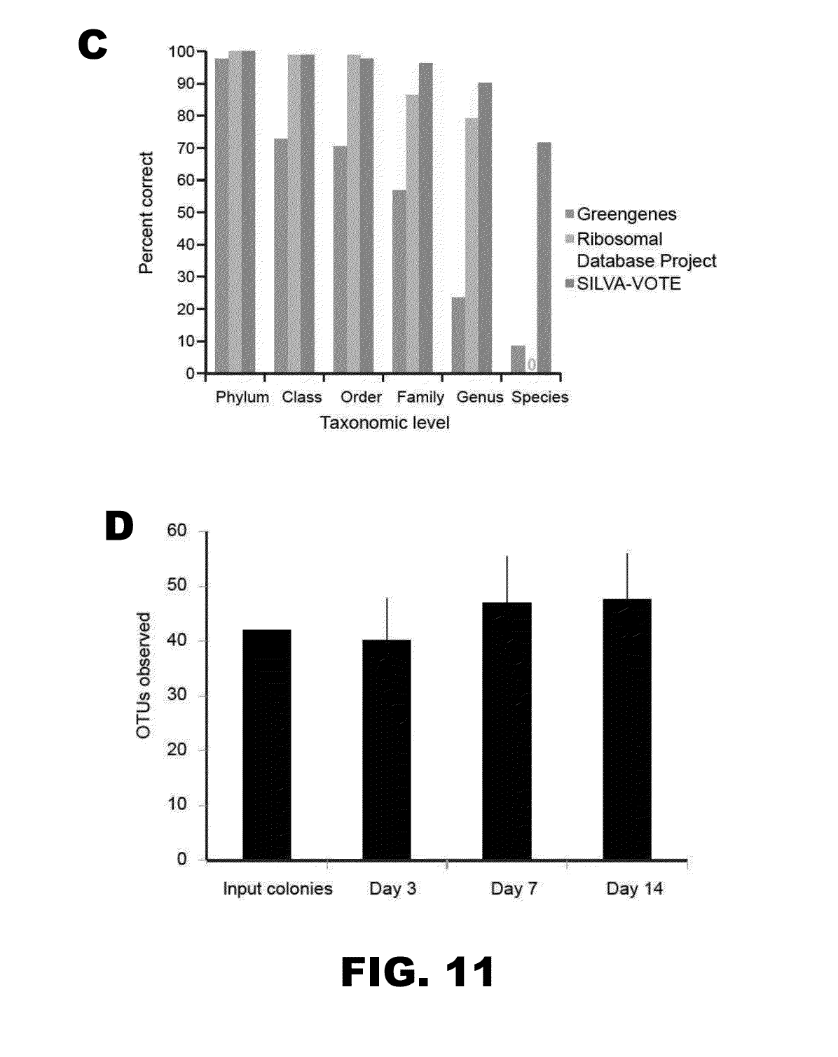

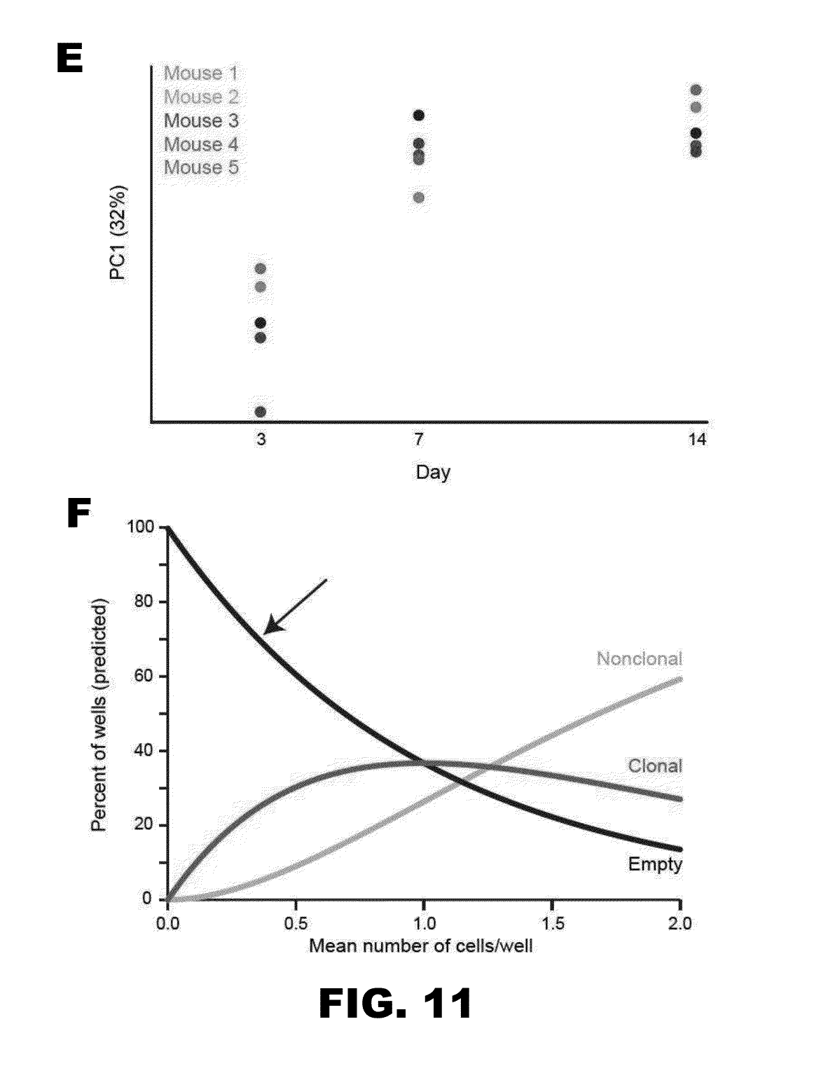

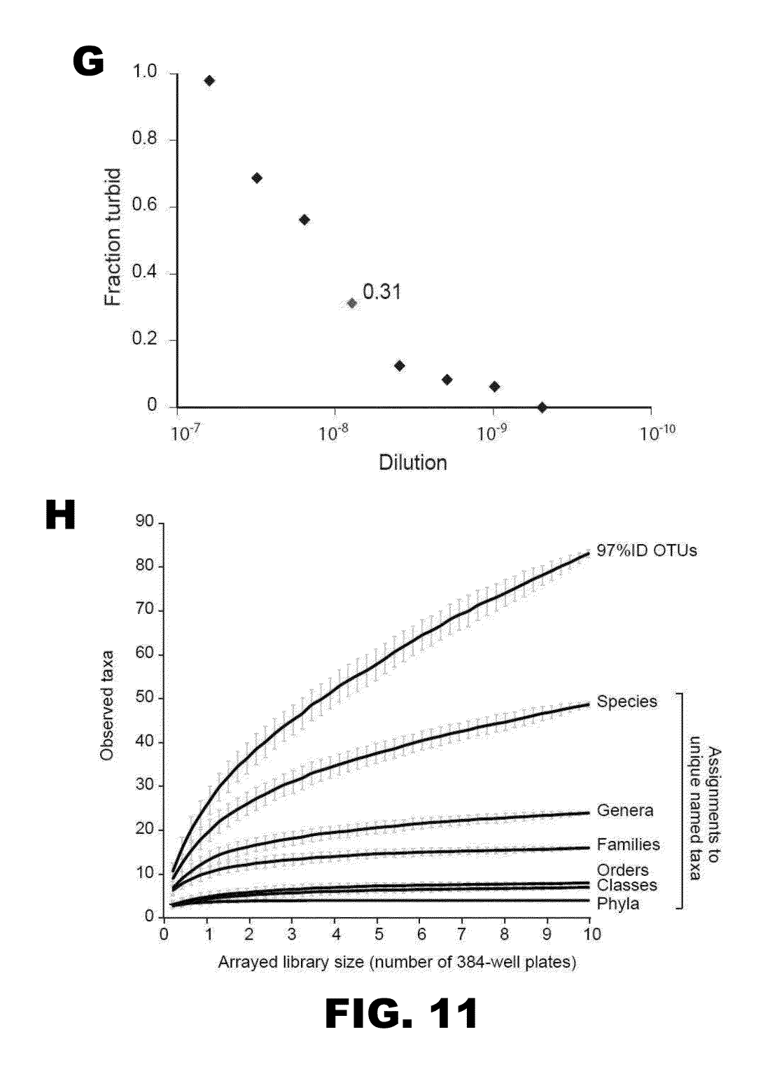

FIG. 11 Experimental parameters for en masse culturing, taxonomic assignment, inoculation of germfree mice, and arrayed strain collections. (A) Most readily cultured OTU in a human fecal sample are observed in six GMM plates. Rarefaction analysis describes the number of new OTU observed with each additional agar plate added to the dataset (10 plates were prepared independently). Points reflect the mean value after 100 iterations (plates sampled in random order without replacement); error bars represent one SD. The mean number of new OTU identified per plate drops below 20 with six or more plates (red). (B) Distributions of percent identities between pairwise comparisons of V2 16S rRNA gene sequences from representatives of bacterial taxa with varying degrees of shared phylogeny. We selected 4,041 16S rRNA sequences from the SILVA database (v102) that contain complete V2 regions and full (species-level) SILVAVOTE annotations. Sequences were aligned using PyNast. After removal of gap-only columns, the % ID between V2 regions was calculated for each pairwise comparison. The resulting % ID distributions are plotted for members of two different species within the same genus (interspecies), two different genera of the same family (intergenus), and so forth. (C) Comparison of three methods for assigning taxonomy to V2 16S rRNA sequences. (D and E) Sequences identified in gnotobiotic mice colonized with a readily cultured human gut microbiota do not reflect nongrowing or dormant cells. (D) Alpha-diversity analysis of fecal microbial communities of mice that had been inoculated with the control sample described in SI Materials and Methods. Diversity is similar at the 7-d and 14-d time points. (E) Time-course beta-diversity analysis of gnotobiotic mice inoculated with the control sample. UniFrac distances were calculated between all samples and represented spatially by PCoA. In this figure, variance along the major coordinate of variance (PC1) is plotted against time; colors represent individual mice. (F) Communities of cultured human gut microbes can be clonally archived in 384-well format by limiting dilution. At a dilution of a human fecal sample that produces 70% empty wells, a Poisson distribution predicts that 25% of wells will contain a clonal population of cells and that 5% of wells will be nonclonal (arrow). (G) Optimization of dilutions for arrayed strain collections. A 1.28×108-fold dilution of the stored fecal aliquot and subsequent inoculation into 384-well trays (0.17 mL per well) produces 70% empty wells. (H) Rarefaction analysis indicates that ten 384-well trays are sufficient to capture nearly all the genus-level diversity that can be retrieved from a single fecal sample using this arrayed culturing method.

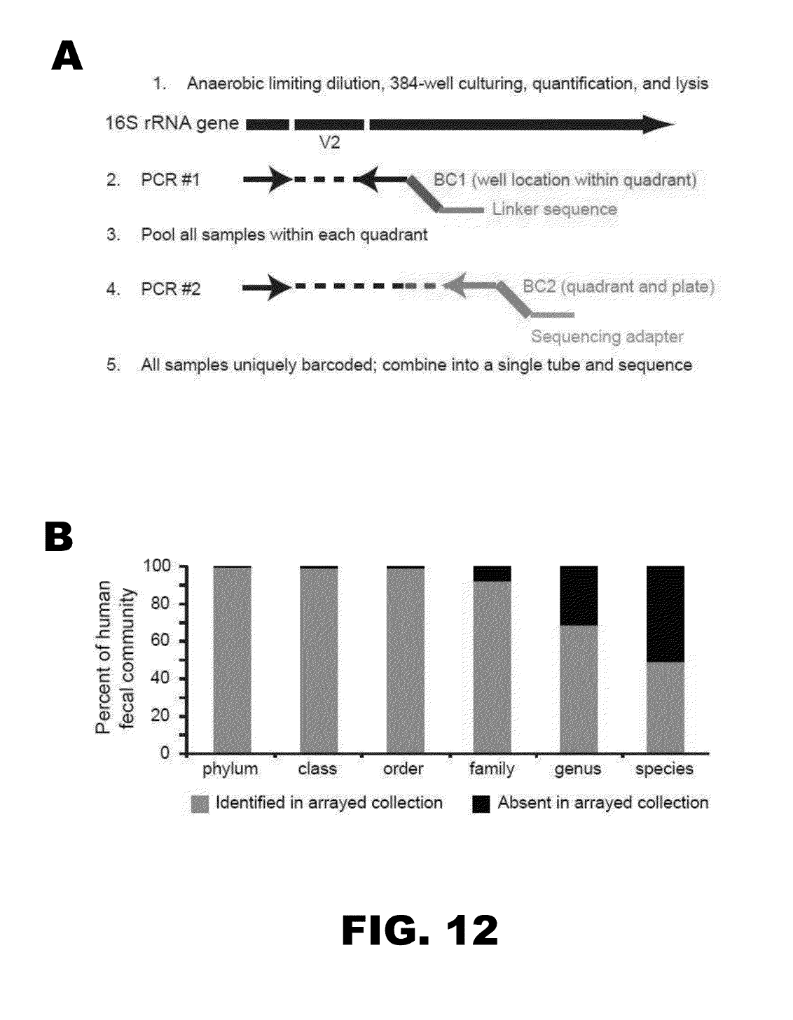

FIG. 12 Personal culture collections archived in a clonally arrayed, taxonomically defined format. (A) After limiting dilution of the sample into 384-well trays to the point at which most turbid wells are clonal, a two-step, barcoded pyrosequencing scheme allows each culture well to be associated with its corresponding bacterial 16S rRNA sequence. In the first round of PCR, one of the V2-directed 16S rRNA primers incorporates 1 of 96 error correcting barcodes (BC1, highlighted in red) that designates the location (row and column) within a quadrant of the 384-well tray where the sample resides. The primer also contains a 12-bp linker (blue). All amplicons generated from all wells in a given quadrant from a single plate then are pooled and subjected to a second round of PCR in which one of primers, which targets the linker sequence, incorporates another error-correcting barcode (BC2; green) that designates the quadrant and plate from which the samples were derived, plus an oligonucleotide (gray) used for 454 pyrosequencing. Amplicons generated from the second round of PCR then are pooled from multiple trays and subjected to multiplex pyrosequencing. This approach allows unambiguous assignment of 16S rRNA reads to well and plate locations using a minimum number of barcodes and primers; e.g., 96 BC1 primers and 96 BC2 primers allow 96² (9,216) wells to be analyzed. (B) Representation of the original (complete) microbial community in the arrayed strain collection.

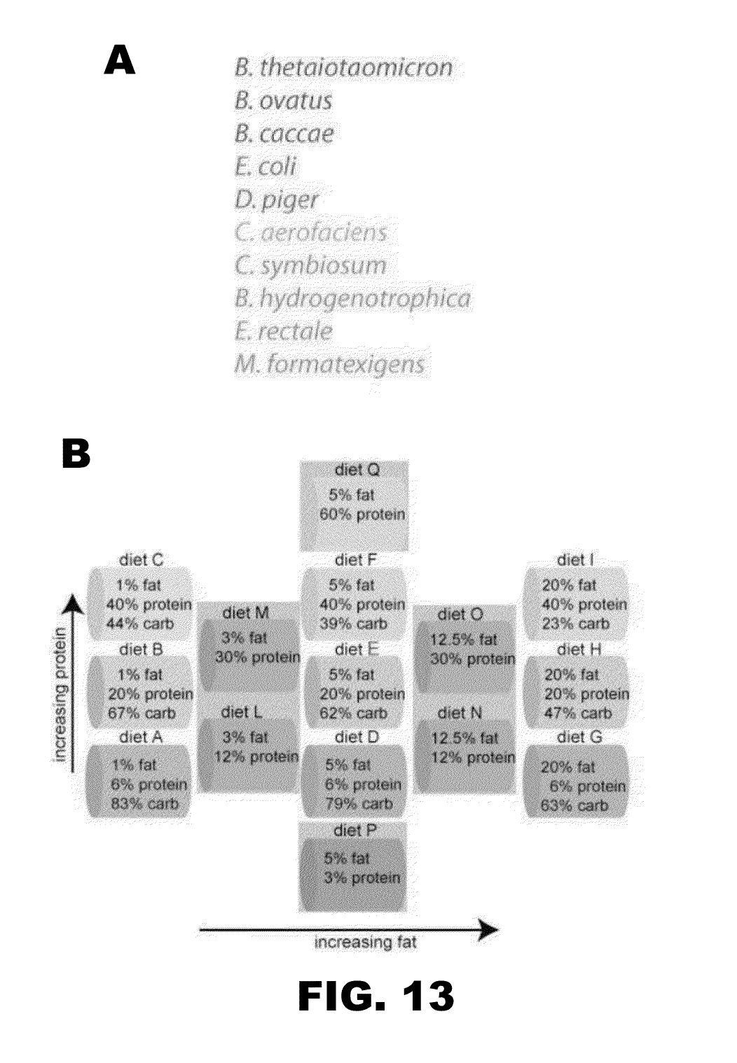

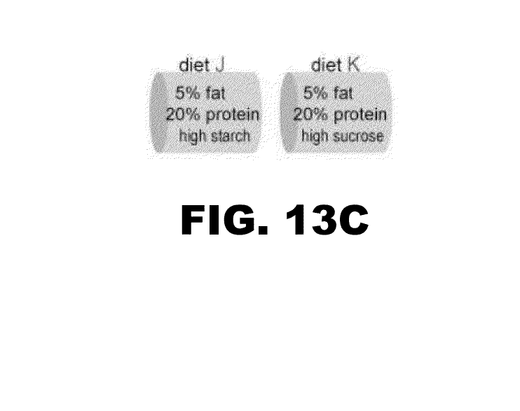

FIG. 13 Study design for refined diets. Two sets of gnotobiotic mice harboring a synthetic microbiota composed of ten sequenced human gut bacterial species were presented a total of 17 diets differing in their concentrations of casein (protein), corn oil (fat), sucrose (simple sugar), and cornstarch (polysaccharide). (A) The model community used for all experiments consisted of sequenced bacterial species from the four most abundant phyla in the adult human gut microbiota: Bacteroidetes (blue), Firmicutes (green), Actinobacteria (yellow), and Proteobacteria (red). (B,C) The first screen consisted of 11 refined diets: 9 of these diets (A-I in panel B) represent all possible combinations of high, medium, and low protein and fat; the two additional diets (J and K in panel C) contained high sucrose/low starch and high starch/low sucrose, respectively.

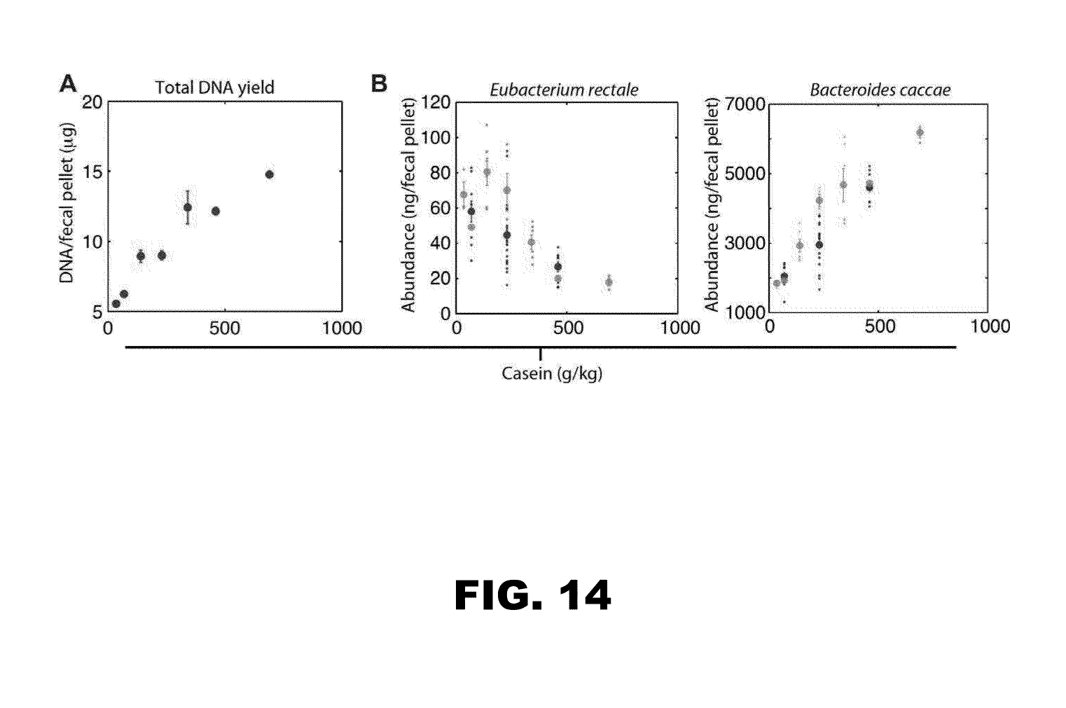

FIG. 14 Total community abundance (biomass) and the abundance of each community member can best be explained by changes in casein. (A) The total DNA yield per fecal pellet increased as the amount of casein in the host diet increased (shown are mean±S.E.M. for each tested concentration of casein). (B) Changes in species abundance as a function of changes in the concentration of casein in the host diet were also apparent for all 10 species; 7 species were positively correlated with casein concentration (e.g., B. caccae, right panel) while the remaining three species were negatively correlated with casein concentration (e.g. E. rectale, left panel). Data points from the first and second set of mice given the refined diets (see Table 9 for explanation) are shown in purple and green, respectively, while the mean and standard error for all diets at a given concentration of casein are shown in red and tan, respectively.

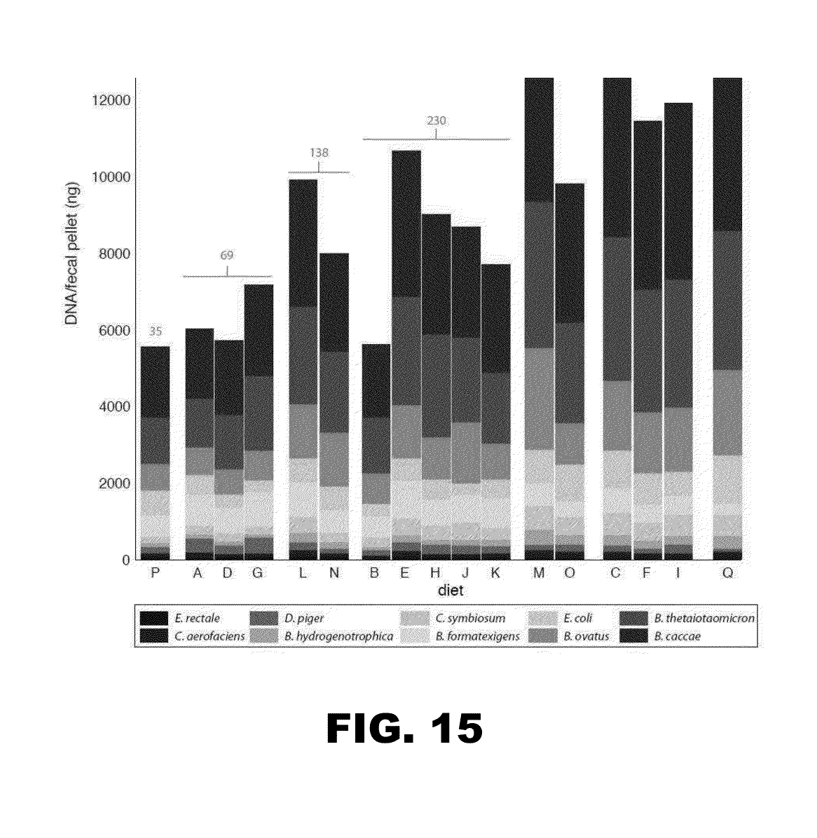

FIG. 15 Mean community member abundance for each diet. The height of each bar indicates the total DNA yield/biomass for a given diet. Casein concentrations (g/kg) for each diet are displayed in gray above each bar. See FIG. 13 and Table 6 for a description of diets A-Q.

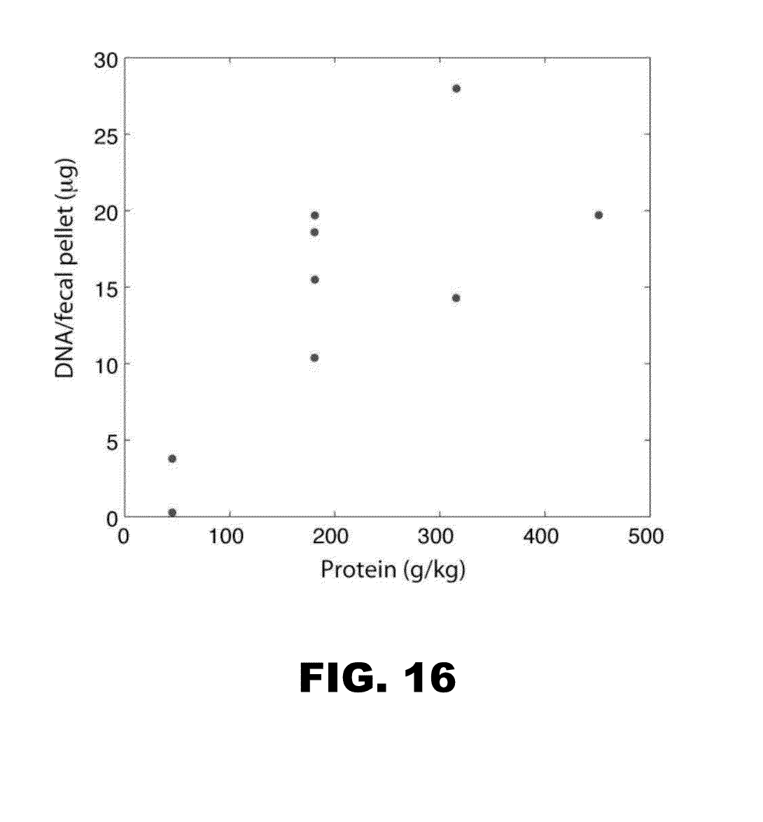

FIG. 16 Total community DNA yield as a function of protein concentration. Nine different 15-week-old gnotobiotic male C57Bl/6J mice harboring the 10-member community were each given a randomly selected diet containing varying amounts of three different refined protein sources (soy, egg white solids, and lactalbumin), and two different refined fat sources (olive oil and lard) (see Table 9 for diet schema). Each mouse was sampled on days 5, 6, and 7 of the diet period. DNA was extracted from each fecal pellet and the three samples from each mouse were averaged to produce the final DNA yield per fecal pellet (see Table 17 for results of DNA measurements).

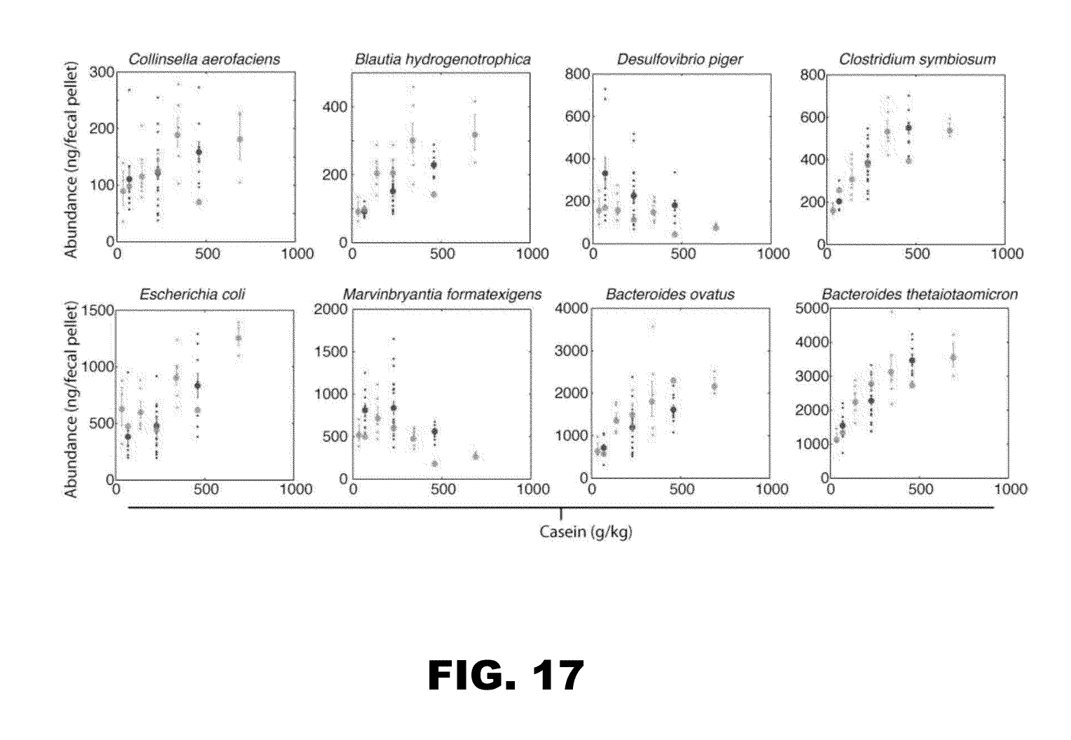

FIG. 17 Changes in species abundance as a function of changes in the concentration of casein in the host diet. Changes are apparent for all species in the model microbiota (note that the responses of E. rectale and B. caccae are shown in FIG. 14B of the main text). Data obtained from the first and second set of mice are shown in blue and green, respectively, while mean values±S.E.M are shown in red and tan, respectively.

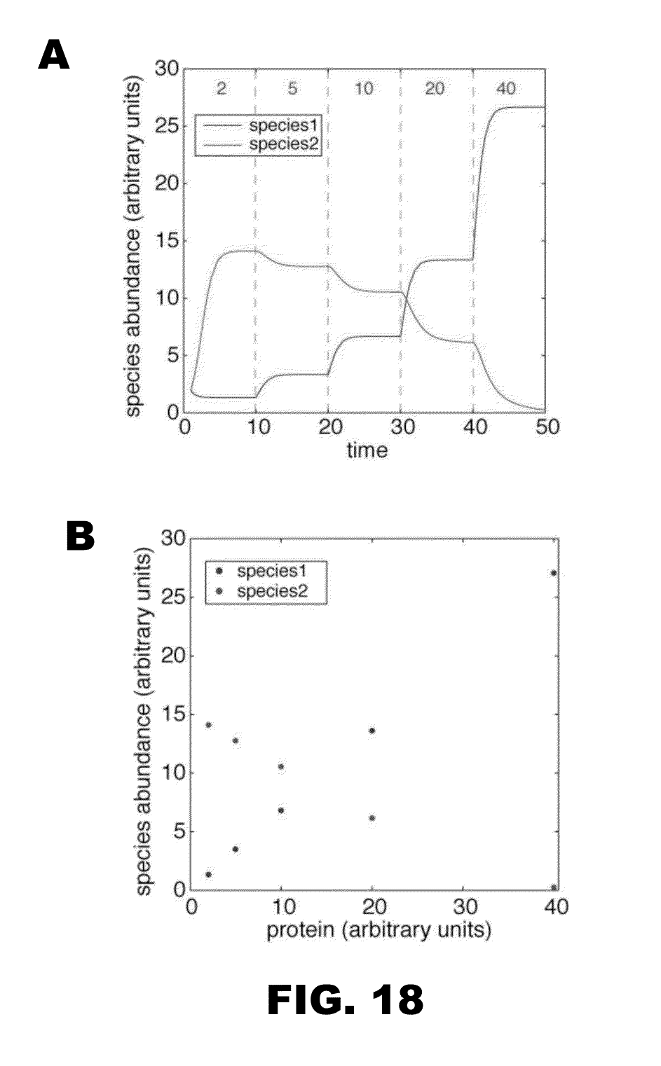

FIG. 18 Simulation of competition for limiting resources. (A) Using equations 4 and 5 for species1 and species2, both species were initialized to a population size of 2 and a diet switch was initiated every ten days, increasing casein abundance at each switch (red numbers indicate % casein for each diet period). (B) The steady-state values of the simulation in panel A mirror the findings in our mouse datasets where the increase in a bacterial species (species1) that is casein limited leads to a decrease in species2 with increasing dietary casein.

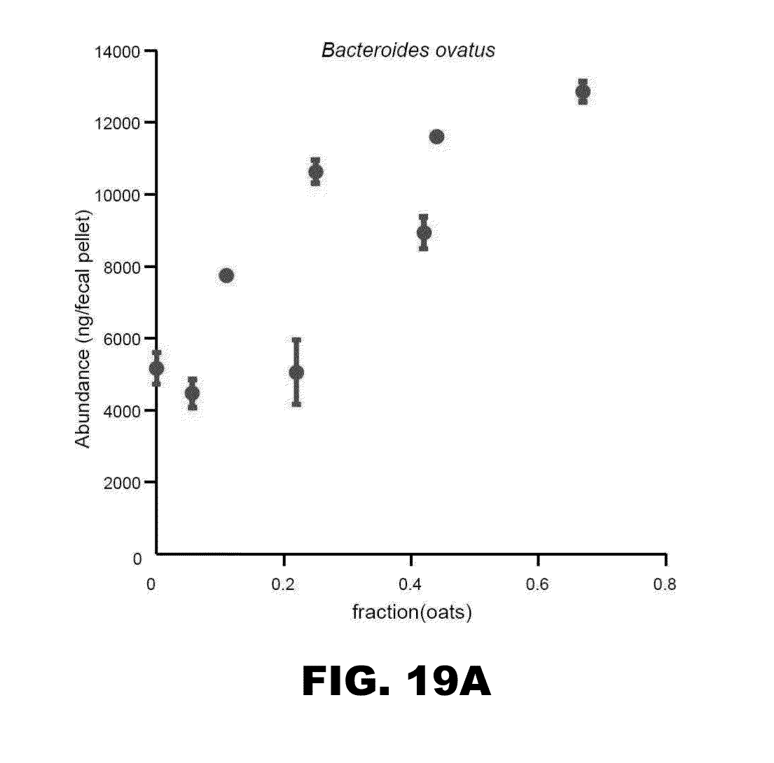

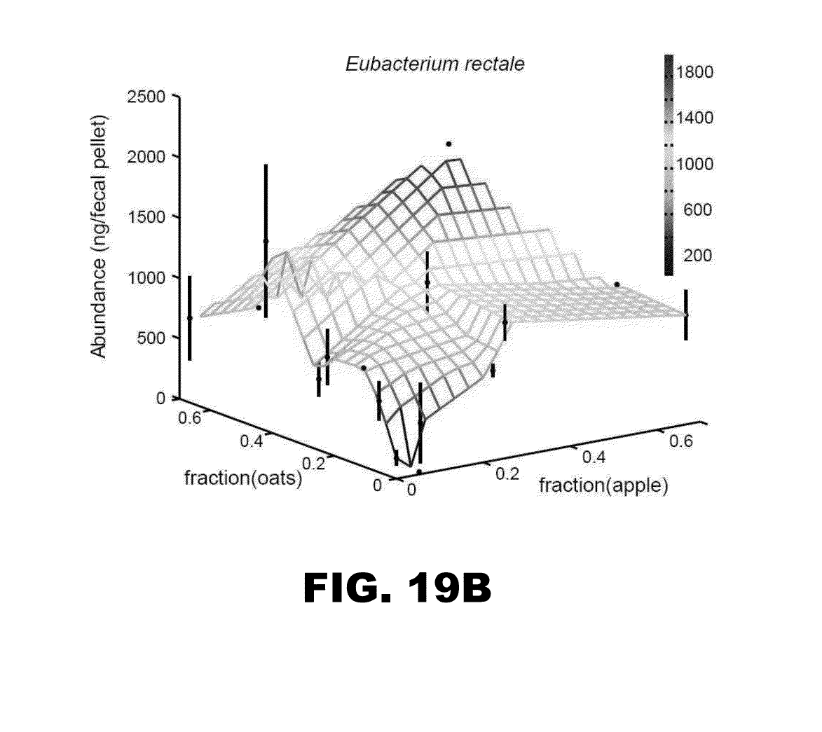

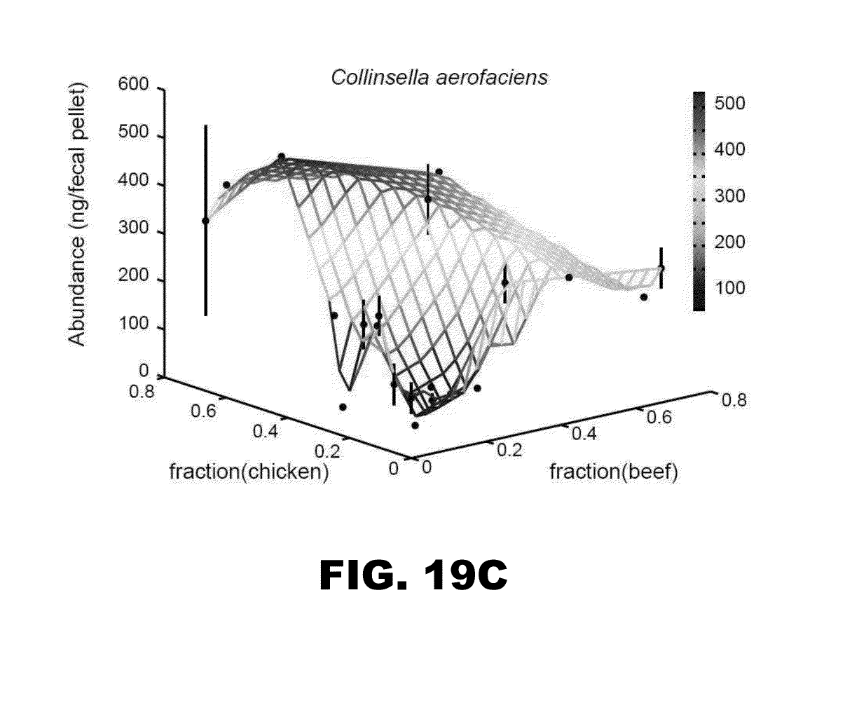

FIG. 19 Example of community member responses to complex human foods. Changes in species abundance as a function of diet ingredients were apparent for all 10 species (Table 16). B. ovatus increased in absolute abundance with increased concentration of oats in the diet (A), while most of the ten bacterial species (including E. rectale and C. aerofaciens) responded to multiple ingredients (B and C). The mean and standard error for all diets are plotted (no error bars are shown when replicate points are not available). The colored z-axis mesh grid on the 3D plots is a triangle-based linear interpolation of the data with color changes corresponding to the values in the color bar on the right.

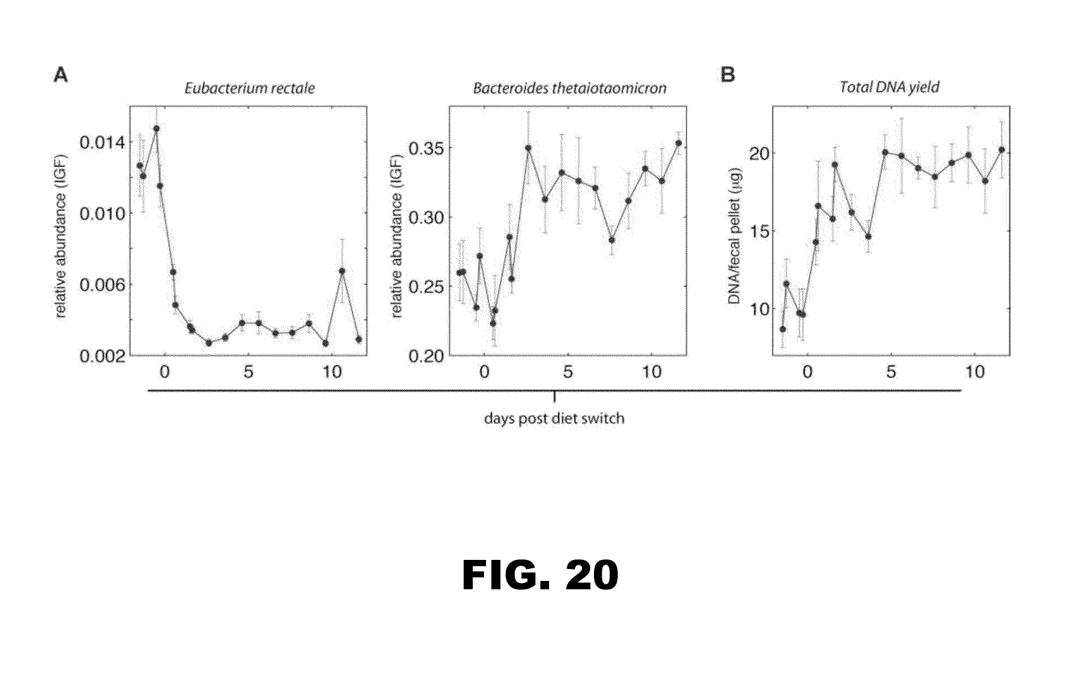

FIG. 20 Estimation of steady state. Nine adult male gnotobiotic mice harboring the ten-member model human gut community were fed a low-fat, low-protein diet (diet A in FIG. 13B) for 7-days and then switched to a high-fat, high-protein (diet I in FIG. 13B) at time-point zero for 13 days. The relative abundance of each of the taxa was subsequently defined using shotgun sequencing of fecal DNA to determine their Informative Genome Fraction (IGF). (A) The relative abundance of each bacterium changes rapidly within hours of a diet switch, reaching steady state levels by the third day (shown are the two species with the greatest increase and decrease respectively in relative abundance). Mean values±SEM are plotted at each time point. (B) Changes in total fecal DNA yield also increased rapidly in the first 24 h after the diet switch, reaching steady state levels on the fourth day.

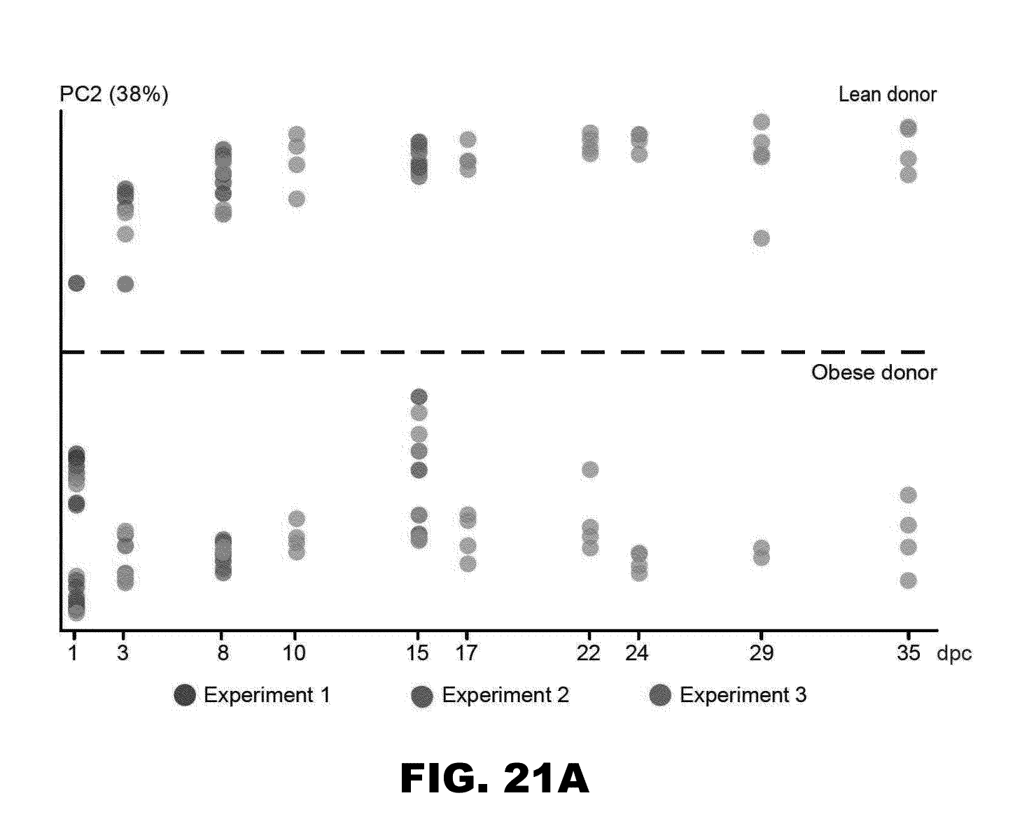

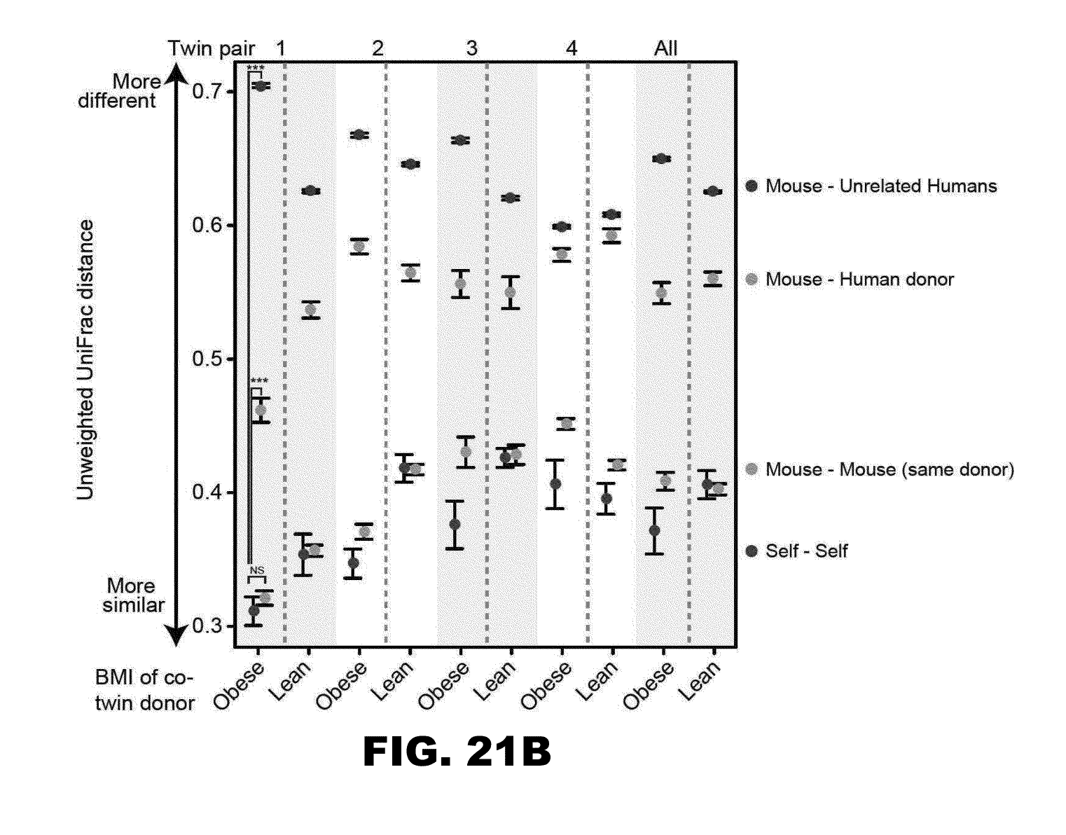

FIG. 21 Reliable replication of human donor microbiota in gnotobiotic mice. (A) Assembly of bacterial communities in mice that had received microbiota transplants from the obese and lean co-twins in DZ pair 1. PCoA plot based on unweighted Unifrac distance matrix and 97% ID OTUs in sampled fecal communities. Spheres correspond to a single fecal sample obtained at a given time point from a given mouse and are colored according to the co-twin microbiota donor, and the experiment (n=3 independent experiments). Note that assembly is reproducible within members of a group of mice that have received a given microbiota, and between experiments. (B) Transplantation of fecal microbiota from human donors to recipient mice is not only reproducible within members of a recipient group but also captures interpersonal differences. Mean values±SEM for pairwise UniFrac distance measurements are plotted. ‘Self-Self’ comparison, same mouse sampled at different time points within a given experiment; ‘Mouse-Mouse (same donor)’, mice colonized with the same human donor's fecal microbiota sample (3-8 mice/donor; 1-4 independent experiments/donor sample); ‘Mouse-Human donor’, comparison of fecal bacterial communities in recipient group of mice versus their human donor's microbiota; ‘Mouse-Unrelated Humans’, comparison of fecal microbiota from recipients of given donor's microbiota compared to the fecal microbiota of all other unrelated individuals (across twin pair comparison). *, p-value<0.05, **, p-value<0.001; Monte Carlo simulation, 100 iterations.

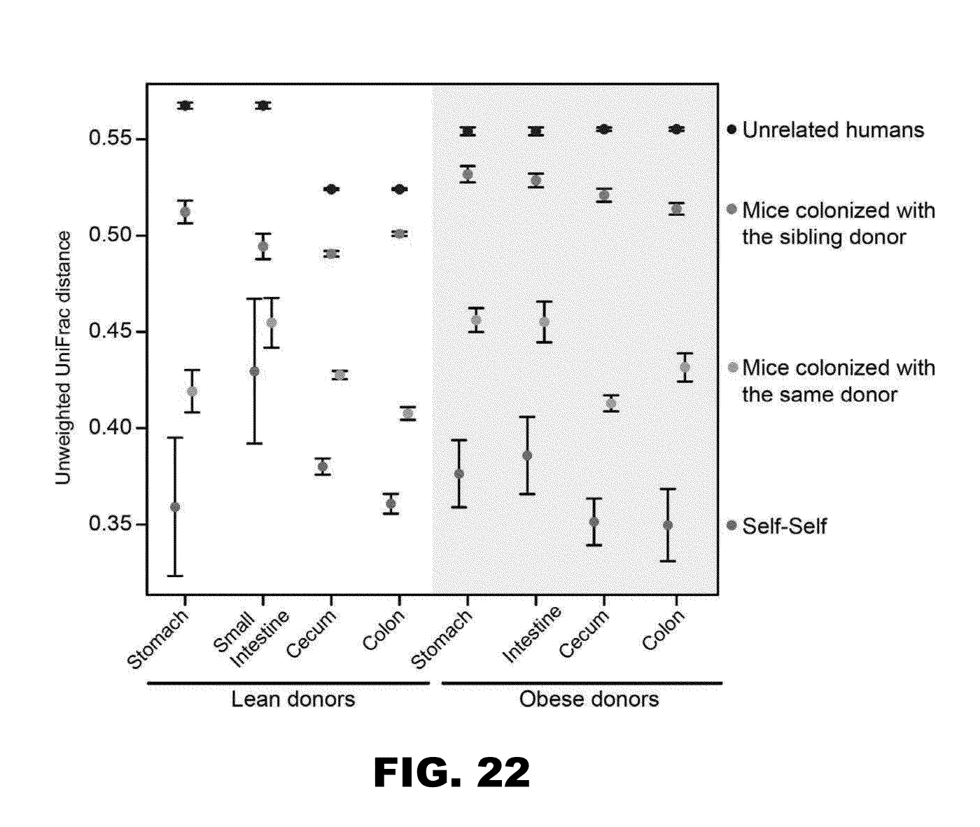

FIG. 22 Unweighted UniFrac analysis of samples collected along the length of the gut. Mean values±SEM for pairwise UniFrac distance measurements are plotted. ‘Self-Self’, comparison of community structures from different regions of the gut (small intestinal segments 1, 2, 5, 9, 13, 15 each analyzed separately with pair wise comparisons of segments). ‘Mice colonized with the same donor’, mice colonized with the same human donor's fecal microbiota sample (3-8 mice/donor; 1-4 independent experiments/donor sample); ‘Mouse colonized with sibling donor’, where the sibling represents the discordant co-twin; ‘Unrelated human donors’, comparison of fecal microbiota from recipients of given donor's microbiota versus fecal microbiota of all other unrelated individuals (across twin pair comparison). *p-value<0.05, ** p-value<0.001; Monte Carlo simulation, 100 iterations.

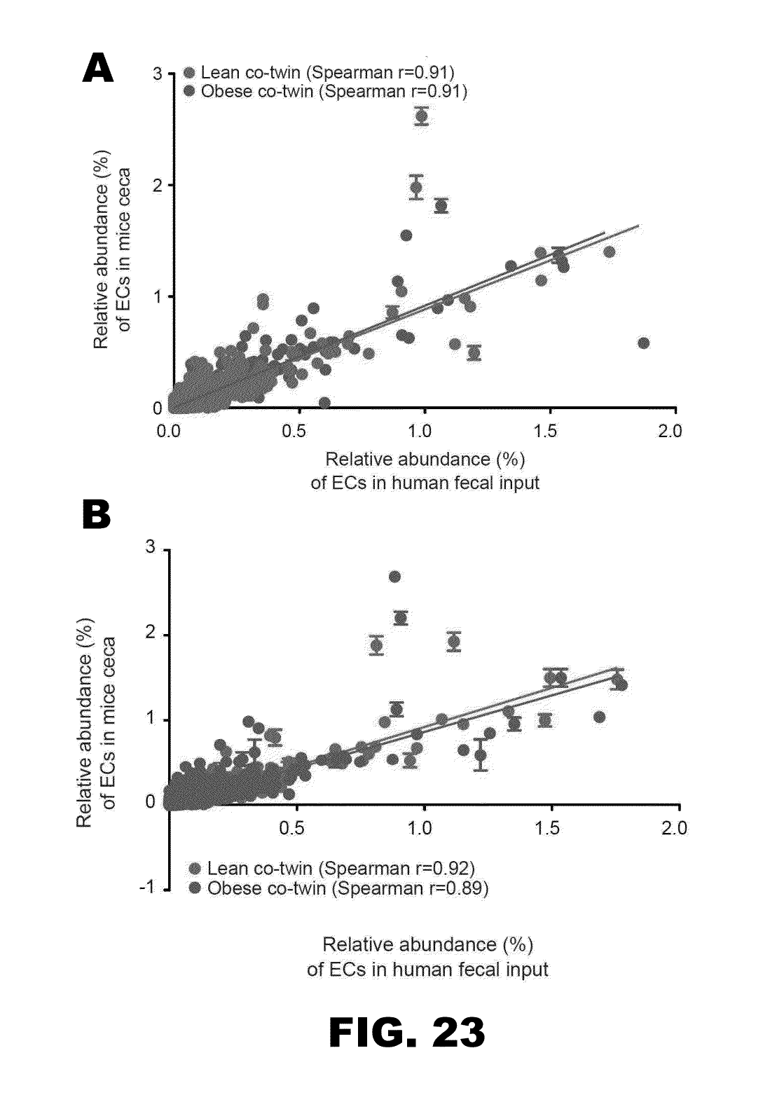

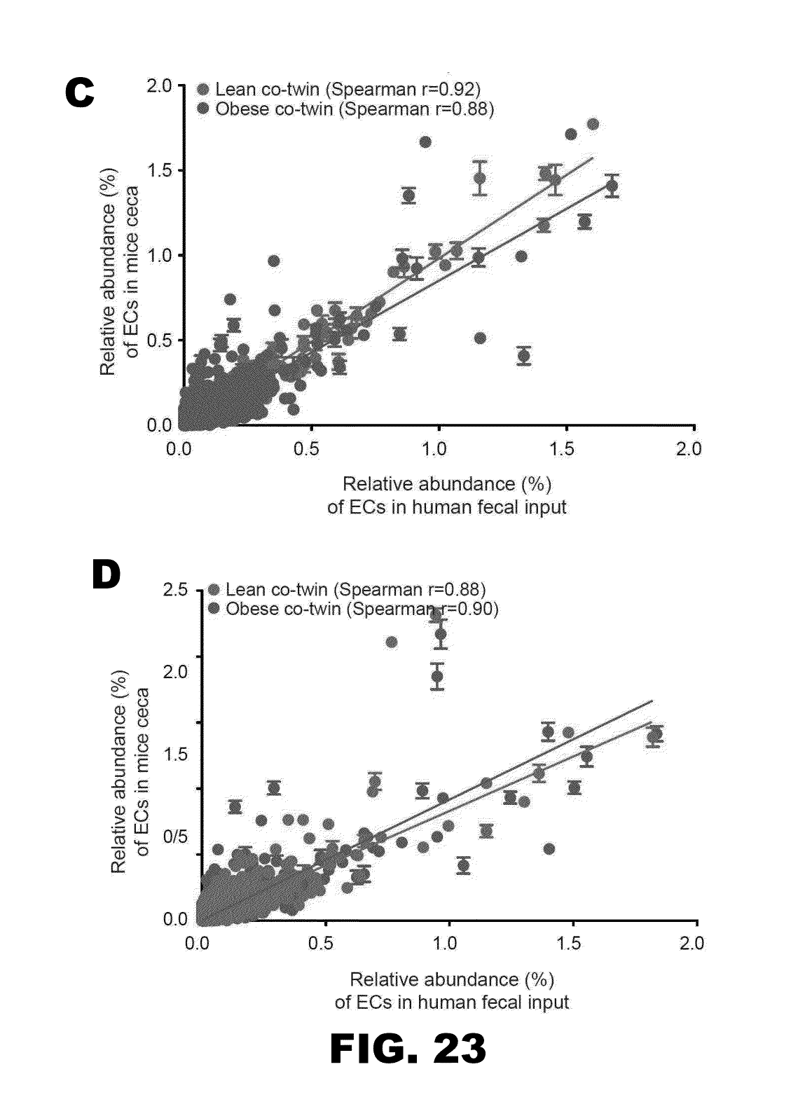

FIG. 23 Correlation between the representation of genes with assigned KEGG EC annotations in human donor 1 (A), human donor 2 (B) human donor 3 (C) and human donor 4 (D) microbiome and their representation in the cecal microbiomes of the corresponding gnotobiotic mouse transplant recipients. Each sphere represents an EC. Mean values±SEM are plotted for each EC in a given group of mice (n=4 recipient mice/human donor). The Spearman correlation r-value is indicated and is significant in all cases (p<0.0001).

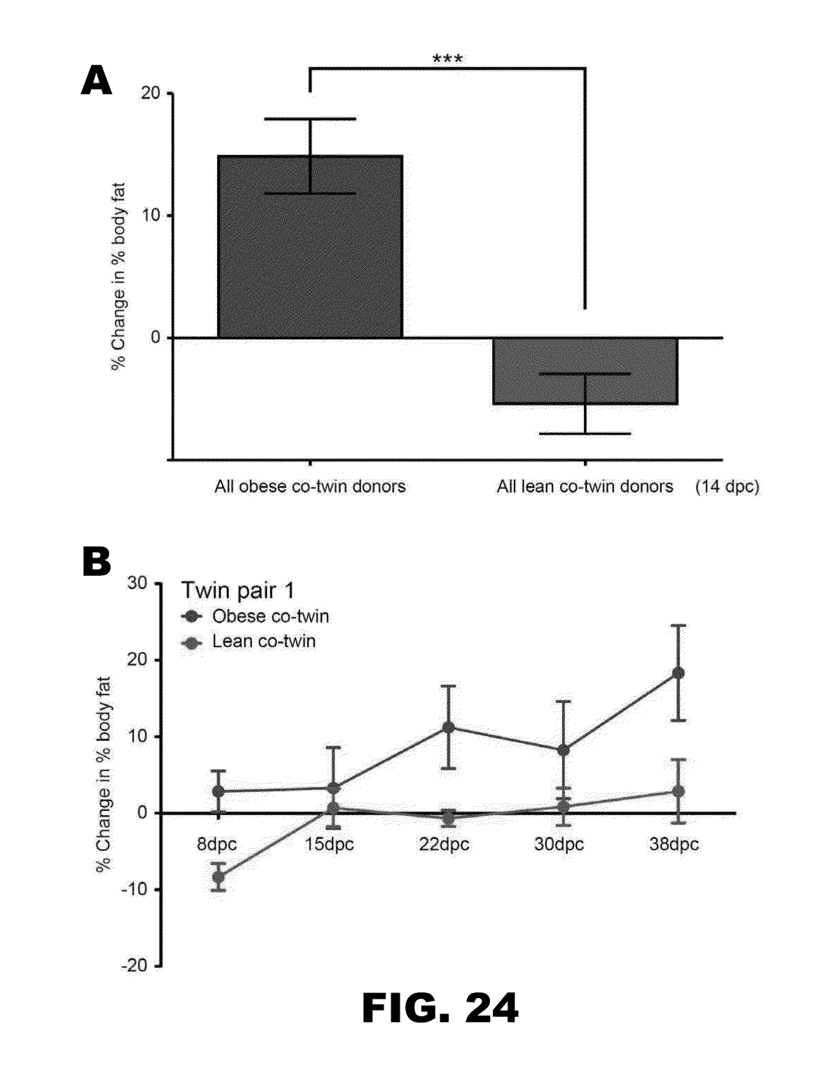

FIG. 24 Transmission of the increased adiposity phenotypes of obese co-twins in discordant pairs by transplantation of their fecal microbiota into gnotobiotic mice. (A) Body composition, defined by quantitative magnetic resonance, performed one day and two weeks post-colonization, of each mouse in each recipient group. Mean values±SEM are plotted of the increase in % body fat over 14 d in all recipient mice for each of the 4 obese co-twins' or lean co-twins' fecal microbiota (n=3-12 animals/donor microbiota; 41 mice/BMI bin; total of 82 mice). ***, p<0.001, as judged by a two-tail Mann-Whitney U-test. (B) More detailed and prolonged time course study for recipients of fecal microbiota from the co-twins in discordant pair 1 (mean values±SEM plotted; n=4 mice/donor microbiota). A linear mixed model revealed that the difference between the gain in adiposity between the two recipient groups of mice is statistically significant (p<0.001).

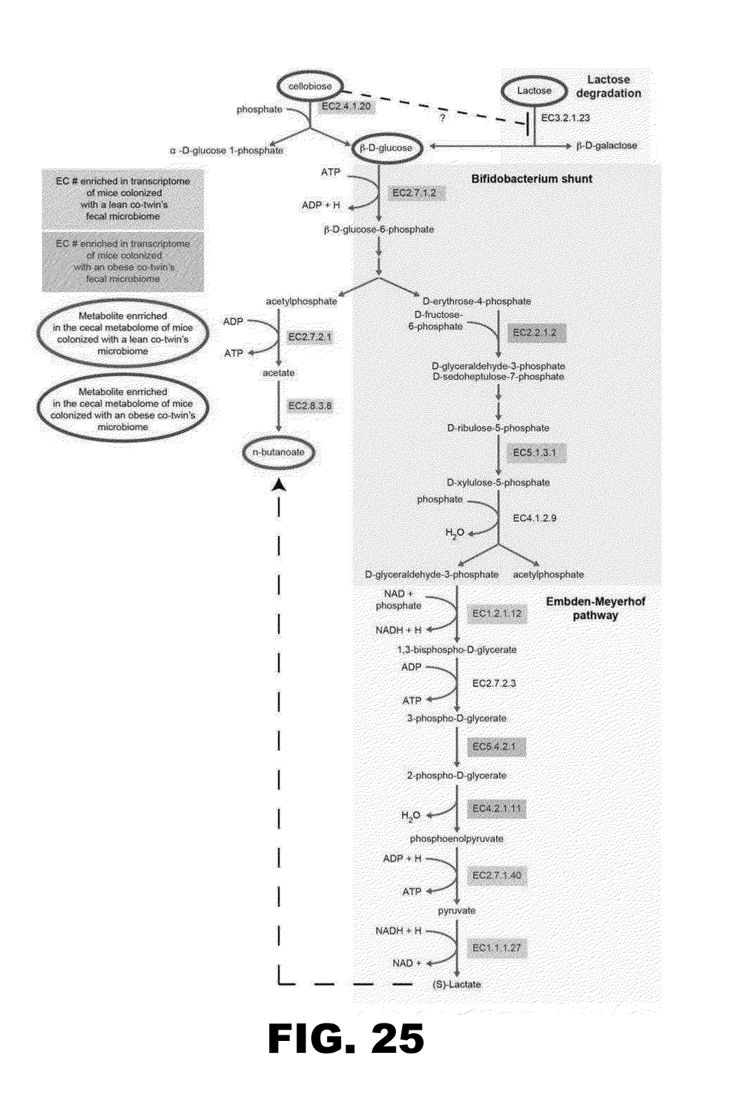

FIG. 25 Pathway maps representing ECs enriched in the fecal meta-transcriptome of mice colonized with an obese compared to lean co-twin's fecal microbiome.

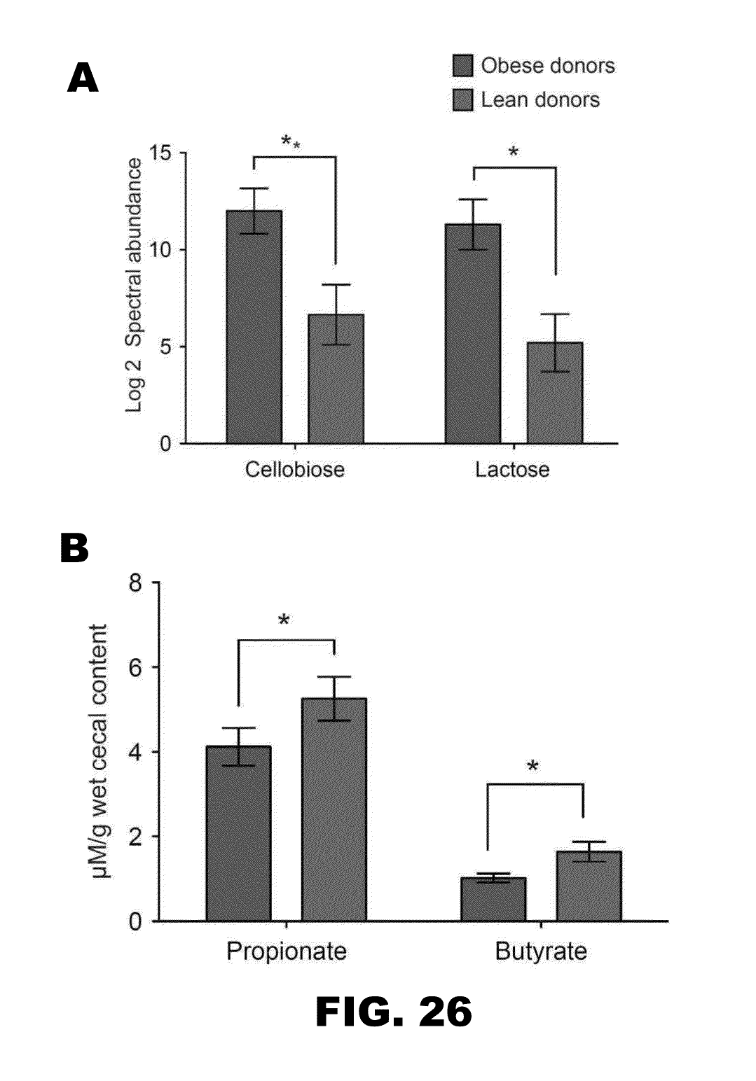

FIG. 26 Metabolites with significant differences in their levels in the ceca of gnotobiotic recipients of obese compared to lean co-twin fecal microbiome transplants. (A) cellobiose and lactose levels as defined by non-targeted GC/MS. (B) Targeted GC/MS analysis of cecal SCFA. *, p<0.05; **, p<0.01 (two-tailed unpaired Student's t-test).

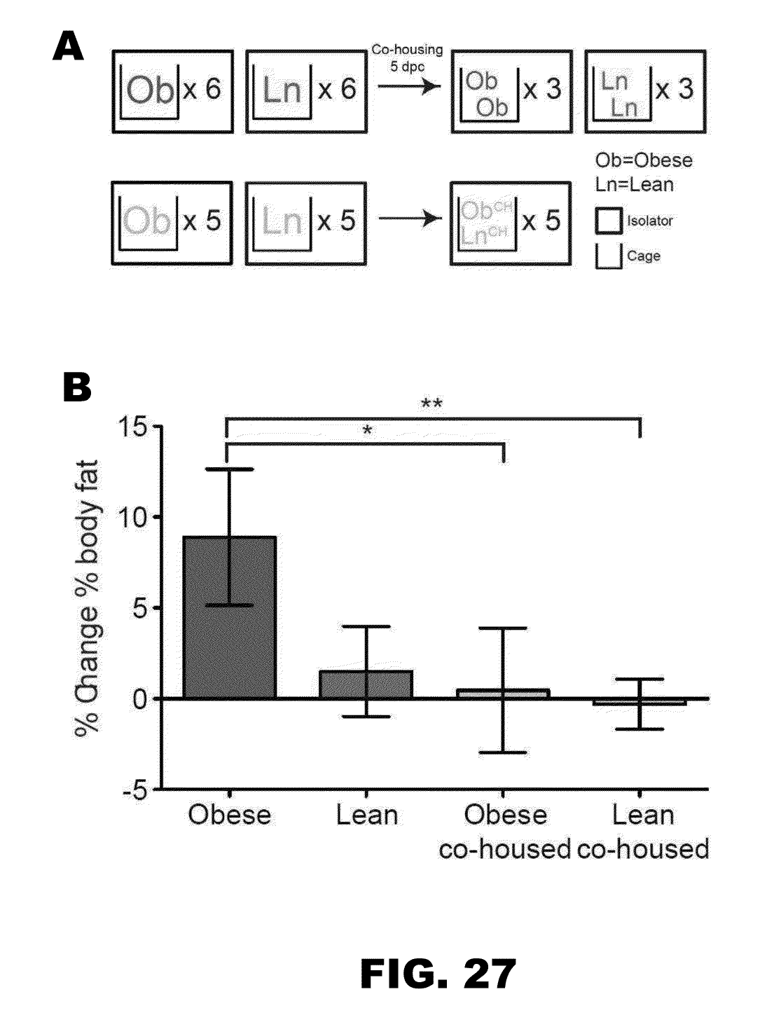

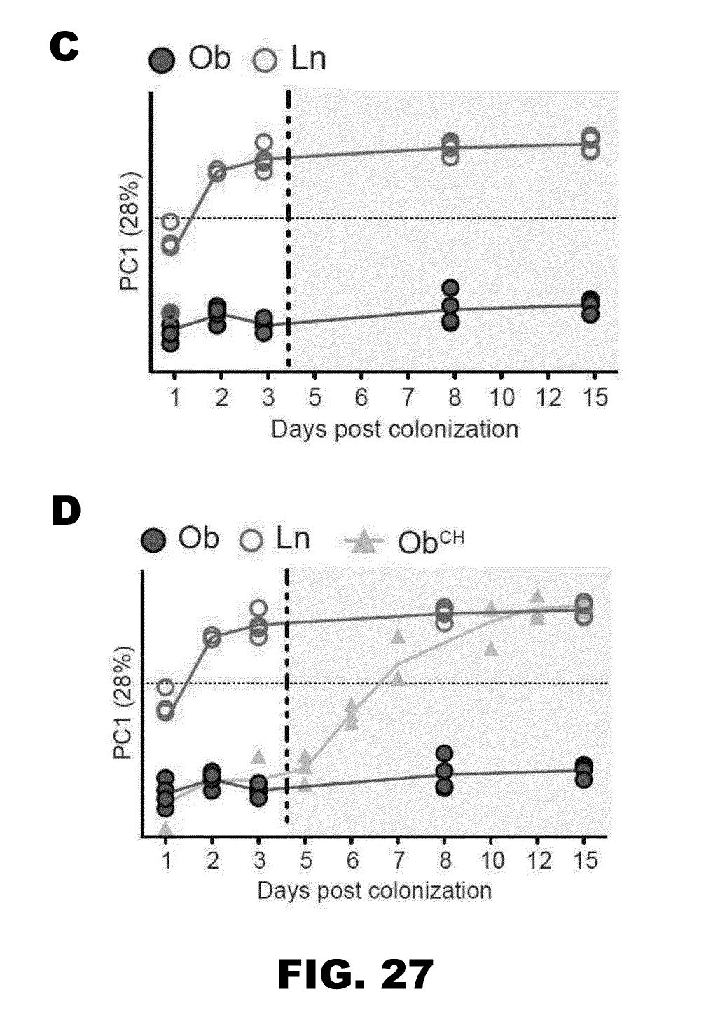

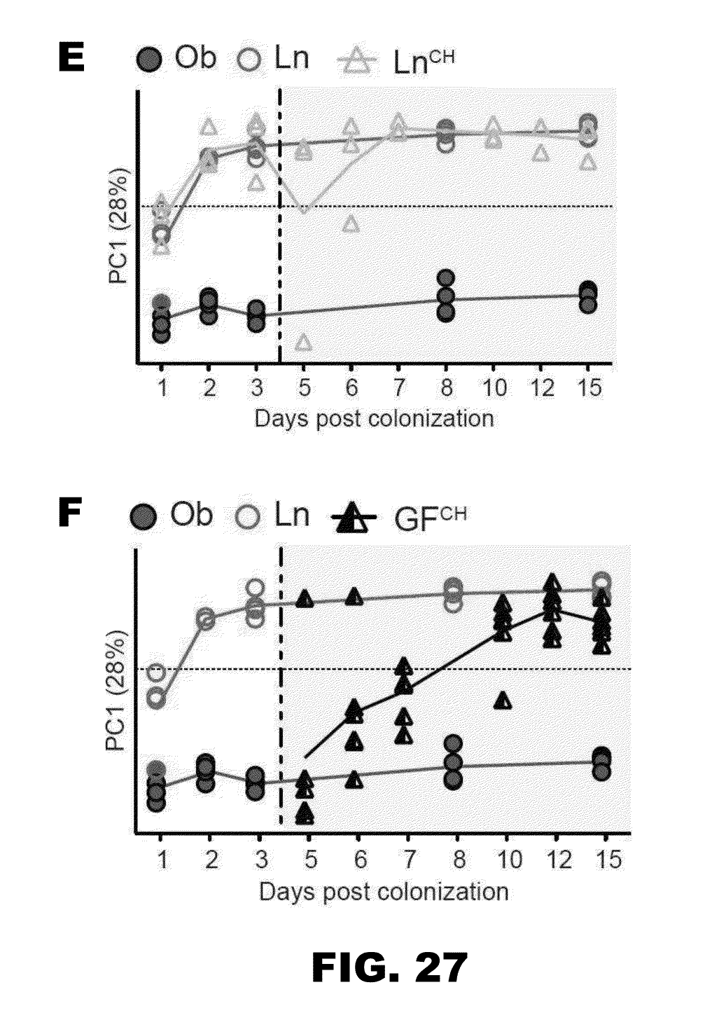

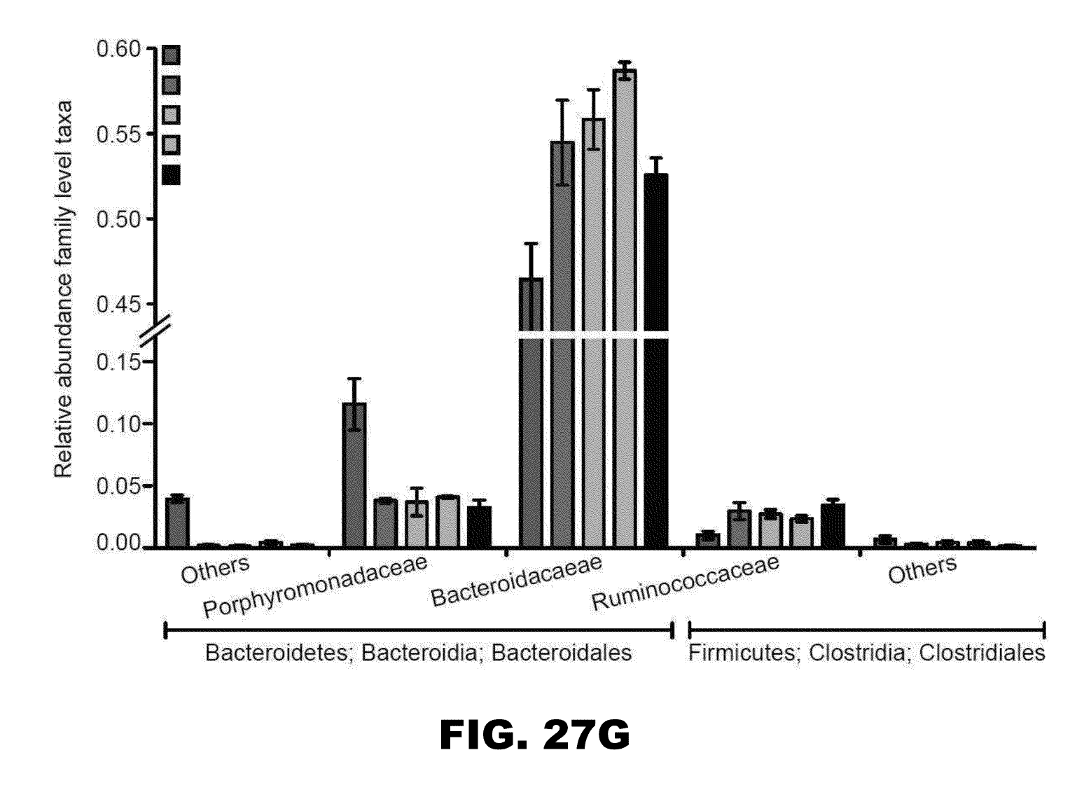

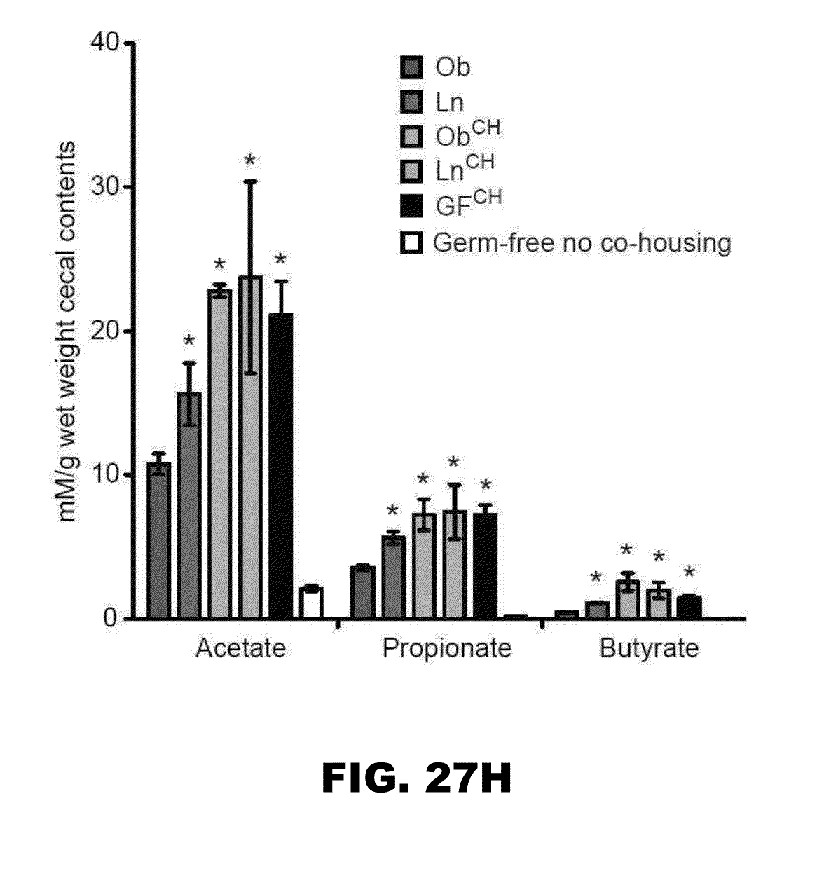

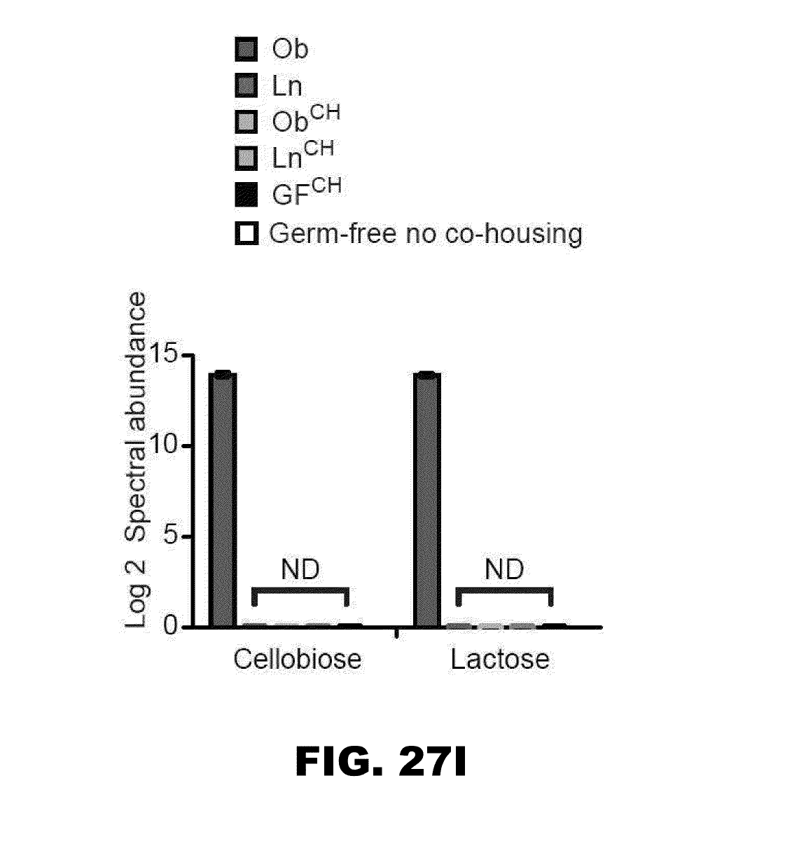

FIG. 27 Transplantation of a bacterial culture collection from an obese co-twin into germ-free mice produces an increased adiposity phenotype that is ameliorated by exposure to co-housed mice harboring a culture collection from her lean co-twin. (A) Design of follow-up co-housing experiment. 8 week-old, male germ-free C57Bl/6J mice received culture collections from the lean (Ln) co-twin or the obese (Ob) co-twin in DZ twin pair 1. Five days post gavage (5dpc) mice were dually co-housed in one of three configurations: control groups consisted of Ob-Ob, or Ln-Ln cagemates; the experimental group consisted of Obch-Lnch cagemates. The number of types of cages in each gnotobiotic isolator is shown. All mice were fed a low-fat, plant polysaccharide-rich diet. Adiposity phenotypes were measured by quantitative magnetic resonance. (B) Adiposity change from dpc1 measured by quantitative MR at dpc15 after 10 d of co-housing. (C-F). PCoA plot of UniFrac distances between transplanted bacterial communities showing the effects of co-housing over time. Also shown are the results obtained when two GF mice were co-housed (GFch) with a Lnch and a Obch mouse (n=3 cages). (G) Family-level taxa whose representation are significantly different between Ob mice and Ln, Obch, Lnch, or GFch mice (p<0.05 after Bonferroni correction) and discriminatory between the Ob and other groups (Random Forests, feature importance score >0.07). (H) GC/MS analysis of levels of short chain fatty acids in the ceca of the indicated groups of mice. Concentrations of acetate, propionate and butyrate were significantly higher in the ceca of control Ln, and Obch, Lnch, and GFch mice compared to Ob controls (*, p<0.05; two tailed Student's t-test). (I) Non-targeted GC/MS reveals that in contrast to Ob controls, cecal levels of cellobiose and lactose are undetectable in Ln mice and in all three groups of co-housed animals.

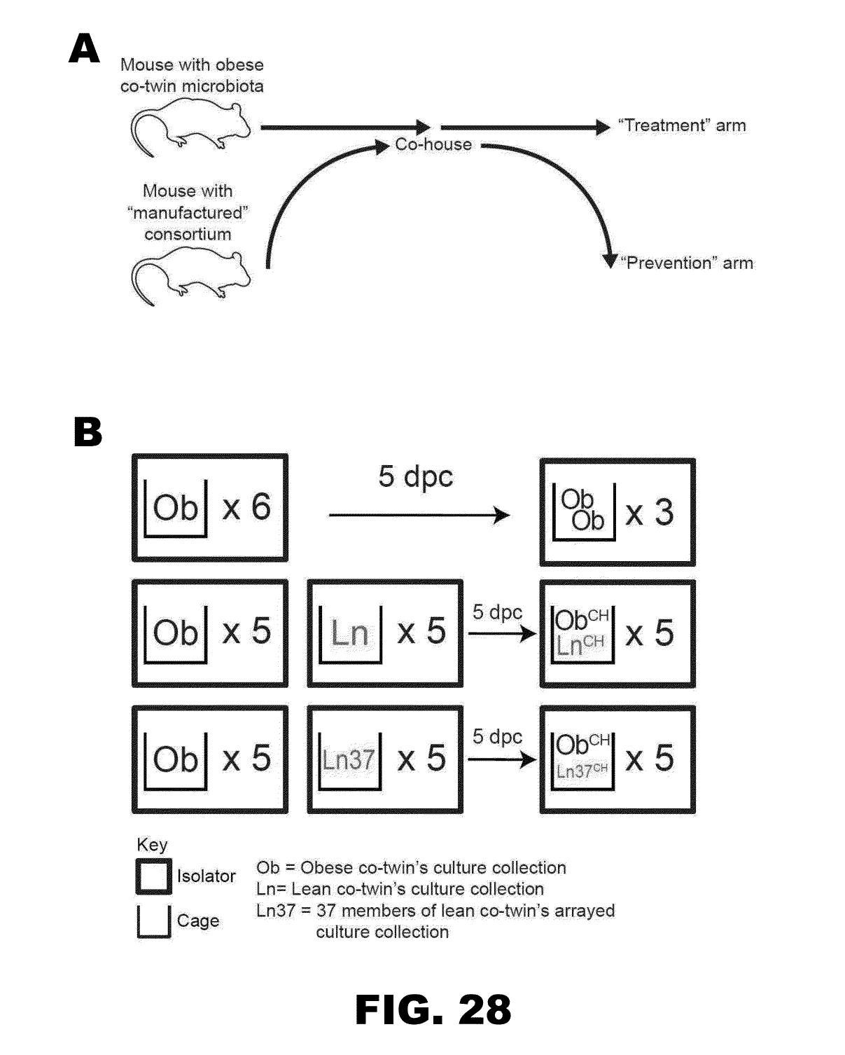

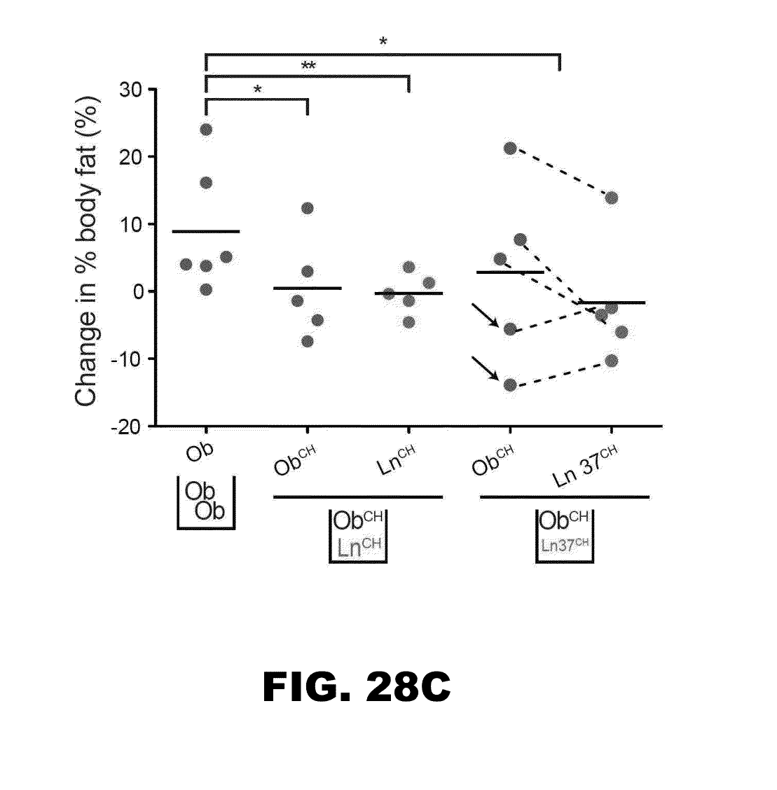

FIG. 28 Co-housing experiments designed to test the effects of a bacterial consortium assembled from the clonally arrayed culture collection from the lean co-twin in DZ pair 1. (A, B) Experimental design. Effects of co-housing Obch and Ln37ch mice on (C) adiposity (note that dashes connect animals that were co-housed, arrows highlight the Obch mice whose adiposity decreased during the period of co-housing, *, p<0.05, **, p<0.01 compared to Ob controls as defined by Mann-Whitney U test).

### DETAILED DESCRIPTION OF THE INVENTION

The present invention discloses in vitro and in vivo cultures of a gut microbial community, models comprising such cultures, and methods of use thereof. Such a culture, while not a complete reproduction of a gut microbial community, maintains a phylotypic composition such that the culture reflects the original gut microbial community it was derived from. The original gut microbial community may be a complete gut microbial community, or a prior culture of a complete gut microbial community. As used herein, a “complete” gut microbial community refers to the natural in vivo composition of the gut microbial community of a given individual. Advantageously, a culture of the invention allows the analysis of the effect of perturbations on a complete gut microbial community by analyzing the effect of the perturbation on a culture derived from the gut microbial community.

The present invention comprises several different in vitro cultures, including a cultured collection of a gut microbial community and a clonally arrayed culture collection of a gut microbial community. Additionally, the invention comprises in vivo cultures, wherein an animal comprises a cultured collection of a gut microbial community or a clonally arrayed culture collection of a gut microbial community. Furthermore, the invention comprises methods of using a cultured collection of a gut microbial community, a clonally arrayed culture collection of a gut microbial community, or an animal comprising a culture of a gut microbial community.

### (I) In Vitro Cultures of a Gut Microbial Community

The present invention encompasses in vitro cultures of a gut microbial community. For instance, the present invention encompasses a cultured collection of a gut microbial community and a clonally arrayed cultured collection of a gut microbial community as detailed below. Generally speaking, an in vitro culture will have a phylotypic composition similar to the original gut microbial community.

### (a) Phylotypic Composition

The term “phylotypic composition,” as used herein, refers to the composition of a gut microbial community as defined by phylotypes. A phylotype is a biological type that classifies an organism by its phylogenetic, e.g. evolutionary, relationship to other organisms. The term phylotype is taxon-neutral, and therefore, may refer to the species composition, genus composition, class composition, etc. or, in alternative embodiments, may refer to organisms with a specified genetic similarity (e.g. 97% similar at a sequence level, or 97% similar at a gene function level).

In some embodiments, an in vitro culture of a gut microbial community may comprise between about 1 and 100% of the phylotypes present in the original gut microbial community. In certain embodiments, an in vitro culture of a gut microbial community may comprise at least about 1, 2, 3, 4, 5, 6, 7, 8, 9, or 10% of the phylotypes present in the original gut microbial community. In other embodiments, an in vitro culture of a gut microbial community may comprise at least about 10, 15, 20, 25, 30, 35, 40, 45, 50, 55, 60, 65, 70, 75, 80, 85, 90, 95 or 100% of the phylotypes present in the original gut microbial community. In still other embodiments, an in vitro culture of a gut microbial community may comprise at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% of the phylotypes present in the original gut microbial community. In yet other embodiments, an in vitro culture of a gut microbial community may comprise at least about 90, 91, 92, 93, 94, 95, 96, 97, 98, 99 or 100% of the phylotypes present in the original gut microbial community. In a preferred embodiment, an in vitro culture of a gut microbial community may comprise at least about 98.0, 98.1, 98.2, 98.3, 98.4, 98.5. 98.6, 98.7, 98.9, 99.0, 99.1, 99.2, 99.3, 99.4, 99.5, 99.6, 99.7, 99.8, or 99.9% of the phylotypes present in the original gut microbial community. In another preferred embodiment, an in vitro culture of a gut microbial community may comprise greater than 99.0% of the phylotypes present in the original gut microbial community.

In exemplary embodiments, an in vitro culture of a gut microbial community may comprise at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% of the phylum, class, order, family, genus or species phylotypes present in the original gut microbial community. In yet other embodiments, an in vitro culture of a gut microbial community may comprise at least about 90, 91, 92, 93, 94, 95, 96, 97, 98, 99 or 100% of the phylum, class, order, family, genus or species phylotypes present in the original gut microbial community. In a preferred embodiment, an in vitro culture of a gut microbial community may comprise at least about 98.0, 98.1, 98.2, 98.3, 98.4, 98.5. 98.6, 98.7, 98.9, 99.0, 99.1, 99.2, 99.3, 99.4, 99.5, 99.6, 99.7, 99.8, or 99.9% of the phylum, class, order, family, genus or species phylotypes present in the original gut microbial community. In another preferred embodiment, an in vitro culture of a gut microbial community may comprise greater than 99.0% of the phylum, class, order, family, genus or species phylotypes present in the original gut microbial community.

In certain exemplary embodiments, an in vitro culture of a gut microbial community may comprise at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% of the metagenome, transcriptome, or proteome of the original gut microbial community. In yet other embodiments, an in vitro culture of a gut microbial community may comprise at least about 90, 91, 92, 93, 94, 95, 96, 97, 98, 99 or 100% of the metagenome, transcriptome, or proteome of the original gut microbial community. In a preferred embodiment, an in vitro culture of a gut microbial community may comprise at least about 98.0, 98.1, 98.2, 98.3, 98.4, 98.5, 98.6, 98.7, 98.9, 99.0, 99.1, 99.2, 99.3, 99.4, 99.5, 99.6, 99.7, 99.8, or 99.9% of the metagenome, transcriptome, or proteome of the original gut microbial community. In another preferred embodiment, an in vitro culture of a gut microbial community may comprise greater than 99.0% of the metagenome, transcriptome, or proteome of the original gut microbial community.

The phylotypic composition of a cultured or complete gut microbial community may be evaluated using several different methods. Non-limiting examples of methods that may be used to evaluate the phylotypic composition of a complete or cultured gut microbial community may include the biological classification of individual isolated microbial colonies, the analysis of the biological functions represented in a sample, and the metagenomic analysis of genetic material isolated from the complete or cultured gut microbial community.

In some embodiments, the phylotypic composition of a gut microbial community may be evaluated by analyzing biological functions represented in a sample of the community. Suitable biological functions may include enzyme functions or drug resistance, such as antibiotic resistance. For instance, the capture and characterization of antibiotic resistance genes may be used to evaluate the biological functions represented in a sample. Non-limiting examples of antibiotics that may be used to capture and characterize antibiotic resistance genes may include amikacin, amoxicillin, carbenicillin, cefdinir, cloramphenicol, ciprofloxacin, cefepime, gentamicin, oxytetracyline, penicillin, piperacillin, piperacillin+Tazobactam, tetracycline, trimethoprim, and rimethoprim+sulfamethoxazole.

In other embodiments, the phylotypic composition of a gut microbial community may be evaluated using metagenomic analysis of genetic material isolated from the gut microbial community. For instance, a conserved region in the composite genomes of the gut microbial community may be sequenced, or the composite genome of a gut microbial community may be shotgun sequenced. In one embodiment, the phylotypic composition of a gut microbial community may be evaluated by sequencing a conserved 16S ribosomal RNA (rRNA) gene of the composite genomes of the gut microbial community. By way of non-limiting example, DNA from a complete or cultured collection of a gut microbial community may be extracted and the variable region 2 (V2) of bacterial 16S rRNA genes may be pyrosequenced.

In yet other embodiments, the phylotypic composition of a gut microbial community may be evaluated by shotgun sequencing of the composite genomes followed by analysis of predicted functions contained in the composite genomes of the gut microbial community. In one embodiment, the phylotypic composition of a gut microbial community may be evaluated by shotgun sequencing of the composite genomes followed by analysis of predicted functions contained in the composite genomes of the gut microbial community by querying against a known database, such as the KEGG Orthology (KO) database.

The phylotypic composition in a gut microbial community may be evaluated at various stages during sample collection, extraction, culture, and storage to produce a profile of diversity in a sample. In some embodiments, the phylotypic composition in the gut microbial community may be evaluated after extraction from the animal host but before culture. In other embodiments, the representation of the taxa in the gut microbial community may be evaluated after culture. In preferred embodiments, the taxa in the gut microbial community may be evaluated both after extraction from the animal host and after culture.

### (b) Cultured Collection of a Gut Microbial Community

One aspect of the present disclosure provides a cultured collection of a gut microbial community. As used herein, a “cultured collection of a gut microbial community” refers to an in vitro collection of cultured microorganisms derived from an original gut microbial community. Cultivation of a cultured collection of a gut microbial community may alter the microbial community structure and representation of members of the original gut microbial community, resulting in a “cultured collection” with a phylotypic composition similar to the original gut microbial community. In an exemplary embodiment, the cultured collection is stable, meaning that over time the members comprising the collection do not substantially change. As used herein, “substantially change” means less than about 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1% difference between the members comprising the cultured collection when it is evaluated at two separate time points. In some embodiments, the cultured collection is stable for 5, 10, 15, 20, 25, 30, 35, 40, 45, 50, 55, 60, 65, 70, 75, 80, 85, 90, or more than 90 days. In other embodiments, the cultured collection is stable for 1, 2, 3, 4, 5, 6, 7, 8, 9, 10, 11, 12, or more than 12 months. In still other embodiments, the cultured collection is stable for 1, 2, 3, 4, 5, 6, 7, 8, 9, 10 or more than 10 years.

Culture conditions may be optimized to maximize the phylotypic composition of members of the original gut microbial community during culture. Non-limiting examples of methods that may be used to optimize culture conditions to maximize the phylotypic composition during culture may include using a low concentration of nutrients to limit growth of aggressive members of the gut microbial community, optimizing plating density to produce dense but distinct colonies, and optimizing the incubation period to balance the growth of aggressive and slow growing members of a gut microbial community.

In some embodiments, low concentrations of a nutrient may be used to limit growth of aggressive members of the gut microbial community to maximize the phylotypic composition of members of a gut microbial community during culture. Non-limiting examples of commonly used nutrients in microbial culture that may be used at low concentrations include glucose, tryptone and yeast extract.

In other embodiments, plating density is optimized to produce dense but distinct colonies to maximize the phylotypic composition of members of a gut microbial community during culture. In a preferred embodiment, a sample of a gut microbial community may be plated at a density of about 4000 to about 6000 colonies per 150 mm diameter culture plate. In another preferred embodiment, a sample of a gut microbial community may be plated at a density of about 2000 to about 7000 colonies per 150 mm diameter culture plate. In an exemplary embodiment, a sample of a gut microbial community may be plated at a density of about 5000 colonies per 150 mm diameter culture plate.

The number of colonies cultured from a sample of a gut microbial community can and will vary depending on the desired phylotypic composition in the resulting cultured collection of the gut microbial community. The number of colonies needed to culture a gut microbial community may be determined using any of the methods used to assess the phylotypic composition of a gut microbial community described above. In some embodiments, the number of colonies cultured from a sample of a gut microbial community is about 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, or more than 20,000. In other embodiments, the number of colonies cultured from a sample of a gut microbial community is about 20,000 to 40,000. In yet other embodiments, the number of colonies cultured from a sample is greater than 40,000. In an exemplary embodiment, the number of colonies cultured from a sample of a gut microbial community is about 30,000 colonies.

The incubation period during the culture of a gut microbial community may be optimized to maximize the phylotypic composition. In some embodiments, plates comprising a gut microbial community may be incubated for about 1, 2, 3, 4, 5, 6, 7, 8, 9, 10, 11, 12, 13, 14 days or more. In one embodiment, plates comprising a gut microbial community may be incubated for about 5 days.

In some embodiments, non-commercially available components that may help increase the phylotypic composition during culture may be used. Non-limiting examples of such components may include sterile rumen or human fecal extracts.

In one embodiment, a gut microbial community isolated from a subject is cultured on solid agar media.

In an exemplary embodiment, a cultured collection of a gut microbial community may comprise at least about 50, 60, 70, 80, 90, 91, 92, 93, 94, 95, 96, 97, 98, 99 or 100% of the phylotypes present in the original gut microbial community. In a preferred embodiment, a cultured collection of a gut microbial community may comprise at least about 98.0, 98.1, 98.2, 98.3, 98.4, 98.5. 98.6, 98.7, 98.9, 99.0, 99.1, 99.2, 99.3, 99.4, 99.5, 99.6, 99.7, 99.8, or 99.9% of the phylotypes present in the original gut microbial community. In another preferred embodiment, a cultured collection of a gut microbial community may comprise greater than 99.0% of the phylotypes present in the original gut microbial community.

In exemplary embodiments, a cultured collection of a gut microbial community may comprise at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% of the phylum, class, order, family, genus or species phylotypes present in the original gut microbial community. In yet other embodiments, a cultured collection of a gut microbial community may comprise at least about 90, 91, 92, 93, 94, 95, 96, 97, 98, 99 or 100% of the phylum, class, order, family, genus or species phylotypes present in the original gut microbial community. In a preferred embodiment, a cultured collection of a gut microbial community may comprise at least about 98.0, 98.1, 98.2, 98.3, 98.4, 98.5. 98.6, 98.7, 98.9, 99.0, 99.1, 99.2, 99.3, 99.4, 99.5, 99.6, 99.7, 99.8, or 99.9% of the phylum, class, order, family, genus or species phylotypes present in the original gut microbial community. In another preferred embodiment, a cultured collection of a gut microbial community may comprise greater than 99.0% of the phylum, class, order, family, genus or species phylotypes present in the original gut microbial community.

In certain exemplary embodiments, a cultured collection of a gut microbial community may comprise at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% of the metagenome, transcriptome, or proteome of the original gut microbial community. In yet other embodiments, a cultured collection of a gut microbial community may comprise at least about 90, 91, 92, 93, 94, 95, 96, 97, 98, 99 or 100% of the metagenome, transcriptome, or proteome of the original gut microbial community. In a preferred embodiment, a cultured collection of a gut microbial community may comprise at least about 98.0, 98.1, 98.2, 98.3, 98.4, 98.5, 98.6, 98.7, 98.9, 99.0, 99.1, 99.2, 99.3, 99.4, 99.5, 99.6, 99.7, 99.8, or 99.9% of the metagenome, transcriptome, or proteome of the original gut microbial community. In another preferred embodiment, a cultured collection of a gut microbial community may comprise greater than 99.0% of the metagenome, transcriptome, or proteome of the original gut microbial community.

### (c) Clonally Arrayed Culture Collection of a Gut Microbial Community

Another aspect of the present disclosure provides a clonally arrayed culture collection of a gut microbial community. A “clonally arrayed culture collection” of a gut microbial community, as used herein, refers to a collection of cultured microbes each derived from a single microbial cell from a gut microbial community.

A clonally arrayed culture collection of a gut microbial community may be derived from a complete gut microbial community isolated from a subject, or from a previously cultured gut microbial community. A complete or previously cultured gut microbial community may be sampled as described in section I(d) below.

In some embodiments, a clonally arrayed culture collection of a gut microbial community may be generated by picking and isolating individual colonies from a gut microbial community cultured on plates. In certain embodiments, a clonally arrayed culture collection of a gut microbial community may be generated using a most probable number (MPN) technique, also known as the method of Poisson zeroes. In essence, the MPN technique allows for creating clonally arrayed species collections by inoculating culture wells with a diluted sample of a gut microbial community so that a certain percentage of the inoculated wells does not receive a microbe.

In some embodiments, a clonally arrayed culture collection of a gut microbial community is generated using a dilution point that yields about 30 to 90% empty wells. In other embodiments, a clonally arrayed culture collection of a gut microbial community is generated using a dilution point that yields about 50, 40, 60, 70, 80, or 90% empty wells. In a preferred embodiment, a clonally arrayed culture collection of a gut microbial community is generated using a dilution point that yields about 70% empty wells. At this dilution point, only about 5% of the wells will receive more than one cell in the inoculum, or non-clonal wells.

Handling, culture and storage conditions for generating a clonally arrayed culture collection of a gut microbial community are under strictly anaerobic conditions as described in section I(d) below. A clonally arrayed culture collection of a gut microbial community may be in multiwell culture plates. Non-limiting examples of multiwell culture plates that may be used for generating and storing a clonally arrayed culture collection of a gut microbial community include 6-well, 12-well, 24-well, 48-well, 96-well and 384-well plates. In an exemplary embodiment, a clonally arrayed culture collection of a gut microbial community may be in 384-well plates.

A clonally arrayed culture collection of a gut microbial community may be taxonomically defined. A two-step barcoded pyrosequencing scheme illustrated in FIG. 12 may be used to allow each culture well to be associated with its corresponding bacterial 16S rRNA sequence. In essence, the two-step barcoded pyrosequencing scheme uses two DNA amplification reactions using barcoded primers. This, combined with pyrosequencing, allows unambiguous assignment of 16S rRNA reads to well and plate locations using a minimum number of barcodes and primers.

In some embodiments, a clonally arrayed culture collection derived from an original gut microbial community contains about 100 to about 5000 taxonomically defined isolates. In other embodiments, a clonally arrayed culture collection may contain about 500, 600, 700, 800, 900, 1000, 2000, 3000, or 4000 to about 5000 taxonomically defined isolates. In still other embodiments, a clonally arrayed culture collection may contain about 800, 900, 1000, or 2000 to about 5000 taxonomically defined isolates. In an exemplary embodiment, the clonally arrayed culture collection may contain about 1,000 taxonomically defined isolates.

In an exemplary embodiment, a clonally arrayed culture collection of a gut microbial community may comprise at least about 90, 91, 92, 93, 94, 95, 96, 97, 98, 99 or 100% of the phylotypes present in the original gut microbial community. In a preferred embodiment, a clonally arrayed culture collection of a gut microbial community may comprise at least about 98.0, 98.1, 98.2, 98.3, 98.4, 98.5. 98.6, 98.7, 98.9, 99.0, 99.1, 99.2, 99.3, 99.4, 99.5, 99.6, 99.7, 99.8, or 99.9% of the phylotypes present in the original gut microbial community. In another preferred embodiment, a clonally arrayed culture collection of a gut microbial community may comprise greater than 99.0% of the phylotypes present in the original gut microbial community.

In exemplary embodiments, a clonally arrayed culture collection of a gut microbial community may comprise at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% of the phylum, class, order, family, genus or species phylotypes present in the original gut microbial community. In yet other embodiments, a clonally arrayed culture collection of a gut microbial community may comprise at least about 90, 91, 92, 93, 94, 95, 96, 97, 98, 99 or 100% of the phylum, class, order, family, genus or species phylotypes present in the original gut microbial community. In a preferred embodiment, a clonally arrayed culture collection of a gut microbial community may comprise at least about 98.0, 98.1, 98.2, 98.3, 98.4, 98.5. 98.6, 98.7, 98.9, 99.0, 99.1, 99.2, 99.3, 99.4, 99.5, 99.6, 99.7, 99.8, or 99.9% of the phylum, class, order, family, genus or species phylotypes present in the original gut microbial community. In another preferred embodiment, a clonally arrayed culture collection of a gut microbial community may comprise greater than 99.0% of the phylum, class, order, family, genus or species phylotypes present in the original gut microbial community.

In certain exemplary embodiments, a clonally arrayed culture collection of a gut microbial community may comprise at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% of the metagenome, transcriptome, or proteome of the original gut microbial community. In yet other embodiments, a clonally arrayed culture collection of a gut microbial community may comprise at least about 90, 91, 92, 93, 94, 95, 96, 97, 98, 99 or 100% of the metagenome, transcriptome, or proteome of the original gut microbial community. In a preferred embodiment, a clonally arrayed culture collection of a gut microbial community may comprise at least about 98.0, 98.1, 98.2, 98.3, 98.4, 98.5, 98.6, 98.7, 98.9, 99.0, 99.1, 99.2, 99.3, 99.4, 99.5, 99.6, 99.7, 99.8, or 99.9% of the metagenome, transcriptome, or proteome of the original gut microbial community. In another preferred embodiment, a clonally arrayed culture collection of a gut microbial community may comprise greater than 99.0% of the metagenome, transcriptome, or proteome of the original gut microbial community.

### (d) Gut Microbial Community and Collection

An in vitro culture of a gut microbial community may be derived from a subject that is a rodent, a human, a livestock animal, a companion animal, or a zoological animal. In one embodiment, a culture of a gut microbial community may be derived from a rodent, e.g. a mouse, a rat, a guinea pig, etc. In another embodiment, an in vitro culture of a gut microbial community may be derived from a livestock animal. Non-limiting examples of suitable livestock animals may include pigs, cows, horses, goats, sheep, llamas and alpacas. In still another embodiment, an in vitro culture of a gut microbial community may be derived from a companion animal. Non-limiting examples of companion animals may include pets such as dogs, cats, rabbits, and birds. In still yet another embodiment, an in vitro culture of a gut microbial community may be derived from a zoological animal. As used herein, a “zoological animal” refers to an animal that may be found in a zoo. Such animals may include non-human primates, large cats, wolves, and bears. In an exemplary embodiment, an in vitro culture of a gut microbial community may be derived from a human.

An in vitro culture of a gut microbial community may be derived from the same subject over a predetermined time period. For instance, in some embodiments, the microbial community may be sampled at an interval of 1, 2, 3, 4, 5, 6, 7, 8, 9, 10, 20, 30, 40, 50, 60, 70, 80, 90, 100, or 200 days.

In some embodiments, an in vitro culture of a gut microbial community may be derived from a subject with an endemic gut microbial community. In other embodiments, an in vitro culture of a gut microbial community may be derived from a sterile subject inoculated with a gut microbial community from another subject. In yet other embodiments, an in vitro culture of a gut microbial community may be derived from a sterile animal inoculated with a previously cultured gut microbial community (e.g. a cultured collection or a clonally arrayed cultured collection as described herein). In other embodiments, an in vitro culture of a gut microbial community may be derived from a sterile animal inoculated with a defined mixture of gut microbes. In yet other embodiments, an in vitro culture of a gut microbial community may be derived from a sterile animal inoculated with a mixture of gut microbes from a clonally arrayed culture collection of a gut microbial community.

The gut environment in a suitable subject is anaerobic. Any prolonged exposure to aerobic conditions may lead to a significant alteration in the gut microbial community structure. Therefore, to reflect the gut microbial community structure in a subject, sample collection, extraction, culture and storage conditions should be maintained under strictly anaerobic conditions upon harvesting the sample from the animal host. Methods for providing anaerobic conditions for sample collection, extraction, culture and storage are known in the art and include performing all operations in anaerobic chambers and incubators.

Anaerobic conditions must also be maintained in sample extraction buffers and growth media. Anaerobic conditions may be maintained in the sample extraction buffers and growth media by using reducing agents. Non-limiting examples of reducing agents may include cysteine.

Methods of collecting a gut microbial community are known in the art. In certain embodiments, a gut microbial community may be extracted from luminal material collected from the gastrointestinal system, such as from the proximal, central, or distal portions of the small intestine, cecum, or colon. In other embodiments, a gut microbial community may be extracted from a freshly excreted fecal sample. Generally speaking, a freshly excreted fecal sample should be transferred to an anaerobic chamber within 1, 2, 3, 4, 5, 6, 7, 8, 9, or 10 minutes of its collection. In one embodiment, a freshly excreted fecal sample is transferred to an anaerobic chamber within 5 minutes of its collection.

Generally speaking, a newly collected sample of a gut microbial community may be suspended in buffer. In a preferred embodiment, the sample is allowed to separate in the buffer, allowing large insoluble particles to settle, thus improving downstream handling steps. In an exemplary embodiment, a gut microbial community may be suspended in pre-reduced PBS buffer.

### (II) In Vivo Culture of a Gut Microbial Community

Yet another aspect of the present disclosure provides an animal comprising a gut microbial community consisting of cultured microbial members. In essence, to generate an animal comprising a gut microbial community consisting of cultured microbial members, a sterile animal may be colonized with a cultured gut microbial community. Such an animal may be referred to as gnotobiotic. Methods of colonizing sterile animals with a gut microbial community are known in the art and consist of introducing an extract comprising a gut microbial community directly into the animal by oral gavage. Oral gavage is the administration of fluids directly into the lower esophagus or stomach using a feeding needle or tube introduced into the mouth and threaded down the esophagus.

In some embodiments, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. The genotype of the sterile animal can and may vary depending on the intended use of the animal. In embodiments where the animal is a mouse, the mouse may be a C57BL/6 mouse, a Balb/c mouse, a 129sv, or any other laboratory strain. In an exemplary embodiment, the mouse is a C57BL/6J mouse. In other embodiments, the animal is a livestock animal, such as swine.

Sterile animal husbandry methods are known in the art. Sterile animals are typically born under aseptic conditions, which may include removal from the mother by Caesarean section. Sterile animals are generally housed in a sterile or microbially-controlled laboratory environment in which they remain free of all microbes such as bacteria, exogenous viruses, fungi, and parasites.

In some embodiments, a sterile animal may be colonized with an in vitro culture of a gut microbial community. An in vitro culture of a gut microbial community may be derived from an animal as described in section I above. In certain embodiments, a sterile animal may be colonized with a cultured collection of a gut microbial community. In other embodiments, a sterile animal may be colonized with a clonally arrayed culture collection of a gut microbial community.

In yet other embodiments, a sterile animal may be colonized with a subset of a clonally arrayed culture collection of a gut microbial community. For instance, a sterile animal may be colonized with one or more clonal members of a clonally arrayed culture collection of a gut microbial community. In one embodiment, a sterile animal may be colonized with 1, 2, 3, 4, 5, 6, 7, 8, 9, 10 or more than 10 clonal members of a clonally arrayed culture collection of a gut microbial community. In another embodiment, a sterile animal may be colonized with about 10, 20, 30, 40, 50, 60, 70, 80, 90, 100 or more than 100 clonal members of a clonally arrayed culture collection of a gut microbial community. In yet another embodiment, a sterile animal may be colonized with about 100, 200, 300, 400, 500, 600, 700, 800, 900 1000 or more than 1000 clonal members of a clonally arrayed culture collection of a gut microbial community. In still another embodiment, a sterile animal may be colonized with about 1000, 2000, 3000 or more clonal members of a clonally arrayed culture collection of a gut microbial community.

In an exemplary embodiment, an in vivo culture of a cultured gut microbial community may comprise at least about 90, 91, 92, 93, 94, 95, 96, 97, 98, 99 or 100% of the phylotypes present in the original gut microbial community. In a preferred embodiment, an in vivo culture of a cultured gut microbial community may comprise at least about 98.0, 98.1, 98.2, 98.3, 98.4, 98.5. 98.6, 98.7, 98.9, 99.0, 99.1, 99.2, 99.3, 99.4, or 99.5% of the phylotypes present in the original gut microbial community. In another preferred embodiment, an in vivo culture of a cultured gut microbial community may comprise greater than 99.0% of the phylotypes present in the original gut microbial community.

In exemplary embodiments, an in vivo culture of a cultured gut microbial community may comprise at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% of the phylum, class, order, family, genus or species phylotypes present in the original gut microbial community. In yet other embodiments, an in vivo culture of a cultured gut microbial community may comprise at least about 90, 91, 92, 93, 94, 95, 96, 97, 98, 99 or 100% of the phylum, class, order, family, genus or species phylotypes present in the original gut microbial community. In a preferred embodiment, an in vivo culture of a cultured gut microbial community may comprise at least about 98.0, 98.1, 98.2, 98.3, 98.4, 98.5. 98.6, 98.7, 98.9, 99.0, 99.1, 99.2, 99.3, 99.4, 99.5, 99.6, 99.7, 99.8, or 99.9% of the phylum, class, order, family, genus or species phylotypes present in the original gut microbial community. In another preferred embodiment, an in vivo culture of a cultured gut microbial community may comprise greater than 99.0% of the phylum, class, order, family, genus or species phylotypes present in the original gut microbial community.

In certain exemplary embodiments, an in vivo culture of a cultured gut microbial community may comprise at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90% of the metagenome, transcriptome, or proteome of the original gut microbial community. In yet other embodiments, an in vivo culture of a cultured gut microbial community may comprise at least about 90, 91, 92, 93, 94, 95, 96, 97, 98, 99 or 100% of the metagenome, transcriptome, or proteome of the original gut microbial community. In a preferred embodiment, an in vivo culture of a cultured gut microbial community may comprise at least about 98.0, 98.1, 98.2, 98.3, 98.4, 98.5, 98.6, 98.7, 98.9, 99.0, 99.1, 99.2, 99.3, 99.4, 99.5, 99.6, 99.7, 99.8, or 99.9% of the metagenome, transcriptome, or proteome of the original gut microbial community. In another preferred embodiment, an in vivo culture of a cultured gut microbial community may comprise greater than 99.0% of the metagenome, transcriptome, or proteome of the original gut microbial community.

### (III) Models and Methods of the Invention

Yet another aspect of the present disclosure provides methods of using an in vitro or in vivo culture of the invention. Importantly, an in vitro or in vivo culture of the invention may be used as a model of a complete gut microbial community. For instance, as described in more detail below, an in vitro or in vivo culture may be used to determine the effect of a perturbation on a gut microbial community or host thereof. As used herein, “perturbation” refers to any compound or condition administered or applied to a gut microbial community. Advantageously, the effect of the perturbation on an in vitro or in vivo culture of the invention or a host thereof may be representative of the effect of the perturbation on the complete gut microbial community that the culture was derived from.

### (a) Perturbations and Effects

As described above, a “perturbation,” as used herein, refers to any compound or condition administered or applied to a gut microbial community. For instance, in one embodiment, a perturbation may be diet related. Non-limiting examples of diet related perturbations may include foods, specific food ingredients, specific nutrients (e.g. vitamin, mineral, protein, carbohydrate, etc.), or combinations thereof.

In another embodiment, a perturbation may be environmentally related. Non-limiting examples of environmentally related perturbations may include temperature, humidity, or other climate related variables, exposure to other gut microbial communities, exposure to other environmental microbial communities, exposure to different living conditions (e.g. different physical conditions, different psychological conditions, different sleeping conditions, different work conditions, etc.), or exposure to pathogens.

In yet another embodiment, a perturbation may be pharmaceutical. Non-limiting examples of a pharmaceutical may be a drug, a prebiotic, a probiotic, or a neutraceutical. In some embodiments where the perturbation is a drug, the drug is an approved drug. In other embodiments where the perturbation is a drug, the drug is undergoing clinical studies or regulatory testing. In certain embodiments, a drug may be a small molecule, a protein, an antibody, a nucleic acid (e.g. antisense, aptamer, miRNA, RNAi, etc.), or other pharmaceutical.

In an alternative embodiment, a perturbation may be genetic.

In an exemplary embodiment, the perturbation is a food or food ingredient. In another exemplary embodiment, the perturbation is a drug, prebiotic, or probiotic.

Non-limiting examples of the types of effects that can be determined using a model and method of the invention include effects of the perturbation on the composition of the gut microbial community, effects of the perturbation on the metabolism of the gut microbial community, and effects of the perturbation on host biology due to changes in the gut microbial community. In one embodiment, a model and method of the invention may be used to determine the effect of a perturbation on the composition of the gut microbial community. In this regard, “composition of the gut microbial community,” may include the phylotypic composition, the metagenomic composition, the transcriptome composition, or the proteome composition of the gut microbial community. In another embodiment, a model and method of the invention may be used to determine the effect of a perturbation on the metabolism of the gut microbial community. In yet another embodiment, a model and method of the invention may be used to determine the effects of the perturbation on host biology. By way of non-limiting examples, the effects may be changes in host metabolism, changes in host transcription, changes in host protein expression, changes in host immune response, and changes in host gut cellular biology.

In an exemplary embodiment, a method of the invention comprises applying the perturbation to the gut microbial community and determining the impact of the perturbation on the spatial and/or functional organization of the gut microbial community and the niches (professions) of its component members, the impact of the perturbation on the capacity of the community to respond to changes in diet, the impact of the perturbation on the ability of component members to forage adaptively on host-derived mucosal substrates, the impact of the perturbation on the physical and functional interactions that occur between the changing microbial communities and the intestinal epithelial barrier, or the impact of the perturbation on the interaction of the gut microbial community and the immune system of the host.

### (b) Diet Related Perturbations

One method of the invention encompasses a method of determining the effect of a diet related perturbation on a gut microbial community or host thereof. Such a method comprises applying the perturbation to the gut microbial community and determining the effect of the perturbation on the gut microbial community or host thereof. As detailed above, the gut microbial community may be an in vitro or in vivo cultured gut microbial community. In exemplary embodiments, differences in the cultured gut community before and after the application of the perturbation advantageously represent the effect of the perturbation on the original gut microbial community or host thereof.

In some embodiments, a method of the invention encompasses a method of determining the effect of a food or food ingredient on a cultured gut microbial community. Such a method comprises evaluating the cultured gut microbial community before and after the perturbation, wherein the difference in the cultured collection represents the effect of the perturbation on the original gut microbial community.

In another embodiment, the invention encompasses a method of evaluating how the nutritional value of a food ingredient varies with the composition of a subject's gut microbial community. The method generally comprises administering a food ingredient (or food) to one or more subjects with varying gut microbial communities and evaluating the differences in nutritional value of the food ingredient between the subjects in conjunction with evaluating the differences in the gut microbial community of the subjects. Nutritional value of a food or food ingredient may be measured using any method known in the art. In certain embodiments, nutritional value may be determined in terms of growth of the host, metabolic activity of the microbiome, metabolic activity of the host, or microbiome biomass. Such methods may provide information on which foods (or food ingredients) provide better nutrition to a particular group of subjects. For instance, it may be determined for a particular population that the nutritional value of one food ingredient is better than a second food ingredient. Hence, such a method may be used to increase the feed efficiency of a particular diet for either agricultural animals, performance animals, or humans.

In an exemplary embodiment of the above method, the gut microbial community is a cultured gut microbial community.

In a further embodiment, for a malnourished population, such a method may be used to determine the best food or food ingredient for ameliorating the malnourishment. As used herein, “malnourishment” refers to the inadequate or excessive consumption of dietary ingredients leading to the development of disease.

In other embodiments, a method of the invention encompasses a method of predicting the variations in the abundance of a member of a gut microbiome of a host in response to a proposed diet. Generally speaking, the method comprises (a) determining the abundance of a member of a gut microbime in a host, (b) determining the amount of the diet ingredients protein, fat, polysaccharide and simple sugar in a proposed diet, (c) determining the linear coefficient for a particular gut microbiome member in relation to protein, fat, polysaccharide, and simple sugar, (d) predicting the absolute abundance of the member of the gut microbiome if the host were to be fed the proposed diet in (b) based on the linear coefficient from (c) for a particular diet ingredient and the amount of the diet ingredient, and determining the difference between (a) and (d), wherein the difference is the predicted variation in the abundance of a gut microbiome member in response to the proposed diet.

In yet other embodiments, a method of the invention encompasses a method of predicting the abundance of a member of a gut microbiome of a host in response to a proposed diet. The method comprises (a) determining the amount of the diet ingredients protein, fat, polysaccharide and simple sugar in a proposed diet, (b) determining the linear coefficient for a particular gut microbiome member in relation to protein, fat, polysaccharide, and simple sugar, and (c) predicting the absolute abundance of the member of the gut microbiome if the host were to be fed the proposed diet in (a) based on the linear coefficient from (b) for a particular diet ingredient and the amount of the diet ingredient.

In still other embodiments, a method of the invention encompasses a method of specifically manipulating the abundance of a member of a gut microbiome of a host to a target level by changing the diet of the host. The method comprises (a) determining the linear coefficient for a particular gut microbiome member in relation to protein, fat, polysaccharide, and simple sugar, (b) determining the amount of protein, fat, polysaccharide and sugar in a diet necessary to achieve the target level of the gut microbiome member based on the linear coefficients from (a), and (c) feeding a diet to the host that contains the amount of protein, fat, polysaccharide and sugar determined in (b).

For each of the above embodiments, methods of determining the abundance of a member of a gut microbiome are known in the art. Similarly, methods of determining the amount of the diet ingredients protein, fat, polysaccharide and simple sugar in a proposed diet are known in the art. The linear coefficient for a particular gut microbiome member for a particular food ingredient may be determined using a model of a human gut microbiome community. The abundance of a member of a gut microbiome may be calculated with the equation yi=β0+βproteinXprotein+βpolysaccharideXpolysaccharide+βsucroseXsucrose+βfatXfat, where yi is the abundance of the member of the gut microbiome, β0 is the calculated parameter for the intercept, X is the amount in g/(kg of total diet) of the diet ingredient, and βprotein, βpolysaccharide, βsucrose, and βfat are the linear coefficients for a particular gut microbiome member for each of the diet components. In some embodiments, β₀ for a particular gut microbiome member for a particular food ingredient may be determined using a gnotobiotic mouse model of a human gut microbiome community.

### (c) Environmental Perturbations

One method of the invention encompasses a method of determining the effect of an environmental perturbation on a gut microbial community or host thereof. Such a method comprises applying the perturbation to the gut microbial community and determining the effect of the perturbation on the gut microbial community or host thereof. As detailed above, the gut microbial community may be an in vitro or in vivo cultured gut microbial community. In exemplary embodiments, differences in the cultured gut community before and after the application of the perturbation advantageously represent the effect of the perturbation on the original gut microbial community or host thereof.

### (d) Pharmaceutical Perturbations

Yet another aspect of the present disclosure provides a method of evaluating the impact of a pharmaceutical on a gut microbial community. The method typically comprises evaluating a culture of a gut microbial community in the presence and absence of the pharmaceutical, and identifying the differences, if any, between the culture exposed to the pharmaceutical, and the culture not exposed to the pharmaceutical. For instance, in one embodiment, an in vivo model (as detailed in section II above) of a particular subject's gut microbial community may be used to determine how a particular pharmaceutical would impact that subject's gut microbial community. Such a method may be used to determine the reaction of the subject's gut microbial community to the pharmaceutical without having to administer the drug or pharmaceutical to the subject itself. Such reactions may include any changes in the composition of the gut microbial community, changes in the metabolism of the gut microbial community, or changes in host biology stemming from a change in the gut microbial community.

(e) Methods of Identifying Agents that Impact a Gut Microbial Community

In some instances the invention encompasses methods of identifying agents that impact a gut microbial community. In one embodiment, such a method may comprise applying a perturbation to a gut microbial community and determining the changes the perturbation evokes in the community. Specifically, in certain embodiments, changes in one or more taxa are identified. These taxa may then be applied to a cultured gut microbial community, either individually or in combinations, to determine their impact on the cultured gut microbial community. In this manner, agents that impact a gut microbial community may be identified.

In one embodiment, the invention encompasses a method of discovering a probiotic. The method generally comprises identifying a microbe that thrives after administration of a particular food or food ingredient to a subject. For instance, an in vitro culture may be created before and after administration of a food or food ingredient to a subject. Differences in the before and after gut microbial cultures may be evaluated to determine a microbe that thrives upon the administration of the particular food or food ingredient. Similarly, a particular food or food ingredient may be administered to a sterile animal comprising a known culture of a gut microbial community. Changes in the gut microbial community may be evaluated to identify a microbe that thrives upon the administration of the particular food or food ingredient. Methods of evaluating a gut microbial community, and method of identifying a microbe that is thriving in a gut microbial community are known in the art, and may include those detailed herein.

### (f) Disease Models

Another aspect of the present disclosure provides a method of creating a disease model. The method generally comprises 1) identifying a gut microbial community that is related to, the cause of, or the result of a particular disease or disorder, and 2) reproducing that gut microbial community in an in vitro or in vivo model as described above.

Yet another aspect of the present disclosure provides a method of treating a disease. The method typically comprises identifying a difference between a normal gut microbial community and a gut microbial community of a subject afflicted with the disease or disorder, and altering the gut microbial community of the subject afflicted with the disease or disorder to more closely resemble a normal gut microbial community.

### (g) Administration of a Specific Cultured Gut Microbiome

Yet another aspect of the present disclosure provides a method of altering the gut microbiome of a subject, the method comprising administering a cultured gut microbiome to the subject. For instance, it may be determined, using a method of the invention, that a particular gut microbiome culture may be advantageous to a subject. Such a microbiome may be administered via oral gavage, as described herein, or in any other manner suitable for administering the cultured collection. By way of non-limiting example, a gut microbiome may be administered to a subject early in its life to form a gut microbiome that is best suited for the growth of the subject in a particular environment. Suitable subjects may include animals (e.g. performance animals, food animals, companion animals, etc.) and humans.

### DEFINITIONS

The term “metagenomics” refers to the application of modern genomic techniques to the study of the composition and operations of communities of microbial organisms sampled directly in their natural environments, by passing the need for isolation and lab cultivation of individual species.

The term “sterile animal” refers to an animal that has no microorganisms living in or on it. In one embodiment, the sterile animal is a sterile mouse.

The term “gnotobiotic animal” refers to an animal in which only certain known strains of bacteria and other microorganisms are present.

### EXAMPLES

The following examples illustrate various embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### Introduction to Examples 1-9

Efforts to dissect the functional interactions between microbial communities and their environmental or animal habitats are complicated by the long-standing observation that, for many of these communities, the great majority of organisms have not been cultured in the laboratory. Methodological differences between culture-independent and culture-based approaches have contributed to the challenge of deriving a realistic appreciation of exactly how much discrepancy exists between the culturable components of a microbial ecosystem and total community diversity. (Table 1 gives examples of these methodological differences.)

The largest microbial community in the human body resides in the gut: Its microbiome contains at least two orders of magnitude more genes than are found in our Homo sapiens genome. Culture-independent metagenomic studies of the human gut microbiota are identifying microbial taxa and genes correlated with host phenotypes, but mechanistic and experimentally demonstrated links between key community members and specific aspects of host biology are difficult to establish with these methods alone. The goals of the examples presented below are (i) to evaluate the representation of readily cultured phylotypes in the human gut microbiota; (ii) to profile the dynamics of these cultured communities in a mammalian gut ecosystem; and (iii) to determine whether a clonally arrayed, personalized strain collection could be constructed to serve as a foundation for reassembling varying elements of a human's gut microbiota in vitro or in vivo.

### Example 1

### Estimating the Abundance of Readily Cultured Bacterial Phylotypes in the Distal Human Gut

To estimate the abundance of readily cultured bacterial phylotypes in the distal human gut, primers were used to amplify variable region 2 (V2) of bacterial 16S ribosomal RNA (rRNA) genes present in eight freshly discarded fecal samples obtained from two healthy, unrelated anonymous donors living in the United States (n=1 complete sample per donor at t=1, 2, 3, and 148 d). Amplicons were subjected to multiplex pyrosequencing, and the results were compared with those generated from DNA prepared from ˜30,000 colonies cultured from each sample, under strict anaerobic conditions and harvested after 7 d at 37° C. on a rich gut microbiota medium (GMM) composed of commercially available ingredients (“cultured” samples; details of the culturing technique are given in Materials and Methods, and a description of GMM is given in Table 2). The resulting 16S rRNA datasets were de-noised to remove sequencing errors, reads were grouped into operational taxonomic units (OTU) of ≧97% nucleotide sequence identity (ID), and chimeric sequences were eliminated (Materials and Methods).

In total, 632 distinct 97% ID OTU were observed in the complete samples, and 316 were identified in the cultured samples. The average abundance of cultured OTU in the complete samples was 0.4%, but the average abundance of uncultured OTU (i.e., those observed in the complete but not the cultured samples) was significantly lower (0.06%; P<10⁻⁶ by an unpaired, two-tailed student's t test, not assuming equal variances).

### Example 2

### Evaluating the Representation of Readily Cultured Taxa in the Human Gut Microbiota

To evaluate the representation of readily cultured taxa in the human gut microbiota at varying phylogenetic levels, taxonomic designations were assigned to each 97% ID OTU (Materials and Methods). Each 16S rRNA read from the complete fecal sample was scored as “cultured” if it had a taxonomic assignment that also was identified in the corresponding cultured population. If a 97% ID OTU in the complete sample could not be placed in any known taxonomic group, it was scored as “cultured” only if the same 97% ID OTU was observed in the cultured sample. This analysis indicated that 99% of the 16S rRNA reads derived from the complete fecal samples from either donor belong to phylum-, class- and order-level taxa that also are present in the corresponding cultured sample; 89±4% of the reads are derived from readily cultured family-level taxa, and 70±5% and 56±4% belong to readily cultured genus- and species-level taxa, respectively (FIG. 1A Upper). Two alternate taxonomic binning methods, the Ribosomal Database Project (RDP) Bayesian classifier v2.0 and an arbitrary % ID cutoff, produced similar results (FIG. 2 A-F). Control experiments described in Materials and Methods indicate that at least 98% of the reads generated from 30,000 pooled colonies are not derived from nongrowing or lysed bacteria (the percentage of reads from the original fecal samples that is derived from dead cells is unknown).

Unsupervised hierarchical clustering of the complete and cultured microbial communities, across the two donors and four time points, revealed that cultured samples cluster separately from those that had not been cultured. Both phylogenetic and nonphylogenetic metrics segregate cultured samples by donor, suggesting that the distinctiveness of each donor's microbiota is preserved in their collections of readily cultured representatives (FIGS. 2 G and H).

Shotgun DNA pyrosequencing was performed to determine the degree to which predicted functions contained in the composite genomes of the complete human fecal microbial communities were represented in the corresponding collection of cultured microbes [n=4 samples (one complete and one cultured from each of two donors); 119,842±43,086 high-quality shotgun reads per microbiome; average read length, 366 nt]. On average, 90% of the 2,302 distinct KEGG Orthology (KO) annotations identified in the two uncultured samples also were observed in the cultured communities (FIG. 1B, FIGS. 3 A and B). This high percentage of functional representation also was observed when the microbiomes were subjected to alternate annotation schemes: On average, 94% of 929 enzyme commission (EC) assignments and 95% of 216 level 2 KEGG pathways associated with the complete fecal samples also were detected in the cultured communities (FIG. 3 C—F).

### Example 3

### Comparing Functions Represented in the Complete and Cultured Microbiota

To compare further the functions represented in the complete and cultured microbiota independent of annotation, antibiotic-resistance genes were captured from their microbiomes in expression vectors in Escherichia coli. Each E. coli library contained ˜1 GB of 1.5- to 4-kB fragments of microbiome DNA subcloned into an expression vector and was screened against a panel of 15 antibiotics and clinically relevant antibiotic combinations (Table 3). Genes encoding resistance to the same 14 antibiotics were captured in libraries prepared from complete and cultured fecal samples (FIG. 3G and Table 4). In one example, a screen for DNA fragments that confer resistance to the aminoglycoside amikacin produced candidate genes from the microbiomes of both complete and cultured microbial communities from Donor 1 but not from Donor 2. Two genes conferring amikacin resistance (either the 16S rRNA methylase rmtD or the aminoglycoside phosphotransferase aphA-3) were identified in 70% of the DNA fragments captured in selections for this phenotype. Direct culturing of the original fecal communities in the presence of amikacin confirmed that this resistance function is significantly enriched in the readily cultured microbiota of Donor 1 compared with Donor 2 (P<0.005 based on triplicate samples; unpaired, two-tailed student's t test assuming equal variances) (FIG. 3H); and PCR analysis showed that many of the amikacin resistant fecal strains harbor rmtD or aphA-3. Sequencing the 16S rRNA genes of a subset of these isolates indicated that rmtD is present in strains of Bacteroides uniformis, B. caccae, and B. thetaiotaomicron in this donor (although, notably, not in the sequenced type strains of these species) and that aphA-3 is contained in the genome of a member of the genus Desulfotomaculum (order Clostridiales).

### Example 4

### Determining Whether an Individual's Readily Cultured Community Exhibits Behavior In Vivo Mirroring that of the Individual's Complete Microbial Community

To determine whether a community composed of an individual's readily cultured bacteria exhibits behavior in vivo mirroring that of the individual's complete microbial community, 9-wk-old C57B16/J germfree mice were colonized with a complete or cultured microbiota from each of the two human donors (n=5 recipient mice per sample type). A fecal sample from each donor was divided after collection, and one aliquot was gavaged directly into one group of recipient mice; the other aliquot was cultured on GMM plates for 7 d, as above, harvested, and introduced into a second group of recipient animals. Mice were maintained on a standard autoclaved low-fat, plant polysaccharide-rich (LF/PP) chow diet before and 4 wk after gavage. 16S rRNA analysis of fecal samples collected from these mice at the end of the 4-wk period indicated that the complete and the cultured communities were influenced similarly by host selection: 91±3% of the 16S rRNA reads identified from mice colonized with a human donor's complete fecal microbiota were derived from genus-level taxa that also were identified in the mice colonized with the cultured microbial community from the same donor (FIG. 1A Lower). Importantly, control experiments demonstrated that the harvested, actively growing colonies gavaged into each germfree mouse are able to exclude nongrowing species that might be present on GMM plates from establishing themselves in recipient animals (details are given in Materials and Methods).

Luminal material was collected from the proximal, central, and distal portions of the small intestine, cecum, and colon of mice colonized with either the complete or cultured communities from each of the two human donors. V2-directed bacterial 16S rRNA sequencing revealed similar geographic variations in community structures (FIG. 1C and FIG. 4 A-C).

### Example 5

### Determining Whether the Similarities in Community Composition In Vivo Extend to Similarities in Community Gene Content

To determine whether the similarities in community composition in vivo extend to similarities in community gene content, the same fecal DNA samples that had been prepared from these mice after 4 wk on the LF/PP diet for 16S rRNA analyses were subjected to shotgun pyrosequencing (n=4 samples; 87,357±30,710 reads per sample). As with the 16S rRNA analysis, comparisons of the representation of KOs in the various microbiome samples revealed an even greater correlation between complete and cultured communities after they had been subjected to in vivo selection than before their introduction into mice (FIG. 1B, FIG. 3 A-F).

### Example 6

### Assessing Whether a Complex Community of Cultured Microbes could Restore Epididymal Fat Pad Weights to the Levels Associated with Complete Microbial Communities

Previous comparisons of adult germfree mice with those that harbor gut microbial communities (either conventionally raised animals or formerly germfree animals colonized from mouse or human donors) have shown that the presence of a complete gut microbiota is associated with increased adiposity. In comparison, colonization of germfree mice with a single, readily cultured, prominent human gut symbiont (Bacteroides thetaiotaomicron) or with a defined community of 12 bacterial species prominently represented in the distal human gut is insufficient to restore epididymal fat pad stores to levels observed in conventionally raised animals (data not shown). To assess whether a complex community of cultured microbes could restore epididymal fat pad weights to the levels associated with complete microbial communities, we evaluated mice colonized with the complete or the cultured fecal communities from the two human donors. All animals displayed significantly greater fat pad to body weight ratios than germfree controls, and no significant difference was observed in adiposity between mice colonized with the donors' complete or cultured microbiota (FIG. 4D).

### Example 7

### Testing Whether a Microbial Community Consisting Only of Cultured Members Recapitulates Known Diet-Induced Changes in Microbial Community Structure In Vivo

Mice colonized with a complete human microbiota undergo drastic changes in microbial community structure (even after a single day) when shifted from LF/PP chow to a high-fat, high-sugar Western diet. To test whether a microbial community consisting only of cultured members recapitulates this behavior in vivo, the four groups of gnotobiotic mice colonized with the complete or cultured microbes from two unrelated human donors were monitored by fecal sampling before, during, and after a 2-wk period when they were placed on the Western diet (samples were collected at days 4, 7, and 14 of the first LF/PP phase, then 1 d before and 3, 7, and 14 d after initiation of the Western diet phase, and finally 1, 3, 8, and 15 d after the return to the LF/PP diet). 16S rRNA-based comparisons of fecal communities were performed using both phylogenetic and nonphylogenetic distance metrics. With either metric, principal coordinates analysis (PCoA) revealed that mice colonized with the complete or cultured samples maintain communities that cluster first by donor (principal coordinate 1; PC1) and that the complete and cultured communities from both donors respond to the diet shift in a similar manner [principal coordinate 2 (PC2); FIG. 5A-C and FIGS. 6A and B]. Like the transplanted complete microbiota examined here and in previous reports, the cultured microbiota responded to this Western diet by increasing the relative proportion of representatives of one class of Firmicutes (the Erysipilotrichi) and decreasing the relative proportion of the Bacteroidia class (FIG. 6C). Notably, of the 18 species-level phylotypes significantly affected by diet shift in the mice containing the complete microbiota of both human donors, 14 were detected and demonstrated the same statistically significant response in mice colonized with readily cultured taxa (FIG. 5D and FIG. 7).

The fecal microbiomes of LF/PP-fed mice harboring complete or cultured communities from each of the two unrelated donors were compared with microbiomes sampled after these mice consumed the Western diet for 14 d (101,222±24,271 reads per sample). The representation of level 2 KEGG pathway functions was highly concordant on both diets, with one exception: Genes encoding phosphotransferase system (PTS) pathways for carbohydrate transport were significantly overrepresented in Western diet-fed mice harboring complete or cultured communities from either donor (FIG. 5E and FIG. 8A-D). Higher-resolution KO level annotations confirmed that the diet-based PTS pathway enrichment reflected increased representation of multiple carbohydrate transporters (FIGS. 8E and F). These results emphasize that the similar taxonomic restructuring of complete and cultured communities in response to diet is accompanied by similar changes in community gene content.

### Example 8

### Using Gnotobiotic Mice as Biological Filters to Recover Collections of Readily Cultured Microbes

Because human gut communities composed of readily cultured members exhibit responses to host diet that mirror those characteristic of a complete microbiota, the possibility that gnotobiotic mice can be used as biological filters to recover collections of readily cultured microbes, obtained from selected human hosts, that are enriched for certain properties [e.g., the ability to prosper (bloom) when exposed to specific foods or food ingredients] was explored. To this end, fecal samples from mice colonized with the complete or corresponding cultured human gut microbial communities from the two unrelated donors and fed the LF/PP or Western diets were collected directly into anaerobic medium and then plated on prereduced GMM plates (FIG. 9A). After 7-d incubation, V2-directed 16S rRNA profiling of these plated microbial collections confirmed that these populations of cultured microbes can be reshaped deliberately in vivo and then recovered in vitro (FIG. 9B and FIG. 10). On either diet, cultured populations showed significantly greater resemblance to the in vivo communities from mice consuming the same diet than to the in vivo communities from the same mice consuming the alternate diet (P<10-11, unpaired two-tailed student's t test of within donor distances shown in FIG. 9B, assuming equal variances).

### Example 9

### Creating Arrayed Species Collections Representing the Bacterial Diversity of the Gut Microbiota

The strict anaerobic techniques used here, compounded with highly diverse colony morphologies across taxa, complicate efforts to pick and isolate individual colonies at a scale sufficient to capture the bacterial diversity represented on the culture plates. Therefore, a most probable number (MPN) technique was used for creating arrayed species collections in a multiwall format without colony picking. First, the dilution point for a fecal sample that yields 70% empty wells (no detectable growth) after inoculation into 384-well trays and 7-d anaerobic incubation was empirically determined. Assuming that the distribution of cells into the wells follows a Poisson distribution, a dilution that leaves 70% of wells empty should yield nonclonal wells (that is, wells that received more than one cell in the inoculum) only 5% of the time; the remainder should be clonal (FIGS. 11F and G). At this dilution, ten 384-well trays should yield ˜1,000 clonal wells. The two-step barcoded pyrosequencing scheme outlined in FIG. 12A was developed to assign a 16S rRNA sequence to the isolate(s) present in each turbid well.

This approach was used to create an archived, personalized culture collection of ten 384-well trays from one of the human donors. 16S rRNA sequences could be assigned to more than 99% of growth-positive wells. One advantage of clonally arrayed collections is that the effects of 16S rRNA primer bias encountered using DNA templates prepared from complex microbial populations are minimized when wells contain a single taxon. This point is illustrated by the known bias of most commonly used primers against Bifidobacteria spp.: Members of this genus were better represented among the set of 16S rRNA genes produced from individual wells than among those observed in complex communities harvested from GMM plates.

After the archived trays had been frozen under anaerobic conditions and stored at −80° C. for 7 mo, recovery of organisms from wells exceeded 60%. Full-length 16S rRNA sequences generated from these recovered strains matched the assignments from the barcoded pyrosequencing data in every case, suggesting that the dilutions did follow a Poisson distribution as predicted. Like 16S rRNA-based community profiling, such collections may miss rare, but important, members of the microbiota; seeding additional 384-well trays with the diluted sample will capture additional phylotypes (FIG. 11H). In total, this individual's culture collection contained 1,172 taxonomically defined isolates from four different phyla, seven classes, eight orders, 15 families, 23 genera, and 48 named bacterial species. Novel isolates were encountered at the family-, genus-, and species-levels, and 69% of the complete community had a genus-level representative in the arrayed collection (FIG. 12B). As a frame of reference, we identified a total of 159 human fecal or gut bacterial species from humans worldwide (including pathogens) in the German Resource Centre for Biological Material (DSMZ) culture collection (Materials and Methods). As such, personalized microbiota collections can complement those of international repositories by capturing strains that coexist in a shared habitat where community structure and host parameters can be measured.

The ability to capture this level of diversity after MPN dilution in these arrayed collections indicates that it is unlikely that interspecies syntrophic relationships by themselves are sufficient to explain the diversity observed on the GMM agar plates. On the other hand, these personalized arrayed culture collections should help identify obligate syntrophic relationships (e.g., by analyzing the patterns of co-occurrence of taxa in wells harboring more than one phylotype or by comparing arrayed collections in which one set of trays contains a candidate syntroph deliberately added to all wells).

### Discussion for Examples 1-9.

The Examples presented above show that it is possible to capture a remarkable proportion of a person's fecal microbiota using straightforward anaerobic culturing conditions and easily obtained reagents. Variations in culturing conditions, including components that are not commercially available (e.g., sterile rumen or human fecal extracts) and other approaches for more closely approximating a native gut habitat, undoubtedly will allow additional members of the human gut microbiota to be cultured in vitro. These personal culture collections can be generated from humans representing diverse cultural traditions and various physiologic or pathophysiologic states. A key opportunity is provided when anaerobic culture initiatives are combined with gnotobiotic mouse models, thereby allowing culture collections to be characterized and manipulated in mice with defined (including engineered) genotypes who are fed diets comparable to those of the human donor, or diets with systematically manipulated ingredients. Temporal and spatial studies of these communities can be used to identify readily cultured microbes that thrive in certain physiological and nutritional contexts, creating a discovery pipeline for new probiotics and for preclinical evaluation of the nutritional value of food ingredients. Based on their in vivo responses, clonally archived cultured representatives of a person's microbiota can be selected for complete genome sequencing (including multiple strains of a given species-level phylotype) to identify potential functional variations that exist or evolve within a species occupying a given host's body habitat. Coinciding with the introduction of yet another generation of massively parallel DNA sequencers, this approach also should allow vast scaling of current sequencing efforts directed at characterizing human (gut) microbial genome diversity, evolution, and function. Recovered organisms also could be used as source material for functional metagenomic screens (bio-prospecting). Guided by the results of metagenomic studies of human microbiota donors, components of a personalized collection that have coevolved in a single host can be reunited in varying combinations in gnotobiotic mice, potentially after genome-wide transposon mutagenesis of selected taxa of interest, for further mechanistic studies of their interactions and impact on host phenotypes.

### Materials and Methods for Examples 1-9.

Culturing of Fecal Microbiota.

Freshly discarded fecal samples from two anonymous unrelated human donors were transferred into an anaerobic chamber (Coy Laboratory Products) within 5 min of their collection. Samples were placed in prereduced PBS with 0.1% cysteine (PBSC) 15 mL⁻¹ g⁻¹ feces. The fecal material was suspended by vortexing for 5 min, and the suspension was allowed to stand at room temperature for 5 min to permit large insoluble particles to settle to the bottom of the tube. 16S ribosomal RNA (rRNA) sequencing of the starting material and of the supernatant obtained after the settling step showed no significant differences in community composition. This settling step dramatically increased the reproducibility of subsequent dilutions. Diluted (10⁻⁴) samples were plated on plates 150 mm in diameter containing prereduced, nonselective Out Microbiota Medium (GMM) (Table 2) so that colonies were dense but distinct (˜5,000 colonies per plate) after a 7-d incubation at 37° C. under an atmosphere of 75% N₂, 20% CO₂, and 5% H₂. GMM is modified from tryptone-yeast-glucose agar with 6% NaCl (TYGS) medium and contains only commercially available components; to discourage colony overgrowth, the concentration of glucose, tryptone, and yeast extract is reduced fivefold compared with TYGS. Colonies were harvested en masse from each of six plates by scraping with a cell scraper (BD Falcon) into 10 mL of prereduced PBSC. Stocks were generated by adding prereduced glycerol containing 0.1% cysteine to the fecal or cultured samples (final concentration of glycerol, 20%). Stocks were stored in anaerobic glass vials in a standard −80° C. freezer.

To determine the optimal number of plates to be surveyed for each fecal sample, a freshly discarded sample from one of the anonymous human donors was processed as above, and the 10⁻⁴ dilution was plated on 10 prereduced GMM plates. De-noised, chimera-checked variable region 2 (V2)-directed 16S rRNA reads generated from the pooled colonies obtained from each plate separately after a 7-d incubation were assigned to operational taxonomic units (OTU) at 97% nucleotide sequence identity (ID) using QIIME v1.1. Each additional plate contributed new OTU, although, as shown by the rarefaction curves plotted in FIG. 11A, the contribution of each added plate fell to <20 new OTU after approximately six plates. For this reason, each subsequent cultured sample reflects pooled scraped material from six plates.

Gnotobiotic Mouse Husbandry.

Germfree adult male C57BL/6J mice were maintained in plastic gnotobiotic isolators. Mice were housed under a strict 12-h light/dark cycle and fed a standard, autoclaved low-fat/plant polysaccharide-rich (LF/PP) chow diet (B&K Universal.) ad libitum. Mice were colonized by gavage (0.2 mL of the resuspended fecal material or pooled cultured organisms recovered from GMM after 7-d incubation as above, per germfree recipient). Animals receiving different microbial inoculations were placed in separate gnotobiotic isolators before gavage; once gavaged, they were caged individually.

After 4 wk of acclimatization on the LF/PP diet, mice were transitioned to Western diet (Harlan-Teklad TD96132) ad libitum for 2 wk and then were returned to the LF/PP diet for 2 wk. 16S rRNA analysis of fecal samples collected 1, 4, 7, and 14 d after gavage indicated that both complete and cultured communities reached a steady state well before the diet transition. During the initial LF/PP diet phase, fecal samples were collected at postgavage days 4, 7, 14, and either on day 32 when the input community was a complete microbiota or on day 25 in the case of an input cultured community. Mice were sampled on days 1, 3, 7, and 14 after the shift to the Western diet and on days 1, 3, 8, and 15 upon return to LF/PP chow. The animals then were fasted for 24 h and returned to the LF/PP diet for 1 wk before they were killed.

Fecal samples for subsequent culture were collected from each mouse directly into BBL thioglycollate medium (BD), transported to an anaerobic chamber within 30 min, then diluted and plated on GMM as above. Fecal samples from fasted mice were not cultured because 16S rRNA analysis did not show significant changes in community composition at either the 12-h or 24-h fasting time points. After mice were killed, the intestine was subdivided into 16 segments of equivalent length numbered from 1 (proximal) to 16 (distal). Contents from small intestine segments 2, 5, and 13, plus cecum and colon contents, were snap frozen in liquid nitrogen and stored at −80° C.

DNA Extraction and Purification.

Fecal samples (0.1 g) were resuspended into 710 μL of 200 mM NaCl, 200 mM Tris, 20 mM EDTA (Buffer A) plus 6% SDS). After the addition of 0.5 mL of 0.1-mm zirconia/silica beads (BioSpec Products) and 0.5 mL of phenol/chloroform/isoamyl alcohol, pH 7.9 (Ambion), cells were lysed by mechanical disruption with a bead-beater (BioSpec Products) for 3 min. Samples were centrifuged for 3 min at 6,800×g, and the aqueous phase was collected and subjected to a second phenol-chloroform-isoamyl alcohol extraction using Phase Lock Gel tubes (5 Prime). DNA in the aqueous phase was precipitated by the addition of an equal volume of isopropanol and 0.1 volumes of 3 M sodium acetate, pH 5.5 (Ambion). After overnight incubation at −20° C., samples were centrifuged for 20 min at 4° C. at 18,000×g, and the supernatant was removed. Pelleted DNA was washed once with 0.5 mL of 100% ethanol, and dried using a vacuum evaporator. DNA pellets then were resuspended in 0.2 mL of Tris-EDTA containing 4 μg RNAseA (Qiagen). Crude DNA extracts were column-purified using the Rapid PCR Purification Kit (Marligen Biosciences). DNA concentrations were adjusted to 50 ng/μL for subsequent 16S rRNA or shotgun pyrosequencing.

16S rRNA Sequencing.

The V2 region of bacterial 16S rRNA genes was subjected to PCR amplification. PCR reactions were carried out in triplicate using 2.5× Master Mix (5 Prime), forward primer FLX-8F; 5′-GCCTTGCCAGCCCGCTCAGTCAGAGTTTGATCCTGGCTCAG-3′ (SEQ ID NO 1); (the 54 FLX Amplicon primer B sequence is underlined, and the 16S rRNA primer sequence 8F is italicized), barcoded reverse primer FLX-BC-338R; 5′-GCCTCCCTCGCGCCATCAGNNNNNNNNNNNNCA TGCTGCCTCCCGTAGGAGT-3′; (SEQ ID NO 2) (the 454 FLX Amplicon primer A sequence is underlined, “Ns” indicate the barcode sequence, and the 16S rRNA primer sequence 338R is shown in italics), and 50 ng input DNA purified as described above. Reactions were incubated for 2 min at 95° C., followed by 30 cycles of 20 s at 95° C., 20 s at 52° C., and 1 min at 65° C. Triplicate reactions were pooled, inspected by gel electrophoresis, and purified on AMPure beads (Agencourt Biosciences). For each barcoded primer, negative control reactions lacking input DNA were conducted in parallel. Purified samples were combined at equimolar concentrations and sequenced with FLX chemistry on a 454 pyrosequencer (Roche).

16S rRNA Sequence Analysis.

Metadata for all 500 samples, including barcodes, are provided in Table 26. For 16S rRNA sequence analysis, sequences were preprocessed to remove reads with low-quality scores (sliding window set to 50 bp), ambiguous characters, and incorrect lengths (<200 or >300 bp). Reads passing these criteria were assigned to specific samples based on their error-corrected barcode sequence, de-noised using default parameters, grouped into OTU at 97% ID, and a representative sequence was selected from each OTU using default parameters in QIIME v.1.1. These representative sequences then were filtered for possible chimeric sequences using ChimeraSlayer (microbiomeutil.sourceforge.net) with default parameters (sequences designated “unknown” were not discarded). Filtered datasets were subsampled to 1,000 sequences per sample (the only exceptions were the datasets from the human-derived complete and cultured samples collected at the day 148 time point shown in FIG. 1A, which were subsampled to 5,000 sequences). Beta-diversity calculations were conducted using Jaccard (nonphylogenetic) and UniFrac (phylogenetic) metrics in QIIME v1.1.

Weighted Taxonomic Analysis.

To quantify the representation of cultured and uncultured lineages in microbial communities, the presence or absence of each phylum-, class-, order-, family-, genus-, or species-level phylotype assigned to sequences in the complete sample(s) was determined in the cultured sample(s). Analysis of full-length and V2-region delimited sequences from the 16S rRNA genes of taxonomically defined bacteria indicated that discrete % ID cutoffs do not correspond closely to established taxonomic levels: Histograms of the distributions of % ID values of 16S rRNA sequences between representatives of two species in the same genus, two genera in the same family, and so forth, overlap to a large extent. (FIG. 11B shows comparisons of 16S V2 regions, which are used commonly in multiplex pyrosequencing studies, between 4,041 bacterial species selected from the SILVA database v102.) For this reason, a taxonomic assignment method was primarily used to compare uncultured and cultured communities across taxonomic levels rather than approximating taxonomic groups by selecting arbitrary % ID cutoffs to represent each taxonomic level. (For example, FIG. 11B illustrates that there is no clear % ID cutoff that distinguishes species-level from genus-level groups or family-level from order-level groups.) As an alternate, taxonomy-independent approach, de-noised 16S rRNA sequences were grouped into OTU at a range of % ID cutoffs (80%, 90%, 95%, 97%) using uclust in QIIME v1.1. At each % ID cutoff, OUT were filtered for chimeras as above. The proportion of reads in the uncultured sample that belonged to OTU also identified in the corresponding cultured sample were determined as in FIG. 1A (see FIG. 2A-F for a comparison of results obtained from different assignment methods).

SILVA-VOTE: A Computational Pipeline for Improved Accuracy in Taxonomic Assignments of V2 16S rRNA Sequences.

Commonly used tools for taxonomy assignment often failed to assign correctly V2 16S rRNA sequences derived from known human gut microbes. To generate a nonredundant, curated 16S rRNA database for taxonomy assignment, the v102 SILVA database was downloaded prefiltered for redundancy at a 99% ID (SSURef_{—}102_SILVA_NR_{—}99.fasta;://www.arb-silva.de). This database is composed of 262,092 full-length sequences from the small subunit rRNAs of Eukaryotes, Bacteria, and Archaea. A total of 297 sequences whose accession numbers had been removed from or modified by GenBank or were not associated with a complete National Center for Biotechnology Information (NCBI) taxonomy (i.e., phylum, class, order, family, genus, and species designations) were excluded. The remaining sequences were aligned using PyNast as implemented in QIIME v1.1: 224,899 sequences were aligned successfully and contained more than 90% of the V2 region. These V2 sequences were filtered for redundancy by clustering and selection of a representative sequence from each cluster, using uclust at a 99% identity. To assign consensus taxonomies to the representative sequences, we applied a 75% majority voting scheme: For each taxonomic level, the representative sequence was assigned a taxonomic designation if more than 75% of the sequences within the cluster shared the same assignment; otherwise, the cluster was labeled “unknown” at that taxonomic level. Taxonomic designations of sequences within a cluster that included the nonunique identifiers “unknown,” “uncultured,” “candidatus,” or “bacterium” were not considered in the 75% majority vote for taxonomy assignment of the representative sequence. Species-level annotations with numbers or decimal points (which in almost all cases refer to strains rather than species) also were excluded. After removal of sequence clusters with little or no consensus taxonomy (i.e., with 50% or more of the taxonomic levels labeled “unknown” after the voting analysis), 34,181 nonredundant, annotated, bacterial 16S rRNA V2 sequences remained and were designated as our reference database.

To assign taxonomy to the 16S rRNA V2-amplicon pyrosequencing reads, significant matches to the 34,181-sequence reference database were identified by BLAST (the top 100 hits with an e-value cutoff of 0-30 were retained). All the BLAST hits with a score within 10% of the score of the best BLAST hit were considered for the taxonomy assignment. Taxonomy was assigned for each phylogenetic level independently by using a majority voting scheme: A read was assigned a taxonomic designation if 50% or more of the selected reference sequences (whose BLAST scores were within 10% of the top score for that query sequence) shared the same taxonomic assignment. As above, sequences designated “unknown” were not taken into account for the voting. When no assignment was conserved in >50% of the selected BLAST hits, the query sequence was labeled as “nonidentified” at that taxonomic level. Data were normalized by the abundance of each taxonomic group in the original (uncultured) sample. For analysis of microbial communities from mice, taxonomic groups observed in fewer than two replicate animals were omitted.

To test this method, 16S rRNA V2 sequences were extracted from the genomes of 66 human gut microbes. Taxonomy was assigned to these test sequences in QIIMEv.1.1 using three methods: (i) the Ribosomal Database Project Bayesian classifier (v.2.0); (ii) a BLAST top hit-based query of the Greengenes core sequence set [composed of 4,938 sequences; downloaded Sep. 15, 2009; (greengenes.lbl.gov/Download/Sequence_Data/Fasta_data_files/core_set_aligned_fast a. imputed) (7)]; and (iii) with SILVA-VOTE. Comparison of these results suggests that SILVA-VOTE yields a significantly increased number of correct taxonomic assignments, particularly at the genus and species levels (FIG. 11C).

Control Experiments to Address the Influence of Lysed or Nongrowing Cells on 16S rRNA Datasets from Colonies Collected from Agar Plates.

Although each average-sized colony among the ˜30,000 colonies obtained from each cultured sample likely contributed ˜10⁹ cells to the pooled population, in theory genetic material from lysed or nongrowing cells also could contribute to the sequences obtained from the plated samples. To test this possibility directly, fecal samples were diluted to the same level as above and plated onto GMM and also onto plates containing ingredients that should not support growth of bacteria and thus represent the background expected if 100% of the plated material was nongrowing or lysed [control PARC plates contained Phosphate buffer, noble Agar, Resazurin (oxygen indicator), and Cysteine (reducing agent) Table 5]. After a 7-d anaerobic incubation, no colonies were detected on the PARC plates. Twenty randomly selected single colonies from the GMM plates were picked, an aliquot was reserved for 16S rRNA gene sequencing, and the remainder was pooled with the scraped surfaces of the PARC plates. Sequencing this pool revealed that >98% of the 16S rRNA reads could be attributed to the 20 colonies from the GMM plates; among the remainder, none belonged to OTU represented by more than two reads per 1,000. Together, these findings suggest that at least 98% of the reads generated from 30,000 pooled colonies are not derived from nongrowing or lysed bacteria.

Testing the Possible Contribution of Lysed or Nongrowing Cells to Microbial Communities in Gnotobiotic Mice Gavaged with a Readily Cultured Human Gut Microbiota.

As noted in the main text, the initial cultured inoculum was prepared by scraping GMM plates en masse. In theory, this input could include cells that did not actually grow in these conditions but instead remained dormant, below the limit of detection by 16S rRNA sequencing, over the 7-d in vitro incubation period. To determine whether such non-growing taxa contributed to the distal gut communities of mice that received a readily cultured microbiota, a control sample containing material harvested from PARC plates and pooled with 20 visible colonies picked directly from GMM plates was introduced into five age-matched, individually caged, germfree mice fed a LF/PP diet. Fecal samples were collected at 3, 7, and 14 d postgavage and were subjected to V2-targeted 16S rRNA pyrosequencing. Community composition, as determined both by alpha-diversity and beta-diversity metrics, was stable after the 7-d time point (average UniFrac distance within 7-d or 14-d samples=0.319; average distance between 7-d and 14-d samples=0.321; P>0.89 based on unpaired, two-tailed student's t test; FIG. 11D-E). When 16S rRNA sequences obtained from the 14-d fecal samples were compared with sequences obtained from the 20 picked colonies, it was discovered that the mice harbored only two OTU, both mapping to Akkermansia muciniphila, that could not be attributed to the 20 colonies and that were not observed on GMM plates in any other experiments. Akkermansia muciniphila type strain ATCC BAA-835 contains three 16S rRNA genes and grows readily on GMM. This species was a minor component in the fecal microbiota of the two donors (no or one read per 1,000 reads from eight samples collected over time); in fecal communities sampled from mice that received the readily cultured component of either donor's microbiota, abundance averaged 1.8% across all time points. These findings indicate that just 20 actively growing colonies are able to exclude virtually all nongrowing species that may be present on GMM plates from colonizing germfree mice.

Shotgun Pyrosequencing.

Five hundred-nanogram aliquots of DNA prepared from selected complete and cultured microbiota were sheared and ligated to the default 454 Titanium multiplex identifiers (MIDs; Roche Rapid Library Preparation Method Manual, GS FLX Titanium Series, October 2009). 16S rRNA sequencing of samples from individually caged mice colonized with the same community indicated a high degree of similarity between individual animals; for this reason, fecal DNAs from replicate mice were pooled (n=3-5 mice per pool), and the 12 pooled samples, each labeled with a unique MID, were sequenced in a single 454 Titanium run.

Shotgun pyrosequencing reads were parsed by MID and filtered to remove short sequences (<60 bp), low-quality sequences (three or more N bases in the sequence or two continuous N bases), and replicate sequences (>97% ID over the length of the read, with identical sequences over the first 20 bases). Reads reflecting host DNA contamination were identified by BLAST (against the mouse genome for samples isolated from mice and against the human genome for all other samples) and were removed in silico (≧75% identify, E-value≦10⁻⁵, bitscore≧50). Remaining sequences were queried against the KEGG Orthology (KO) database (v52) with a Blastx e-value cutoff of 10⁻⁵. KO assignments were mapped further to Enzyme Classification (EC) and KEGG pathway annotations.

Bio-Prospecting for Antibiotic-Resistance Genes in Uncultured and Readily Cultured Microbiota.

DNA fragments from complete and cultured communities were cloned into an expression vector, electroporated into Escherichia coli, and screened for their ability to confer resistance to 15 different antibiotics. To this end, 10 μg of DNA purified from the two human donors' fecal microbiota, from the derived cultured communities, plus pooled contents of the arrayed strain collection were sheared to 1.5- to 4-kB fragments (Bioruptor XL), followed by size selection (1% agarose gel electrophoresis). Sheared DNA then was endrepaired (Epicentre Endlt Kit), column-purified (Qiagen Qia-Quick PCR Purification Kit), and concentrated in a vacuum evaporator (SpeedVac). The expression vector pZE21-MCS1 was prepared by PCR amplification using primers flanking the HincII site [pZE21_{—}126_{—}146FOR, 5′-GACGGTATCGATAAGCTTGAT-3′ (SEQ ID NO 3); pZE21_{—}111_{—}123rcREV, 5′-GACCTCGAGGGGGGG-3′ (SEQ ID NO 4)] to linearize the vector, gel-purification of the linear product, dephosphorylation (calf intestinal phosphatase), and column purification. Approximately 500 ng of the DNA fragments were ligated to 100 ng of the linearized vector in an overnight ligation reaction (Epicentre FastLink Ligation Kit). The ligation reaction was desalted by dialysis in double-distilled H₂O and electroporated into E. coli MegaX DH10B T1R cells (Invitrogen). After 1 h of recovery, a small (1 μL) aliquot of the library was titered with serial dilutions onto LB agar plates containing 50 μg/mL kanamycin (to select for pZE21 transformants) and incubated at 37° C. for 16 h. The insert size distribution for each library was characterized by gel electrophoresis of amplicons obtained using primers flanking the HincII site in the multiple cloning site of pZE21 MCS1. The total size of each library was estimated by multiplying average insert size by the number of cfu in a given library. The remainder of the recovered cells was inoculated into 10 mL LB containing 50 μg/mL kanamycin and grown overnight, with shaking, at 27° C. for ˜16 h. The culture subsequently was diluted with an equal volume of LB medium containing 30% glycerol and stored at −80° C. before screening.

For functional selections, 100 μL of each library freezer stock (corresponding to 0.5-1×10⁸ cfu) was plated on an LB agar plate containing kanamycin (50 μg/mL) plus one of 15 different antibiotics (Table 3). The total number of cells plated on each antibiotic represented ˜10 copies of each original unique transformant. Antibiotic-resistant colonies were scored after plates had been incubated at 37° C. for 16 h. Inserts contained in colonies with amikacin-, piperacillin- and piperacillin/tazobactam-resistant phenotypes were subjected to bidirectional Sanger sequencing (Beckman Coulter Genomics) using primers pZE21_{—}81_{—}104_{—}57C (5′-GAATTCATTAAAGAGGAGAAA GGT-3′; SEQ ID NO 5) and pZE21_{—}151_{—}174rc_{—}58C (5′-TTTCGTTTTATTTGATGCCTCTAG-3′; SEQ ID NO 6). Resulting reads were trimmed to remove low-quality and vector sequences and subjected to within-library contig assembly (≧200 bp of 97% ID sequence required). Contigs and unassembled reads were mapped by BLAST to the National Center for Biotechnology Information (NCBI) nonredundant database and to a custom database of 122human gutmicrobial genomes. All sequence datasets have been deposited in the NCBI Sequence Read Archive (SRA) under accession no. SRA026271.

Amikacin-resistant strains were quantified from each donor, in triplicate, by plating diluted fecal samples on GMM with and without amikacin (4,100 μg/mL; lower concentrations produced high background). Amikacin-resistant colonies were quantified after 5-d incubation under anaerobic conditions, and colony counts were normalized to the total number of colonies obtained in the absence of the antibiotic. A total of 48 fecal isolates (12 from the GMM+amikacin selection and 12 from the nonselective plates, from each of two donors) were chosen for a PCR-based survey for the amikacin-resistance genes captured in the E. coli libraries described above and for 16S rRNA sequencing.

Preparation of an Arrayed Species Collection.

A single vial of the −80° C. anaerobic glycerol stock containing an aliquot of a fecal sample from Donor 2 was diluted into prereduced TYGs medium lacking resazurin in an anaerobic chamber and was dispensed into prereduced 384-well flat-bottomed trays (0.17 mL per well). To determine the dilution at which a high percentage of wells received a single viable cell in the initial inoculation (FIG. 11F), two- and fourfold serial dilutions were performed (from 10-6 to 10-10) in a trial inoculation (48 wells per dilution; 0.17 mL per well). Trays were sealed with sterile foil lids and incubated anaerobically at 37° C. for 5 d; the dilution at which ˜30% of wells were turbid (OD₆₃₀>0.2) was chosen for the subsequent large-scale culturing (FIG. 11G). To this end, a second vial of the frozen anaerobic glycerol stock from the same donor was added to 500 mL of prereduced TYGS medium lacking resazurin at the calculated dilution and dispensed into ten 384-well culture trays (170 μL per well). Trays were sealed and incubated as above. Cells then were resuspended in each well of each tray by pipetting, and 25 μL aliquots were transferred to each of two archive trays containing 25 μL prereduced TYGS (resazurin included) plus 40% glycerol per well. The arrayed archive trays were sealed with aluminum foil, frozen on dry ice inside the anaerobic chamber, and transported on dry ice to a conventional −80° C. freezer for storage. Cultures stored in this fashion remain anaerobic, as judged colorimetrically using resazurin in the medium and by recovery of strict anaerobes (as long as they are transported frozen, on dry ice, into an anaerobic chamber for strain recovery). Another 50-μL aliquot from the culture trays (not from the archive trays) was measured by OD₆₃₀ and stored at −80° C. for PCR amplification.

Taxonomies were assigned to each strain in the 3,840-well collection by two-step barcoded 454 FLX pyrosequencing. The V2 16S rRNA region of the DNA present in each well was amplified with an invariant V2-directed forward primer and 1 of 96 barcoded V2-directed reverse primers. A 1-μL aliquot from each well was transferred to a new tray, and cells were lysed in 10 μL of lysis buffer (25 mM NaOH, 0.2 mM EDTA; incubation for 30 min at 95° C.) followed by the addition of 10 μL of neutralization buffer (40 mM Tris-HCl). To reduce the amplification of background DNA present from dead or lysed cells, the neutralized lysate was diluted 1:10 into EB buffer (Qiagen). To barcode each bacterial strain uniquely before amplicon sequencing, the V2 region of their 16S rRNA gene was targeted for PCR using 2× Master Mix (Phusion HF), 2 μL input DNA, primers 454_{—}16S_{—}8F and 1 of 96 barcoded (Roche Multiplex Identifiers) reverse primers (454_{—}16S_{—}338R_barcode1) that include a 12-bp tail sequence in a 10-μL reaction (384-well format). Duplicate reactions were incubated for 30 s at 98° C., followed by 30 cycles of 10 s at 98° C., 30 s at 61° C., and 30 s at 72° C.

Reactions were combined so that each pool contained one representative associated with each barcode (four pools per 384-well tray), passed over a PCR cleanup column (Qiagen), and diluted to 0.5 ng/μL. Pools then were subjected to a second round of PCR amplification with 0.5 ng of pool DNA in a 25-μL reaction. Reactions were incubated for 30 s min at 98° C., followed by five cycles of 10 s at 98° C., 30 s at 54° C., and 30 s at 72° C., followed by an additional 25 cycles of 10 s at 98° C. and 30 s at 70° C. In this second PCR, the reverse primers were replaced with a second barcoded linker primer (454_linker_barcode2) specific to the 12-bp tail sequence added in the first PCR. In this way, the 16S rRNA V2 regions of the bacterial genomes in each initial well were associated with a unique twobarcode pointer sequence (FIG. 12A and legend). After the second-round PCR, reactions were pooled and run over a PCR cleanup column (Qiagen), and DNA in the expected size range (200-300 bp) was gel purified (Qiagen). The final product was quantified and subjected to multiplex 454 FLX pyrosequencing at a depth expected to yield 250 sequences per well (25% of one sequencing run).

The resulting reads were assigned to wells in the archive trays based on their associated barcodes. Of the 3,840 wells, 1,181 (30.8%) were turbid as defined by OD₆₃₀≧0.2; of the turbid wells, 1,172 (99.2%) had at least a single sequence with the correct barcode combination. Barcode combinations mapping to wells with culture OD₆₃₀<0.2 also were identified in the sequencing dataset. However, the total number of reads that mapped to these wells was much lower than to turbid wells (51,925 turbid versus 14,427 nonturbid), and the percentage of mapped wells was much lower for the nonturbid subset (62.2% vs. 99.2%).

The taxonomy of the most abundant sequence associated with each barcode combination was assigned using SILVA-VOTE. These most abundant sequences also were clustered into 97% ID OTU using uclust in QIIME 1.1. To evaluate the diversity captured at varying taxonomic levels, representative 97% ID OUT sequences were assigned taxonomy using SILVA-VOTE. Reads designated “nonidentified” by SILVA-VOTE were not considered to represent an additional taxonomic group unless they were associated with a distinct higher-order taxonomic classification (e.g., sequences annotated as “Family Clostridiaceae; Genus nonidentified” were scored as representing a different genus-level group than sequences annotated as “Family Ruminococcaceae; Genus nonidentified”). Rarefaction analysis of the number of additional taxa added with each additional 384-well culture tray is shown in FIG. 11H. Mapping was verified by recovery of strains from the archive trays, colony purification, and full-length Sanger sequencing of their 16S rRNA gene.

Identification of Human Gut Isolates in the German Resource Centre for Biological Material Culture Collection.

The German Resource Centre for Biological Material (DSMZ) bacterial culture collection (www.dsmz.de/microorganisms/bacteria_catalogue. php; Oct. 14, 2010) was searched under the terms “gut,” “faeces,” “feces,” “fecal,” and “stool.” Search results were filtered to exclude strains from nonhuman sources. Strains that matched the search terms without host species information were included, as were noncommensals (i.e., pathogens).

### Tables for Examples 1 to 9

TABLE 1
Common methodological differences between culture-independent and
culture-based surveys of human gut microbial diversity.

| | Culture-Independent | |
|---|---|---|
| | Metagenomic Methods | Culture-Based Methods |
| Survey depth | Thousands to millions of 16S | Tens to hundreds of isolates cultured |
| | rRNA gene sequences | per sample |
| | generated per microbial | |
| | community sample | |
| Accuracy of | 454 FLX (FLX standard) | Taxonomy typically defined from full |
| (bacterial) 16S | pyrosequencing platform: 10x-100x | length 16S rRNA genes using the |
| rRNA gene | over-estimate of species- | more accurate Sanger method of |
| assigments | level diversity due to artifacts | dideoxy-chain termination |
| | (sequencing errors, chimeras) | sequencing, or from high-coverage |
| | Illumina sequencing platform: | assemblies of isolate genomes |
| | ≧100x over-estimate of species- | |
| | level diversity due to artifacts | |
| Documentation | “Culturability” determined by | Many readily cultured taxa not |
| | matching 16S rRNA reads to | documented in public databases (in |
| | public databases | one study, 64%) |

TABLE 2
Gut microbiota medium (GMM)

| Component | Amount/L | | Concentration | Comments |
|---|---|---|---|---|
| Tryptone | 2 | g | 0.2% | |
| Peptone | | | | |
| Yeast Extract | 1 | g | 0.1% | |
| D-glucose | 0.4 | g | 2.2 mM | |
| L-cysteine | 0.5 | g | 3.2 mM | |
| Cellobiose | 1 | g | 2.9 mM | |
| Maltose | 1 | g | 2.8 mM | |
| Fructose | 1 | g | 2.2 mM | |
| Meat Extract | 5 | g | 0.5% | |
| KH₂PO4 | 100 | mL | 100 mM | 1M stock solution pH 7.2 |
| MgSO₄—7H₂0 | 0.002 | g | 0.008 mM | |
| NaHCO₃ | 0.4 | g | 4.8 mM | |
| NaCl₂ | 0.08 | g | 1.37 mM | |
| CaCl2 | 1 | mL | 0.80% | 0.8 g/100 mL stock |
| Vitamin K | 1 | mL | 5.8 mM | 1 mg/mL stock solution |
| (menadione) | | | | |
| FeSO₄ | 1 | mL | 1.44 mM | 0.4 mg FeSO₄/mL stock |
| | | | | solution |
| Histidine | 1 | mL | 0.1% | 1.2 mg hematin/mL in |
| Hematin | | | | 0.2M histidine |
| Solution | | | | |
| Tween 80 | 2 | mL | 0.05% | 25% stock solution |
| ATCC Vitamin | 10 | mL | 1% | |
| Mix | | | | |
| ATCC Trace | 10 | mL | 1% | |
| Mineral Mix | | | | |
| Acetic acid | 1.7 | mL | 30 mM | |
| Isovaleric acid | 0.1 | mL | 1 mM | |
| Propionic acid | 2 | mL | 8 mM | |
| Butyric acid | 2 | mL | 4 mM | |
| Resazurin | 4 | mL | 4 mM | 0.25 mg/mL stock solution |
| Noble Agar | 12 | g | 1.2% | |

TABLE 3
Antibiotics used in functional metagenomic selections

| Name | Class | Abbreviation | MIC (mg/mL) |
|---|---|---|---|
| Amikacin | Aminoglycoside | AM | 64 |
| Amoxicillin | βlactam | AX | 16 |
| Carbenicillin | βlactam | CA | 64 |
| Cefdinir | Cephalosporin | CF | 2 |
| Cloramphenicol | Amphenicol | CH | 8 |
| Ciprofloxacin | Flouroquinolone | CI | 4 |
| Cefepime | Cephalosporin | CP | 8 |
| Gentamicin | Aminoglycoside | GE | 16 |
| Oxytetracyline | Tetracyline | OX | 8 |
| Penicillin | βlactam | PE | 128 |
| Piperacillin | βlactam | PI | 16 |
| Piperacillin + | βlactam + | PI + TZ | 16(PI), 4(TZ) |
| Tazobactam | βlactamase | | |
| | inhibitor | | |
| Tetracyline | Tetracycline | TE | 8 |
| Trimethoprim | Pyrimidine derivative | TR | 8 |
| Trimethoprim + | Pyrimidine | TR + SX | 2(TR), 38(SX) |
| Sulfamethoxazole | derivative + | | |
| | Sulfonamide | | |

TABLE 4
BLAST analysis of metagenomic sequences identified by selection in E. coli
E. coli libraries containing microbiome fragments were selected on each of 15 antibiotics and antibiotic
combinations. Microbiome DNA fragments identified from three of these selections (amikacin, piperacillin,
piperacillin plus tazobactam) were subjecte

| Antibiotic | Gene identified | Donor | Complete/Cultured |
|---|---|---|---|
| Amikacin | rmtD; 16S rRNA methylase rmtD | 1 | Complete* |
| Amikacin | aphA-3; aminoglycoside phosphotransferase type III | 1 | Complete, Cultured |
| Amikacin | bcrA; bacitracin transport ATP-binding protein | 1 | Cultured |
| Amikacin | marR locus; multiple antibiotic resistance repressor | 1 | Cultured |
| Pipericillin | beta-lactamase; non-experimental evidence, no additional details | 1, 2 | Complete, Cultured |
| | recorded | | |
| Pipericillin | Megamonas hypermegale beta-lactamase class D | 1 | Cultured |
| Pipericillin | Bacteroides uniformis beta-lactamase cblA | 1, 2 | Cultured |
| Pipericillin | beta-lactamase/D-alanine carboxypeptidase ampC | 2 | Cultured |
| Pipericillin | Beta-lactamase precursor ampC | 2 | Cultured |
| Pipericillin | Clostridium nexile beta-lactamase class A | 2 | Cultured |
| Pipericillin | ABC-type multidrug transport system | 2 | Cultured |
| Pipericillin + | beta-lactamase; non-experimental evidence, no additional details | 1 | Complete, Cultured |
| Tazobactam | recorded | | |
| Pipericillin + | Clostridium bolteae beta-lactamase class A | 1 | Cultured |
| Tazobactam | | | |
| Pipericillin + | Megamonas hypermegale beta-lactamase class D | 1 | Cultured |
| Tazobactam | | | |
| Pipericillin + | Bacteroides uniformis Multi Antimicrobial Exclusion family | 1 | Cultured |
| Tazobactam | | | |
| Pipericillin + | Bactreroides caccae beta-lactamase class A | 1 | Cultured |
| Tazobactam | | | |
| Pipericillin + | Bacteroides uniformis beta-lactamase cblA | 1 | Cultured |
| Tazobactam | | | |
| Pipericillin + | beta-lactamase/D-alanine carboxypeptidase ampC | 2 | Cultured |
| Tazobactam | | | |

*rmtD sequences were found in readily cultured amikacin-resistant fecal strains from Donor 1

TABLE 5
Control PARC medium

| | Component | Amount/L | Concentration | Comments |
|---|---|---|---|---|
| P | KH2PO4 | 100 mL | 100 mM | 1M stock solution pH 7.2 |
| A | Noble Agar | 12 g | 1.2% | |
| R | Resazurin | 4 mL | 4 mM | 0.25 mg/mL stock |
| | | | | solution |
| C | L-cysteine | 0.5 g | 3.2 mM | |

### Example 10

### Modeling the Response of a Microbiota to Changes in Host Diets

Owing to its many roles in human health, there is great interest in deciphering the principles that govern the operations of an individual's gut microbiota. Current estimates indicate that each of us harbors several hundred bacterial species in our intestine and different diets lead to large and rapid changes in the composition of the microbiota. Given the dynamic interrelationship between diet, the configuration of the microbiota, and the partitioning of nutrients in food to the host, inferring the rules that govern the microbiota's responses to dietary ingredients represents a challenge.

Gnotobiotic mice colonized with simple, defined collections of sequenced representatives of the various phylotypes present in the human gut microbiota provide a simplified in vivo model system where metabolic niches, host-microbe, and microbemicrobe interactions can be examined using a variety of techniques. These studies have focused on small communities exposed to a few perturbations. In this example, gnotobiotic mice harboring a 10-member community of sequenced human gut bacteria were used to model the response of a microbiota to changes in host diet. The aim was to predict the absolute abundance of each species in this microbiota based on knowledge of the composition of the host diet. Another aim was to gain insights into the niche preferences of members of the microbiota, and to discover how much of the response of the community was a reflection of their phenotypic plasticity.

The ten bacterial species were introduced into germ-free mice to create a model community with representatives of the four most prominent bacterial phyla in the healthy human gut microbiota (FIG. 13A). Their genomes encode major metabolic functions that have been identified in anaerobic food webs, including the ability to break down complex dietary polysaccharides not accessible to the host (Bacteroides thetaiotaomicron, Bacteroides ovatus and Bacteroides caccae), consume oligosaccharides and simple sugars (Eubacterium rectale, Marvinbryantia formatexigens, Collinsella aerofaciens, Escherichia coli), and ferment amino acids (Clostridium symbiosum, E. coli). Two species capable of removing the end products of fermentation were also included: a H₂-consuming, sulfate-reducing bacterium (Desulfovibrio piger) and a H₂-consuming acetogen (Blautia hydrogenotrophica).

To perturb this community, a series of refined diets were used where each ingredient represented the sole source of a given macronutrient (casein=protein, corn oil=fat, cornstarch=polysaccharide, and sucrose=simple sugar) and where the concentrations of these four ingredients were systematically varied (FIG. 13B, C and Table 6). Each individually caged male C57Bl/6J mouse was fed a randomly selected diet with diet switches occurring every two-weeks (n=13 animals; Table 7 shows the variation of diet presentation between animals). 13 gnotobiotic mice harboring the 10-member community were each fed a randomly selected diet every two weeks for eight weeks (i.e., four total diets per mouse). Shotgun sequencing of total fecal DNA allowed the determination of the absolute abundance of each community member, based on assignment of reads to the various species' genomes, in samples obtained from each mouse on days 1, 2, 4, 7, and 14 of a given diet period. Analyses of the shotgun sequencing data revealed that steady state levels of community members were achieved within 24 h of a diet change. Therefore, the values from all five time points sampled within a diet period were averaged to obtain the mean absolute abundance of each community member for each of the refined diet periods.

TABLE 7
Variation of diet presentation between animals.

| mouse: | m1 | m2 | m3 | m4 | m5 | m6 | m7 | m8 | m9 | m10 | m11 | m12 | m13 |
|---|---|---|---|---|---|---|---|---|---|---|---|---|---|
| 1^{st} diet period | E | E | E | E | E | E | E | E | E | E | E | E | E |
| 2^{nd} diet period | B | G | E | K | F | J | H | D | J | A | C | I | E |
| 3^{rd} diet period | B | G | C | J | F | I | K | A | K | H | E | D | E |
| 4^{th} diet period | I | F | D | H | E | J | C | G | J | A | K | B | E |

To predict the abundance of each species in the model human gut microbiome given only knowledge of the concentration of each of the four perturbed diet ingredients, a linear model was used,

yᵢ=β₀+β_{casein}X_{casein}+β_{starch}X_{starch}+β_{sucrose}X_{sucrose}+βₒᵢₗXₒᵢₗ

where yᵢ; is the absolute abundance of species i, X_{casein}, X_{starch}, X_{sucrose}, and Xₒᵢₗ are the amounts (in g/kg of mouse diet) of casein, corn starch, sucrose, and corn oil respectively in a given host diet, β₀ is the estimated parameter for the intercept, and β_{casein}, β_{starch}, β_{sucrose}, and βₒᵢₗ are the estimated parameters for each of the perturbed diet components. Since each mouse underwent a sequence of three diet permutations presented in different order, and each of the diet periods covered all of the 11 possible diets (Table 7), it was possible to use two of these three diet intervals to fit the model for the equation (13 mice×2 diets per mouse=26 samples per bacterial species) and then the ability to predict the abundance of each bacterial species for the 13 samples was measured in the remaining (third) diet. Averaging this cross-validation from all three subsets, the model explained over 61% of the variance in the abundance of the community members (abundance weighted mean R²=0.61; see Table 8 for species-specific R²).

### Example 11

### Predicting Response of Microbiota to a Diet

Although the cross-validation provided evidence that the response of this microbiota was predictable from knowledge of these diet ingredients, a more conclusive validation of the model would be its ability to make predictions for new diets. Therefore, six additional diets were designed with new combinations of the four refined ingredients. Using a design similar to the first experiment, eight different 10-week-old gnotobiotic male C57Bl/6J mice harboring the 10-member community were each given a randomized sequence of diets selected from the six new diets (shaded diets L-Q in FIG. 13B), or one of the previous diets (Table 9). Fitting the model parameters with the data from the first experiment, we were able to explain 61% of the variance in the abundance of the community members on the new diets, showing virtually equivalent results to the cross validation procedure (see Table 8).

TABLE 9
Variation of diet presentation between animals.

| mouse: | m1 | m2 | m3 | m4 | m5 | m6 | m7 | m8 |
|---|---|---|---|---|---|---|---|---|
| 1^{st} diet period | Q | M | P | N | L | E | E | O |
| 2^{nd} diet period | N | D | O | M | E | Q | P | L |
| 3^{rd} diet period | P | N | M | O | F | E | L | Q |

These results indicate that the linear model explains the majority of the variation in abundance of each organism using only a knowledge of the species in the community and the concentrations of casein, cornstarch, sucrose, and corn oil in the diet, without having to explicitly consider the effects of microbe-microbe or microbe-host interactions, or diet order. As described in Materials and Methods below, several other models were also tested including adding interactions between the variables, quadratic terms, and interactions with quadratic terms. After correcting for the number of parameters in the model using Akaike information criterion, the linear model was still the best performing.

### Example 12

### Inferring the Association of Ingredients with the Abundance of Each Community Member

To further dissect the community response to these diet perturbations, we need to infer which set of diet ingredients is associated with the abundance of each community member. Feature selection algorithms assume that the response variable (in this case, the abundance of each organism) is potentially affected by only a fraction of the variables in the model, and use statistical methods to choose the subset of variables that most informatively predict the abundance of each species. Using stepwise regression as a feature selection procedure with the equation above, all species in the 10-member community had the diet variable X_{casein} significantly associated with their abundance (Table 10).

E. coli and C. symbiosum were the only bacteria with more than one variable significantly associated with their abundance (casein and sucrose for E. coli and casein and starch for C. symbiosum). Further exploring this finding, we found casein highly correlated with the yield of total DNA per fecal pellet across all diets (FIG. 14A and FIG. 15). A component of casein, presumably amino acids and/or nitrogen, limits the biomass of the community: this resource limitation was observed even for combinations of three additional refined protein and two additional fat sources (soy, lactalbumin, egg-white solids, olive oil and lard; n=9 different diets given to another group of 9 C57Bl/6J male mice; FIG. 16; Table 11). However, the observed changes in species abundance are not a simple consequence of a constant relative abundance of each community member that is scaled upwards as casein is increased: three community members, E. rectale, D. piger, and M. formatexigens, decreased in absolute abundance by 1.4-2.4-fold from the low casein to high casein diets even though total community biomass tripled (FIG. 14B, 17; Table 12). Similar changes in species abundance and total community DNA levels were observed when casein concentrations were altered in gnotobiotic mice harboring a 9-member or an 8-member subset of the original community (minus B. hydrogenotrophica or minus D. piger and B. hydrogenotrophica) (Table 13).

### Example 13

### Correlation of Diet Ingredients with mRNA Expression

Microbial RNA-Seq was used on fecal RNA samples, prepared from mice on each diet (mean=2.1±0.7 replicates per diet; Table 14), to determine if perturbations in diet ingredients correlated with underlying changes in mRNA expression by community members. Each of the 36 RNA-Seq datasets was composed of 36 nt-long reads (3.20±1.35×10⁶ mRNA reads/sample). Transcript abundances were normalized for each of the 10 species to reads per million per kilobase (RPKM). After correcting for multiple-hypotheses, no statistically significant changes in gene expression were found within a given bacterial species as a function of any of the diet perturbations. While community members do not appear to significantly alter their gene expression, they do respond by increasing or decreasing their absolute abundances (FIG. 15), thereby adjusting the total available transcript pool in the microbiota for processing dietary components. For example, as casein levels are increased across the diets, B. caccae increases its contribution to the gene pool/community transcriptome; so the number of transcripts per unit of casein remains roughly constant.

Since RNA-Seq provides accurate estimates of absolute transcript levels, transcript abundance information was used as a proxy to predict the major metabolic niche occupied by each community member. For species positively correlated with casein, it was found that high expression of mRNAs predicted to be involved in pathways using amino acids as substrates for nitrogen, as energy and/or as carbon sources. By contrast, the three species that negatively correlated with dietary casein concentration showed no clear evidence of high levels of expression of genes involved in catabolism of amino acids. The changes in abundance of the negatively correlated species (e.g., E. rectale) can be explained by competition with another member of the community that increases with casein (see FIG. 18).

### Example 14

### Use of the Modeling Framework with Typically Consumed Human Diets

The power of the refined diets used lies in the capacity to precisely control individual diet variables and to aid data interpretation from more complex diets. To test if the modeling framework used here generalizes to diets containing food more typically consumed in human diets, 48 meals were created consisting of random combinations and concentrations of four ingredients selected from a set of eight pureed human baby foods (apples, peaches, peas, sweet potatoes, beef, chicken, oats, and rice; Table 15). The meals were administered for periods of 7 d to the same eight gnotobiotic mice used for the follow-up refined diet experiments described above and in FIG. 13E. Each mouse received a sequence of 6 baby food diets. The order of presentation of the baby food diets was varied between animals (see Table 15). The absolute abundance of each bacterial community member was measured on days 1, 5, 6, and 7 for each diet. Using the linear modeling approach described above, over half of the variation in species abundance could be explained using only knowledge of the concentrations of the pureed foods present in each meal (R²=0.62). Stepwise regression was used to identify the type of pureed food(s) present in a given mixed meal that was most significantly associated with changes in each bacterial species (Table 16; FIG. 19).

Defining the interrelationship between diet and the structure and operations of the human gut microbiome is key to advancing understanding of the nutritional value of food, for creating new guidelines for feeding humans at various stages of their lifespan, for improving global human health, and for developing new ways to manipulate the properties of the microbiota to prevent or treat various diseases. The experiments and model described above highlight the extent to which host diet can explain the configuration of the microbiota, both for refined diets where all of the perturbed diet components are digestible by the host, and for human diets whose ingredients are only partially known. These models can now be tested using larger defined gut microbial communities representing those of humans living in different cultural settings, and with more complex diets, including various combinations of food ingredients that they consume.

### Materials and Methods for Examples 10 to 14.

### Assembling a Model Human Microbiota in Gnotobiotic Mice

B. caccae ATCC 43185 (GenBank genome accession number NZ_AAVM00000000), B. ovatus ATCC 8483 (NZ_AAXF00000000), B. thetaiotaomicron VPI-5482 (NC_{—}004663), B. hydrogenotrophica DSM 10507 (NZ_ACBZ00000000), M. formatexigens DSM 14469 (NZ_ACCL00000000), C. symbiosum ATCC 14940, C. aerofaciens ATCC 25986 (NZ_AAVN00000000), E. coli str. K-12 substr. MG1655 (NC_{—}000913), and E. rectale ATCC 33656 (NC_{—}012781) were obtained from public strain repositories (ATCC or DSMZ). A draft genome assembly for C. symbiosum ATCC 25986 is available at the Washington University Genome Center public web site (genome.wustl.edu/pub/organism/Microbes/Human_Gut_Microbiome/Clostridium_symbi osum/assembly/Clostridium_symbiosum-1.0/output/). D. piger GOR1 was isolated from a healthy human by plating serial dilutions of freshly voided feces under strictly anaerobic conditions (80% H₂/20% CO₂ at 15 psi) onto plates containing medium with the following components (quantities expressed per liter): K₂HPO₄ (0.3 g); KHPO₄ (0.3 g); (NH₄)SO₄ (0.3 g); NaCl (0.6 g); MgSO₄.7H₂O (0.13 g); CaCl₂.2H₂O (0.008 g), yeast extract (0.5 g); NH₄Cl (1.0 g); NaHCO₃ (5.0 g); dithiothreitol (0.5 g); sodium formate (3.0 g); Noble agar (10 g); 5 ml of a 0.2% (w/v) solution of Fe(NH₄)₂(SO₄)₂.6H₂O, 1 ml of a 0.2% (w/v) solution of resazurin; cysteine (1 g), 10 ml of trace mineral solution (ATCC), and 10 ml of a vitamin solution (ATCC). The genome sequence of D. piger was determined by 454 FLX and FLX Titanium pyrosequencing. For both C. symbiosum and D. piger, genes were identified using Glimmer3.0, tRNAScan 1.23, and RNAmmer 1.2. All 10 genomes were annotated using PFAM v23; and String COG version 7.1. Annotations for all 40,669 predicted protein-coding genes in the 10 genomes can be found at gordonlab.wustl.edu/modeling_microbiota/.

Each community member was grown anaerobically in 5 ml of TYGS medium in Balch tubes. Inoculation times were staggered so that all organisms reached stationary phase within a 24 h window. Just prior to gavage, equal volumes (1 ml) of each culture were pooled and mixed regardless of the final stationary phase density reached by each mono-culture (OD₆₀₀ values ranged from 0.4 to >2.0). Each germ-free mouse was subsequently gavaged with 300 μl of the pooled cultures.

### Refined Diet Composition, Experimental Design, and Data Processing

A set of eleven diets was initially designed (FIG. 13B,C and Table 6), each differing in their concentrations of casein (protein), corn oil (fat), cornstarch (polysaccharide), and sucrose (simple sugar). Nine of the diets consisted of all possible combinations of high, medium, and low casein and corn oil, with a fixed amount of cornstarch and the remainder as sucrose (FIG. 13B; diets A-I). Using sucrose as the ‘remainder’ for these initial nine diets generated a negative correlation between sucrose concentration and casein/fat concentration. Therefore, two diets, one with high starch and low sucrose and the other with low starch and high sucrose, were designed to lessen this negative correlation (see diets J and K in FIG. 13C).

Initially, all mice were co-housed and given the diet labeled ‘E’ in Table 7 (5% fat, 20% protein, 62% carbohydrate). Mice were then individually caged in the gnotobiotic isolator, and every two weeks each animal received another randomly selected diet (second, third and fourth diet periods in Table 7). Mouse 13 received only control diet E to determine if there was any ‘drift’ in steady state over the 8-week period.

The steady state mean absolute abundance of each community member was estimated for each of the 36 mouse/diet combinations for the second, third, and fourth diet periods shown in Table 7. To do so, DNA was isolated from fecal samples taken from each mouse on days 1, 2, 4, 7, and 14 of a diet and analyzed by COmmunity PROfiling by sequencing (COPRO-Seq). This generally applicable method relies on the massive number of short reads generated by the Illumina GA-II instrument during shotgun sequencing of total community DNA. Briefly, “informative” tags are identified that map uniquely to a single location in one species' genome. These tags are then summed to generate raw “counts” of each species' abundance. To account for non-unique matches, species-specific counts are normalized by the “Informative Genome Fraction” of each genome (defined as the fraction of all possible k-mers a genome can produce that are unique). Up to 16 barcoded fecal DNA samples were pooled in each sequencing lane: a minimum of 50,000 reads per sample were generated so that all organisms comprising ≧0.02% of the community could be detected (for a mouse colonized at 10¹² cfu/ml cecal contents or feces, this represents ˜10⁸ cfu/ml; at this sequencing depth, all species were detected in all samples). Total DNA yield per fecal pellet was used as a proxy for community biomass and multiplied the relative abundance of each species by the mean total DNA yield per fecal pellet for a particular diet to estimate the absolute abundance of each species in units of nanograms per fecal pellet. The absolute abundance N_{impd} of each species i in mouse m on diet period p on day d was calculated N_{impd}=F_{impd}Tⱼ where F_{impd} is the Informative Genome Fraction adjusted fraction of species i in mouse m on diet period p on day d as measured by COPRO-Seq and Tⱼ is the mean total DNA yield per fecal pellet for all samples taken from mice on diet j. Fecal pellets were used because they reflect overall microbiota composition in the gut and they provide the only means to sample each mouse over time. Mice were weighed during each diet period (Table 17). Although there was a trend towards increased weight gain as levels of casein and corn oil were increased (Table 18), there were no significant correlations between any of the diet perturbations and weight gain.

### Model Description and Performance Evaluation

Population growth can be modeled as exponential growth with a carrying capacity:

$$\frac{d N}{d t} = rN ( 1 - \frac{N}{K} ) \qquad Equation 1$$

where r is the growth rate, N is the population size, and K is the carrying capacity. Extending the above equation to include multiple species (i) and multiple diets (j), the model becomes:

$$\frac{d {N}_{i}}{d t} = {r}_{ij} {N}_{i} ( 1 - \frac{{N}_{i}}{{K}_{ij}} ) \qquad Equation 2$$

where Kᵢⱼ is the carrying capacity of species ion diet j (i.e. the steady-state level). We were interested in predicting the steady-state abundance of each species in the synthetic community as a function of the ingredients in the host diet. Thus, we can ignore the time-specific abundance of each community member Nᵢ(t) on each diet and the growth rate r, assuming it is sufficiently large to allow each community member to reach their carrying capacity for each diet within the period that a given mouse was consuming the diet.

To predict the steady-state levels (Kᵢⱼ) for each community member i given each diet j, we measured the abundance of each community member for each mouse and diet combination (FIG. S1D,E). The absolute abundance K_{impd} for each community member (i) in a specific mouse (m) for a specific diet period (p) for a given day (d) was calculated as described above. These abundances were averaged across all available time points for each mouse after the microbiota had reached steady-state (dₛ) for a specific diet period (i.e., the cells in the diet/mouse matrices in Tables 7 and 9; see below for estimation of steady-state). On average, 2.7 samples were available per mouse per diet period to give the steady-state abundance of each species (i) in the fecal microbiota for a given mouse (m) and diet period (p) combination in Tables 7 and 9.

yᵢₘₚ=mean(K_{impd}) where d>=dₛ Equation 3

These yᵢₘₚ values served as the data for the linear model described in the Examples above.

### Scoring Model Performance

All of the models used in this study were linear. Therefore, model could be scored by using R², which for linear models represent the proportion of variance in the system that is explained by the model. The R² was used for each species in the community separately to calculate a weighted mean R², where the weights represent the fraction of total fecal DNA content represented by each species (i.e., the R² for abundant taxa are given more weight than those of less abundant taxa). By using this weighted scoring schema, the final R² metric represents the amount of the total variation in species DNA content that can be explained by the model. An alternative method is to weight each species' R² equally, which produces similar albeit slightly worse results (Table 8).

### Estimating Steady-State

Since the model assumes the microbiota is at steady-state, values of species abundance were only included from time points after the microbiota reaches steady-state for a given diet period (i.e. dₛ in Equation 3 needs to be define). To determine the time required by the microbiota to reach steady-state after a diet switch, nine 22-week-old C57Bl/6J mice were fed a low protein/low fat diet for 7 d, followed by a switch to the high protein/high fat diet for 13 d (FIG. 13B diets A and I respectively). All mice were sampled approximately once every 24 h with twice-daily sampling around the time of the diet switch. To estimate whether the community had stabilized at a given time point, (i) the total microbial community biomass using DNA yield (ng/fecal pellet) as a marker, and (ii) the relative abundance of each species using the Informative Genome Fraction estimated from Illumina DNA sequencing were measured. It was found that the biomass of the gut microbiota was stable at the end of the first diet, highly variable during the initial time points after the diet transition, and then stable again by the fourth day after the diet switch (although even by 24 h after the switch, the mean yield largely reflects final steady-state abundance on the new diet). Similar results were found for the relative abundance data, although the relative abundance of each species appeared to stabilize faster than biomass (FIG. 20A compared with FIG. 20B). Given the results for both the biomass and relative abundance metrics, the number of days required by the microbiota to reach steady-state was set to four (i.e. dₛ=4).

### Comparing More Complex Models

Although the linear model performed well (see examples above and Table 8), more complex linear and nonlinear models could perhaps yield even better predictive ability. Therefore the cross validation procedure was repeated for the casein, corn oil, sucrose, starch diet combinations (see Examples above) using models that allowed for interactions between variables, quadratic terms, and interactions with quadratic terms:

yᵢ=β₀+β_{A}X_{A}+β_{B}X_{B}, Linear

yᵢ=β₀+β_{A}X_{A}+β_{B}X_{B}+β_{AB}X_{A}X_{B}, Interaction

yᵢ=β₀+β_{A}X_{A}+β_{B}X_{B}+β_{AA}X_{A}X_{A}+β_{BB}X_{B}XB, Quadratic

yᵢ=β₀+β_{A}X_{A}+β_{B}X_{B}+β_{AA}X_{A}X_{A}+β_{BB}X_{B}X_{B}+β_{AB}X_{A}X_{B}, Pure Quadratic

Akaike information criterion (AIC) was used as the scoring metric to allow for comparisons between these models with varying numbers of parameters and found the linear model performed best overall (Table 8). Given the slightly asymptotic behavior of the microbiota at extremely low and high casein concentrations (FIG. 14), it was also attempted to fit a nonlinear logistic function to the data to account for these saturation points. However, it was found that the lack of data at and beyond the asymptote made the nonlinear regression difficult to reliably fit. While it is interesting to question whether the microbiota will reach and maintain an asymptotic behavior with sampling at extreme concentrations of these ingredients, moving beyond the current maximum and minimum casein values would be unrealistic in terms of modern human eating habits and would be unhealthy for the animals.

### Transcriptional Responses of the Microbiota to Host Diet Perturbations

To deplete total microbial community RNA of 16S, 23S, 5S rRNA and tRNA species prior to synthesis of cDNA with random hexanucleotide primers, each fecal RNA preparation was subjected to column-based size-selection and hybridization to custom biotinylated oligonucleotides directed at conserved regions of bacterial rRNA genes present in human gut communities, followed by streptavidin-bead based capture of the hybridized RNA sequences. RNA-Seq data were normalized as described previously. After normalization, the list was filtered to remove all transcripts whose total number of counts (log₂) summed across all 36 RNA-Seq expression profiles was <64 (2⁶). This threshold was chosen to be as inclusive as possible while still requiring a sufficient number of reads so that a dynamic range of roughly 5-fold could be detected across the 17 sampled diets. For example, if a transcript linearly increases 5-fold in response to diets with a 20-fold range in their casein concentration, with the lowest concentration yielding a number of reads that was just below level of detection for both replicates and the highest casein concentration yielding 5 reads for that transcript per replicate, 55 reads would be require. After normalization and filtering, a list of 26,643 genes across the 10 species remained (64±20% of the annotated genes in each species were detected as ‘expressed’). For each of these genes, the correlation and the p-value of the correlation were calculated between (i) each of the four perturbed refined diet ingredients and (ii) the log₂(gene expression) in reads per million per kilobase (RPKM). Multiple hypotheses correction was performed using the Storey procedure.

### Highly Expressed Transcripts for Each Species

the highest 10% expressed genes in each community member were examined (gordonlab.wustl.edu/modeling_microbiota/), as major metabolic activities of gut microbes have consistently been identified among the abundant genes. Among the most highly expressed genes in B. thetaiotaomicron, were those encoding components of glycolysis/gluconeogenesis pathways (e.g. BT1658-1660, BT1672, 1691), the pentose phosphate pathway (e.g. BT3946-3950), plus members of polysaccharide utilization loci (PULs), including one PUL predicted to act on O-glycan containing mucins (BT0317-0319; S12), and another PUL involved in the degradation of fructans (BT1757-1763 and BT1765; 26). In addition to several peptidases (BT2522, BT2706, BT3926, BT4583), genes predicted to be involved in the metabolism of glutamate (glutamate dehydrogenase (BT1973); glutamate decarboxylase (BT2570)), glutamine (glutaminase (BT2571)), serine (L-serine dehydrate (BT4678)), aspartate (aspartate ammonia lyase (BT2755)), asparagine (L-asparaginase (BT2757)), and branched-chain amino acids (branched-chain alpha-keto acid dehydrogenase (BT0311-12)) were highly expressed. Similar results were observed in B. caccae and B. ovatus. Although the ability of colonic Bacteroides to access protein has not been extensively explored, there is evidence that members of this genus have extracellular proteolytic activity, and can incorporate amino acids into cellular components other than proteins. This feature, combined with their ability to use complex polysaccharides not accessible to other members in the community (including host glycans), may explain why they benefit from increased levels of dietary protein (casein).

Among the most highly expressed genes in C. symbiosum were components of the hydroxyglutarate pathway for degradation of glutamate (Csym2026-2031), the most abundant amino acid in casein (25.3% w/w), and a sodium/glutamate transporter (Csym3971). This pathway yields crotonyl-CoA, which is metabolized to butyrate, acetate, H2 and ATP. Genes encoding components of the pathway for butyrate production (Csym1328-1334) were also among the highest expressed.

Another Firmicute that grows on amino acids is the acetogenic bacterium B. hydrogenotrophica. Genes predicted to encode key enzymes of the acetyl-CoA pathway involved in the reductive assimilation of CO₂ were among the most highly expressed in this species (e.g., carbon monoxide dehydrogenase (Rumhyd0314-0320)), as were genes involved in fermentation of aliphatic (Rumhyd0546-0555) and aromatic amino acids (Rumhyd1109-1113), and the metabolism of ribose (Rumhyd2245-2256).

E. coli also benefited from higher levels of protein; among its most highly expressed genes were components of a cytochrome d terminal oxidase involved in the consumption of oxygen (b0733-0734), genes involved in the utilization of simple sugars (e.g., b2092-2097 (galactitol), b2416-2417 (glucose), b2801-2803 (fucose)) and several genes involved in the metabolism of tryptophan (b3708-b3709), aspartate (b1439) asparagine (b2957), and threonine (b3114-3117).

C. aerofaciens expressed high levels of transcripts encoding proteins predicted to be involved in the catabolism of arginine (COLAER0352-356, COLAER1230), plus components of several phosphotransferase systems (a predicted sucrose-specific PTS (COLAER0919-0921), a predicted mannose/fructose/N-acetylgalactosamine-specific PTS (COLAER1259-1260) and a predicted mannitol/fructose PTS (COLAER0058-0061)).

Levels of E. rectale and M. formatexigens decreased as protein increased. Inspection of their most highly expressed genes suggested that they focus on catabolism of carbohydrates. For example, among the most highly expressed genes in M. formatexigens were components of several ABC transporters with predicted specificities for monosaccharides/oligosaccharides (e.g., BRYFOR5076-BRYFOR5080, BRYFOR06841-06843), and genes encoding key enzymes of the acetyl-CoA pathway (e.g., BRYFOR06355-06360). There was no clear evidence of genes involved in catabolism of amino acids being highly expressed.

D. piger also decreased as casein levels increased. D. piger is fairly restricted in its metabolism: it can use a few substrates (e.g., lactate, H₂, succinate) to reduce different forms of sulfur to H₂S and generate energy, and it can oxidize lactate and pyruvate incompletely to acetate. Among its most highly expressed genes were components of the sulfate reducing pathway (DpigGOR12316-18, DpigGOR110789-10794), a C4-dicarboxylate transport system (DpigGOR12113-2115), subunits of a Ni—Fe hydrogenase, and several genes predicted to be involved in lactate metabolism (DpigGOR11071-1075). Three predicted transporters of amino acids were highly expressed, but there was no evidence of further metabolism of these amino acids, which likely indicates that they are used for protein biosynthesis.

Simulation of Negatively Correlated Species with Constant Behavioral Responses

Using Equation 2 above, a simulated 2-member community was created where one member (species1=N₁) is casein limited (C) and the second member (species2=N₂) is negatively influenced in proportion to the abundance of species1 (α₁₂) (e.g., species1 could consume a limiting resource of species2, produce an inhibitory compound, or act through apparent competition). It was assumed that both species have the same growth constant (r₁=r₂) on all diets and that species1 is able to convert a proportion (s) of the casein into increases in population size (K₁=sC; note that 1.3, 2/3, 0, 2/3, and 15 were used for constants r, s, α₂₁, α₁₂, and K₂ respectively, but this choice of values is arbitrary and similar results can be obtained over a wide-range of stable values):

$$\begin{matrix}\frac{d {N}_{1}}{d t} = {r}_{1} {N}_{1} ( 1 - \frac{{N}_{1} + {α}_{21} {N}_{2}}{{K}_{1}} ) & Equation 4 \\ \frac{d {N}_{2}}{d t} = {r}_{2} {N}_{2} ( 1 - \frac{{N}_{2} + {α}_{12} {N}_{1}}{{K}_{2}} ) & Equation 5\end{matrix}$$

Simulating the above equations, where every ten days we change the amount of casein (Cⱼ) from 2, 5, 10, 20, and 40% respectively, yields the result shown in FIG. 18 where species1 increases with increased casein while species2 decreases, during which time both maintain the same behaviors.

This type of behavior at the transcriptional level of a microbial community resembles similar phenomena observed in macro-ecology. For example, if two species of naturally co-occurring grasshoppers, one that eats almost exclusively grasses (Ageneotettix deorum) and the other that eats both grasses and forbs (Melanoplus sanguinipes), are co-housed to compete in environments with different dietary contexts, the final population size of each grasshopper species is dependent not only on the ability of A. deorum to compete for grass (i.e. its essential resource), but also M. sanguinipes' ability to utilize both grass and forbs. Thus, if the amount of grass available to the grasshoppers is held constant while the amount of forbs is increased, the population of A. deorum decreases even though it maintains the constant behavioral response of exclusively eating grass.

### Design, Administration, and Modeling of Complex Diets

The following commercially available eight pureed human baby foods were used as the source ingredients to construct a set of 48 meals: peaches (Gerber 3^{rd} foods®; Gerber Products Company); apple sauce (Gerber 3^{rd} foods); peas (Gerber 2^{nd} foods®), sweet potatoes (Gerber 3^{rd} foods); chicken (Gerber 2^{nd} foods); beef (Gerber 2^{nd} foods), oats (Gerber Single Grain with VitaBlocks®); and rice (Gerber Single Grain with VitaBlocks). Oats and rice were purchased dry and mixed with dH₂O in a 1:5 ratio prior to use (e.g., a meal with 6 g of oats contained 1 g dried oats and 5 g dH₂O). Each meal consisted of four ingredients randomly selected from the set of eight total pureed human foods, with different concentrations of the four ingredients used in different diet periods. Meals were autoclaved and each mouse was fed a sequence of 5 different diets, with each diet provided for 1 week. The order of presentation of the 48 diets to the 8 gnotobiotic mice is described in Table 15. The table shows how a 1 week period of consumption of one of the 17 diets composed of refined ingredients was interposed, between each 1 week period of administration of a given pureed baby food meal, to ensure mice obtained adequate amounts of vitamins and minerals.

Absolute abundance of each bacterial community member was measured on days 1, 5, 6, and 7 of each human baby food diet. As before, the abundance values (yᵢₘₚ) were calculated from the mean of all samples within a given diet period. However, day 1 was excluded in case the microbiota had not yet reached steady state by 24 h. To cover more of the potential “meal” space, the schema for the complex diets used less replication than was used for the refined diets, so there were fewer fecal pellets available to estimate the DNA yield data used to calculate absolute abundance of each species. Therefore, to estimate DNA yields for each diet, a nearest neighbor smoothing procedure was used where the DNA yield for each sample was calculated as a weighted average of the ten nearest samples with weights corresponding to the Euclidean distance from the true sample to the Nth-nearest sample (e.g., the nearest samples would be exact replicates and have a weight of 1.0).

The modeling performance was estimated with a species abundance weighted R² as described above using the following equation:

yᵢ=β₀+βₐₚₚₗₑXₐₚₚₗₑ+β_{peach}X_{peach}+βₚₑₐXₚₑₐ+β_{sweetpotato}X_{sweetpotato}+β_{chicken}X_{chicken}+β_{beef}X_{beef}+βₒₐₜₛXₒₐₜₛ+β_{rice}X_{rice},

where the variables correspond to the concentration of each pureed ingredient in each meal. The final performance metric was the mean of ten replicates of 10-fold crossvalidation on the 48 samples. The performance when training on the larger set (n˜43) and testing on the smaller set (n˜5) was similar to training and testing using the larger set only (weighted R²=0.62 and R²=0.66 respectively).

### Tables for Examples 10 to 14

TABLE 6A
Composition of refined diets: seventeen perturbations to casein, sucrose, corn starch, and corn oil concentrations.

| | First set of diets | | | | | | | | | | |
|---|---|---|---|---|---|---|---|---|---|---|---|
| | Diet ID: | | | | | | | | | | |
| | A | B | C | D | E | F | G | H | I | J | K |
| | Harlan Teklad Diet Number: | | | | | | | | | | |
| | TD.09049 | TD. 09050 | TD.09051 | TD.09052 | TD.09053 | TD.09054 | TD.09055 | TD.09056 | TD.09057 | TD.09058 | TD.09059 |
| | g/kg | g/kg | g/kg | g/kg | g/kg | g/kg | g/kg | g/kg | g/kg | g/kg | g/kg |
| Casein | 69 | 230 | 460 | 69 | 230 | 460 | 69 | 230 | 460 | 230 | 230 |
| L-Cystine | 0.9 | 3 | 6 | 0.9 | 3 | 6 | 0.9 | 3 | 6 | 3 | 3 |
| Sucrose | 675.88 | 514.13 | 283.08 | 633.46 | 471.66 | 240.66 | 473.98 | 312.38 | 81.38 | 171.66 | 571.66 |
| Corn Starch | 100 | 100 | 100 | 100 | 100 | 100 | 100 | 100 | 100 | 400 | 0 |
| Maltodextrin, | 50 | 50 | 50 | 50 | 50 | 50 | 50 | 50 | 50 | 50 | 50 |
| Lo-Dex 10 | | | | | | | | | | | |
| Cellulose (Fiber) | 50 | 50 | 50 | 50 | 50 | 50 | 50 | 50 | 50 | 50 | 50 |
| Corn Olil | 10 | 10 | 10 | 50 | 50 | 50 | 200 | 200 | 200 | 50 | 50 |
| 79055 Mineral | 12.73 | 12.73 | 12.73 | 13.4 | 13.4 | 13.4 | 16.08 | 16.08 | 16.08 | 13.4 | 13.4 |
| Mix, Ca-p | | | | | | | | | | | |
| Deficient | | | | | | | | | | | |
| Calcium | 2.6 | 6.25 | 11.3 | 2.6 | 6.4 | 11.3 | 3 | 6.5 | 11.6 | 6.4 | 6.4 |
| Carbonate | | | | | | | | | | | |
| Calcium | 12.5 | 7.5 | 0.5 | 13.4 | 8.3 | 1.4 | 16.4 | 11.4 | 4.3 | 8.3 | 8.3 |
| Phosphate | | | | | | | | | | | |
| 40077 | 14.25 | 14.25 | 14.25 | 15 | 15 | 15 | 18 | 18 | 18 | 15 | 15 |
| Vitamin mix | | | | | | | | | | | |
| Choline | 2.1 | 2.1 | 2.1 | 2.2 | 2.2 | 2.2 | 2.6 | 2.6 | 2.6 | 2.2 | 2.2 |
| Bitratrate | | | | | | | | | | | |
| Ethoxyquin | 0.04 | 0.04 | 0.04 | 0.04 | 0.04 | 0.04 | 0.04 | 0.04 | 0.04 | 0.04 | 0.04 |
| (Liquid) | | | | | | | | | | | |
| Protein | 6.1 | 20.3 | 40.6 | 6.1 | 20.3 | 40.6 | 6.1 | 20.3 | 40.6 | 20.3 | 20.3 |
| (% by weight) | | | | | | | | | | | |
| Protein | 6.7 | 22.7 | 46.5 | 6.3 | 21.5 | 44.1 | 5.3 | 18.0 | 36.8 | 22.2 | 21.3 |
| (% of kcal) | | | | | | | | | | | |
| Carbohydrate | 82.7 | 66.6 | 43.5 | 78.6 | 62.4 | 39.3 | 62.9 | 46.8 | 23.7 | 59.4 | 63.4 |
| (% by weight) | | | | | | | | | | | |
| Carbohydrate | 90.7 | 74.3 | 49.7 | 81.8 | 66.0 | 42.6 | 55.1 | 41.5 | 21.4 | 64.9 | 66.4 |
| (% by kcal) | | | | | | | | | | | |
| Fat | 1.1 | 1.2 | 1.5 | 5.1 | 5.2 | 5.5 | 20.1 | 20.2 | 20.5 | 5.2 | 5.2 |
| (% by weight) | | | | | | | | | | | |
| Fat (% by kcal) | 2.6 | 3.1 | 3.8 | 11.9 | 12.5 | 13.3 | 39.6 | 40.4 | 41.7 | 12.9 | 12.3 |
| kcal/g diet | 3.6 | 3.6 | 3.5 | 3.8 | 3.8 | 3.7 | 4.6 | 4.5 | 4.4 | 3.7 | 3.8 |

TABLE 6B
Composition of refined diets: seventeen perturbations to casein, sucrose,
corn starch, and corn oil concentrations.
Second set of diets

| L | M | N | O | P | Q |
|---|---|---|---|---|---|
| TD.09620 | TD.09621 | TD.09622 | TD.09623 | TD.09624 | TD.09625 |
| g/kg | g/kg | g/kg | g/kg | g/kg | g/kg |
| 138 | 345 | 138 | 345 | 35 | 690 |
| 1.8 | 4.5 | 1.8 | 4.5 | 45 | 9 |
| 585.425 | 377.525 | 484.52 | 276.42 | 667.61 | 108.11 |
| 100 | 100 | 100 | 100 | 100 | 0 |
| 50 | 50 | 50 | 50 | 50 | 50 |
| 50 | 50 | 50 | 50 | 50 | 50 |
| 30 | 30 | 125 | 125 | 50 | 50 |
| 13.06 | 13.06 | 14.74 | 14.74 | 13.4 | 13.4 |
| 4.2 | 8.8 | 4.5 | 9 | 1.9 | 12.25 |
| 10.7 | 4.3 | 12.5 | 6.4 | 14.4 | 0 |
| 14.525 | 14.525 | 16.5 | 16.5 | 15 | 15 |
| 2.15 | 2.15 | 2.4 | 2.4 | 2.2 | 2.2 |
| 0.04 | 0.04 | 0.04 | 0.04 | 0.04 | 0.04 |
| 12.2 | 30.5 | 12.2 | 30.5 | 3.1 | 60.9 |
| 13.1 | 33.5 | 11.7 | 29.7 | 3.2 | 67.1 |
| 73.7 | 52.9 | 63.8 | 43 | 82 | 17 |
| 79.4 | 58.1 | 61.1 | 41.9 | 85.1 | 18.7 |
| 3.1 | 3.4 | 12.6 | 12.9 | 5 | 5.7 |
| 7.5 | 8.4 | 27.2 | 28.3 | 11.7 | 14.1 |
| 3.7 | 3.6 | 4.2 | 4.1 | 3.9 | 3.6 |

Calcium carbonate and calcium phosphate were used to maintain calcium and phosphorus levels at 0.5% and 0.35%, respectively, across all diets with the exception of the diet with the highest level of protein (TD.09621) where the phosphorus present in casein brings its level to 0.48%. Vitamin and mineral mixes were adjusted based on the caloric density of each diet.
Custom diets designed for this study are now commercial available from Harlan Teklad using the Harlan Teklad Diet Number above.

TABLE 8
Model performance measurements for individual species in experiments involving variations
in dietary casein, corn oil, corn starch and sucrose concentrations.
A. R² performance measurements for linear model

| | cross validation | prediction on new diets |
|---|---|---|
| Eubacterium rectale ATCC 33656 | 0.25 | 0.68 |
| Collinsella aerofaciens ATCC 25986 | 0.08 | 0.42 |
| Blautia hydrogenotrophica DSM 10507 | 0.70 | 0.63 |
| Desulfovibrio piger GOR1 | 0.08 | 0.13 |
| Clostridium symbiosum ATCC 14940 | 0.70 | 0.70 |
| Escherichia coli str. K-12 substr. MG1655 | 0.22 | 0.61 |
| Marvinbryantia formatexigens DSM 14469 | 0.08 | 0.43 |
| Bacteroides ovatus ATCC 8483 | 0.69 | 0.54 |
| Bacteroides thetaiotaomicron VPI-5482 | 0.69 | 0.50 |
| Bacteroides caccae ATCC 43185 | 0.78 | 0.80 |
| mean | 0.43 | 0.54 |
| weighted mean (weighted by species abundance) | 0.61 | 0.61 |

B. Cross validation comparison of AIC scores across different models

| | linear | interaction | quadratic | pure quadratic |
|---|---|---|---|---|
| Eubacterium rectate ATCC 33656 | 121.3 | 132.9 | 140.9 | 129.5 |
| Collinsella aerofaciens ATCC 25986 | 161.5 | 174.9 | 182.9 | 170.7 |
| Blautia hydrogenotrophica DSM 10507 | 137.0 | 150.8 | 158.8 | 146.3 |
| Desulfovibrio piger GOR1 | 193.3 | 205.9 | 213.9 | 201.8 |
| Clostridium symbiosum ATCC 14940 | 169.7 | 181.2 | 189.7 | 177.2 |
| Escherichia coli str. K-12 substr. MG1655 | 200.0 | 213.3 | 221.3 | 209.4 |
| Marvinbryantia formatexigens DSM 14469 | 199.4 | 210.6 | 218.6 | 206.7 |
| Bacteroides ovatus ATCC 8483 | 204.9 | 216.5 | 224.5 | 212.4 |
| Bacteroides thetaiotaomicron VPI-5482 | 211.6 | 222.0 | 229.7 | 218.2 |
| Bacteroides caccae ATCC 43185 | 206.8 | 220.8 | 228.8 | 216.3 |

TABLE 10
Stepwise regression selection of diet ingredients significantly associated with
changes in species abundance.

| | Casein | Sucrose | Corn Starch | Corn Oil |
|---|---|---|---|---|
| Eubacterium rectale ATCC 33656 | 1.10E−07 | 0.43 | 0.35 | 1.00 |
| Collinsella aerofaciens ATCC 25986 | 3.13E−03 | 0.21 | 0.49 | 0.26 |
| Blautia hydrogenotrophica DSM 10507 | 1.51E−08 | 0.24 | 0.65 | 0.18 |
| Desulfovibrio piger GOR1 | 1.13E−02 | 0.31 | 0.90 | 0.12 |
| Clostridium symbiosum ATCC 14940 | 2.63E−15 | 0.64 | 2.44E−03 | 0.65 |
| Escherichia coli str. K-12 substr. MG1655 | 1.57E−07 | 9.38E−03 | 0.55 | 0.56 |
| Marvinbryantia formatexigens DSM 14469 | 1.31E−03 | 0.65 | 0.97 | 0.48 |
| Bacteroides ovatus ATCC 8483 | 1.36E−07 | 0.32 | 0.06 | 0.36 |
| Bacteroides thetaiotaomicron VPI-5482 | 3.29E−12 | 0.43 | 0.74 | 0.38 |
| Bacteroides caccae ATCC 43185 | 4.48E−19 | 0.47 | 0.52 | 0.76 |

Significant p-values from regression are shown in boldface

TABLE 11
Composition of nine diets with combinations of three refined protein sources and two refined fat sources.

| | Mouse | | | | | | | | |
|---|---|---|---|---|---|---|---|---|---|
| | m1 | m2 | m3 | m4 | m5 | m6 | m7 | m8 | m9 |
| | Harlan Teklad Diet Number: | | | | | | | | |
| | TD.10321 | TD.10309 | TD.10314 | TD.10316 | TD.10311 | TD.10319 | TD.10310 | TD.10307 | TD.10308 |
| Ingredients | g/kg | g/kg | g/kg | g/kg | g/kg | g/kg | g/kg | g/kg | g/kg |
| Isolated Soy Protein | 172 | 17.2 | 17.2 | 172 | 17.2 | 172 | 17.2 | 17.2 | 17.2 |
| Egg White Solids, spray-dried | 187.5 | 187.5 | 18.8 | 187.5 | 18.8 | 18.8 | 18.8 | 18.8 | 187.5 |
| Lactalbumin | 172 | 17.2 | 17.2 | 17.2 | 172 | 172 | 172 | 17.2 | 17.2 |
| Sucrose | 15.5 | 322.3 | 682.4 | 264.5 | 336.2 | 278.4 | 528.1 | 585.3 | 514.2 |
| Maltodextrin | 100 | 100 | 100 | 100 | 100 | 100 | 100 | 100 | 100 |
| Corn Starch | 50 | 50 | 50 | 50 | 50 | 50 | 50 | 50 | 50 |
| Cellulose (Fiber) | 50 | 50 | 50 | 50 | 50 | 50 | 50 | 50 | 50 |
| 79055 Mineral mix, Ca—P Deficient | 16.1 | 16.1 | 12.7 | 14.7 | 16.1 | 14.7 | 12.7 | 14.7 | 12.7 |
| Calcium Carbonate | 6.3 | 2.3 | 1.7 | 5.5 | 2.6 | 5.5 | 2.3 | 1.8 | 1.9 |
| Calcium Phosphate Dibasic | 10.0 | 16.8 | 13.7 | 9.6 | 16.5 | 9.6 | 12.6 | 16.1 | 13.0 |
| 40077 Vitamin mix | 18.0 | 18.0 | 14.3 | 16.5 | 18.0 | 16.5 | 14.3 | 16.5 | 14.3 |
| Choline Bitartrate | 2.6 | 2.6 | 2.1 | 2.4 | 2.6 | 2.4 | 2.1 | 2.4 | 2.1 |
| Biotin | 0.004 | 0.004 | 0.004 | 0.004 | 0.004 | 0.004 | 0.004 | 0.004 | 0.004 |
| Olive Oil | 100 | 100 | 10 | 100 | 100 | 10 | 10 | 10 | 10 |
| Lard | 100 | 100 | 10 | 10 | 100 | 100 | 10 | 100 | 10 |
| TBHQ (antioxidant) | 0.04 | 0.04 | 0.01 | 0.02 | 0.04 | 0.02 | 0.01 | 0.02 | 0.01 |
| Protein, g/kg | 451 | 181 | 45 | 316 | 181 | 315 | 181 | 45 | 181 |
| CHO, g/kg | 177 | 480 | 837 | 423 | 496 | 438 | 684 | 742 | 668 |
| Fat, g/kg | 215 | 201 | 21 | 118 | 208 | 125 | 28 | 111 | 21 |
| Fiber, g/kg | 50 | 50 | 50 | 50 | 50 | 50 | 50 | 50 | 50 |

TABLE 12
Correlation of species abundance with mouse diet casein concentration.

| | correlation with | |
|---|---|---|
| | casein | p-value |
| Eubacterium rectale ATCC 33656 | −0.61 | 1.1E−07 |
| Collinsella aerofaciens ATCC 25986 | 0.37 | 3.1E−03 |
| Blautia hydrogenotrophica DSM 10507 | 0.64 | 1.5E−08 |
| Desulfovibrio piger GOR1 | −0.32 | 1.1E−02 |
| Clostridium symbiosum ATCC 14940 | 0.77 | 1.3E−13 |
| Escherichia coli str. K-12 substr. MG1655 | 0.61 | 8.5E−08 |
| Marvinbryantia formatexigens DSM 14469 | −0.40 | 1.3E−03 |
| Bacteroides ovatus ATCC 8483 | 0.61 | 1.4E−07 |
| Bacteroides thetaiotaomicron VPI-5482 | 0.74 | 3.3E−12 |
| Bacteroides caccae ATCC 43185 | 0.86 | 4.5E−19 |

TABLE 13
Responses of 8-member and 9-member subset communities to low and
high casein.
A. Increase in total community DNA from low casein to high casein

| | mean totat DNA yield (ng/fecal pellet) | | percent |
|---|---|---|---|
| Community | diet 6 (tow casein) | diet I (high casein) | increase |
| 8-member | 8568 | 13800 | 61% |
| 9-member | 12448 | 16875 | 36% |
| 10-member | 7692 | 11718 | 52% |

8-member and 9-member community samples were from 13 to 14-week
old NMRI mice
10-member community samples were from 10 to 16-week old
C57BI/6J mice
B. Species-level responses ro changes in casein concentration

| Species | 8-member | 9-member | 10-member |
|---|---|---|---|
| Bacteroides caccae ATCC 43185 | p | p | p |
| Clostridium symbiosum | p | p | p |
| ATCC 14940 | | | |
| Bacteroides thetaiotaomicron | p | p | p |
| VPI-5482 | | | |
| Blautia hydrogenotrophica | — | — | p |
| DSM 10507 | | | |
| Escherichia coli str. K-12 | p | p | p |
| substr. MG1655 | | | |
| Eubacterium rectale ATCC 33656 | n | n | n |
| Bacteroides ovatus ATCC 8483 | p | p | p |
| Marvinbryantia formatexigens | n | n | n |
| DSM 14469 | | | |
| Collinsella aerofaciens | n | p | p |
| ATCC 25986 | | | |
| Desulfovibrio piger GOR1 | — | n | n |

species are sorted by the p-value of the correlation between casein and
species abundance for the 10-member community.
n = negatively correlated with casein concentration.
p = positively correlated with casein concentration.
— = not present in community.

TABLE 14
Number of expressed genes/species with ≧64 sequencing reads.

| | | Total | % |
|---|---|---|---|
| | Genes | Genes in | Observed |
| Species | Observed | Genome | Genes |
| Eubacterium rectale ATCC 33656 | 453 | 3621 | 13% |
| Collinsella aerofaciens ATCC 25986 | 1779 | 2367 | 75% |
| Blautia hydrogenotrophica | 2612 | 3869 | 68% |
| DSM 10507 | | | |
| Desulfovibrio piger GOR1 | 1660 | 2487 | 67% |
| Clostridium symbiosum ATCC 14940 | 3141 | 5128 | 61% |
| Escherichia coli str. K-12 substr. | 2969 | 4132 | 72% |
| MG1655 | | | |
| Marvinbryantia formatexigens | 3173 | 4896 | 65% |
| DSM 14469 | | | |
| Bacteroides ovatus ATCC 8483 | 3785 | 5536 | 68% |
| Bacteroides thetaiotaomicron | 3696 | 4778 | 77% |
| VPI-5482 | | | |
| Bacteroides caccae ATCC 43185 | 3375 | 3855 | 88% |

TABLE 15
Composition of and experimental design for complex diets composed of pureed baby foods.

| | mouse 1 | mouse 2 | mouse 3 | mouse 4 | mouse 5 | mouse 6 | mouse 7 | mouse 8 | g/kg | |
|---|---|---|---|---|---|---|---|---|---|---|
| week 1 | apple sauce | peas | sweet | peaches | oatmeal | sweet | rice | beef | 666.7 | BABY FOOD |
| | | | potatoes | | | potatoes | | | | MEAL 1 |
| | peaches | peaches | chicken | peas | rice | apple sauce | apple sauce | peaches | 222.2 | |
| | chicken | chicken | beef | oatmeal | beef | beef | sweet | sweet | 55.6 | |
| | | | | | | | potatoes | potatoes | | |
| | sweet | oatmeal | peas | rice | sweet | peaches | beef | rice | 55.6 | |
| | potatoes | | | | potatoes | | | | | |
| week 2 | TD.09052 | TD.09623 | TD.09621 | TD.09622 | TD.09620 | TD.09624 | TD.09625 | TD.09054 | | Refined Diet |
| week 3 | apple sauce | peas | sweet | peaches | oatmeal | sweet | rice | beef | 666.7 | BABY FOOD |
| | | | potatoes | | | potatoes | | | | MEAL 2 |
| | peaches | peaches | chicken | peas | rice | apple sauce | apple sauce | peaches | 222.2 | |
| | chicken | chicken | beef | oatmeal | beef | beef | sweet | sweet | 55.6 | |
| | | | | | | | potatoes | potatoes | | |
| | sweet | oatmeal | peas | rice | sweet | peaches | beef | rice | 55.6 | |
| | potatoes | | | | potatoes | | | | | |
| week 4 | TD.09620 | TD.09623 | TD.09621 | TD.09622 | TD.09625 | TD.09624 | TD.09052 | TD.09054 | | Refined Diet |
| week 5 | sweet | chicken | beef | apple sauce | apple sauce | apple sauce | peas | peaches | 666.7 | BABY FOOD |
| | potatoes | | | | | | | | | MEAL 3 |
| | oatmeal | peaches | rice | oatmeal | peaches | beef | apple sauce | oatmeal | 222.2 | |
| | peaches | beef | peas | chicken | chicken | chicken | sweet | sweet | 55.6 | |
| | | | | | | | potatoes | potatoes | | |
| | peas | rice | chicken | beef | rice | peaches | peaches | chicken | 55.6 | |
| week 6 | TD.09049 | TD.09053 | TD.09050 | TD.09056 | TD.09058 | TD.09053 | TD.09051 | TD.09059 | | Refined Diet |
| week 7 | peas | chicken | peaches | peaches | peas | chicken | sweet | peas | 666.7 | BABY FOOD |
| | | | | | | | potatoes | | | MEAL 4 |
| | rice | sweet | peas | peas | chicken | peaches | rice | rice | 222.2 | |
| | | potatoes | | | | | | | | |
| | apple sauce | rice | rice | apple sauce | oatmeal | rice | peaches | sweet | 55.6 | |
| | | | | | | | | potatoes | | |
| | beef | oatmeal | beef | chicken | sweet | oatmeal | beef | peaches | 55.6 | |
| | | | | | potatoes | | | | | |
| week 8 | TD.09053 | TD.09051 | TD.09059 | TD.09053 | TD.09050 | TD.09049 | TD.09058 | TD.09056 | | Refined Diet |
| week 9 | oatmeal | oatmeal | sweet | peaches | sweet | peas | peaches | chicken | 421.1 | BABY FOOD |
| | | | potatoes | | potatoes | | | | | MEAL 5 |
| | rice | apple sauce | rice | oatmeal | oatmeal | chicken | beef | beef | 421.1 | |
| | chicken | beef | chicken | sweet | chicken | rice | oatmeal | peas | 105.3 | |
| | | | | potatoes | | | | | | |
| | apple sauce | rice | peas | beef | rice | peaches | rice | apple sauce | 52.6 | |
| week 10 | TD.09053 | TD.09053 | TD.09053 | TD.09055 | TD.09057 | TD.09055 | TD.09057 | TD.09053 | | Refined Diet |
| week 11 | peas | apple sauce | peaches | sweet | sweet | beef | apple sauce | apple sauce | 250 | BABY FOOD |
| | | | | potatoes | potatoes | | | | | MEAL 6 |
| | oatmeal | rice | oatmeal | beef | apple sauce | peas | oatmeal | rice | 250 | |
| | beef | oatmeal | rice | peaches | peaches | chicken | rice | peaches | 250 | |
| | chicken | beef | sweet | rice | beef | oatmeal | beef | chicken | 250 | |
| | | | potatoes | | | | | | | |

Custom Harlan Teklad Diet Numbers are provided for the weeks mice were on refined diets.

TABLE 16
Stepwise regression selection of complex diet ingredients significantly associated with changes in species abundance.

| | Apple | Beef | Chicken | Oat | Pea | Peach | Rice | Sweet Potato |
|---|---|---|---|---|---|---|---|---|
| Desulfovibrio piger GOR1 | 0.37 | 0.18 | 1.26E−04 | 6.08E−03 | 0.81 | 0.10 | 2.27E−06 | 0.17 |
| Collinsella aerofaciens ATCC 25986 | 0.06 | 4.49E−05 | 5.55E−07 | 0.26 | 0.10 | 0.91 | 0.08 | 0.19 |
| Blautia hydrogenotrophica DSM 10507 | 0.14 | 1.73E−03 | 5.62E−06 | 0.99 | 0.14 | 0.21 | 0.50 | 0.57 |
| Clostridium symbiosum | 1.14E−03 | 7.29E−04 | 2.30E−04 | 0.25 | 0.16 | 4.96E−04 | 0.35 | 0.84 |
| Escheria coli str. K-12 substr. MG1655 | 4.30E−04 | 1.42E−03 | 8.63E−05 | 0.85 | 0.28 | 0.38 | 7.74E−05 | 0.70 |
| Bryantella formatexigens DSM 14469 | 0.48 | 0.07 | 0.87 | 0.38 | 0.98 | 0.10 | 0.96 | 0.18 |
| Eubacterium rectale ATCC 33656 | 3.77E−05 | 0.27 | 0.07 | 6.27E−06 | 0.13 | 2.76E−03 | 0.68 | 0.58 |
| Bacteroides caccae ATCC 43185 | 0.28 | 7.56E−06 | 2.00E−05 | 0.37 | 0.57 | 0.76 | 2.46E−03 | 3.72E−02 |
| Bacteroides thetaiotaomicron VPI-5482 | 2.31E−05 | 0.78 | 0.12 | 3.51E−03 | 1.94E−02 | 6.52E−04 | 0.31 | 0.43 |
| Bacteroides ovatus ATCC 8483 | 0.20 | 0.46 | 1.00 | 1.43E−08 | 0.92 | 0.11 | 0.16 | 0.34 |

TABLE 17
Mean Weight Gain (g/diet period).

| mouse | m1 | m2 | m3 | m4 | m5 | m6 | m7 | m8 | m9 | m10 | m11 | m12 | m13 |
|---|---|---|---|---|---|---|---|---|---|---|---|---|---|
| 2^{nd} diet period | 0.891 | 1.400 | 1.909 | 2.291 | 1.527 | −1.909 | −1.655 | −1.145 | 2.927 | 0.636 | 0.255 | 2.291 | 0.636 |
| 3^{rd} diet period | 0.800 | 0.000 | 0.600 | 1.200 | 0.300 | 0.600 | 0.100 | 1.400 | −0.100 | 1.900 | 1.200 | −0.700 | 1.000 |
| 4^{th} diet period | 1.400 | 2.800 | 0.700 | 2.300 | 1.100 | 2.400 | 1.300 | 2.700 | 3.300 | 0.300 | 1.100 | 1.900 | 2.700 |

TABLE 18
Weight gain as a function casein and corn oil concentration.

| | |
|---|---|
| Mean Weight Gain (g/diet period) | SEM |

A. Weight gain per diet period as a function of Casein concentration

| | | |
|---|---|---|
| % Casein | | |
| 7% | 0.589 | 0.386 |
| 23% | 1.233 | 0.295 |
| 46% | 1.233 | 0.294 |

B. Weight gain per diet period as a function of Corn oil concentration

| | | |
|---|---|---|
| % Corn Oil | | |
| 1% | 0.900 | 0.180 |
| 5% | 1.110 | 0.301 |
| 20% | 1.211 | 0.463 |

TABLE 19
Fecal DNA yields from diverse protein diets.

| mouse | diet | nanograms (mean ± SEM) |
|---|---|---|
| m1 | TD.10321 | 19716 ± 5689 |
| m2 | TD.10309 | 15488 ± 7023 |
| m3 | TD.10314 | 3781 ± 2906 |
| m4 | TD.10316 | 27989 ± 5462 |
| m5 | TD.10311 | 10378 ± 1515 |
| m6 | TD.10319 | 14283 ± 3203 |
| m7 | TD.10310 | 18604 ± 1007 |
| m8 | TD.10307 | 259 ± 75 |
| m9 | TD.10308 | 19684 ± 9045 |

### Example 15

### Intact and Cultured Gut Microbial Communities from Twins Discordant for Obesity Transplanted into Gnotobiotic Mice

Substantial interpersonal differences in microbial community configurations normally exist between unrelated individuals, creating a challenge in designing surveys of sufficient power to determine whether observed differences between healthy versus disease-associated microbiomes are significantly different from normal interpersonal variation. Microbiome configurations are influenced by early environmental exposures and are generally more similar among family members. In the case of same-sex twins discordant for a disease phenotype, the genetically related, healthy co-twin provides a valuable reference control for characterizing the disease-associated co-twin's microbiome. However, while each discordant pair can provide a vignette about the potential role of the microbiome in disease pathogenesis, the comparison is fundamentally descriptive and does not establish causality. Transplanting a fecal sample obtained from each co-twin in a discordant pair into multiple recipient mice provides an opportunity to conduct a virtual clinical trial designed to identify structural and functional differences between their communities, to generate and test hypotheses about the impact of these differences on host biology, and to directly test the effects of manipulating the representation of microbial taxa in the community.

A number of studies of obese and lean humans have revealed compositional differences in their gut microbiomes. Mono- or dizygotic twins discordant for obesity provide an attractive study paradigm for studies of the contributions of the gut microbiome to differences in body mass index (BMI). Two Finnish twin cohort studies have provided much of the published data about BMI discordance for obesity among monozygotic (MZ) twin pairs. In one cohort, with participants aged 35-60 years at the time of data collection, 1.3% of MZ twin pairs were defined as discordant for obesity [body mass index (BMI) difference ≧3 kg/m2 with one twin >27 kg/m² and the other <25 kg/m²]. In the other study, with participants aged 22-27 years at data collection, 2.16% of MZ twin pairs had a BMI difference ≧4 kg/m². Data collected at the 5th wave of assessment from 1539, 21-32 year-old female twin pairs enrolled in the Missouri Adolescent Female Twin Study (MOAFTS) was surveyed. Four discordant twin pairs with a BMI difference ≧6 kg/m² were recruited for the present study (n=1 MZ; 3 DZ pairs; Table 20).

TABLE 20
Features of the 4 discordant twin pairs.

| | Twin Pair | | | | | | | |
|---|---|---|---|---|---|---|---|---|
| | 1 | | 2 | | 3 | | 4 | |
| BMI (kg/m²) | 23 | 32 | 25.5 | 31 | 19.5 | 30.7 | 24 | 33 |

| | | | | |
|---|---|---|---|---|
| Zygosity | DZ | DZ | DZ | MZ |

Comparisons of the input human fecal microbiota, and ‘output’ mouse fecal communities surveyed two weeks after transplantation revealed that 77.8±7.4% (SD) of genus-level bacterial taxa in the human donor microbiota were represented in the microbiota of gnotobiotic mouse recipients (n=3-12 animals analyzed/microbiota; Table 21). The UniFrac metric measures the overall degree of phylogenetic similarity of any two bacterial communities by comparing the degree of branch length they share on a Bacterial tree of life. V2-16S rRNA reads sharing 97% nucleotide sequence identity were considered to represent a given species-level operational taxonomic unit (OTU). Principal Components Analysis (PCoA) of unweighted UniFrac distance matrices based on the 97% ID OTU datasets revealed that transplanted microbial communities achieved a stable configuration in recipients within 3 d. This configuration was sustained for at least 38 d. Importantly, the overall phylogenetic architecture of the transplanted community evolved in a reproducible way between singly housed recipient mice within given experiment for a given co-twin microbiota, and between replicate experiments (FIG. 21B). Pairwise UniFrac-based comparisons of fecal samples and of communities sampled along the length of the gut of transplant recipients also demonstrated a significantly higher similarity among recipients colonized with the same human donor, and a greater similarity to their human donor compared to mice colonized with unrelated human donor microbiota (FIG. 21C; FIG. 22).

TABLE 21
Fecal samples obtained from American female twin pairs discordant for obesity and used as donor samples for gnotobiotic mice.
Percent recapitulation at each taxonomical level based on pyrosequencing data from the V2 region of the 16S rRNA gene.

| | Twin Pair ID | | | | | | | | |
|---|---|---|---|---|---|---|---|---|---|
| | 1 | | 2 | | 3 | | 4 | | All |
| BMI of the | Lean | Obese | Overweight | Obese | Lean | Obese | Lean | Obese | |
| donor (kg/m²) | (23) | (32) | (25.5) | (31) | (19.5) | (30.7) | (24) | (33) | |
| Twin ID | TSDC17 | TSDC16 | TSDC19 | TSDC20 | TSDC22 | TSDC23 | TSDC7 | TSDC8 | |
| Phyla (%) | 100 | 75 | 100 | 80 | 100 | 100 | 100 | 100 | 94.4 ± 10.5 |
| Class (%) | 80 | 80 | 100 | 83.3 | 85.7 | 85.7 | 60 | 85.7 | 82.6 ± 11.1 |
| Order (%) | 57.1 | 66.7 | 80 | 57.1 | 87.5 | 66.6 | 70 | 66.6 | 69.0 ± 10.4 |
| Family (%) | 62.5 | 63.6 | 83.3 | 66.7 | 77.8 | 83.3 | 82.3 | 68.7 | 73.5 ± 9.1 |
| Genus (%) | 69 | 66.7 | 88 | 76 | 80 | 84.2 | 82.9 | 75.9 | 77.8 ± 7.4 |
| OTU level (%) | 30.80 ± 7.81 | 44.39 ± 3.81 | 27.74 ± 2.59 | 17.13 ± 4.10 | 16.91 ± 2.09 | 21.43 ± 1.32 | 19.41 ± 9.56 | 28.54 ± 7.92 | 25.89 ± 10.33 |

Transplant recipients not only efficiently captured the organismal features of their human donor's microbiota but also the functions encoded by the donor's microbiome, as judged by shotgun pyrosequencing of cecal DNA samples isolated from mice colonized with each of the 8 human fecal microbiota [n=3-8 mice sampled 15 d after transplantation/microbiota; n=45 cecal samples; 90,164±37,526 (mean±SD) reads per sample; 337±62 (SD) nt/read; 9.43±4.12 Mb/sample]. Shotgun reads were functionally annotated with KEGG orthology groups (KO) and Enzyme Commission (E.C.) numbers (KEGG version 58; see Methods). The results disclosed that 99.69±0.2% of donor ECs were captured in recipients. Remarkably, there was a significant correlation between the proportional representation of reads with given assignable EC and KO in donor and recipient microbiomes (Spearman's correlation, p-value<0.0001, Spearman's R≧0.88) (FIG. 23), highlighting the remarkable ability of a human microbiome to reassemble itself in a mouse gut ecosystem.

Body composition was analyzed using quantitative magnetic resonance imaging 1 d, 15 d, and in the case of longer experiments 38 d after transplantation. The increased adiposity phenotype of each obese co-twin in a discordant twin pair was transmissible: the differences in adiposity between mice that received an obese co-twins fecal microbiome was statistically greater than the adiposity of mice receiving her lean co-twins microbiome within an experiment, and was reproducible between experiments (One-tail Mann-Whitney U test, p-value<0.001; n=92 recipients phenotyped) (FIG. 24A, B). Epididymal fat pad weights were also significantly higher in mice colonized with gut communities from obese co-twins (p<0.05, one-tail Mann-Whitney U test). These differences in adiposity were not associated with statistically significant differences in daily chow consumption among recipients of obese versus lean co-twin microbiomes.

Supervised machine learning was performed using Random Forests to identify family-level bacterial phylotypes that differentiate gnotobiotic mice harboring a gut community transplanted from all lean versus all obese co-twins. The estimated generalization error of the trained model was 12.6%, indicating that it could be predicted if a sample came from a mouse colonized with a lean or obese human donor microbiota with 87.4% accuracy using family-level taxonomic classifications. Only three family-level taxa were identified as producing a mean decrease in classification accuracy of ≧5% when they were ignored; all three families were members of the order Clostridiales: Lachnospiraceae, Ruminococcaceae and Veillonellaceae; two of the three (Ruminococcacae and Veillonellaceae) were significantly increased in fecal samples obtained from mice colonized with a lean donor's microbiota (Table 22).

TABLE 22
Discriminatory family level taxa between gnotobiotic mice colonized with the microbiota of human
co-twins discordan for obesity. Feature importance score was calculated using supervised machine learning
(Random Forest algorithm) and represents t

| | Feature importance scores = | | | |
|---|---|---|---|---|
| | Mean decrease in | Relative Abundance (%) | | |
| | accuracy when the feature | Lean donors | Obese donors | p-adjust |
| Feature ID | is ignored | (mean ± SEM) | (mean ± SEM) | (ANOVA, FDR) |
| Firmicutes; Clostridia; Clostridiales; | 10.06% | 1.19 ± 0.47 | 0.00 ± 0.00 | 1.58E−15 |
| Veillonellaceae | | | | |
| Firmicutes; Clostridia; Clostridiales; | 9.45% | 16.02 ± 1.68 | 8.34 ± 3.19 | 7.64E−09 |
| Ruminococcaceae | | | | |
| Firmicutes; Clostridia; Clostridiales; | 7.84% | 21.29 ± 2.29 | 30.82 ± 1.35 | 2.81E−12 |
| Lachnospiraceae | | | | |
| Firmicutes; Clostridia; Clostridiales; | 3.40% | 0.04 ± 0.03 | 0.19 ± 0.10 | 3.28E−04 |
| Eubacteriaceae | | | | |
| Bacteroidetes; Bacteroidia; | 1.62% | 0.57 ± 0.20 | 0.84 ± 0.38 | 7.26E−02 |
| Bacteroidales; Rikenellaceae | | | | |
| Firmicutes; Clostridia; Clostridiales; | 0.70% | 0.74 ± 0.18 | 1.17 ± 0.26 | 8.05E−03 |
| Others | | | | |
| Firmicutes; Clostridia; Clostridiales; | 0.46% | 0.04 ± 0.01 | 0.00 ± 0.00 | 8.61E−03 |
| Clostridiales FamilyXIII. Incertae | | | | |
| Sedis | | | | |
| Tenericutes; Erysipelotrichi; | 0.42% | 1.91 ± 0.68 | 2.61 ± 0.45 | 5.55E−02 |
| Erysipelotrichales; | | | | |
| Erysipelotrichaceae | | | | |
| Firmicutes; Clostridia; Clostridiales; | 0.40% | 0.17 ± 0.11 | 0.34 ± 0.22 | 5.69E−02 |
| Clostridiaceae | | | | |
| Proteobacteria; Deltaproteobacteria; | 0.27% | 0.09 ± 0.05 | 0.16 ± 0.07 | 5.10E−02 |
| Desulfovibrionales; | | | | |
| Desulfovibrionaceae | | | | |
| Actinobacteria; Actinobacteria; | 0.13% | 0.11 ± 0.04 | 0.11 ± 0.05 | 9.63E−01 |
| Coriobacteriales; Coriobacteriaceae | | | | |
| Actinobacteria; Actinobacteria; | 0.12% | 0.00 ± 0.00 | 0.02 ± 0.01 | 1 |
| Coriobacteriales; Others | | | | |
| Verrucomicrobia; Verrucomicrobiae; | 0.12% | 1.05 ± 0.40 | 0.93 ± 0.29 | 7.80E−01 |
| Verrucomicrobiales; | | | | |
| Verrucomicrobiaceae | | | | |
| Firmicutes; Clostridia; Clostridiales; | 0.07% | 0.01 ± 0.00 | 0.05 ± 0.07 | 8.02E−02 |
| Peptococcaceae | | | | |
| Firmicutes; Bacilli; Turicibacterales; | 0.01% | 0.15 ± 0.15 | 0.15 ± 0.07 | 9.41E−01 |
| Turicibacteraceae | | | | |

Estimated generalization error = 0.126050
Error estimation method = Out-of-bag prediction of training data
Number of features used = 15
Parameters
# values of all non-default parameters
# method was “random_forest”
seed:616869664
cross.validation.k:10

ShotgunFunctionalizerR, a software tool designed for metagenomic analysis and based on a Poisson model, was used to identify genes encoding KEGG KOs and ECs whose proportional representation in cecal microbiomes differed significantly between recipients of transplanted obese versus lean co-twin microbiomes (p-value<0.0001). Random Forests is an ensemble classifier, that uses multiple decision trees to identify which features are discriminatory among different class labels, rather than features that are over- or underrepresented. This complementary approach to Shotgun FunctionalizerR identified KOs and ECs that best discriminate transplanted obese and lean microbiomes (relevant discriminatory features defined as those with a feature importance score ≧0.0001). These predictive KOs and ECs were among the most significantly different KOs and ECs as judged by ShotgunFunctionalizeR.

This type of DNA-level analysis provides information about functional capacity, but not about expressed functions. Therefore, the same cecal samples were used to prepare RNA for microbial RNA-Seq characterization of the transplanted microbial communities' meta-transcriptomes. Transcripts were mapped to 127 sequenced human gut genomes and assigned to KEGG KOs and ECs (see Methods). Significant differences in gene expression between transplanted obese and lean co-microbiomes were defined using ShotgunFunctionalizerR and Random Forests.

All transplanted lean co-twin microbiomes exhibited increased relative abundance of transcripts encoding components of the KEGG ‘Starch and Sucrose’ metabolic pathway (cellobiose and pyruvate metabolism) as well as the KEGG ‘Mannose and Fructose’ metabolic pathway (propanoate and butanoate metabolism). ECs involved in the KEGG pathway for ‘Pyrimidine and Purine’ metabolism were also enriched in the transcriptomes expressed by lean co-twin microbiomes, consistent with the significantly greater fecal microbial biomass (defined by fecal DNA content) observed in mice colonized with the gut microbial communities of lean versus obese co-twin donors (p<0.00X, ANOVA). Obese microbiomes had a significant increased in the representation of ECs involved in more futile energy metabolic cycles [e.g. KEGG ‘Pentose phosphate’ pathway) (FIG. 25).

Non-targeted gas chromatography-mass spectrometry (GC/MS) analyses of cecal contents was used to confirm these findings, and to identify other differences in expressed microbiome-associated metabolic activities. A metabolite profile was obtained for each sample using the spectral abundances of all identifiable metabolites (cecal samples from 3-5 mice/microbiome donor). A total of 26 metabolites satisfied a reverse match score cutoff of 65% (for definition, see Methods) and were present in at least 50% of samples representing a given transplanted microbiome (Table 23); 10 were robust discriminatory biomarkers of lean versus obese microbiome donor transplants in all four pairs (two tail Student's t-test, p-value<0.01; Random Forests, importance score >0.0007). Five of these 10 metabolites were mono- and disaccharides [cellobiose, mannose, glucose, lactose, and talose (C2-epimer of galactose)]; each was present at significantly lower levels in the cecal metabolomes of mice colonized with the gut communities of lean co-twins and each can be fermented by members of gut microbiota to short chain fatty acids (SCFA). Targeted GC/MS of cecal SCFA revealed significant increases in propionate and butyrate levels in mice harboring transplanted lean co-twin microbiomes (p<0.05, Student's t-test), consistent with increased carbohydrate fermentation (FIG. 26A, B). Bomb calorimetry revealed a trend towards increased energy content in the feces of mice containing transplanted lean compared to obese microbiomes (2.4±0.2 (SEM) and 1.9±0.3 kcal/mg dry fecal weight, respectively; p=0.07; two tailed Student's t-test; 2 discordant twin pairs surveyed; 7-12 mice/co-twin microbiome). This trend, coupled with the significantly greater biomass and lower SCFA levels associated with transplanted lean co-twin microbiomes, raise the question of whether the transplanted gut communities from lean co-twin donors may be more ‘selfish’, consuming energy for their own use, and less altruistic in distributing energy to their gnotobiotic hosts.

TABLE 23
Metabolites whose levels are significantly different in 29
cecal samples obtained from gnotobiotic recipients of intact fecal
samples from obese versus lean co-twins from four discordant pairs.
Metabolites were identified using non-targeted GC/M

| Metabolite | ttest | p-adjust |
|---|---|---|
| tagatose 1 | 0.165003758 | 0.4158388 |
| D-lyxosylamine | 0.377652896 | 0.61797747 |
| D-altrose | 0.022259913 | 0.08903965 |
| lactose 2 | 0.000758951 | 0.02714539 |
| 3-(3-hydroxyphenyl)propionic acid | 0.908546205 | 0.93858256 |
| cellobiose 1 | 0.001508077 | 0.02714539 |
| D-allose | 0.022259913 | 0.08903965 |
| gluconic acid lactone 1 | 0.010717259 | 0.0654987 |
| talose 1 | 0.016037437 | 0.08247825 |
| Phenylalanine | 0.529530864 | 0.65734866 |
| coniferyl alcohol | 0.347509677 | 0.61797747 |
| maleic acid | 0.055489479 | 0.18160193 |
| fumaric acid | 0.931223792 | 0.93858256 |
| beta-alanine 1 | 0.473120794 | 0.63082773 |
| acetoacetate 2 | 0.173266167 | 0.4158388 |
| cholesterol | 0.450002889 | 0.62308092 |
| oxalic acid | 0.444235043 | 0.62308092 |
| 5-Hydroxylysine | 0.502472019 | 0.64603545 |
| succinic acid | 0.89644695 | 0.93858256 |
| L-alanine 1 | 0.351162848 | 0.61797747 |
| 2-hydroxybutyric acid | 0.338703755 | 0.61797747 |
| glycine | 0.006317688 | 0.0654987 |
| D-malic acid | 0.757829475 | 0.85255816 |
| L-glutamic acid 2 | 0.145769214 | 0.40366859 |
| 5-aminovaleric acid 1 | 0.00890099 | 0.0654987 |
| ribose | 0.660979975 | 0.76758965 |
| D-lyxose | 0.429692899 | 0.62308092 |
| 2-amino-1-phenylethanol [symbol] , Ä† | 0.325820188 | 0.61797747 |
| putrescine | 0.127190686 | 0.38157206 |
| Myristic Acid | 0.267126773 | 0.60103524 |
| L-sorbose 1 | 0.620036783 | 0.74404414 |
| fructose 1 | 0.938582558 | 0.93858256 |
| D-mannose 1 | 0.033391959 | 0.12021105 |
| D-glucose 1 | 0.01091645 | 0.0654987 |
| Myoinositol | 0.364584224 | 0.61797747 |
| inosine | 0.41516831 | 0.62308092 |

GPCRs comprise the largest superfamily of transmembrane signaling proteins encoded in the human genome, and participate in an array of signaling pathways that regulate myriad aspects of host physiology. GPCRs expressed by gut epithelial cell lineages (e.g. enteroendocrine cells) would be in a strategic position to transduce metabolic signals emanating from the microbiota to the host. To determine whether obese versus lean microbiota have differential effects on GPCR signaling, TaqMan assays were used to survey the expression of 350 GPCRs, belonging to 50 subfamilies, in the distal small intestine (ileums) of microbiota transplant recipients (initially 2 discordant pairs; 4 mice/donor microbiota). Three GPCRs satisfied criteria for a consistent >2-fold difference in expression in the distal small intestines of recipients of lean versus obese co-twin microbiota (p-value<0.05; Student's t-test). qRT-PCR was used to confirm the differential patterns of expression of these three GPCRs, Gpr15, Gpr3 and Gabbr2, in gnotobiotic recipients of microbiota from members of all four discordant twin pairs (n=4 mice/microbiota). Gpr15/Bob is abundant at the basal surface of the small intestinal epithelium 0. In the human enterocyte-like cell line HT-29-D4 cells, activation of Gpr15 leads to a 70% decrease in sodium-dependent glucose and lipid transport. Gpr15 down-regulation in mice harboring an obese microbiome would be expected to result in increased glucose and lipid absorption.

Procrustes analysis utilizing a Bray-Curtis distance matrix from the different groups of gnotobiotic recipients revealed significant correlations (p-value<10⁻⁷) between taxonomic structure (V2-16S rRNA family-level bacterial phylotype), functional capacity (EC representation in cecal microbiomes), transcriptional profiles (EC representation in cecal mRNA populations), and metabolic profile (non-targeted GC/MS profiles) (Mantel test with 10,000,000 iterations, p-value<0.001), with separation of groups based on donor microbiota and adiposity phenotype.

These observations were followed up, generated from studies of transplanted intact (uncultured) donor communities, with a set of experiments involving culture collections produced from the fecal microbiota of one of the discordant twin pairs. The goal was to determine whether cultured bacterial members of the co-twins'microbiota could transmit the discordant adiposity phenotypes and metabolic profiles of the corresponding uncultured microbiomes to gnotobiotic recipients.

Collections of cultured anaerobic bacteria were generated from each co-twin in DZ pair 1 (see Methods). The bacterial taxa present in the culturable component of each co-twin's microbiota were defined by sequencing V2-16S rRNA amplicons generated DNA isolated from the entire collection harvested directly from plates. Subsequently, each transplanted collection was harvested from plates directly into separate groups of 8-week old germ-free male C57Bl/6J mice (n=3 independent experiments; 4-6 recipient mice/culture collection/experiment). Capture of the cultured taxa was reproducible with 61±2% and 56±7% of the genus-level phylotypes present in the obese and lean co-twin's intact cultured microbiota retained in gnotobiotic recipients of their culture collection as shown (Table 24) by unweighted UniFrac analysis of V2-16S rRNA datasets (97% ID OTUs generated from cecal community DNA 15 d after gavage of an intact uncultured fecal microbiota or the corresponding non-arrayed culture collection generated from that fecal sample (n=5 mice/treatment group; 4 treatment groups). The culture collections reached a steady state configuration within 3 d after transplantation. Shotgun sequencing of the cecal microbiomes of transplant recipients confirmed that the functional features of the intact (non-cultured) donor microbiomes were efficiently captured and their proportional representation was recapitulated. Remarkably, statistically significantly greater increases in adiposity was documented 15 d after gavage in recipients of the obese co-twin's compared to the lean co-twin's culture collection (p<0.02; Mann Whitney U-test).

TABLE 24
Efficiency of capture of taxa, present in culture
collections prepared from obese and lean co-twins belonging to
twin pair 1, in gnotobiotic mouse recipients.

| | Shared with mice | | | |
|---|---|---|---|---|
| Twin Pair 1 | colonized with an | | Shared with intact | |
| BMI of co- | intact community | | Human donor sample | |
| twin donor | Obese | Lean | Obese | Lean |
| Phyla | 89.7 ± 8.1 | 100 ± 8.7 | 84.0 ± 8.9 | 96.0 ± 8.9 |
| Class | 88.9 ± 8.0 | 88.8 ± 11.6 | 80.0 ± 13.9 | 74.3 ± 12.0 |
| Order | 86.6 ± 7.0 | 87.2 ± 11.9 | 68.6 ± 12.0 | 54.0 ± 8.9 |
| Family | 83.2 ± 3.4 | 86.4 ± 8.2 | 63.1 ± 3.4 | 50.9 ± 7.5 |
| Genus | 73.9 ± 6.6 | 82.0 ± 7.3 | 61 ± 2.2 | 55.6 ± 6.5 |
| OUT-97% ID | 73.8 ± 6.6 | 69.5 ± 7.4 | 41.9 ± 9.3 | 18.1 ± 3.2 |

Given that mice are coprophagic, co-housing was used to determine whether exposure of a mouse harboring a culture collection from the lean co-twin could modify or rescue development of an increased adiposity phenotype in a cagemate colonized with the culture collection generated from her obese co-twin, or vice versa. Five days after gavage, a mouse with the lean co-twin's culture collection was co-housed with a mouse with the obese co-twin's culture collection, with or without two age-matched germ-free animals. Control groups consisted of cages of dually-housed recipients of the lean co-twin culture collection, or dually-housed recipients of the obese co-twin's culture collection (n=3-5 cages/experiment; n=2 independent experiments; each cage in each experiment placed a separate gnotobiotic isolator) (FIG. 27A). All mice were fed a low-fat, plant polysaccharide-rich diet. Adiposity phenotypes were measured by quantitative magnetic resonance imaging 1 and 5 days after transplantation, and after 10 days of co-housing. Fecal samples were collected from all mice on days 1, 2, 3, 5, 6, 7, 8, 10 and 15.

The results revealed that the microbiota of the co-housed mouse harboring the obese co-twin's culture collection (abbreviated Obch) was re-configured so that its phylogenetic composition came to resemble that of the animal with the lean co-twin's culture collection (abbreviated Ln^{ch}). In contrast, the microbiota of the Ln^{ch} mouse remained stable (FIG. 27C-F). Moreover, Ob^{ch} mice exhibited a significantly lower change in adiposity compared to Ob controls that had never been exposed to mice harboring the lean co-twin's culture collection, while Ln^{ch} mice had adiposity phenotypes that were indistinguishable from Ln controls (FIG. 27B). Co-housing experiments that included germ-free members revealed that these animals had adiposity phenotypes that were indistinguishable from Ln^{ch} cagemates (FIG. 27B).

Similar to what was observed with complete community transplants, the ceca of mice colonized with the lean co-twin's culture collection exhibited significantly greater levels of short chain fatty acids, particularly acetate, propionate and butyrate than recipients of the obese co-twin's culture collection (FIG. 27H). The differences with regards to lactose and cellobiose were even more dramatic than with the complete (uncultured) transplants: These metabolites were undetectable in mice with culture collection from the lean co-twin (FIG. 27I). Co-housing not only affected host adiposity phenotypes but also transformed the Ob^{ch} mouse's cecal metabolic profile such that it was indistinguishable from Ln controls (FIG. 27I).

To delineate the contribution of specific members of the lean co-twin's microbiota to these phenotypic changes, limiting dilution was used to produce a clonal arrayed collection of the lean twin's culture collection in replicate 384 well plates (Methods). 16S rRNA sequencing was initially used to identify the bacterial taxa present in wells that exhibited growth (see Methods) and to confirm that the well contained a clonal population of bacterial cells. The collection contained 54 strains representing 23 phylotypes (Table 25). DNA prepared from wells containing a single strain was then sequenced at ≧50× coverage and their genomes annotated. Then, a pool containing 37 was assembled comprising strains whose genomes were sequenced. This pool contained six strains of the known cellobiose fermentor Collinsella aerofaciens (family Coriobacteriaceae) plus 22 members of the family Bacteroidaceae and 11 members of Ruminococcaceae. These latter 33 members were chosen because Random Forests indicated that they discriminated between the transplanted lean and obese co-twin culture collections (feature importance score ≧0.1) and because their abundance increased significantly in the Ob^{ch} gut microbiota during the co-housing experiment described above (ANOVA; p-value<0.05 after Bonferroni correction) (Table 25).

TABLE 25
Components of the arrayed anaerobic bacterial culture collection produced
from the lean co-twin in DZ pair 1.

| | | Lactose | Cellobiose |
|---|---|---|---|
| Strain designation | 37 Selected taxa | degrading | degrading |
| Ruminococcus_bromii_TSDC17.2_V2.2.5 | + | | |
| Ruminococcus_bromii_TSDC17.2_V2.2.2 | + | | |
| Ruminococcus_bromii_TSDC17.2_V2.1.7 | + | | |
| Ruminococcus_albus_TSDC17.2_V2.2.8 | + | | + |
| Ruminococcus_albus_TSDC17.2_V2.1.7 | + | | + |
| Ruminococcus_albus_TSDC17.2_V2.1.6 | + | | + |
| Ruminococcus_albus_TSDC17.2_V2.1.16 | + | | + |
| Ruminococcaceae_TSDC17.2_V2.2.3 | + | | |
| Ruminococcaceae_TSDC17.2_V2.2.1 | + | | |
| Ruminococcaceae_TSDC17.2_V2.2.1 | + | | |
| Ruminococcaceae_TSDC17.2_V2.1.1 | + | | |
| Peptoniphilus_TSDC17.2_V2.1.1 | | | |
| Parabacteroides_distasonis_TSDC17.2_V2.1.2 | | | |
| Odoribacter_splanchnicus_TSDC17.2_V2.1.2 | | | |
| Odoribacter_splanchnicus_TSDC17.2_V2.1.1 | | | |
| final_name | | | |
| Escherichia_coli_TSDC17.2_V2.1.2 | | | |
| Escherichia_coli_TSDC17.2_V2.1.1 | | | |
| Dorea_TSDC17.2_V2.1.1 | | | |
| Coprococcus_comes_TSDC17.2_V2.1.2 | | | |
| Coprococcus_comes_TSDC17.2_V2.1.1 | | | |
| Collinsella_aerofaciens_TSDC17.2_V2.4.22 | + | + | |
| Collinsella_aerofaciens_TSDC17.2_V2.3.23 | + | + | |
| Collinsella_aerofaciens_TSDC17.2_V2.3.20 | + | + | |
| Collinsella_aerofaciens_TSDC17.2_V2.2.24 | + | + | |
| Collinsella_aerofaciens_TSDC17.2_V2.1.9 | + | + | |
| Collinsella_aerofaciens_TSDC17.2_V2.1.10 | + | + | |

TABLE 25
Components of the arrayed anaerobic bacterial culture collection produced from the lean co-twin in
DZ pair 1.

| | | Lactose | Cellobiose |
|---|---|---|---|
| Strain designation | 37 Selected taxa | degrading | degrading |
| Clostridiaceae_TSDC17.2_V2.3.1 | | | |
| Clostridiaceae_TSDC17.2_V2.2.4 | | | |
| Bifidobacterium_pseudocatenulatum_TSDC17.2_V2.1.5 | + | | |
| Bacteroides_vulgatus_TSDC17.2_V2.2.12 | + | | − |
| Bacteroides_vulgatus_TSDC17.2_V2.1.5 | + | | − |
| Bacteroides_vulgatus_TSDC17.2_V2.1.11 | + | | − |
| Bacteroides_thetaiotaomicron_TSDC17.2_V2.3.1 | + | | − |
| Bacteroides_thetaiotaomicron_TSDC17.2_V2.2.5 | + | | − |
| Bacteroides_thetaiotaomicron_TSDC17.2_V2.2.4 | + | | − |
| Bacteroides_thetaiotaomicron_TSDC17.2_V2.1.3 | + | | − |
| Bacteroides_ovatus_TSDC17.2_V2.3.1 | + | | − |
| Bacteroides_ovatus_TSDC17.2_V2.2.2 | + | | − |
| Bacteroides_massiliensis_TSDC17.2_V2.1.3 | + | | − |
| Bacteroides_massiliensis_TSDC17.2_V2.1.2 | + | | − |
| Bacteroides_intestinalis_TSDC17.2_V2.1.9 | + | | − |
| Bacteroides_intestinalis_TSDC17.2_V2.1.7 | + | | − |
| Bacteroides_intestinalis_TSDC17.2_V2.1.5 | + | | − |
| Bacteroides_finegoldii_TSDC17.2_V2.1.4 | + | | − |
| Bacteroides_finegoldii_TSDC17.2_V2.1.2 | + | | − |
| Bacteroides_caccae_TSDC17.2_V2.1.7 | + | | − |
| Bacteroides_caccae_TSDC17.2_V2.1.6 | + | | − |
| Bacteroides_caccae_TSDC17.2_V2.1.3 | + | | − |
| Bacteroides_caccae_TSDC17.2_V2.1.1 | + | | − |
| Bacteroides_acidifaciens_TSDC17.2_V2.1.8 | + | | − |
| Bacteroides_acidifaciens_TSDC17.2_V2.1.3 | + | | − |
| Anaerococcus_TSDC17.2_V2.1.1 | | | |

The experimental design is shown in FIG. 28A, B. Groups of mice were colonized with one of three culture collections: the non-arrayed collection from the obese co-twin; the non-arrayed collection or the assembled 37-member consortium from lean co-twin. In the case of the 37-member consortium, the abundance of members in the non-arrayed collection was not preserved; rather, equivalent numbers of cells/strain were inoculated into recipient mice. Five days after inoculation, mice harboring the non-arrayed culture collection from the obese co-twin were co-housed with one another (negative control), or with mice containing the non-arrayed culture collection from the lean co-twin (positive control), or were co-housed with mice containing the ‘manufactured’ 37 member collection (5 mice/treatment group; total of 26 mice). FIG. 28A emphasizes how mice with 37-member consortium can be viewed as a prevention arm of the experiment: i.e., does the presence of these microbes prevent their host from gaining the level of adiposity achieved in mice harboring the obese co-twin microbiota?). These mice can also be viewed as a treatment arm: i.e., can the 37-member consortia ameliorate the increased adiposity phenotype that develops in mice colonized with the obese co-twin's culture collection? Quantitative MR was performed on days 1, 5, 15 and 19 of the 20 d long experiment revealed that the non-arrayed lean co-twin's culture collection produced a consistent reduction in adiposity in co-housed mice with the obese co-twin's culture collection. In 4 of 5 cages, co-housed mice with 37-member consortia (Ln37^{ch}) did not develop increased adiposity; in two of these 4 cages, the cage-mate with the obese co-twin's culture collection (Ob^{ch}) exhibited a very marked reduction in adiposity compared to the control group (dually housed mice, each with the obese co-twin's culture collection) (see arrows in FIG. 28C).

A similar experiment may be performed to further identify individual microbes from the 37-member consortium that, when inoculated into gnotobiotic mice and cohoused with mice containing the obese co-twin's culture collection, may induce reduced adiposity in the mice containing the obese co-twin's culture collection, and prevent increased adiposity in the mice containing the individual member of the 37-member consortium. In short, wherein individual members of the 37-member consortium may be used to colonize gnotobiotic mice. The mice may then be cohoused with mice containing the obese co-twin's culture collection, and the change in adiposity of all mice may be measured over time as described above. Such an experiment may identify individual members of the 37-member consortium that may induce reduce adiposity in obese mice, or prevent increased adiposity in mice harboring the individual member of the 37-member consortium.

### Example 16

### Discordance for Obesity Among Adult Female Twin Pairs in the MOAFTS Study Cohort

There were 3,427 participants in the MOAFTS wave 5 assessment; height and weight data were available for 3,416 (99.7%). The majority of participants (55.8%) were classified as lean (BMI 18.50-24.99 kg/m2), while 21.9% were classified as overweight (25-29.99 kg/m2), 18.3% as obese (≧30 kg/m2), and 3.98% as underweight (<18.5 kg/m2). African-Americans, who comprised 14.4% of the wave 5 sample, had significantly higher rates of overweight and obesity compared to European-Americans (EA) (32.5% and 36.6% vs. 20.1% and 15.2%, respectively p<0.001).

Height and weight data were available for 1,539 complete twin pairs participating in wave 5. 54.3% of the twin pairs were MZ. The mean difference in BMI between co-twins was 3.53 kg/m2 (SD 3.78 kg/m²). The mean difference in BMI was greater in DZ compared to MZ twin pairs (4.65±4.58 kg/m² versus 2.60±2.57 kg/m²; p<0.001). Using the criteria that one co-twin was obese and the other lean, 5.72% of twin pairs were defined as BMI discordant (mean difference=11.42±4.09 kg/m²). The rate of discordance was substantially lower for MZ pairs compared to DZ pairs (2.3% versus 9.9%; p<0.001) and AA twin pairs were more likely to be discordant than EA pairs (p=0.008). Alternatively, when BMI discordance was defined as a BMI difference ≧8 kg/m2, 18.3% of DZ pairs and 5.2% of MZ pairs were classified as discordant (p<0.001); AA pairs were again more likely to be discordant (21.6% vs. 9.4%; p<0.001).

### Methods for Examples 15 and 16.

Animal Husbandry.

All experiments involving mice were performed using protocols approved by the Washington University Animal Studies Committee. Germ-free adult male C57BL/6J mice were maintained in plastic flexible film gnotobiotic isolators under a strict 12 hr light cycle and fed an autoclaved low-fat, polysaccharide-rich chow diet (B&K diet 7378000) ad libitum.

Collection of Fecal Samples from Twin Pairs Discordant for Obesity and Transplantation of their Uncultured Fecal Microbiota into Germ-Free Mice.

Adult female twin pairs with a BMI discordance ranging from 6-10 kg/m² were recruited for this study. Procedures for obtaining their consent to provide fecal samples were approved by the Washington University Human Studies Committee. A single fecal sample was collected at t=0 and another 2 months later from each subject. Each sample was frozen immediately at −20° C., shipped in a frozen state to a biospecimen repository overseen by one of the authors, and then de-indentified. All samples were subsequently stored at −80° C. until the time of processing.

A given human fecal sample was homogenized with a mortar and pestle packed in dry ice. A 500 mg aliquot of the pulverized material was diluted in 5 mL of reduced PBS (PBS supplemented with 0.1% Resazurin (w/v), 0.05% L-cysteine-HCl), in an anaerobic Coy chamber (atmosphere, 70% N₂, 25% CO₂, 5% H₂), and then vortexed at room temperature for 5 min. The suspension was allowed to settle by gravity for 5 min, after which time the clarified supernatant was transferred to an anaerobic crimped tube that was then transported to the gnotobiotic mouse facility. The surface of the tube was sterilized by exposure for 20 min to chlorine dioxide in the transfer sleeve attached to the gnotobiotic isolator, transferred into the isolator. A 1 mL syringe was used to obtain a 200 μL aliquot of the suspension and was introduced by gavage into each adult C57BL6/J germ-free recipient. Transplant recipients were maintained in separate cages within an isolator dedicated to mice colonized with the same donor microbiota, except in the case of the co-housing experiments described below.

Analysis of Body Composition by Quantitative Magnetic Resonance Imaging (MRI).

Body composition was defined using MRI analysis (EchoMRI-3in1 instrument; EchoMRI, Houston, Tex.). Mice were transported from the gnotobiotic isolator to the MR instrument using in a HEPA filter capped glass vessel. Fat, lean and tissue-free body water were measured 1 d after gavage, and weekly for up to 5 weeks.

Sample Collection from Gnotobiotic Mice.

Fecal samples were collected at defined times after gavage from the mouse. At the time of sacrifice, luminal contents were collected as described in the Examples above at defined positions along the length of the gut (stomach, small intestinal segments 1, 2, 5, 9, 13 and 15 after its division into 16 equal-sized segments, cecum, and proximal and distal halves of the colon). Blood was harvested by retro-orbital phlebotomy into capillary blood collection tubes (BD), which were then centrifuged at 5,000×g at 4° C. for 5 min. The supernatant (serum) was frozen in liquid nitrogen for subsequent GC/MS analyses. Urine, also obtained at the time of sacrifice, was flash frozen in liquid nitrogen for metabolomic analysis. Both epididymal fat pads were recovered from each animal, by dissection, and weighed.

Multiplex Pyrosequencing of Amplicons Generated from Bacterial 16S rRNA Genes.

Genomic DNA was extracted from feces and gut samples using a bead-beating protocol. Briefly, a ˜500 mg aliquot of each pulverized frozen human fecal sample, or mouse fecal pellets (˜50 mg), or stomach, small intestinal, cecal or colonic contents (˜20 mg each); were re-suspended in a solution containing 500 μL of extraction buffer [200 mM Tris (pH 8.0), 200 mM NaCl, 20 mM EDTA], 210 μL of 20% SDS, 500 μL of phenol:chloroform:isoamyl alcohol (pH 7.9, 25:24:1, Ambion) and 500 μL of a slurry of 0.1-mm diameter zirconia/silica beads. Cells were then mechanically disrupted using a bead beater (Biospec, maximum setting; 3 min at room temperature), followed by extraction with phenol:chloroform:isoamyl alcohol and precipitation with isopropanol.

Amplicons of ˜330 bp, spanning variable region 2 (V2) of the 16S rRNA gene, were generated by using modified primers 8F and 338R incorporating sample specific barcodes as described in the Examples above and subjected to multiplex pyrosequencing (454 FLX Standard or Titanium chemistry). V2-16S rRNA sequences from Titanium chemistry were trimmed to FLX standard length and, together with the sequenced generated using FLX chemistry, filtered for low quality reads and assigned to a particular pyrosequencing bin according to their sample-specific barcodes. Sequencing errors were corrected using OTUpipe (QIIME v1.3) and classified into 97% ID OTUs using UCLUST. A representative OTU set was created using the most-abundant OTU from each bin. Reads were aligned using PyNAST. Taxonomy was assigned using RDP classifier.

Samples were rarefied at a depth of 815 OTUs/sample for time series studies of the fecal microbiota of gnotobiotic recipients of human microbiota and for the donor fecal microbiota) and 800 OTUs/sample in the case of the gut biogeography datasets. Data analysis (beta-diversity calculations, PCoA clustering) was performed using QIIME v1.3 and Vegan R package v 1.17-4 for pairwise distance analysis.

Shotgun Pyrosequencing of Total Community DNA.

For multiplex pyrosequencing (454 FLX Titanium chemistry), each of the 45 cecal DNA samples was randomly fragmented by nebulization to 500-800 bp and subsequently labeled with one of 12 MIDs (Multiplex Identifier; Roche) using the MID manufacturer's protocol (Rapid Library preparation for FLX Titanium). Equivalent amounts of up to 12 MID-labeled samples were pooled prior to each sequencer run. Shotgun reads were filtered to remove all reads <60 nt long, LR70 reads with at least one degenerate base (N), or reads with two continuous and/or three total degenerate bases, plus all duplicates (defined as sequences whose initial 20 nt were identical and shared an overall identity of >97% throughout the length of the shortest read). In the case of human fecal DNAs, all sequences with significant similarity to human reference genomes (BLASTN with e-value<10-5, bitscore>50, percent identity>75%) were removed. Comparable filtering against the mouse genome was performed for reads produced from samples obtained from recipient gnotobiotic animals.

All resulting filtered sequences were queried against the KEGG database (v58) using BLASTX. Sequences were annotated as the best hit in the database if (i) they had an E-value<10⁻⁵; (ii) the bit score was >50; and (iii) the query and subject were at least 50% identical after being aligned. If two entries were assigned as the best BLAST hit, the read was annotated with both entries. KO, E.C., and KEGG Pathway assignments were made using the “ko” file provided by KEGG. A matrix containing the counts for each KEGG annotation for each sample was generated for analysis with ShotgunFunctionalizeR (R package version 1.2-8).

Microbial RNA-Seq.

Each fecal pellet (˜50 mg) collected 15 or 17 days after colonization, was suspended while frozen in 1 ml of RNAprotect bacteria reagent (Qiagen), vortexed for 5 min at room temperature and centrifuged (10 min; 5,000×g; 4° C.). After decanting the supernatant, pelleted cells were suspended in 500 μL of extraction buffer [200 mM NaCl, 20 mM EDTA], 210 μl of 20% SDS, 500 μL of phenol:choloroform:isoamyl alcohol (pH 4.5, 125:24:1, Ambion), and 250 μL of acid-washed glass beads (Sigma-Aldrich, 212-300 μm diameter). Microbial cells were lysed by mechanical disruption using a bead beater (Biospec, maximum setting; 5 min at room temperature), followed by phenol:chloroform:isoamyl alcohol extraction and precipitation with isopropanol. RNA was treated with RNAse-free TURBO-DNAse (Ambion) and 5S rRNA and tRNAs were removed using MEGAClear columns (Ambion). A second DNAse treatment was performed (Baseline-ZERO DNAse; Epicenter). rRNA was initially depleted using MICROBexpress kit (Ambion) followed by a second MEGAClear purification. In addition, custom biotinylated oligonucleotides, directed against conserved regions of sequenced human gut bacterial rRNA genes were employed for streptavidin bead-based pulldowns. cDNA was synthesized using SuperScript II (Invitrogen), followed by second strand synthesis with RNAseH, E. coli DNA polymerase (NEB) and E. coli DNA ligase (NEB). Samples were sheared using a BioRuptor XL sonicator (Diagenode) and 150-200 bp fragments gel selected and prepared for sequencing.

Multiplexed microbial cDNA sequencing was performed using Illumina Hi-Seq2000 instruments to generate 23.7±6.5 million unidirectional 101 nt reads/sample. Reads were split according to 4-bp barcodes used to label each of four samples pooled together. After dividing sequences by barcode, reads were mapped to genes in a custom database of 127 sequenced human gut microbial genomes using the ssaha2 algorithm. A minimum score threshold of 42 was selected for ssaha, based on the distribution of scores for 101 nt barcoded reads. If a read mapped to more than one location in a genome or multiple genomes, the counts for each gene were added to the gene according to the gene's fraction of unique-match counts. Pseudo counts were added (i.e. added 1 count) to each gene prior to normalization to account for different sampling depths (i.e. normalized to reads/kb/million mapped reads).

Culturing Fecal Microbiota.

Each human fecal sample was pulverized while frozen and resuspended in pre-reduced PBS (0.1% Resazurin, 0.05% Cysteine/HCl; 15 mL/g feces). Samples were subsequently vortexed for 5 min and allowed to settle by gravity for 5 min to permit large, insoluble particles to settle. The supernatant was diluted 1000-fold in pre-reduced PBS and plated on 150 mm diameter plates containing pre-reduced, non-selective Gut Microbiota Medium (GMM, Goodman et al., 2011). Plates were incubated in a Coy chamber under anaerobic conditions for 7 d at 37° C. Colonies were subsequently harvested en masse from six plates by scraping (10 mL of pre-reduced PBS/plate). Glycerol (30%)/PBS stocks were stored in anaerobic glass vials at −80° C. 200 μL of the non-arrayed culture collection was used to gavage each germ-free recipient.

Creating a Clonally Arrayed Taxonomically Defined Sequenced Culture Collection.

Methods for creating clonally arrayed culture collections from frozen fecal samples were as described in the Examples above. A set of interfaces was also created for a Precision XS robot (BioTek) so that picking, arraying, and archiving of fecal bacterial culture collections can be done with speed and economy within a Coy anaerobic chamber. Taxonomies were assigned to each strain in an arrayed collection by 454 Titanium V2-16S rRNA pyrosequencing.

For a given culture collection, most strains (unique V2-16S rRNA sequence) are found in more than one well across the arrayed library. Therefore, several replicate wells of each strain were picked robotically from the 384-well plate, and streaked onto 8-well TYGS-agar plates. Plates were incubated under anaerobic conditions for 3 d at 37° C. in a Coy chamber. A single colony from each agar well was picked, grown in TYGS and archived as a TYGs/15% glycerol stock at −80° C. A small aliquot of each stock was taken for DNA extraction and subjected to multiplex genome sequencing with an Illumina HiSeq 2000 instrument [fold coverage 119±66 (mean±SD) coverage; range, 35-289].

TABLE 26
Metadata for 16S sequencing

| | | | | | Primer | Split | |
|---|---|---|---|---|---|---|---|
| Sample | Barcode | | | 454 run | Plate | Lib | |
| ID | Sequence | Linker Primer Sequence | 454 run IDs | Dates | Well | Reads | Donor |
| 2 | CTGTTCGTAGAG, | CATGCTGCCTCCCGTAGGAGT | GK_run_4.1 | Mar. 19, 2010 | 4_A1 | 1231 | 2 |
| | GTCACGACTATT | | | | | | |
| 3 | GACAGGAGATAG, | CATGCTGCCTCCCGTAGGAGT | GK_run_4.1 | Mar. 19, 2010 | 4_B1 | 2833 | 2 |
| | GTCTGACAGTTG | | | | | | |
| 5 | GACTCACTCAAT, | CATGCTGCCTCCCGTAGGAGT | GK_run_4.1 | Mar. 19, 2010 | 4_C1 | 1252 | 1 |
| | GTGTGCTATCAG | | | | | | |
| 6 | GAGCATTCTCTA, | CATGCTGCCTCCCGTAGGAGT | GK_run_4.1 | Mar. 19, 2010 | 4_D1 | 2753 | 1 |
| | TACAGATGGCTC | | | | | | |
| 7 | ACAGAGTCGGCT | CATGCTGCCTCCCGTAGGAGT | GK_run_4.2 | Mar. 25, 2010 | 1_B3 | 1634 | 2 |
| 8 | ACCGCAGAGTCA | CATGCTGCCTCCCGTAGGAGT | GK_run_4.2 | Mar. 25, 2010 | 1_C3 | 1518 | 2 |
| 9 | ACGGTGAGTGTC | CATGCTGCCTCCCGTAGGAGT | GK_run_4.2 | Mar. 25, 2010 | 1_D3 | 1493 | 2 |
| 10 | ACTCGATTCGAT | CATGCTGCCTCCCGTAGGAGT | GK_run_4.2 | Mar. 25, 2010 | 1_E3 | 1834 | 2 |
| 11 | AGACTGCGTACT | CATGCTGCCTCCCGTAGGAGT | GK_run_4.2 | Mar. 25, 2010 | 1_F3 | 1932 | 2 |
| 12 | AGCAGTCGCGAT | CATGCTGCCTCCCGTAGGAGT | GK_run_4.2 | Mar. 25, 2010 | 1_G3 | 1491 | 1 |
| 14 | AAGAGATGTCGA | CATGCTGCCTCCCGTAGGAGT | GK_run_4.2 | Mar. 25, 2010 | 1_A4 | 1857 | 1 |
| 15 | ACAGCAGTGGTC | CATGCTGCCTCCCGTAGGAGT | GK_run_4.2 | Mar. 25, 2010 | 1_B4 | 1904 | 1 |
| 16 | ACCTCGATCAGA | CATGCTGCCTCCCGTAGGAGT | GK_run_4.2 | Mar. 25, 2010 | 1_C4 | 1885 | 1 |
| 17 | ACGTACTCAGTG | CATGCTGCCTCCCGTAGGAGT | GK_run_4.2 | Mar. 25, 2010 | 1_D4 | 1854 | 2 |
| 18 | ACTCGCACAGGA | CATGCTGCCTCCCGTAGGAGT | GK_run_4.2 | Mar. 25, 2010 | 1_E4 | 1734 | 2 |
| 19 | AGAGAGCAAGTG | CATGCTGCCTCCCGTAGGAGT | GK_run_4.2 | Mar. 25, 2010 | 1_F4 | 1444 | 2 |
| 20 | AGCATATGAGAG | CATGCTGCCTCCCGTAGGAGT | GK_run_4.2 | Mar. 25, 2010 | 1_G4 | 1938 | 1 |
| 21 | AGGCTACACGAC | CATGCTGCCTCCCGTAGGAGT | GK_run_4.2 | Mar. 25, 2010 | 1_H4 | 1396 | 1 |
| 22 | AAGCTGCAGTCG | CATGCTGCCTCCCGTAGGAGT | GK_run_4.2 | Mar. 25, 2010 | 1_A5 | 2936 | 1 |
| 23 | ACAGCTAGCTTG | CATGCTGCCTCCCGTAGGAGT | GK_run_4.2 | Mar. 25, 2010 | 1_B5 | 2561 | 1 |
| 24 | ACCTGTCTCTCT | CATGCTGCCTCCCGTAGGAGT | GK_run_4.2 | Mar. 25, 2010 | 1_C5 | 2830 | 1 |
| 25 | AGTGTTCGATCG | CATGCTGCCTCCCGTAGGAGT | GK_run_4.3 | Mar. 30, 2010 | 2_A5 | 4245 | 2 |
| 26 | ATATCGCTACTG | CATGCTGCCTCCCGTAGGAGT | GK_run_4.3 | Mar. 30, 2010 | 2_B5 | 2842 | 2 |
| 27 | ATCTCTGGCATA | CATGCTGCCTCCCGTAGGAGT | GK_run_4.3 | Mar. 30, 2010 | 2_C5 | 3899 | 2 |
| 28 | ATGGATACGCTC | CATGCTGCCTCCCGTAGGAGT | GK_run_4.3 | Mar. 30, 2010 | 2_D5 | 3353 | 2 |
| 29 | CAACTCATCGTA | CATGCTGCCTCCCGTAGGAGT | GK_run_4.3 | Mar. 30, 2010 | 2_E5 | 3213 | 2 |
| 30 | CACTACTGTTGA | CATGCTGCCTCCCGTAGGAGT | GK_run_4.3 | Mar. 30, 2010 | 2_F5 | 2970 | 1 |
| 31 | CAGCGGTGACAT | CATGCTGCCTCCCGTAGGAGT | GK_run_4.3 | Mar. 30, 2010 | 2_G5 | 2532 | 1 |
| 32 | CATATACTCGCA | CATGCTGCCTCCCGTAGGAGT | GK_run_4.3 | Mar. 30, 2010 | 2_H5 | 3384 | 1 |
| 33 | AGTTAGTGCGTC | CATGCTGCCTCCCGTAGGAGT | GK_run_4.3 | Mar. 30, 2010 | 2_A6 | 4685 | 1 |
| 34 | ATATGCCAGTGC | CATGCTGCCTCCCGTAGGAGT | GK_run_4.3 | Mar. 30, 2010 | 2_B6 | 3926 | 1 |
| 35 | ATCTGAGCTGGT | CATGCTGCCTCCCGTAGGAGT | GK_run_4.3 | Mar. 30, 2010 | 2_C6 | 3573 | 2 |
| 36 | ATGGCAGCTCTA | CATGCTGCCTCCCGTAGGAGT | GK_run_4.3 | Mar. 30, 2010 | 2_D6 | 3211 | 2 |
| 37 | CAAGATCGACTC | CATGCTGCCTCCCGTAGGAGT | GK_run_4.3 | Mar. 30, 2010 | 2_E6 | 3509 | 2 |
| 38 | CACTCAACAGAC | CATGCTGCCTCCCGTAGGAGT | GK_run_4.3 | Mar. 30, 2010 | 2_F6 | 2617 | 1 |
| 39 | CAGCTAGAACGC | CATGCTGCCTCCCGTAGGAGT | GK_run_4.3 | Mar. 30, 2010 | 2_G6 | 3000 | 1 |
| 40 | CATATCGCAGTT | CATGCTGCCTCCCGTAGGAGT | GK_run_4.3 | Mar. 30, 2010 | 2_H6 | 2264 | 1 |
| 41 | AGTTCAGACGCT | CATGCTGCCTCCCGTAGGAGT | GK_run_4.3 | Mar. 30, 2010 | 2_A7 | 3821 | 1 |
| 42 | ATCACGTAGCGG | CATGCTGCCTCCCGTAGGAGT | GK_run_4.3 | Mar. 30, 2010 | 2_B7 | 3316 | 1 |
| 43 | ATCTGGTGCTAT | CATGCTGCCTCCCGTAGGAGT | GK_run_4.3 | Mar. 30, 2010 | 2_C7 | 3703 | 2 |
| 44 | ATGGCGTGCACA | CATGCTGCCTCCCGTAGGAGT | GK_run_4.3 | Mar. 30, 2010 | 2_D7 | 2682 | 2 |
| 45 | CAAGTGAGAGAG | CATGCTGCCTCCCGTAGGAGT | GK_run_4.3 | Mar. 30, 2010 | 2_E7 | 3429 | 2 |
| 46 | CACTCTGATTAG | CATGCTGCCTCCCGTAGGAGT | GK_run_4.3 | Mar. 30, 2010 | 2_F7 | 3117 | 2 |
| 47 | CAGGTGCTACTA | CATGCTGCCTCCCGTAGGAGT | GK_run_4.3 | Mar. 30, 2010 | 2_G7 | 2917 | 2 |
| 48 | CATCAGCGTGTA | CATGCTGCCTCCCGTAGGAGT | GK_run_4.3 | Mar. 30, 2010 | 2_H7 | 3119 | 1 |
| 49 | AGTTCTACGTCA | CATGCTGCCTCCCGTAGGAGT | GK_run_4.3 | Mar. 30, 2010 | 2_A8 | 4706 | 1 |
| 50 | ATCACTAGTCAC | CATGCTGCCTCCCGTAGGAGT | GK_run_4.3 | Mar. 30, 2010 | 2_B8 | 3644 | 1 |
| 51 | ATCTTAGACTGC | CATGCTGCCTCCCGTAGGAGT | GK_run_4.3 | Mar. 30, 2010 | 2_C8 | 2541 | 1 |
| 52 | ATGGTCTACTAC | CATGCTGCCTCCCGTAGGAGT | GK_run_4.3 | Mar. 30, 2010 | 2_D8 | 3143 | 1 |
| 53 | CACACGTGAGCA | CATGCTGCCTCCCGTAGGAGT | GK_run_4.3 | Mar. 30, 2010 | 2_E8 | 3225 | 2 |
| 54 | CACTGGTATATC | CATGCTGCCTCCCGTAGGAGT | GK_run_4.3 | Mar. 30, 2010 | 2_F8 | 3485 | 2 |
| 55 | CAGTACGATCTT | CATGCTGCCTCCCGTAGGAGT | GK_run_4.3 | Mar. 30, 2010 | 2_G8 | 2971 | 2 |
| 56 | CATCATGAGGCT | CATGCTGCCTCCCGTAGGAGT | GK_run_4.3 | Mar. 30, 2010 | 2_H8 | 3508 | 1 |
| 57 | ATAATCTCGTCG | CATGCTGCCTCCCGTAGGAGT | GK_run_4.3 | Mar. 30, 2010 | 2_A9 | 5065 | 1 |
| 58 | ATCAGGCGTGTG | CATGCTGCCTCCCGTAGGAGT | GK_run_4.3 | Mar. 30, 2010 | 2_B9 | 3503 | 1 |
| 59 | ATGACCATCGTG | CATGCTGCCTCCCGTAGGAGT | GK_run_4.3 | Mar. 30, 2010 | 2_C9 | 4798 | 1 |
| 60 | ATGTACGGCGAC | CATGCTGCCTCCCGTAGGAGT | GK_run_4.3 | Mar. 30, 2010 | 2_D9 | 3642 | 1 |
| 61 | GATCAGAAGATG | CATGCTGCCTCCCGTAGGAGT | GK_run_4.1 | Mar. 19, 2010 | 4_E1 | 2146 | 2 |
| 62 | GCAATAGCTGCT | CATGCTGCCTCCCGTAGGAGT | GK_run_4.1 | Mar. 19, 2010 | 4_F1 | 2757 | 2 |
| 63 | GCATATAGTCTC | CATGCTGCCTCCCGTAGGAGT | GK_run_4.1 | Mar. 19, 2010 | 4_G1 | 2347 | 2 |
| 64 | CTTAGCACATCA | CATGCTGCCTCCCGTAGGAGT | GK_run_4.1 | Mar. 19, 2010 | 4_A2 | 3189 | 2 |
| 65 | GACAGTTACTGC | CATGCTGCCTCCCGTAGGAGT | GK_run_4.1 | Mar. 19, 2010 | 4_B2 | 2423 | 2 |
| 66 | GACTCGAATCGT | CATGCTGCCTCCCGTAGGAGT | GK_run_4.1 | Mar. 19, 2010 | 4_C2 | 2105 | 1 |
| 67 | GAGCTGGCTGAT | CATGCTGCCTCCCGTAGGAGT | GK_run_4.1 | Mar. 19, 2010 | 4_D2 | 1986 | 1 |
| 68 | GATCCGACACTA | CATGCTGCCTCCCGTAGGAGT | GK_run_4.1 | Mar. 19, 2010 | 4_E2 | 2738 | 1 |
| 69 | GCACATCGAGCA | CATGCTGCCTCCCGTAGGAGT | GK_run_4.1 | Mar. 19, 2010 | 4_F2 | 2227 | 1 |
| 70 | GCATCGTCAACA | CATGCTGCCTCCCGTAGGAGT | GK_run_4.1 | Mar. 19, 2010 | 4_G2 | 3092 | 1 |
| 71 | GCGTTACACACA | CATGCTGCCTCCCGTAGGAGT | GK_run_4.1 | Mar. 19, 2010 | 4_H2 | 2686 | 2 |
| 72 | CTTGATGCGTAT | CATGCTGCCTCCCGTAGGAGT | GK_run_4.1 | Mar. 19, 2010 | 4_A3 | 2552 | 2 |
| 73 | GACATCGGCTAT | CATGCTGCCTCCCGTAGGAGT | GK_run_4.1 | Mar. 19, 2010 | 4_B3 | 2489 | 2 |
| 74 | GACTGATCATCT | CATGCTGCCTCCCGTAGGAGT | GK_run_4.1 | Mar. 19, 2010 | 4_C3 | 3026 | 1 |
| 75 | GAGGCTCATCAT | CATGCTGCCTCCCGTAGGAGT | GK_run_4.1 | Mar. 19, 2010 | 4_D3 | 2671 | 1 |
| 76 | GATCGCAGGTGT | CATGCTGCCTCCCGTAGGAGT | GK_run_4.1 | Mar. 19, 2010 | 4_E3 | 2940 | 1 |
| 77 | GCACGACAACAC | CATGCTGCCTCCCGTAGGAGT | GK_run_4.1 | Mar. 19, 2010 | 4_F3 | 2811 | 1 |
| 78 | GCATGTGCATGT | CATGCTGCCTCCCGTAGGAGT | GK_run_4.1 | Mar. 19, 2010 | 4_G3 | 3283 | 1 |
| 79 | TATTCGTGTCAG | CATGCTGCCTCCCGTAGGAGT | GK_run_4.4 | Apr. 7, 2010 | 6_A9 | 3865 | 2 |
| 80 | TCAGGACTGTGT | CATGCTGCCTCCCGTAGGAGT | GK_run_4.4 | Apr. 7, 2010 | 6_B9 | 2719 | 2 |
| 81 | TCCTGAGATACG | CATGCTGCCTCCCGTAGGAGT | GK_run_4.4 | Apr. 7, 2010 | 6_C9 | 3072 | 2 |
| 82 | TCGTACGTCATA | CATGCTGCCTCCCGTAGGAGT | GK_run_4.4 | Apr. 7, 2010 | 6_D9 | 3757 | 2 |
| 83 | TCTCTAGAGCAT | CATGCTGCCTCCCGTAGGAGT | GK_run_4.4 | Apr. 7, 2010 | 6_E9 | 3255 | 2 |
| 84 | GCTAAGAGAGTA | CATGCTGCCTCCCGTAGGAGT | GK_run_4.1 | Mar. 19, 2010 | 4_H3 | 3353 | 2 |
| 85 | TGAGACGTGCTT | CATGCTGCCTCCCGTAGGAGT | GK_run_4.4 | Apr. 7, 2010 | 6_F9 | 2567 | 2 |
| 86 | TGCATTACGCAT | CATGCTGCCTCCCGTAGGAGT | GK_run_4.4 | Apr. 7, 2010 | 6_G9 | 2460 | 2 |
| 87 | TGGATATGCGCT | CATGCTGCCTCCCGTAGGAGT | GK_run_4.4 | Apr. 7, 2010 | 6_H9 | 3075 | 2 |
| 88 | TCAACAGCATCG | CATGCTGCCTCCCGTAGGAGT | GK_run_4.4 | Apr. 7, 2010 | 6_A10 | 3998 | 2 |
| 89 | TCAGTACGAGGC | CATGCTGCCTCCCGTAGGAGT | GK_run_4.4 | Apr. 7, 2010 | 6_B10 | 4152 | 2 |
| 90 | CTTGTGTCGATA | CATGCTGCCTCCCGTAGGAGT | GK_run_4.1 | Mar. 19, 2010 | 4_A4 | 3128 | 2 |
| 91 | TCGAATCACAGC | CATGCTGCCTCCCGTAGGAGT | GK_run_4.4 | Apr. 7, 2010 | 6_C10 | 2934 | 2 |
| 92 | TCGTCGATAATC | CATGCTGCCTCCCGTAGGAGT | GK_run_4.4 | Apr. 7, 2010 | 6_D10 | 3590 | 2 |
| 93 | TCTCTCCGTCGA | CATGCTGCCTCCCGTAGGAGT | GK_run_4.4 | Apr. 7, 2010 | 6_E10 | 2537 | 2 |
| 94 | TGAGAGAGCATA | CATGCTGCCTCCCGTAGGAGT | GK_run_4.4 | Apr. 7, 2010 | 6_F10 | 2656 | 2 |
| 95 | TGCGCGAATACT | CATGCTGCCTCCCGTAGGAGT | GK_run_4.4 | Apr. 7, 2010 | 6_G10 | 3900 | 2 |
| 96 | GACCACTACGAT | CATGCTGCCTCCCGTAGGAGT | GK_run_4.1 | Mar. 19, 2010 | 4_B4 | 3207 | 2 |
| 97 | TGGCTCTACAGA | CATGCTGCCTCCCGTAGGAGT | GK_run_4.4 | Apr. 7, 2010 | 6_H10 | 6179 | 2 |
| 98 | TCAATCTAGCGT | CATGCTGCCTCCCGTAGGAGT | GK_run_4.4 | Apr. 7, 2010 | 6_A11 | 2758 | 2 |
| 102 | GACTGCATCTTA | CATGCTGCCTCCCGTAGGAGT | GK_run_4.1 | Mar. 19, 2010 | 4_C4 | 3461 | 2 |
| 108 | GAGTAGCTCGTG | CATGCTGCCTCCCGTAGGAGT | GK_run_4.1 | Mar. 19, 2010 | 4_D4 | 2931 | 2 |
| 110 | TCGAGACGCTTA | CATGCTGCCTCCCGTAGGAGT | GK_run_4.4 | Apr. 7, 2010 | 6_C12 | 2488 | 1 |
| 111 | TCGTGTCTATAG | CATGCTGCCTCCCGTAGGAGT | GK_run_4.4 | Apr. 7, 2010 | 6_D12 | 3204 | 1 |
| 112 | TCTGCGTACTAA | CATGCTGCCTCCCGTAGGAGT | GK_run_4.4 | Apr. 7, 2010 | 6_E12 | 3109 | 1 |
| 113 | TGAGCGATTCTG | CATGCTGCCTCCCGTAGGAGT | GK_run_4.4 | Apr. 7, 2010 | 6_F12 | 2739 | 1 |
| 114 | GATCGTCCAGAT | CATGCTGCCTCCCGTAGGAGT | GK_run_4.1 | Mar. 19, 2010 | 4_E4 | 1893 | 1 |
| 115 | TGCGTCAGTTAG | CATGCTGCCTCCCGTAGGAGT | GK_run_4.4 | Apr. 7, 2010 | 6_G12 | 3190 | 1 |
| 116 | TGTACACGGCGA | CATGCTGCCTCCCGTAGGAGT | GK_run_4.4 | Apr. 7, 2010 | 6_H12 | 3369 | 1 |
| 120 | GCACTCGTTAGA | CATGCTGCCTCCCGTAGGAGT | GK_run_4.1 | Mar. 19, 2010 | 4_F4 | 3832 | 1 |
| 126 | GCATTGCGTGAG | CATGCTGCCTCCCGTAGGAGT | GK_run_4.1 | Mar. 19, 2010 | 4_G4 | 1654 | 1 |
| 132 | GCTAGATGCCAG | CATGCTGCCTCCCGTAGGAGT | GK_run_4.1 | Mar. 19, 2010 | 4_H4 | 4293 | 1 |
| 144 | CATCGTATCAAC | CATGCTGCCTCCCGTAGGAGT | GK_run_4.5 | Apr. 21, 2010 | 2_H9 | 2199 | 2 |
| 150 | GACTGTCATGCA | CATGCTGCCTCCCGTAGGAGT | GK_run_4.1 | Mar. 19, 2010 | 4_C5 | 3645 | 2 |
| 156 | GAGTATGCAGCC | CATGCTGCCTCCCGTAGGAGT | GK_run_4.1 | Mar. 19, 2010 | 4_D5 | 3804 | 2 |
| 162 | GATCTATCCGAG | CATGCTGCCTCCCGTAGGAGT | GK_run_4.1 | Mar. 19, 2010 | 4_E5 | 2547 | 1 |
| 168 | GCACTGAGACGT | CATGCTGCCTCCCGTAGGAGT | GK_run_4.1 | Mar. 19, 2010 | 4_F5 | 2587 | 1 |
| 174 | GCCACTGATAGT | CATGCTGCCTCCCGTAGGAGT | GK_run_4.1 | Mar. 19, 2010 | 4_G5 | 5182 | 1 |
| 180 | GCTAGTCTGAAC | CATGCTGCCTCCCGTAGGAGT | GK_run_4.1 | Mar. 19, 2010 | 4_H5 | 4795 | 1 |
| 186 | GAACTGTATCTC | CATGCTGCCTCCCGTAGGAGT | GK_run_4.1 | Mar. 19, 2010 | 4_A6 | 4916 | 1 |
| 187 | ACGTCTGTAGCA | CATGCTGCCTCCCGTAGGAGT | GK_run_4.2 | Mar. 25, 2010 | 1_D5 | 2958 | 2 |
| 188 | ACTCTTCTAGAG | CATGCTGCCTCCCGTAGGAGT | GK_run_4.2 | Mar. 25, 2010 | 1_E5 | 2558 | 2 |
| 189 | AGAGCAAGAGCA | CATGCTGCCTCCCGTAGGAGT | GK_run_4.2 | Mar. 25, 2010 | 1_F5 | 2598 | 2 |
| 190 | AGCCATACTGAC | CATGCTGCCTCCCGTAGGAGT | GK_run_4.2 | Mar. 25, 2010 | 1_G5 | 2597 | 2 |
| 191 | AGGTGTGATCGC | CATGCTGCCTCCCGTAGGAGT | GK_run_4.2 | Mar. 25, 2010 | 1_H5 | 2756 | 2 |
| 192 | AATCAGTCTCGT | CATGCTGCCTCCCGTAGGAGT | GK_run_4.2 | Mar. 25, 2010 | 1_A6 | 2861 | 1 |
| 193 | ACAGTGCTTCAT | CATGCTGCCTCCCGTAGGAGT | GK_run_4.2 | Mar. 25, 2010 | 1_B6 | 2886 | 1 |
| 194 | ACGACGTCTTAG | CATGCTGCCTCCCGTAGGAGT | GK_run_4.2 | Mar. 25, 2010 | 1_C6 | 2451 | 1 |
| 195 | ACGTGAGAGAAT | CATGCTGCCTCCCGTAGGAGT | GK_run_4.2 | Mar. 25, 2010 | 1_D6 | 2718 | 1 |
| 196 | ACTGACAGCCAT | CATGCTGCCTCCCGTAGGAGT | GK_run_4.2 | Mar. 25, 2010 | 1_E6 | 2845 | 1 |
| 197 | AGAGTAGCTAAG | CATGCTGCCTCCCGTAGGAGT | GK_run_4.2 | Mar. 25, 2010 | 1_F6 | 3027 | 2 |
| 198 | AGCGACTGTGCA | CATGCTGCCTCCCGTAGGAGT | GK_run_4.2 | Mar. 25, 2010 | 1_G6 | 2666 | 2 |
| 199 | AGTACGCTCGAG | CATGCTGCCTCCCGTAGGAGT | GK_run_4.2 | Mar. 25, 2010 | 1_H6 | 2054 | 2 |
| 200 | AATCGTGACTCG | CATGCTGCCTCCCGTAGGAGT | GK_run_4.2 | Mar. 25, 2010 | 1_A7 | 2368 | 1 |
| 201 | ACAGTTGCGCGA | CATGCTGCCTCCCGTAGGAGT | GK_run_4.2 | Mar. 25, 2010 | 1_B7 | 2191 | 1 |
| 202 | ACGAGTGCTATC | CATGCTGCCTCCCGTAGGAGT | GK_run_4.2 | Mar. 25, 2010 | 1_C7 | 3087 | 1 |
| 203 | ACGTGCCGTAGA | CATGCTGCCTCCCGTAGGAGT | GK_run_4.2 | Mar. 25, 2010 | 1_D7 | 1995 | 1 |
| 204 | ACTGATCCTAGT | CATGCTGCCTCCCGTAGGAGT | GK_run_4.2 | Mar. 25, 2010 | 1_E7 | 2715 | 1 |
| 205 | CACAGCTCGAAT | CATGCTGCCTCCCGTAGGAGT | GK_run_4.3 | Mar. 30, 2010 | 2_E9 | 3780 | 2 |
| 207 | CAGTCACTAACG | CATGCTGCCTCCCGTAGGAGT | GK_run_4.3 | Mar. 30, 2010 | 2_G9 | 4148 | 2 |
| 208 | CATCGTATCAAC | CATGCTGCCTCCCGTAGGAGT | GK_run_4.3 | Mar. 30, 2010 | 2_H9 | 4183 | 2 |
| 209 | ATACACGTGGCG | CATGCTGCCTCCCGTAGGAGT | GK_run_4.3 | Mar. 30, 2010 | 2_A10 | 4825 | 2 |
| 210 | ATCCGATCACAG | CATGCTGCCTCCCGTAGGAGT | GK_run_4.3 | Mar. 30, 2010 | 2_B10 | 3543 | 1 |
| 211 | ATGACTCATTCG | CATGCTGCCTCCCGTAGGAGT | GK_run_4.3 | Mar. 30, 2010 | 2_C10 | 2816 | 1 |
| 212 | ATGTCACCGTGA | CATGCTGCCTCCCGTAGGAGT | GK_run_4.3 | Mar. 30, 2010 | 2_D10 | 3590 | 1 |
| 213 | CACAGTGGACGT | CATGCTGCCTCCCGTAGGAGT | GK_run_4.3 | Mar. 30, 2010 | 2_E10 | 3382 | 1 |
| 214 | CAGACATTGCGT | CATGCTGCCTCCCGTAGGAGT | GK_run_4.3 | Mar. 30, 2010 | 2_F10 | 3892 | 1 |
| 215 | CAGTCGAAGCTG | CATGCTGCCTCCCGTAGGAGT | GK_run_4.3 | Mar. 30, 2010 | 2_G10 | 2712 | 2 |
| 216 | CATCTGTAGCGA | CATGCTGCCTCCCGTAGGAGT | GK_run_4.3 | Mar. 30, 2010 | 2_H10 | 4157 | 2 |
| 217 | ATACAGAGCTCC | CATGCTGCCTCCCGTAGGAGT | GK_run_4.3 | Mar. 30, 2010 | 2_A11 | 4694 | 2 |
| 218 | ATCCTCAGTAGT | CATGCTGCCTCCCGTAGGAGT | GK_run_4.3 | Mar. 30, 2010 | 2_B11 | 3396 | 1 |
| 219 | ATGAGACTCCAC | CATGCTGCCTCCCGTAGGAGT | GK_run_4.3 | Mar. 30, 2010 | 2_C11 | 3888 | 1 |
| 220 | ATGTGCACGACT | CATGCTGCCTCCCGTAGGAGT | GK_run_4.3 | Mar. 30, 2010 | 2_D11 | 3586 | 1 |
| 221 | CACATCTAACAC | CATGCTGCCTCCCGTAGGAGT | GK_run_4.3 | Mar. 30, 2010 | 2_E11 | 3484 | 1 |
| 222 | CAGACTCGCAGA | CATGCTGCCTCCCGTAGGAGT | GK_run_4.3 | Mar. 30, 2010 | 2_F11 | 3945 | 1 |
| 223 | CAGTGATCCTAG | CATGCTGCCTCCCGTAGGAGT | GK_run_4.3 | Mar. 30, 2010 | 2_G11 | 3838 | 2 |
| 224 | CATGAGTGCTAC | CATGCTGCCTCCCGTAGGAGT | GK_run_4.3 | Mar. 30, 2010 | 2_H11 | 3414 | 2 |
| 225 | ATACGTCTTCGA | CATGCTGCCTCCCGTAGGAGT | GK_run_4.3 | Mar. 30, 2010 | 2_A12 | 5041 | 2 |
| 226 | ATCGATCTGTGG | CATGCTGCCTCCCGTAGGAGT | GK_run_4.3 | Mar. 30, 2010 | 2_B12 | 4240 | 2 |
| 227 | ATGATCGAGAGA | CATGCTGCCTCCCGTAGGAGT | GK_run_4.3 | Mar. 30, 2010 | 2_C12 | 4558 | 2 |
| 228 | ATGTGTCGACTT | CATGCTGCCTCCCGTAGGAGT | GK_run_4.3 | Mar. 30, 2010 | 2_D12 | 3653 | 1 |
| 229 | CACATTGTGAGC | CATGCTGCCTCCCGTAGGAGT | GK_run_4.3 | Mar. 30, 2010 | 2_E12 | 3431 | 1 |
| 230 | CAGAGGAGCTCT | CATGCTGCCTCCCGTAGGAGT | GK_run_4.3 | Mar. 30, 2010 | 2_F12 | 4546 | 1 |
| 231 | CAGTGCATATGC | CATGCTGCCTCCCGTAGGAGT | GK_run_4.3 | Mar. 30, 2010 | 2_G12 | 3572 | 1 |
| 232 | CATGCAGACTGT | CATGCTGCCTCCCGTAGGAGT | GK_run_4.3 | Mar. 30, 2010 | 2_H12 | 3448 | 1 |
| 233 | TAGTTGCGAGTC | CATGCTGCCTCCCGTAGGAGT | GK_run_4.3 | Mar. 30, 2010 | 6_A1 | 1448 | 2 |
| 234 | TCACGATTAGCG | CATGCTGCCTCCCGTAGGAGT | GK_run_4.3 | Mar. 30, 2010; | 6_B1 | 2084 | 2 |
| | | | GK_run_5.1 | Jul. 6, 2010 | | | |
| 235 | TCATCGCGATAT | CATGCTGCCTCCCGTAGGAGT | GK_run_4.3 | Mar. 30, 2010 | 6_C1 | 1143 | 2 |
| 236 | TCGAGCGAATCT | CATGCTGCCTCCCGTAGGAGT | GK_run_4.3 | Mar. 30, 2010 | 6_D1 | 1160 | 1 |
| 238 | TCACTATGGTCA | CATGCTGCCTCCCGTAGGAGT | GK_run_4.3 | Mar. 30, 2010 | 6_B2 | 1650 | 1 |
| 240 | TCGAGGACTGCA | CATGCTGCCTCCCGTAGGAGT | GK_run_4.3 | Mar. 30, 2010; | 6_D2 | 2005 | 1 |
| | | | GK_run_5.1 | Jul. 6, 2010 | | | |
| 261 | GACGAGTCAGTC | CATGCTGCCTCCCGTAGGAGT | GK_run_4.1 | Mar. 19, 2010 | 4_B6 | 2652 | 2 |
| 262 | GACTTCAGTGTG | CATGCTGCCTCCCGTAGGAGT | GK_run_4.1 | Mar. 19, 2010 | 4_C6 | 3091 | 2 |
| 263 | GAGTCTGAGTCT | CATGCTGCCTCCCGTAGGAGT | GK_run_4.1 | Mar. 19, 2010 | 4_D6 | 2839 | 2 |
| 265 | GCAGCACGTTGA | CATGCTGCCTCCCGTAGGAGT | GK_run_4.1 | Mar. 19, 2010; | 4_F6 | 1367 | 1 |
| | | | GK_run_5.1 | Jul. 6, 2010 | | | |
| 266 | GCCAGAGTCGTA | CATGCTGCCTCCCGTAGGAGT | GK_run_4.1 | Mar. 19, 2010 | 4_G6 | 2358 | 1 |
| 267 | GCTATCACGAGT | CATGCTGCCTCCCGTAGGAGT | GK_run_4.1 | Mar. 19, 2010 | 4_H6 | 3173 | 1 |
| 268 | GAAGAGTGATCA | CATGCTGCCTCCCGTAGGAGT | GK_run_4.1 | Mar. 19, 2010 | 4_A7 | 2451 | 1 |
| 269 | GACGATATCGCG | CATGCTGCCTCCCGTAGGAGT | GK_run_4.1 | Mar. 19, 2010 | 4_B7 | 3617 | 2 |
| 270 | GAGAATACGTGA | CATGCTGCCTCCCGTAGGAGT | GK_run_4.1 | Mar. 19, 2010 | 4_C7 | 2850 | 2 |
| 271 | GAGTGAGTACAA | CATGCTGCCTCCCGTAGGAGT | GK_run_4.1 | Mar. 19, 2010 | 4_D7 | 4874 | 2 |
| 272 | GATCTTCAGTAC | CATGCTGCCTCCCGTAGGAGT | GK_run_4.1 | Mar. 19, 2010 | 4_E7 | 3025 | 1 |
| 273 | GCAGCCGAGTAT | CATGCTGCCTCCCGTAGGAGT | GK_run_4.1 | Mar. 19, 2010 | 4_F7 | 2346 | 1 |
| 274 | GCCTATACTACA | CATGCTGCCTCCCGTAGGAGT | GK_run_4.1 | Mar. 19, 2010 | 4_G7 | 3861 | 1 |
| 275 | GCTATTCGACAT | CATGCTGCCTCCCGTAGGAGT | GK_run_4.1 | Mar. 19, 2010 | 4_H7 | 2016 | 1 |
| 276 | GAAGCTACTGTC | CATGCTGCCTCCCGTAGGAGT | GK_run_4.1 | Mar. 19, 2010 | 4_A8 | 4143 | 1 |
| 277 | GACGCAGTAGCT | CATGCTGCCTCCCGTAGGAGT | GK_run_4.1 | Mar. 19, 2010 | 4_B8 | 3776 | 2 |
| 278 | GAGACAGCTTGC | CATGCTGCCTCCCGTAGGAGT | GK_run_4.1 | Mar. 19, 2010 | 4_C8 | 3315 | 2 |
| 279 | GAGTGGTAGAGA | CATGCTGCCTCCCGTAGGAGT | GK_run_4.1 | Mar. 19, 2010 | 4_D8 | 4263 | 2 |
| 280 | GATGATCGCCGA | CATGCTGCCTCCCGTAGGAGT | GK_run_4.1 | Mar. 19, 2010 | 4_E8 | 4051 | 2 |
| 281 | GCAGGATAGATA | CATGCTGCCTCCCGTAGGAGT | GK_run_4.1 | Mar. 19, 2010 | 4_F8 | 1899 | 2 |
| 282 | GCGACTTGTGTA | CATGCTGCCTCCCGTAGGAGT | GK_run_4.1 | Mar. 19, 2010 | 4_G8 | 2026 | 1 |
| 284 | GAAGTCTCGCAT | CATGCTGCCTCCCGTAGGAGT | GK_run_4.1 | Mar. 19, 2010 | 4_A9 | 2928 | 1 |
| 285 | GACGCTAGTTCA | CATGCTGCCTCCCGTAGGAGT | GK_run_4.1 | Mar. 19, 2010 | 4_B9 | 1767 | 1 |
| 287 | ATGTCACCGTGA | CATGCTGCCTCCCGTAGGAGT | GK_run_4.5 | Apr. 21, 2010 | 2_D10 | 2764 | 2 |
| 288 | GATGCATGACGC | CATGCTGCCTCCCGTAGGAGT | GK_run_4.1 | Mar. 19, 2010 | 4_E9 | 3009 | 2 |
| 289 | GCAGGCAGTACT | CATGCTGCCTCCCGTAGGAGT | GK_run_4.1 | Mar. 19, 2010 | 4_F9 | 3693 | 2 |
| 290 | GCGAGATCCAGT | CATGCTGCCTCCCGTAGGAGT | GK_run_4.1 | Mar. 19, 2010 | 4_G9 | 3696 | 1 |
| 291 | GCTCGCTACTTC | CATGCTGCCTCCCGTAGGAGT | GK_run_4.1 | Mar. 19, 2010 | 4_H9 | 2907 | 1 |
| 292 | GAATGATGAGTG | CATGCTGCCTCCCGTAGGAGT | GK_run_4.1 | Mar. 19, 2010 | 4_A10 | 3714 | 1 |
| 293 | GACGTTGCACAG | CATGCTGCCTCCCGTAGGAGT | GK_run_4.1 | Mar. 19, 2010 | 4_B10 | 3562 | 1 |
| 294 | GAGAGCTCTACG | CATGCTGCCTCCCGTAGGAGT | GK_run_4.1 | Mar. 19, 2010 | 4_C10 | 2135 | 1 |
| 295 | AGAGTCCTGAGC | CATGCTGCCTCCCGTAGGAGT | GK_run_4.2 | Mar. 25, 2010 | 1_F7 | 3232 | 2 |
| 296 | AGCGAGCTATCT | CATGCTGCCTCCCGTAGGAGT | GK_run_4.2 | Mar. 25, 2010 | 1_G7 | 2868 | 2 |
| 297 | AGTACTGCAGGC | CATGCTGCCTCCCGTAGGAGT | GK_run_4.2 | Mar. 25, 2010 | 1_H7 | 2498 | 2 |
| 298 | ACACACTATGGC | CATGCTGCCTCCCGTAGGAGT | GK_run_4.2 | Mar. 25, 2010 | 1_A8 | 2242 | 2 |
| 299 | ACATCACTTAGC | CATGCTGCCTCCCGTAGGAGT | GK_run_4.2 | Mar. 25, 2010 | 1_B8 | 2942 | 2 |
| 300 | ACGATGCGACCA | CATGCTGCCTCCCGTAGGAGT | GK_run_4.2 | Mar. 25, 2010 | 1_C8 | 2635 | 1 |
| 301 | ACGTTAGCACAC | CATGCTGCCTCCCGTAGGAGT | GK_run_4.2 | Mar. 25, 2010 | 1_D8 | 2630 | 1 |
| 302 | ACTGTACGCGTA | CATGCTGCCTCCCGTAGGAGT | GK_run_4.2 | Mar. 25, 2010 | 1_E8 | 2806 | 1 |
| 303 | AGATACACGCGC | CATGCTGCCTCCCGTAGGAGT | GK_run_4.2 | Mar. 25, 2010 | 1_F8 | 2814 | 1 |
| 304 | AGCGCTGATGTG | CATGCTGCCTCCCGTAGGAGT | GK_run_4.2 | Mar. 25, 2010 | 1_G8 | 2181 | 1 |
| 305 | AGTAGTATCCTC | CATGCTGCCTCCCGTAGGAGT | GK_run_4.2 | Mar. 25, 2010 | 1_H8 | 1951 | 2 |
| 306 | CAGTCGAAGCTG | CATGCTGCCTCCCGTAGGAGT | GK_run_4.5 | Apr. 21, 2010 | 2_G10 | 1452 | 2 |
| 307 | CATCTGTAGCGA | CATGCTGCCTCCCGTAGGAGT | GK_run_4.5 | Apr. 21, 2010 | 2_H10 | 1764 | 2 |
| 308 | ACGCAACTGCTA | CATGCTGCCTCCCGTAGGAGT | GK_run_4.2 | Mar. 25, 2010 | 1_C9 | 5485 | 1 |
| 309 | ACTACAGCCTAT | CATGCTGCCTCCCGTAGGAGT | GK_run_4.2 | Mar. 25, 2010 | 1_D9 | 6799 | 1 |
| 310 | ACTGTCGAAGCT | CATGCTGCCTCCCGTAGGAGT | GK_run_4.2 | Mar. 25, 2010 | 1_E9 | 5436 | 1 |
| 311 | AGATCGGCTCGA | CATGCTGCCTCCCGTAGGAGT | GK_run_4.2 | Mar. 25, 2010 | 1_F9 | 7218 | 1 |
| 312 | AGCGTAGGTCGT | CATGCTGCCTCCCGTAGGAGT | GK_run_4.2 | Mar. 25, 2010 | 1_G9 | 5252 | 1 |
| 313 | TGATGCTAACTC | CATGCTGCCTCCCGTAGGAGT | GK_run_4.4 | Apr. 7, 2010 | 6_G6 | 3297 | 2 |
| 314 | TGCTCGTAGGAT | CATGCTGCCTCCCGTAGGAGT | GK_run_4.4 | Apr. 7, 2010 | 6_H6 | 2848 | 2 |
| 315 | TATGCGAGGTCG | CATGCTGCCTCCCGTAGGAGT | GK_run_4.4 | Apr. 7, 2010 | 6_A7 | 2502 | 2 |
| 316 | TCAGCCATGACA | CATGCTGCCTCCCGTAGGAGT | GK_run_4.4 | Apr. 7, 2010 | 6_B7 | 2074 | 2 |
| 317 | TCCTAGCAGTGA | CATGCTGCCTCCCGTAGGAGT | GK_run_4.4 | Apr. 7, 2010 | 6_C7 | 15122 | 2 |
| 318 | TCGCTAGTGAGG | CATGCTGCCTCCCGTAGGAGT | GK_run_4.4 | Apr. 7, 2010 | 6_D7 | 223 | 1 |
| 319 | TCTCCGCATGTC | CATGCTGCCTCCCGTAGGAGT | GK_run_4.4 | Apr. 7, 2010 | 6_E7 | 2269 | 1 |
| 321 | TGATGTGTGACC | CATGCTGCCTCCCGTAGGAGT | GK_run_4.4 | Apr. 7, 2010 | 6_G7 | 2047 | 1 |
| 323 | TATGGCACACAC | CATGCTGCCTCCCGTAGGAGT | GK_run_4.4 | Apr. 7, 2010 | 6_A8 | 2898 | 2 |
| 324 | TCAGCTCAACTA | CATGCTGCCTCCCGTAGGAGT | GK_run_4.4 | Apr. 7, 2010 | 6_B8 | 2070 | 2 |
| 325 | TCCTCTGTCGAC | CATGCTGCCTCCCGTAGGAGT | GK_run_4.4 | Apr. 7, 2010 | 6_C8 | 1996 | 2 |
| 326 | TCGGCTACAGAG | CATGCTGCCTCCCGTAGGAGT | GK_run_4.4 | Apr. 7, 2010 | 6_D8 | 2003 | 1 |
| 328 | TGACGGACATCT | CATGCTGCCTCCCGTAGGAGT | GK_run_4.4 | Apr. 7, 2010 | 6_F8 | 2864 | 1 |
| 329 | TGCAGAGCTCAG | CATGCTGCCTCCCGTAGGAGT | GK_run_4.4 | Apr. 7, 2010 | 6_G8 | 1111 | 1 |
| 330 | TGCTGTGAGCTA | CATGCTGCCTCCCGTAGGAGT | GK_run_4.4 | Apr. 7, 2010 | 6_H8 | 15062 | 1 |
| 332 | TCTGTTGCTCTC | CATGCTGCCTCCCGTAGGAGT | GK_run_4.3 | Mar. 30, 2010; | 6_F2 | 2453 | 2 |
| | | | GK_run_5.1 | Jul. 6, 2010 | | | |
| 333 | TGAGTCACTGGT | CATGCTGCCTCCCGTAGGAGT | GK_run_4.3 | Mar. 30, 2010; | 6_G2 | 3264 | 2 |
| | | | GK_run_5.1 | Jul. 6, 2010 | | | |
| 334 | TGCTACCATGAG | CATGCTGCCTCCCGTAGGAGT | GK_run_4.3 | Mar. 30, 2010; | 6_H2 | 2765 | 2 |
| | | | GK_run_5.1 | Jul. 6, 2010 | | | |
| 335 | TATCAGGTGTGC | CATGCTGCCTCCCGTAGGAGT | GK_run_4.3 | Mar. 30, 2010; | 6_A3 | 2150 | 2 |
| | | | GK_run_5.1 | Jul. 6, 2010 | | | |
| 338 | TCACTGGCAGTA | CATGCTGCCTCCCGTAGGAGT | GK_run_4.3 | Mar. 30, 2010 | 6_B3 | 3399 | 1 |
| 339 | TCATGGTACACT | CATGCTGCCTCCCGTAGGAGT | GK_run_4.3 | Mar. 30, 2010; | 6_C3 | 2604 | 1 |
| | | | GK_run_5.1 | Jul. 6, 2010 | | | |
| 340 | TCGATACTTGTG | CATGCTGCCTCCCGTAGGAGT | GK_run_4.3 | Mar. 30, 2010; | 6_D3 | 3308 | 1 |
| | | | GK_run_5.1 | Jul. 6, 2010 | | | |
| 341 | TCTACTCGTAAG | CATGCTGCCTCCCGTAGGAGT | GK_run_4.3 | Mar. 30, 2010 | 6_E3 | 1747 | 2 |
| 342 | TCTTAGACGACG | CATGCTGCCTCCCGTAGGAGT | GK_run_4.3 | Mar. 30, 2010; | 6_F3 | 3081 | 2 |
| | | | GK_run_5.1 | Jul. 6, 2010 | | | |
| 343 | TGAGTTCGCTAT | CATGCTGCCTCCCGTAGGAGT | GK_run_4.3 | Mar. 30, 2010 | 6_G3 | 1233 | 2 |
| 344 | TGCTAGTCATAC | CATGCTGCCTCCCGTAGGAGT | GK_run_4.3 | Mar. 30, 2010; | 6_H3 | 2169 | 1 |
| | | | GK_run_5.1 | Jul. 6, 2010 | | | |
| 345 | TATCGCGCGATA | CATGCTGCCTCCCGTAGGAGT | GK_run_4.3 | Mar. 30, 2010 | 6_A4 | 1368 | 1 |
| 347 | TCCACGTCGTCT | CATGCTGCCTCCCGTAGGAGT | GK_run_4.3 | Mar. 30, 2010; | 6_C4 | 2467 | 1 |
| | | | GK_run_5.1 | Jul. 6, 2010 | | | |
| 348 | TCGATGAACTCG | CATGCTGCCTCCCGTAGGAGT | GK_run_4.3 | Mar. 30, 2010 | 6_D4 | 1063 | 1 |
| 349 | TCTAGCGTAGTG | CATGCTGCCTCCCGTAGGAGT | GK_run_4.4 | Apr. 7, 2010 | 6_E4 | 3880 | 2 |
| 350 | TGAACGCTAGCT | CATGCTGCCTCCCGTAGGAGT | GK_run_4.4 | Apr. 7, 2010 | 6_F4 | 3359 | 2 |
| 351 | TGATAGTGAGGA | CATGCTGCCTCCCGTAGGAGT | GK_run_4.4 | Apr. 7, 2010 | 6_G4 | 4397 | 2 |
| 352 | TGCTATATCTGG | CATGCTGCCTCCCGTAGGAGT | GK_run_4.4 | Apr. 7, 2010 | 6_H4 | 2412 | 2 |
| 353 | TATCTCGAACTG | CATGCTGCCTCCCGTAGGAGT | GK_run_4.4 | Apr. 7, 2010 | 6_A5 | 1464 | 2 |
| 354 | TCAGACAGACCG | CATGCTGCCTCCCGTAGGAGT | GK_run_4.4 | Apr. 7, 2010 | 6_B5 | 2501 | 1 |
| 355 | TCCAGTGCGAGA | CATGCTGCCTCCCGTAGGAGT | GK_run_4.4 | Apr. 7, 2010 | 6_C5 | 3236 | 1 |
| 356 | TCGCATGAAGTC | CATGCTGCCTCCCGTAGGAGT | GK_run_4.4 | Apr. 7, 2010 | 6_D5 | 2745 | 1 |
| 357 | TCTAGTTAGTCG | CATGCTGCCTCCCGTAGGAGT | GK_run_4.4 | Apr. 7, 2010 | 6_E5 | 2956 | 1 |
| 358 | TGACATCAGCGG | CATGCTGCCTCCCGTAGGAGT | GK_run_4.4 | Apr. 7, 2010 | 6_F5 | 2763 | 1 |
| 359 | TGATCAGAAGAG | CATGCTGCCTCCCGTAGGAGT | GK_run_4.4 | Apr. 7, 2010 | 6_G5 | 2997 | 2 |
| 360 | TGCTCAGTATGT | CATGCTGCCTCCCGTAGGAGT | GK_run_4.4 | Apr. 7, 2010 | 6_H5 | 2927 | 2 |
| 361 | TATGCACCAGTG | CATGCTGCCTCCCGTAGGAGT | GK_run_4.4 | Apr. 7, 2010 | 6_A6 | 2951 | 2 |
| 362 | TCAGATCCGATG | CATGCTGCCTCCCGTAGGAGT | GK_run_4.4 | Apr. 7, 2010; | 6_B6 | 4486 | 1 |
| | | | GK_run_5.1 | Jul. 6, 2010 | | | |
| 363 | TCCGTCGTCTGT | CATGCTGCCTCCCGTAGGAGT | GK_run_4.4 | Apr. 7, 2010; | 6_C6 | 4602 | 1 |
| | | | GK_run_5.1 | Jul. 6, 2010 | | | |
| 364 | TCGCGTATTAGT | CATGCTGCCTCCCGTAGGAGT | GK_run_4.4 | Apr. 7, 2010; | 6_D6 | 4390 | 1 |
| | | | GK_run_5.1 | Jul. 6, 2010 | | | |
| 365 | TCTCACTAGGTA | CATGCTGCCTCCCGTAGGAGT | GK_run_4.4 | Apr. 7, 2010 | 6_E6 | 3685 | 1 |
| 366 | TGACCATATCGT | CATGCTGCCTCCCGTAGGAGT | GK_run_4.4 | Apr. 7, 2010 | 6_F6 | 3512 | 1 |
| 367 | GATAGCTGTCTT | CATGCTGCCTCCCGTAGGAGT | GK_run_4.1 | Mar. 19, 2010 | 4_D10 | 2567 | 2 |
| 369 | GCAGTATCACTG | CATGCTGCCTCCCGTAGGAGT | GK_run_4.1 | Mar. 19, 2010 | 4_F10 | 1714 | 2 |
| 370 | GCGATATATCGC | CATGCTGCCTCCCGTAGGAGT | GK_run_4.1 | Mar. 19, 2010 | 4_G10 | 2385 | 2 |
| 371 | GCTGATGAGCTG | CATGCTGCCTCCCGTAGGAGT | GK_run_4.1 | Mar. 19, 2010 | 4_H10 | 2805 | 2 |
| 372 | GACACTCGAATC | CATGCTGCCTCCCGTAGGAGT | GK_run_4.1 | Mar. 19, 2010 | 4_A11 | 2511 | 1 |
| 373 | GACTAACGTCAC | CATGCTGCCTCCCGTAGGAGT | GK_run_4.1 | Mar. 19, 2010 | 4_B11 | 2274 | 1 |
| 374 | GAGATGCCGACT | CATGCTGCCTCCCGTAGGAGT | GK_run_4.1 | Mar. 19, 2010 | 4_C11 | 1609 | 1 |
| 375 | GATAGTGCCACT | CATGCTGCCTCCCGTAGGAGT | GK_run_4.1 | Mar. 19, 2010 | 4_D11 | 2282 | 1 |
| 376 | GATGTGAGCGCT | CATGCTGCCTCCCGTAGGAGT | GK_run_4.1 | Mar. 19, 2010 | 4_E11 | 2557 | 1 |
| 377 | GCAGTTCATATC | CATGCTGCCTCCCGTAGGAGT | GK_run_4.1 | Mar. 19, 2010 | 4_F11 | 2944 | 2 |
| 378 | GCGGATGTGACT | CATGCTGCCTCCCGTAGGAGT | GK_run_4.1 | Mar. 19, 2010 | 4_G11 | 2793 | 2 |
| 379 | GCTGCTGCAATA | CATGCTGCCTCCCGTAGGAGT | GK_run_4.1 | Mar. 19, 2010 | 4_H11 | 2359 | 2 |
| 380 | GACAGCGTTGAC | CATGCTGCCTCCCGTAGGAGT | GK_run_4.1 | Mar. 19, 2010 | 4_A12 | 2942 | 1 |
| 381 | GACTAGACCAGC | CATGCTGCCTCCCGTAGGAGT | GK_run_4.1 | Mar. 19, 2010 | 4_B12 | 2840 | 1 |
| 382 | GAGCAGATGCCT | CATGCTGCCTCCCGTAGGAGT | GK_run_4.1 | Mar. 19, 2010 | 4_C12 | 2397 | 1 |
| 383 | GATATGCGGCTG | CATGCTGCCTCCCGTAGGAGT | GK_run_4.1 | Mar. 19, 2010 | 4_D12 | 1625 | 1 |
| 384 | GATTAGCACTCT | CATGCTGCCTCCCGTAGGAGT | GK_run_4.1 | Mar. 19, 2010 | 4_E12 | 2688 | 1 |
| 385 | GCATAGTAGCCG | CATGCTGCCTCCCGTAGGAGT | GK_run_4.1 | Mar. 19, 2010 | 4_F12 | 2655 | 2 |
| 386 | GCGTACAACTGT | CATGCTGCCTCCCGTAGGAGT | GK_run_4.1 | Mar. 19, 2010 | 4_G12 | 1886 | 2 |
| 387 | GCTGGTATCTGA | CATGCTGCCTCCCGTAGGAGT | GK_run_4.1 | Mar. 19, 2010 | 4_H12 | 2759 | 2 |
| 388 | AACGCACGCTAG | CATGCTGCCTCCCGTAGGAGT | GK_run_4.1 | Mar. 19, 2010; | 1_A1 | 2596 | 2 |
| | | | GK_run_5.1 | Jul. 6, 2010 | | | |
| 389 | ACACTGTTCATG | CATGCTGCCTCCCGTAGGAGT | GK_run_4.1 | Mar. 19, 2010 | 1_B1 | 1176 | 2 |
| 390 | ACCAGACGATGC | CATGCTGCCTCCCGTAGGAGT | GK_run_4.1 | Mar. 19, 2010 | 1_C1 | 1290 | 1 |
| 391 | ACGCTCATGGAT | CATGCTGCCTCCCGTAGGAGT | GK_run_4.1 | Mar. 19, 2010 | 1_D1 | 1180 | 1 |
| 392 | ACTCACGGTATG | CATGCTGCCTCCCGTAGGAGT | GK_run_4.1 | Mar. 19, 2010; | 1_E1 | 3017 | 1 |
| | | | GK_run_5.1 | Jul. 6, 2010 | | | |
| 393 | AGACCGTCAGAC | CATGCTGCCTCCCGTAGGAGT | GK_run_4.1 | Mar. 19, 2010; | 1_F1 | 2347 | 1 |
| | | | GK_run_5.1 | Jul. 6, 2010 | | | |
| 394 | AACTCGTCGATG | CATGCTGCCTCCCGTAGGAGT | GK_run_4.1 | Mar. 19, 2010; | 1_A2 | 2185 | 1 |
| | | | GK_run_5.1 | Jul. 6, 2010 | | | |
| 395 | ACAGACCACTCA | CATGCTGCCTCCCGTAGGAGT | GK_run_4.1 | Mar. 19, 2010 | 1_B2 | 1159 | 2 |
| 396 | ACCAGCGACTAG | CATGCTGCCTCCCGTAGGAGT | GK_run_4.1 | Mar. 19, 2010; | 1_C2 | 3200 | 2 |
| | | | GK_run_5.1 | Jul. 6, 2010 | | | |
| 397 | ACGGATCGTCAG | CATGCTGCCTCCCGTAGGAGT | GK_run_4.1 | Mar. 19, 2010; | 1_D2 | 2392 | 2 |
| | | | GK_run_5.1 | Jul. 6, 2010 | | | |
| 398 | ACTCAGATACTC | CATGCTGCCTCCCGTAGGAGT | GK_run_4.1 | Mar. 19, 2010 | 1_E2 | 1181 | 1 |
| 399 | AGACGTGCACTG | CATGCTGCCTCCCGTAGGAGT | GK_run_4.1 | Mar. 19, 2010; | 1_F2 | 2872 | 1 |
| | | | GK_run_5.1 | Jul. 6, 2010 | | | |
| 400 | AGCAGCACTTGT | CATGCTGCCTCCCGTAGGAGT | GK_run_4.1 | Mar. 19, 2010; | 1_G2 | 1894 | 1 |
| | | | GK_run_5.1 | Jul. 6, 2010 | | | |
| 401 | AGCTTGACAGCT | CATGCTGCCTCCCGTAGGAGT | GK_run_4.1 | Mar. 19, 2010; | 1_H2 | 2327 | 1 |
| | | | GK_run_5.1 | Jul. 6, 2010 | | | |
| 402 | AACTGTGCGTAC | CATGCTGCCTCCCGTAGGAGT | GK_run_4.1 | Mar. 19, 2010; | 1_A3 | 4056 | 1 |
| | | | GK_run_5.1 | Jul. 6, 2010 | | | |
| 403 | CAGATCGGATCG | CATGCTGCCTCCCGTAGGAGT | GK_run_4.2 | Mar. 25, 2010; | 2_G2 | 4392 | 2 |
| | | | GK_run_5.1 | Jul. 6, 2010 | | | |
| 404 | CATACCAGTAGC | CATGCTGCCTCCCGTAGGAGT | GK_run_4.2 | Mar. 25, 2010; | 2_H2 | 3733 | 2 |
| | | | GK_run_5.1 | Jul. 6, 2010 | | | |
| 405 | AGTGGATGCTCT | CATGCTGCCTCCCGTAGGAGT | GK_run_4.2 | Mar. 25, 2010; | 2_A3 | 4140 | 2 |
| | | | GK_run_5.1 | Jul. 6, 2010 | | | |
| 406 | ATAGCTCCATAC | CATGCTGCCTCCCGTAGGAGT | GK_run_4.2 | Mar. 25, 2010; | 2_B3 | 5023 | 2 |
| | | | GK_run_5.1 | Jul. 6, 2010 | | | |
| 407 | ATCGTACAACTC | CATGCTGCCTCCCGTAGGAGT | GK_run_4.2 | Mar. 25, 2010; | 2_C3 | 2418 | 2 |
| | | | GK_run_5.1 | Jul. 6, 2010 | | | |
| 408 | ATGCCTGAGCAG | CATGCTGCCTCCCGTAGGAGT | GK_run_4.2 | Mar. 25, 2010; | 2_D3 | 2404 | 1 |
| | | | GK_run_5.1 | Jul. 6, 2010 | | | |
| 409 | CAACACGCACGA | CATGCTGCCTCCCGTAGGAGT | GK_run_4.2 | Mar. 25, 2010; | 2_E3 | 2338 | 1 |
| | | | GK_run_5.1 | Jul. 6, 2010 | | | |
| 410 | CACGTCGATGGA | CATGCTGCCTCCCGTAGGAGT | GK_run_4.2 | Mar. 25, 2010; | 2_F3 | 2766 | 1 |
| | | | GK_run_5.1 | Jul. 6, 2010 | | | |
| 411 | CAGCACTAAGCG | CATGCTGCCTCCCGTAGGAGT | GK_run_4.2 | Mar. 25, 2010; | 2_G3 | 3609 | 1 |
| | | | GK_run_5.1 | Jul. 6, 2010 | | | |
| 412 | CATAGACGTTCG | CATGCTGCCTCCCGTAGGAGT | GK_run_4.2 | Mar. 25, 2010; | 2_H3 | 3321 | 1 |
| | | | GK_run_5.1 | Jul. 6, 2010 | | | |
| 413 | AGTGTCACGGTG | CATGCTGCCTCCCGTAGGAGT | GK_run_4.2 | Mar. 25, 2010; | 2_A4 | 4793 | 2 |
| | | | GK_run_5.1 | Jul. 6, 2010 | | | |
| 414 | ATAGGCGATCTC | CATGCTGCCTCCCGTAGGAGT | GK_run_4.2 | Mar. 25, 2010; | 2_B4 | 4093 | 2 |
| | | | GK_run_5.1 | Jul. 6, 2010 | | | |
| 416 | ATGCGTAGTGCG | CATGCTGCCTCCCGTAGGAGT | GK_run_4.2 | Mar. 25, 2010; | 2_D4 | 2956 | 1 |
| | | | GK_run_5.1 | Jul. 6, 2010 | | | |
| 417 | CAACTATCAGCT | CATGCTGCCTCCCGTAGGAGT | GK_run_4.2 | Mar. 25, 2010; | 2_E4 | 3557 | 1 |
| | | | GK_run_5.1 | Jul. 6, 2010 | | | |
| 418 | CACGTGACATGT | CATGCTGCCTCCCGTAGGAGT | GK_run_4.2 | Mar. 25, 2010; | 2_F4 | 6453 | 1 |
| | | | GK_run_5.1 | Jul. 6, 2010 | | | |
| 419 | CAGCATGTGTTG | CATGCTGCCTCCCGTAGGAGT | GK_run_4.2 | Mar. 25, 2010 | 2_G4 | 49 | 1 |
| 420 | CATAGCGAGTTC | CATGCTGCCTCCCGTAGGAGT | GK_run_4.2 | Mar. 25, 2010; | 2_H4 | 5822 | 1 |
| | | | GK_run_5.1 | Jul. 6, 2010 | | | |
| 421 | AGTCACATCACT | CATGCTGCCTCCCGTAGGAGT | GK_run_4.2 | Mar. 25, 2010 | 1_H9 | 5301 | 2 |
| 422 | ACACGAGCCACA | CATGCTGCCTCCCGTAGGAGT | GK_run_4.2 | Mar. 25, 2010 | 1_A10 | 5245 | 2 |
| 423 | ACATGTCACGTG | CATGCTGCCTCCCGTAGGAGT | GK_run_4.2 | Mar. 25, 2010 | 1_B10 | 4861 | 2 |
| 424 | ACGCGATACTGG | CATGCTGCCTCCCGTAGGAGT | GK_run_4.2 | Mar. 25, 2010 | 1_C10 | 4704 | 2 |
| 425 | ACTACGTGTGGT | CATGCTGCCTCCCGTAGGAGT | GK_run_4.2 | Mar. 25, 2010 | 1_D10 | 3767 | 2 |
| 426 | ACTGTGACTTCA | CATGCTGCCTCCCGTAGGAGT | GK_run_4.2 | Mar. 25, 2010 | 1_E10 | 4999 | 1 |
| 427 | AGATCTCTGCAT | CATGCTGCCTCCCGTAGGAGT | GK_run_4.2 | Mar. 25, 2010 | 1_F10 | 5880 | 1 |
| 428 | AGCTATCCACGA | CATGCTGCCTCCCGTAGGAGT | GK_run_4.2 | Mar. 25, 2010 | 1_G10 | 5964 | 1 |
| 429 | AGTCCATAGCTG | CATGCTGCCTCCCGTAGGAGT | GK_run_4.2 | Mar. 25, 2010 | 1_H10 | 4127 | 1 |
| 430 | ACACGGTGTCTA | CATGCTGCCTCCCGTAGGAGT | GK_run_4.2 | Mar. 25, 2010 | 1_A11 | 3610 | 1 |
| 431 | ACATTCAGCGCA | CATGCTGCCTCCCGTAGGAGT | GK_run_4.2 | Mar. 25, 2010 | 1_B11 | 4799 | 2 |
| 432 | ACGCGCAGATAC | CATGCTGCCTCCCGTAGGAGT | GK_run_4.2 | Mar. 25, 2010 | 1_C11 | 3223 | 2 |
| 433 | ACTAGCTCCATA | CATGCTGCCTCCCGTAGGAGT | GK_run_4.2 | Mar. 25, 2010 | 1_D11 | 4541 | 2 |
| 434 | ACTTGTAGCAGC | CATGCTGCCTCCCGTAGGAGT | GK_run_4.2 | Mar. 25, 2010 | 1_E11 | 4676 | 1 |
| 435 | AGATGTTCTGCT | CATGCTGCCTCCCGTAGGAGT | GK_run_4.2 | Mar. 25, 2010 | 1_F11 | 5414 | 1 |
| 436 | AGCTCCATACAG | CATGCTGCCTCCCGTAGGAGT | GK_run_4.2 | Mar. 25, 2010 | 1_G11 | 4879 | 1 |
| 437 | AGTCTACTCTGA | CATGCTGCCTCCCGTAGGAGT | GK_run_4.2 | Mar. 25, 2010 | 1_H11 | 2988 | 1 |
| 438 | ACACTAGATCCG | CATGCTGCCTCCCGTAGGAGT | GK_run_4.2 | Mar. 25, 2010 | 1_A12 | 4897 | 1 |
| 439 | ACCACATACATC | CATGCTGCCTCCCGTAGGAGT | GK_run_4.2 | Mar. 25, 2010 | 1_B12 | 4214 | 2 |
| 440 | ACGCTATCTGGA | CATGCTGCCTCCCGTAGGAGT | GK_run_4.2 | Mar. 25, 2010 | 1_C12 | 3336 | 2 |
| 441 | ACTATTGTCACG | CATGCTGCCTCCCGTAGGAGT | GK_run_4.2 | Mar. 25, 2010 | 1_D12 | 2357 | 2 |
| 442 | AGAACACGTCTC | CATGCTGCCTCCCGTAGGAGT | GK_run_4.2 | Mar. 25, 2010 | 1_E12 | 2645 | 2 |
| 443 | AGCACACCTACA | CATGCTGCCTCCCGTAGGAGT | GK_run_4.2 | Mar. 25, 2010 | 1_F12 | 2079 | 2 |
| 444 | AGCTCTCAGAGG | CATGCTGCCTCCCGTAGGAGT | GK_run_4.2 | Mar. 25, 2010 | 1_G12 | 1493 | 1 |
| 445 | AGTCTCGCATAT | CATGCTGCCTCCCGTAGGAGT | GK_run_4.2 | Mar. 25, 2010 | 1_H12 | 1350 | 1 |
| 446 | AGTGAGAGAAGC | CATGCTGCCTCCCGTAGGAGT | GK_run_4.2 | Mar. 25, 2010; | 2_A1 | 3610 | 1 |
| | | | GK_run_5.1 | Jul. 6, 2010 | | | |
| 447 | ATACTATTGCGC | CATGCTGCCTCCCGTAGGAGT | GK_run_4.2 | Mar. 25, 2010; | 2_B1 | 4784 | 1 |
| | | | GK_run_5.1 | Jul. 6, 2010 | | | |
| 448 | ATCGCGGACGAT | CATGCTGCCTCCCGTAGGAGT | GK_run_4.2 | Mar. 25, 2010; | 2_C1 | 4650 | 1 |
| | | | GK_run_5.1 | Jul. 6, 2010 | | | |
| 449 | ATGCACTGGCGA | CATGCTGCCTCCCGTAGGAGT | GK_run_4.2 | Mar. 25, 2010; | 2_D1 | 3538 | 2 |
| | | | GK_run_5.1 | Jul. 6, 2010 | | | |
| 450 | ATTATCGTGCAC | CATGCTGCCTCCCGTAGGAGT | GK_run_4.2 | Mar. 25, 2010; | 2_E1 | 3876 | 2 |
| | | | GK_run_5.1 | Jul. 6, 2010 | | | |
| 451 | AGTGCGATGCGT | CATGCTGCCTCCCGTAGGAGT | GK_run_4.2 | Mar. 25, 2010; | 2_A2 | 3747 | 2 |
| | | | GK_run_5.1 | Jul. 6, 2010 | | | |
| 452 | ATACTCACTCAG | CATGCTGCCTCCCGTAGGAGT | GK_run_4.2 | Mar. 25, 2010; | 2_B2 | 3353 | 1 |
| | | | GK_run_5.1 | Jul. 6, 2010 | | | |
| 453 | ATCGCTCGAGGA | CATGCTGCCTCCCGTAGGAGT | GK_run_4.2 | Mar. 25, 2010; | 2_C2 | 3304 | 1 |
| | | | GK_run_5.1 | Jul. 6, 2010 | | | |
| 454 | ATGCAGCTCAGT | CATGCTGCCTCCCGTAGGAGT | GK_run_4.2 | Mar. 25, 2010; | 2_D2 | 4065 | 1 |
| | | | GK_run_5.1 | Jul. 6, 2010 | | | |
| 455 | ATTCTGTGAGCG | CATGCTGCCTCCCGTAGGAGT | GK_run_4.2 | Mar. 25, 2010; | 2_E2 | 4245 | 1 |
| | | | GK_run_5.1 | Jul. 6, 2010 | | | |
| 456 | CACGGACTATAC | CATGCTGCCTCCCGTAGGAGT | GK_run_4.2 | Mar. 25, 2010; | 2_F2 | 4068 | 1 |
| | | | GK_run_5.1 | Jul. 6, 2010 | | | |
| 457 | AACGCACGCTAG | CATGCTGCCTCCCGTAGGAGT | GK_run_4.5 | Apr. 21, 2010 | 1_A1 | 6941 | 2 |
| 458 | ACACTGTTCATG | CATGCTGCCTCCCGTAGGAGT | GK_run_4.5 | Apr. 21, 2010 | 1_B1 | 7024 | 2 |
| 459 | ACCAGACGATGC | CATGCTGCCTCCCGTAGGAGT | GK_run_4.5 | Apr. 21, 2010 | 1_C1 | 7007 | 2 |
| 460 | ACGCTCATGGAT | CATGCTGCCTCCCGTAGGAGT | GK_run_4.5 | Apr. 21, 2010 | 1_D1 | 5334 | 2 |
| 461 | ACTCACGGTATG | CATGCTGCCTCCCGTAGGAGT | GK_run_4.5 | Apr. 21, 2010 | 1_E1 | 4648 | 2 |
| 462 | AGACCGTCAGAC | CATGCTGCCTCCCGTAGGAGT | GK_run_4.5 | Apr. 21, 2010 | 1_F1 | 6188 | 1 |
| 463 | AGCACGAGCCTA | CATGCTGCCTCCCGTAGGAGT | GK_run_4.5 | Apr. 21, 2010 | 1_G1 | 8077 | 1 |
| 465 | AACTCGTCGATG | CATGCTGCCTCCCGTAGGAGT | GK_run_4.5 | Apr. 21, 2010 | 1_A2 | 5968 | 1 |
| 466 | ACAGACCACTCA | CATGCTGCCTCCCGTAGGAGT | GK_run_4.5 | Apr. 21, 2010 | 1_B2 | 6100 | 1 |
| 467 | ACCAGCGACTAG | CATGCTGCCTCCCGTAGGAGT | GK_run_4.5 | Apr. 21, 2010 | 1_C2 | 5769 | 2 |
| 468 | ACGGATCGTCAG | CATGCTGCCTCCCGTAGGAGT | GK_run_4.5 | Apr. 21, 2010 | 1_D2 | 4274 | 2 |
| 469 | ACTCAGATACTC | CATGCTGCCTCCCGTAGGAGT | GK_run_4.5 | Apr. 21, 2010 | 1_E2 | 9537 | 2 |
| 470 | AGACGTGCACTG | CATGCTGCCTCCCGTAGGAGT | GK_run_4.5 | Apr. 21, 2010 | 1_F2 | 5685 | 1 |
| 471 | AGCAGCACTTGT | CATGCTGCCTCCCGTAGGAGT | GK_run_4.5 | Apr. 21, 2010 | 1_G2 | 6851 | 1 |
| 472 | AGCTTGACAGCT | CATGCTGCCTCCCGTAGGAGT | GK_run_4.5 | Apr. 21, 2010 | 1_H2 | 6656 | 1 |
| 473 | AACTGTGCGTAC | CATGCTGCCTCCCGTAGGAGT | GK_run_4.5 | Apr. 21, 2010 | 1_A3 | 10052 | 1 |
| 474 | ACAGAGTCGGCT | CATGCTGCCTCCCGTAGGAGT | GK_run_4.5 | Apr. 21, 2010 | 1_B3 | 5010 | 1 |
| 493 | ACTCTTCTAGAG | CATGCTGCCTCCCGTAGGAGT | GK_run_4.5 | Apr. 21, 2010 | 1_E5 | 4958 | 2 |
| 494 | AGAGCAAGAGCA | CATGCTGCCTCCCGTAGGAGT | GK_run_4.5 | Apr. 21, 2010 | 1_F5 | 2101 | 2 |
| 495 | AGCCATACTGAC | CATGCTGCCTCCCGTAGGAGT | GK_run_4.5 | Apr. 21, 2010 | 1_G5 | 5489 | 2 |
| 496 | AGGTGTGATCGC | CATGCTGCCTCCCGTAGGAGT | GK_run_4.5 | Apr. 21, 2010 | 1_H5 | 4575 | 2 |
| 498 | ACAGTGCTTCAT | CATGCTGCCTCCCGTAGGAGT | GK_run_4.5 | Apr. 21, 2010 | 1_B6 | 3493 | 1 |
| 499 | ACGACGTCTTAG | CATGCTGCCTCCCGTAGGAGT | GK_run_4.5 | Apr. 21, 2010 | 1_C6 | 6218 | 1 |
| 500 | ACGTGAGAGAAT | CATGCTGCCTCCCGTAGGAGT | GK_run_4.5 | Apr. 21, 2010 | 1_D6 | 4192 | 1 |
| 501 | ACTGACAGCCAT | CATGCTGCCTCCCGTAGGAGT | GK_run_4.5 | Apr. 21, 2010 | 1_E6 | 3052 | 1 |
| 502 | AGAGTAGCTAAG | CATGCTGCCTCCCGTAGGAGT | GK_run_4.5 | Apr. 21, 2010 | 1_F6 | 3287 | 1 |
| 503 | AGCGACTGTGCA | CATGCTGCCTCCCGTAGGAGT | GK_run_4.5 | Apr. 21, 2010 | 1_G6 | 5799 | 2 |
| 505 | AATCGTGACTCG | CATGCTGCCTCCCGTAGGAGT | GK_run_4.5 | Apr. 21, 2010 | 1_A7 | 3504 | 2 |
| 506 | ACAGTTGCGCGA | CATGCTGCCTCCCGTAGGAGT | GK_run_4.5 | Apr. 21, 2010 | 1_B7 | 4162 | 1 |
| 507 | ACGAGTGCTATC | CATGCTGCCTCCCGTAGGAGT | GK_run_4.5 | Apr. 21, 2010 | 1_C7 | 3582 | 1 |
| 509 | ACTGATCCTAGT | CATGCTGCCTCCCGTAGGAGT | GK_run_4.5 | Apr. 21, 2010 | 1_E7 | 3784 | 1 |
| 510 | AGAGTCCTGAGC | CATGCTGCCTCCCGTAGGAGT | GK_run_4.5 | Apr. 21, 2010 | 1_F7 | 4672 | 1 |
| 511 | AGCGAGCTATCT | CATGCTGCCTCCCGTAGGAGT | GK_run_4.5 | Apr. 21, 2010 | 1_G7 | 5618 | 2 |
| 512 | AGTACTGCAGGC | CATGCTGCCTCCCGTAGGAGT | GK_run_4.5 | Apr. 21, 2010 | 1_H7 | 4820 | 2 |
| 513 | ACACACTATGGC | CATGCTGCCTCCCGTAGGAGT | GK_run_4.5 | Apr. 21, 2010 | 1_A8 | 4098 | 2 |
| 514 | ACATCACTTAGC | CATGCTGCCTCCCGTAGGAGT | GK_run_4.5 | Apr. 21, 2010 | 1_B8 | 4915 | 2 |
| 515 | ACGATGCGACCA | CATGCTGCCTCCCGTAGGAGT | GK_run_4.5 | Apr. 21, 2010 | 1_C8 | 3875 | 2 |
| 517 | ACTGTACGCGTA | CATGCTGCCTCCCGTAGGAGT | GK_run_4.5 | Apr. 21, 2010 | 1_E8 | 4062 | 1 |
| 518 | AGATACACGCGC | CATGCTGCCTCCCGTAGGAGT | GK_run_4.5 | Apr. 21, 2010 | 1_F8 | 4990 | 1 |
| 519 | AGCGCTGATGTG | CATGCTGCCTCCCGTAGGAGT | GK_run_4.5 | Apr. 21, 2010 | 1_G8 | 5519 | 1 |
| 520 | AGTAGTATCCTC | CATGCTGCCTCCCGTAGGAGT | GK_run_4.5 | Apr. 21, 2010 | 1_H8 | 4261 | 1 |
| 521 | ACACATGTCTAC | CATGCTGCCTCCCGTAGGAGT | GK_run_4.5 | Apr. 21, 2010 | 1_A9 | 6732 | 2 |
| 522 | ACATGATCGTTC | CATGCTGCCTCCCGTAGGAGT | GK_run_4.5 | Apr. 21, 2010 | 1_B9 | 4845 | 2 |
| 523 | ACGCAACTGCTA | CATGCTGCCTCCCGTAGGAGT | GK_run_4.5 | Apr. 21, 2010 | 1_C9 | 5789 | 2 |
| 524 | ACTACAGCCTAT | CATGCTGCCTCCCGTAGGAGT | GK_run_4.5 | Apr. 21, 2010 | 1_D9 | 4604 | 1 |
| 525 | ACTGTCGAAGCT | CATGCTGCCTCCCGTAGGAGT | GK_run_4.5 | Apr. 21, 2010 | 1_E9 | 4773 | 1 |
| 526 | AGATCGGCTCGA | CATGCTGCCTCCCGTAGGAGT | GK_run_4.5 | Apr. 21, 2010 | 1_F9 | 3210 | 1 |
| 527 | AGCGTAGGTCGT | CATGCTGCCTCCCGTAGGAGT | GK_run_4.5 | Apr. 21, 2010 | 1_G9 | 3990 | 1 |
| 547 | ACGCTATCTGGA | CATGCTGCCTCCCGTAGGAGT | GK_run_4.5 | Apr. 21, 2010 | 1_C12 | 4539 | 2 |
| 548 | ACTATTGTCACG | CATGCTGCCTCCCGTAGGAGT | GK_run_4.5 | Apr. 21, 2010 | 1_D12 | 4432 | 2 |
| 549 | AGAACACGTCTC | CATGCTGCCTCCCGTAGGAGT | GK_run_4.5 | Apr. 21, 2010 | 1_E12 | 4431 | 2 |
| 550 | AGCACACCTACA | CATGCTGCCTCCCGTAGGAGT | GK_run_4.5 | Apr. 21, 2010 | 1_F12 | 5001 | 2 |
| 551 | AGCTCTCAGAGG | CATGCTGCCTCCCGTAGGAGT | GK_run_4.5 | Apr. 21, 2010 | 1_G12 | 5141 | 2 |
| 552 | AGTCTCGCATAT | CATGCTGCCTCCCGTAGGAGT | GK_run_4.5 | Apr. 21, 2010 | 1_H12 | 6246 | 1 |
| 553 | AGTGAGAGAAGC | CATGCTGCCTCCCGTAGGAGT | GK_run_4.5 | Apr. 21, 2010 | 2_A1 | 1985 | 1 |
| 554 | ATACTATTGCGC | CATGCTGCCTCCCGTAGGAGT | GK_run_4.5 | Apr. 21, 2010 | 2_B1 | 1930 | 1 |
| 555 | ATCGCGGACGAT | CATGCTGCCTCCCGTAGGAGT | GK_run_4.5 | Apr. 21, 2010 | 2_C1 | 1727 | 1 |
| 557 | ATTATCGTGCAC | CATGCTGCCTCCCGTAGGAGT | GK_run_4.5 | Apr. 21, 2010 | 2_E1 | 2954 | 2 |
| 558 | CACGACAGGCTA | CATGCTGCCTCCCGTAGGAGT | GK_run_4.5 | Apr. 21, 2010 | 2_F1 | 2126 | 2 |
| 559 | CAGATACACTTC | CATGCTGCCTCCCGTAGGAGT | GK_run_4.5 | Apr. 21, 2010 | 2_G1 | 2754 | 2 |
| 560 | CAGTGTCAGGAC | CATGCTGCCTCCCGTAGGAGT | GK_run_4.5 | Apr. 21, 2010 | 2_H1 | 1691 | 1 |
| 561 | AGTGCGATGCGT | CATGCTGCCTCCCGTAGGAGT | GK_run_4.5 | Apr. 21, 2010 | 2_A2 | 2213 | 1 |
| 562 | ATACTCACTCAG | CATGCTGCCTCCCGTAGGAGT | GK_run_4.5 | Apr. 21, 2010 | 2_B2 | 2106 | 1 |
| 563 | ATCGCTCGAGGA | CATGCTGCCTCCCGTAGGAGT | GK_run_4.5 | Apr. 21, 2010 | 2_C2 | 1525 | 1 |
| 583 | CAGCATGTGTTG | CATGCTGCCTCCCGTAGGAGT | GK_run_4.5 | Apr. 21, 2010 | 2_G4 | 288 | 2 |
| 584 | CATAGCGAGTTC | CATGCTGCCTCCCGTAGGAGT | GK_run_4.5 | Apr. 21, 2010 | 2_H4 | 1839 | 2 |
| 587 | ATCTCTGGCATA | CATGCTGCCTCCCGTAGGAGT | GK_run_4.5 | Apr. 21, 2010 | 2_C5 | 1881 | 2 |
| 588 | ATGGATACGCTC | CATGCTGCCTCCCGTAGGAGT | GK_run_4.5 | Apr. 21, 2010 | 2_D5 | 3537 | 1 |
| 589 | CAACTCATCGTA | CATGCTGCCTCCCGTAGGAGT | GK_run_4.5 | Apr. 21, 2010 | 2_E5 | 1777 | 1 |
| 591 | CAGCGGTGACAT | CATGCTGCCTCCCGTAGGAGT | GK_run_4.5 | Apr. 21, 2010 | 2_G5 | 1509 | 1 |
| 592 | CATATACTCGCA | CATGCTGCCTCCCGTAGGAGT | GK_run_4.5 | Apr. 21, 2010 | 2_H5 | 1686 | 1 |
| 593 | AGTTAGTGCGTC | CATGCTGCCTCCCGTAGGAGT | GK_run_4.5 | Apr. 21, 2010; | 2_A6 | 2178 | 2 |
| | | | GK_run_5.1 | Jul. 6, 2010 | | | |
| 594 | ATATGCCAGTGC | CATGCTGCCTCCCGTAGGAGT | GK_run_4.5 | Apr. 21, 2010 | 2_B6 | 1751 | 2 |
| 595 | ATCTGAGCTGGT | CATGCTGCCTCCCGTAGGAGT | GK_run_4.5 | Apr. 21, 2010 | 2_C6 | 2451 | 2 |
| 596 | ATGGCAGCTCTA | CATGCTGCCTCCCGTAGGAGT | GK_run_4.5 | Apr. 21, 2010 | 2_D6 | 1984 | 1 |
| 597 | CAAGATCGACTC | CATGCTGCCTCCCGTAGGAGT | GK_run_4.5 | Apr. 21, 2010 | 2_E6 | 1965 | 1 |
| 598 | CACTCAACAGAC | CATGCTGCCTCCCGTAGGAGT | GK_run_4.5 | Apr. 21, 2010 | 2_F6 | 1667 | 1 |
| 599 | CAGCTAGAACGC | CATGCTGCCTCCCGTAGGAGT | GK_run_4.5 | Apr. 21, 2010 | 2_G6 | 2507 | 1 |
| 600 | CATATCGCAGTT | CATGCTGCCTCCCGTAGGAGT | GK_run_4.5 | Apr. 21, 2010; | 2_H6 | 1927 | 1 |
| | | | GK_run_5.1 | Jul. 6, 2010 | | | |
| 601 | AGTTCAGACGCT | CATGCTGCCTCCCGTAGGAGT | GK_run_4.5 | Apr. 21, 2010 | 2_A7 | 1996 | 2 |
| 602 | ATCACGTAGCGG | CATGCTGCCTCCCGTAGGAGT | GK_run_4.5 | Apr. 21, 2010 | 2_B7 | 2261 | 2 |
| 603 | ATCTGGTGCTAT | CATGCTGCCTCCCGTAGGAGT | GK_run_4.5 | Apr. 21, 2010 | 2_C7 | 2300 | 2 |
| 604 | ATGGCGTGCACA | CATGCTGCCTCCCGTAGGAGT | GK_run_4.5 | Apr. 21, 2010 | 2_D7 | 3097 | 2 |
| 605 | CAAGTGAGAGAG | CATGCTGCCTCCCGTAGGAGT | GK_run_4.5 | Apr. 21, 2010 | 2_E7 | 3208 | 2 |
| 606 | CACTCTGATTAG | CATGCTGCCTCCCGTAGGAGT | GK_run_4.5 | Apr. 21, 2010 | 2_F7 | 2974 | 1 |
| 607 | CAGGTGCTACTA | CATGCTGCCTCCCGTAGGAGT | GK_run_4.5 | Apr. 21, 2010 | 2_G7 | 3147 | 1 |
| 608 | CATCAGCGTGTA | CATGCTGCCTCCCGTAGGAGT | GK_run_4.5 | Apr. 21, 2010 | 2_H7 | 3555 | 1 |
| 609 | AGTTCTACGTCA | CATGCTGCCTCCCGTAGGAGT | GK_run_4.5 | Apr. 21, 2010 | 2_A8 | 2425 | 1 |
| 610 | ATCACTAGTCAC | CATGCTGCCTCCCGTAGGAGT | GK_run_4.5 | Apr. 21, 2010 | 2_B8 | 2533 | 1 |
| 611 | ATCTTAGACTGC | CATGCTGCCTCCCGTAGGAGT | GK_run_4.5 | Apr. 21, 2010 | 2_C8 | 1678 | 2 |
| 612 | ATGGTCTACTAC | CATGCTGCCTCCCGTAGGAGT | GK_run_4.5 | Apr. 21, 2010 | 2_D8 | 2063 | 2 |
| 613 | CACACGTGAGCA | CATGCTGCCTCCCGTAGGAGT | GK_run_4.5 | Apr. 21, 2010 | 2_E8 | 2638 | 2 |
| 614 | CACTGGTATATC | CATGCTGCCTCCCGTAGGAGT | GK_run_4.5 | Apr. 21, 2010 | 2_F8 | 3166 | 1 |
| 615 | CAGTACGATCTT | CATGCTGCCTCCCGTAGGAGT | GK_run_4.5 | Apr. 21, 2010 | 2_G8 | 1642 | 1 |
| 616 | CATCATGAGGCT | CATGCTGCCTCCCGTAGGAGT | GK_run_4.5 | Apr. 21, 2010 | 2_H8 | 1550 | 1 |
| 617 | ATAATCTCGTCG | CATGCTGCCTCCCGTAGGAGT | GK_run_4.5 | Apr. 21, 2010 | 2_A9 | 1278 | 1 |
| 618 | ATCAGGCGTGTG | CATGCTGCCTCCCGTAGGAGT | GK_run_4.5 | Apr. 21, 2010 | 2_B9 | 1185 | 1 |
| 1001 | GAGAATACGTGA | CATGCTGCCTCCCGTAGGAGT | GK_run_1.1 | Jul. 16, 2009; | 4_C7 | 5862 | 2 |
| | | | GK_run_3.1 | Oct. 30, 2009 | | | |
| 1002 | GACGATATCGCG | CATGCTGCCTCCCGTAGGAGT | GK_run_1.1 | Jul. 16, 2009; | 4_B7 | 4352 | 2 |
| | | | GK_run_3.1 | Oct. 30, 2009 | | | |
| 1003 | GAAGAGTGATCA | CATGCTGCCTCCCGTAGGAGT | GK_run_1.1 | Jul. 16, 2009; | 4_A7 | 7940 | 2 |
| | | | GK_run_3.1 | Oct. 30, 2009 | | | |
| 2000 | TCAACAGCATCG | CATGCTGCCTCCCGTAGGAGT | GK_run_2.1 | Aug. 17, 2009 | 6_A10 | 3417 | 2 |
| 2001 | TCAGTACGAGGC | CATGCTGCCTCCCGTAGGAGT | GK_run_2.1 | Aug. 17, 2009; | 6_B10 | 2740 | 2 |
| | | | GK_run_3.1 | Oct. 30, 2009 | | | |
| 2002 | TCGAATCACAGC | CATGCTGCCTCCCGTAGGAGT | GK_run_2.1 | Aug. 17, 2009; | 6_C10 | 3010 | 2 |
| | | | GK_run_3.1 | Oct. 30, 2009 | | | |
| 2003 | TCGTCGATAATC | CATGCTGCCTCCCGTAGGAGT | GK_run_2.1 | Aug. 17, 2009; | 6_D10 | 2387 | 2 |
| | | | GK_run_3.1 | Oct. 30, 2009 | | | |
| 2004 | TCTCTCCGTCGA | CATGCTGCCTCCCGTAGGAGT | GK_run_2.1 | Aug. 17, 2009; | 6_E10 | 2351 | 2 |
| | | | GK_run_3.1 | Oct. 30, 2009 | | | |
| 2005 | TGAGAGAGCATA | CATGCTGCCTCCCGTAGGAGT | GK_run_2.1 | Aug. 17, 2009; | 6_F10 | 2724 | 2 |
| | | | GK_run_3.1 | Oct. 30, 2009 | | | |
| 2006 | TGCGCGAATACT | CATGCTGCCTCCCGTAGGAGT | GK_run_2.1 | Aug. 17, 2009; | 6_G10 | 2557 | 2 |
| | | | GK_run_3.1 | Oct. 30, 2009 | | | |
| 2007 | TGGCTCTACAGA | CATGCTGCCTCCCGTAGGAGT | GK_run_2.1 | Aug. 17, 2009; | 6_H10 | 2284 | 2 |
| | | | GK_run_3.1 | Oct. 30, 2009 | | | |
| 2008 | TCAATCTAGCGT | CATGCTGCCTCCCGTAGGAGT | GK_run_2.1 | Aug. 17, 2009; | 6_A11 | 2534 | 2 |
| | | | GK_run_3.1 | Oct. 30, 2009 | | | |
| 2009 | TCAGTCGACGAG | CATGCTGCCTCCCGTAGGAGT | GK_run_2.1 | Aug. 17, 2009; | 6_B11 | 2719 | 2 |
| | | | GK_run_3.1 | Oct. 30, 2009 | | | |
| 2010 | TCGACTCCTCGT | CATGCTGCCTCCCGTAGGAGT | GK_run_2.1 | Aug. 17, 2009 | 6_C11 | 2410 | 1 |
| 2011 | TCGTGATGTGAC | CATGCTGCCTCCCGTAGGAGT | GK_run_2.1 | Aug. 17, 2009 | 6_D11 | 3470 | 1 |
| 2012 | TCTGAGTCTGAG | CATGCTGCCTCCCGTAGGAGT | GK_run_2.1 | Aug. 17, 2009 | 6_E11 | 2229 | 1 |
| 2013 | TGAGCACACACG | CATGCTGCCTCCCGTAGGAGT | GK_run_2.1 | Aug. 17, 2009 | 6_F11 | 2769 | 1 |
| 2014 | TGCGTATAGTGC | CATGCTGCCTCCCGTAGGAGT | GK_run_2.1 | Aug. 17, 2009 | 6_G11 | 2382 | 1 |
| 2015 | TGGTCATCACTA | CATGCTGCCTCCCGTAGGAGT | GK_run_2.1 | Aug. 17, 2009 | 6_H11 | 2396 | 1 |
| 2016 | TCACAGATCCGA | CATGCTGCCTCCCGTAGGAGT | GK_run_2.1 | Aug. 17, 2009 | 6_A12 | 2361 | 1 |
| 2017 | TCAGTGACGTAC | CATGCTGCCTCCCGTAGGAGT | GK_run_2.1 | Aug. 17, 2009 | 6_B12 | 2255 | 1 |
| 2018 | TCGAGACGCTTA | CATGCTGCCTCCCGTAGGAGT | GK_run_2.1 | Aug. 17, 2009 | 6_C12 | 2400 | 1 |
| 2019 | TCGTGTCTATAG | CATGCTGCCTCCCGTAGGAGT | GK_run_2.1 | Aug. 17, 2009 | 6_D12 | 2675 | 1 |
| 3001 | CCGATGTCAGAT | CATGCTGCCTCCCGTAGGAGT | GK_run_3.1 | Oct. 30, 2009 | 3_A10 | 3103 | 2 |
| 3002 | CGAGTTGTAGCG | CATGCTGCCTCCCGTAGGAGT | GK_run_3.1 | Oct. 30, 2009 | 3_B10 | 3266 | 2 |
| 3003 | CGCGTAACTGTA | CATGCTGCCTCCCGTAGGAGT | GK_run_3.1 | Oct. 30, 2009 | 3_C10 | 4179 | 2 |
| 3004 | CGTCACGACTAA | CATGCTGCCTCCCGTAGGAGT | GK_run_3.1 | Oct. 30, 2009 | 3_D10 | 3499 | 2 |
| 3005 | CTACACAAGCAC | CATGCTGCCTCCCGTAGGAGT | GK_run_3.1 | Oct. 30, 2009 | 3_E10 | 2039 | 2 |
| 3006 | CTATCTAGCGAG | CATGCTGCCTCCCGTAGGAGT | GK_run_3.1 | Oct. 30, 2009 | 3_F10 | 3584 | 2 |
| 3007 | CTCTGAAGTCTA | CATGCTGCCTCCCGTAGGAGT | GK_run_3.1 | Oct. 30, 2009 | 3_G10 | 2929 | 2 |
| 3008 | CTGTCTCTCCTA | CATGCTGCCTCCCGTAGGAGT | GK_run_3.1 | Oct. 30, 2009 | 3_H10 | 3741 | 2 |
| 3009 | CCTAGTACTGAT | CATGCTGCCTCCCGTAGGAGT | GK_run_3.1 | Oct. 30, 2009 | 3_A11 | 3439 | 2 |
| 3010 | CGATAGATCTTC | CATGCTGCCTCCCGTAGGAGT | GK_run_3.1 | Oct. 30, 2009 | 3_B11 | 3261 | 2 |
| 3011 | CGCTAGAACGCA | CATGCTGCCTCCCGTAGGAGT | GK_run_3.1 | Oct. 30, 2009 | 3_C11 | 0 | 2 |
| 3012 | CGTCAGACGGAT | CATGCTGCCTCCCGTAGGAGT | GK_run_3.1 | Oct. 30, 2009 | 3_D11 | 3504 | 2 |
| 4000 | GCTGTAGTATGC | CATGCTGCCTCCCGTAGGAGT | GK_run_5.1 | Jul. 6, 2010 | 5_A1 | 1614 | 1 |
| 4001 | GGTCACTGACAG | CATGCTGCCTCCCGTAGGAGT | GK_run_5.1 | Jul. 6, 2010 | 5_B1 | 1478 | 1 |
| 4002 | GTAGTGTCTAGC | CATGCTGCCTCCCGTAGGAGT | GK_run_5.1 | Jul. 6, 2010 | 5_C1 | 1803 | 1 |
| 4003 | GTCGCTGTCTTC | CATGCTGCCTCCCGTAGGAGT | GK_run_5.1 | Jul. 6, 2010 | 5_D1 | 1732 | 1 |
| 4004 | GTGAGGTCGCTA | CATGCTGCCTCCCGTAGGAGT | GK_run_5.1 | Jul. 6, 2010 | 5_E1 | 1449 | 1 |
| 4005 | GTTGACGACAGC | CATGCTGCCTCCCGTAGGAGT | GK_run_5.1 | Jul. 6, 2010 | 5_F1 | 1656 | 1 |
| 4006 | TACGCGCTGAGA | CATGCTGCCTCCCGTAGGAGT | GK_run_5.1 | Jul. 6, 2010 | 5_G1 | 1564 | 1 |
| 4007 | TAGATCCTCGAT | CATGCTGCCTCCCGTAGGAGT | GK_run_5.1 | Jul. 6, 2010 | 5_H1 | 2002 | 1 |
| 4008 | GCTGTGTAGGAC | CATGCTGCCTCCCGTAGGAGT | GK_run_5.1 | Jul. 6, 2010 | 5_A2 | 1951 | 1 |
| 4009 | GGTCGTAGCGTA | CATGCTGCCTCCCGTAGGAGT | GK_run_5.1 | Jul. 6, 2010 | 5_B2 | 2229 | 1 |
| 4010 | GTATATCCGCAG | CATGCTGCCTCCCGTAGGAGT | GK_run_5.1 | Jul. 6, 2010 | 5_C2 | 1933 | 1 |
| 4011 | GTCGTAGCCAGA | CATGCTGCCTCCCGTAGGAGT | GK_run_5.1 | Jul. 6, 2010 | 5_D2 | 1391 | 1 |
| 4012 | GTGATAGTGCCG | CATGCTGCCTCCCGTAGGAGT | GK_run_5.1 | Jul. 6, 2010 | 5_E2 | 1780 | 1 |
| 4013 | GTTGTATACTCG | CATGCTGCCTCCCGTAGGAGT | GK_run_5.1 | Jul. 6, 2010 | 5_F2 | 1555 | 1 |
| 4014 | TACGGTATGTCT | CATGCTGCCTCCCGTAGGAGT | GK_run_5.1 | Jul. 6, 2010 | 5_G2 | 1234 | 1 |
| 4015 | TAGCACACCTAT | CATGCTGCCTCCCGTAGGAGT | GK_run_5.1 | Jul. 6, 2010 | 5_H2 | 2106 | 1 |
| 4016 | GCTTACATCGAG | CATGCTGCCTCCCGTAGGAGT | GK_run_5.1 | Jul. 6, 2010 | 5_A3 | 1172 | 1 |
| 4017 | GGTGCGTGTATG | CATGCTGCCTCCCGTAGGAGT | GK_run_5.1 | Jul. 6, 2010 | 5_B3 | 1324 | 1 |

| | Donor | | | Date human | | Date human | |
|---|---|---|---|---|---|---|---|
| Sample | Mouse | Source | Sample | sample | Date human | sample plate | |
| ID | Sample | Material | Type | collection | sample plating | collection | Mouse ID |
| 2 | Donor | direct | fc | Dec. 10, 2009 | N/A | N/A | N/A |
| 3 | Donor | cultured | pt | Dec. 10, 2009 | Dec. 10, 2009 | Dec. 17, 2009 | N/A |
| 5 | Donor | direct | fc | Dec. 10, 2009 | N/A | N/A | N/A |
| 6 | Donor | cultured | pt | Dec. 10, 2009 | Dec. 10, 2009 | Dec. 17, 2009 | N/A |
| 7 | Mouse | direct | fc | Dec. 10, 2009 | N/A | N/A | GK_mouse_4.1 |
| 8 | Mouse | direct | fc | Dec. 10, 2009 | N/A | N/A | GK_mouse_4.2 |
| 9 | Mouse | direct | fc | Dec. 10, 2009 | N/A | N/A | GK_mouse_4.3 |
| 10 | Mouse | direct | fc | Dec. 10, 2009 | N/A | N/A | GK_mouse_4.4 |
| 11 | Mouse | direct | fc | Dec. 10, 2009 | N/A | N/A | GK_mouse_4.5 |
| 12 | Mouse | direct | fc | Dec. 10, 2009 | N/A | N/A | GK_mouse_4.6 |
| 14 | Mouse | direct | fc | Dec. 10, 2009 | N/A | N/A | GK_mouse_4.8 |
| 15 | Mouse | direct | fc | Dec. 10, 2009 | N/A | N/A | GK_mouse_4.9 |
| 16 | Mouse | direct | fc | Dec. 10, 2009 | N/A | N/A | GK_mouse_4.10 |
| 17 | Mouse | cultured | fc | Dec. 10, 2009 | Dec. 10, 2009 | Dec. 17, 2009 | GK_mouse_4.11 |
| 18 | Mouse | cultured | fc | Dec. 10, 2009 | Dec. 10, 2009 | Dec. 17, 2009 | GK_mouse_4.13 |
| 19 | Mouse | cultured | fc | Dec. 10, 2009 | Dec. 10, 2009 | Dec. 17, 2009 | GK_mouse_4.15 |
| 20 | Mouse | cultured | fc | Dec. 10, 2009 | Dec. 10, 2009 | Dec. 17, 2009 | GK_mouse_4.16 |
| 21 | Mouse | cultured | fc | Dec. 10, 2009 | Dec. 10, 2009 | Dec. 17, 2009 | GK_mouse_4.17 |
| 22 | Mouse | cultured | fc | Dec. 10, 2009 | Dec. 10, 2009 | Dec. 17, 2009 | GK_mouse_4.18 |
| 23 | Mouse | cultured | fc | Dec. 10, 2009 | Dec. 10, 2009 | Dec. 17, 2009 | GK_mouse_4.19 |
| 24 | Mouse | cultured | fc | Dec. 10, 2009 | Dec. 10, 2009 | Dec. 17, 2009 | GK_mouse_4.20 |
| 25 | Mouse | direct | fc | Dec. 10, 2009 | N/A | N/A | GK_mouse_4.1 |
| 26 | Mouse | direct | fc | Dec. 10, 2009 | N/A | N/A | GK_mouse_4.2 |
| 27 | Mouse | direct | fc | Dec. 10, 2009 | N/A | N/A | GK_mouse_4.3 |
| 28 | Mouse | direct | fc | Dec. 10, 2009 | N/A | N/A | GK_mouse_4.4 |
| 29 | Mouse | direct | fc | Dec. 10, 2009 | N/A | N/A | GK_mouse_4.5 |
| 30 | Mouse | direct | fc | Dec. 10, 2009 | N/A | N/A | GK_mouse_4.6 |
| 31 | Mouse | direct | fc | Dec. 10, 2009 | N/A | N/A | GK_mouse_4.7 |
| 32 | Mouse | direct | fc | Dec. 10, 2009 | N/A | N/A | GK_mouse_4.8 |
| 33 | Mouse | direct | fc | Dec. 10, 2009 | N/A | N/A | GK_mouse_4.9 |
| 34 | Mouse | direct | fc | Dec. 10, 2009 | N/A | N/A | GK_mouse_4.10 |
| 35 | Mouse | cultured | fc | Dec. 10, 2009 | Dec. 10, 2009 | Dec. 17, 2009 | GK_mouse_4.11 |
| 36 | Mouse | cultured | fc | Dec. 10, 2009 | Dec. 10, 2009 | Dec. 17, 2009 | GK_mouse_4.13 |
| 37 | Mouse | cultured | fc | Dec. 10, 2009 | Dec. 10, 2009 | Dec. 17, 2009 | GK_mouse_4.15 |
| 38 | Mouse | cultured | fc | Dec. 10, 2009 | Dec. 10, 2009 | Dec. 17, 2009 | GK_mouse_4.16 |
| 39 | Mouse | cultured | fc | Dec. 10, 2009 | Dec. 10, 2009 | Dec. 17, 2009 | GK_mouse_4.17 |
| 40 | Mouse | cultured | fc | Dec. 10, 2009 | Dec. 10, 2009 | Dec. 17, 2009 | GK_mouse_4.18 |
| 41 | Mouse | cultured | fc | Dec. 10, 2009 | Dec. 10, 2009 | Dec. 17, 2009 | GK_mouse_4.19 |
| 42 | Mouse | cultured | fc | Dec. 10, 2009 | Dec. 10, 2009 | Dec. 17, 2009 | GK_mouse_4.20 |
| 43 | Mouse | direct | fc | Dec. 10, 2009 | N/A | N/A | GK_mouse_4.1 |
| 44 | Mouse | direct | fc | Dec. 10, 2009 | N/A | N/A | GK_mouse_4.2 |
| 45 | Mouse | direct | fc | Dec. 10, 2009 | N/A | N/A | GK_mouse_4.3 |
| 46 | Mouse | direct | fc | Dec. 10, 2009 | N/A | N/A | GK_mouse_4.4 |
| 47 | Mouse | direct | fc | Dec. 10, 2009 | N/A | N/A | GK_mouse_4.5 |
| 48 | Mouse | direct | fc | Dec. 10, 2009 | N/A | N/A | GK_mouse_4.6 |
| 49 | Mouse | direct | fc | Dec. 10, 2009 | N/A | N/A | GK_mouse_4.7 |
| 50 | Mouse | direct | fc | Dec. 10, 2009 | N/A | N/A | GK_mouse_4.8 |
| 51 | Mouse | direct | fc | Dec. 10, 2009 | N/A | N/A | GK_mouse_4.9 |
| 52 | Mouse | direct | fc | Dec. 10, 2009 | N/A | N/A | GK_mouse_4.10 |
| 53 | Mouse | cultured | fc | Dec. 10, 2009 | Dec. 10, 2009 | Dec. 17, 2009 | GK_mouse_4.11 |
| 54 | Mouse | cultured | fc | Dec. 10, 2009 | Dec. 10, 2009 | Dec. 17, 2009 | GK_mouse_4.13 |
| 55 | Mouse | cultured | fc | Dec. 10, 2009 | Dec. 10, 2009 | Dec. 17, 2009 | GK_mouse_4.15 |
| 56 | Mouse | cultured | fc | Dec. 10, 2009 | Dec. 10, 2009 | Dec. 17, 2009 | GK_mouse_4.16 |
| 57 | Mouse | cultured | fc | Dec. 10, 2009 | Dec. 10, 2009 | Dec. 17, 2009 | GK_mouse_4.17 |
| 58 | Mouse | cultured | fc | Dec. 10, 2009 | Dec. 10, 2009 | Dec. 17, 2009 | GK_mouse_4.18 |
| 59 | Mouse | cultured | fc | Dec. 10, 2009 | Dec. 10, 2009 | Dec. 17, 2009 | GK_mouse_4.19 |
| 60 | Mouse | cultured | fc | Dec. 10, 2009 | Dec. 10, 2009 | Dec. 17, 2009 | GK_mouse_4.20 |
| 61 | Mouse | direct | fc | Dec. 10, 2009 | N/A | N/A | GK_mouse_4.1 |
| 62 | Mouse | direct | fc | Dec. 10, 2009 | N/A | N/A | GK_mouse_4.2 |
| 63 | Mouse | direct | fc | Dec. 10, 2009 | N/A | N/A | GK_mouse_4.3 |
| 64 | Mouse | direct | fc | Dec. 10, 2009 | N/A | N/A | GK_mouse_4.4 |
| 65 | Mouse | direct | fc | Dec. 10, 2009 | N/A | N/A | GK_mouse_4.5 |
| 66 | Mouse | direct | fc | Dec. 10, 2009 | N/A | N/A | GK_mouse_4.6 |
| 67 | Mouse | direct | fc | Dec. 10, 2009 | N/A | N/A | GK_mouse_4.7 |
| 68 | Mouse | direct | fc | Dec. 10, 2009 | N/A | N/A | GK_mouse_4.8 |
| 69 | Mouse | direct | fc | Dec. 10, 2009 | N/A | N/A | GK_mouse_4.9 |
| 70 | Mouse | direct | fc | Dec. 10, 2009 | N/A | N/A | GK_mouse_4.10 |
| 71 | Mouse | cultured | fc | Dec. 10, 2009 | Dec. 10, 2009 | Dec. 17, 2009 | GK_mouse_4.11 |
| 72 | Mouse | cultured | fc | Dec. 10, 2009 | Dec. 10, 2009 | Dec. 17, 2009 | GK_mouse_4.13 |
| 73 | Mouse | cultured | fc | Dec. 10, 2009 | Dec. 10, 2009 | Dec. 17, 2009 | GK_mouse_4.15 |
| 74 | Mouse | cultured | fc | Dec. 10, 2009 | Dec. 10, 2009 | Dec. 17, 2009 | GK_mouse_4.16 |
| 75 | Mouse | cultured | fc | Dec. 10, 2009 | Dec. 10, 2009 | Dec. 17, 2009 | GK_mouse_4.17 |
| 76 | Mouse | cultured | fc | Dec. 10, 2009 | Dec. 10, 2009 | Dec. 17, 2009 | GK_mouse_4.18 |
| 77 | Mouse | cultured | fc | Dec. 10, 2009 | Dec. 10, 2009 | Dec. 17, 2009 | GK_mouse_4.19 |
| 78 | Mouse | cultured | fc | Dec. 10, 2009 | Dec. 10, 2009 | Dec. 17, 2009 | GK_mouse_4.20 |
| 79 | Mouse | direct | pt | Dec. 10, 2009 | N/A | N/A | GK_mouse_4.1 |
| 80 | Mouse | direct | pt | Dec. 10, 2009 | N/A | N/A | GK_mouse_4.1 |
| 81 | Mouse | direct | pt | Dec. 10, 2009 | N/A | N/A | GK_mouse_4.1 |
| 82 | Mouse | direct | pt | Dec. 10, 2009 | N/A | N/A | GK_mouse_4.1 |
| 83 | Mouse | direct | pt | Dec. 10, 2009 | N/A | N/A | GK_mouse_4.1 |
| 84 | Mouse | direct | pt | Dec. 10, 2009 | N/A | N/A | GK_mouse_4.1 |
| 85 | Mouse | direct | pt | Dec. 10, 2009 | N/A | N/A | GK_mouse_4.2 |
| 86 | Mouse | direct | pt | Dec. 10, 2009 | N/A | N/A | GK_mouse_4.2 |
| 87 | Mouse | direct | pt | Dec. 10, 2009 | N/A | N/A | GK_mouse_4.2 |
| 88 | Mouse | direct | pt | Dec. 10, 2009 | N/A | N/A | GK_mouse_4.2 |
| 89 | Mouse | direct | pt | Dec. 10, 2009 | N/A | N/A | GK_mouse_4.2 |
| 90 | Mouse | direct | pt | Dec. 10, 2009 | N/A | N/A | GK_mouse_4.2 |
| 91 | Mouse | direct | pt | Dec. 10, 2009 | N/A | N/A | GK_mouse_4.3 |
| 92 | Mouse | direct | pt | Dec. 10, 2009 | N/A | N/A | GK_mouse_4.3 |
| 93 | Mouse | direct | pt | Dec. 10, 2009 | N/A | N/A | GK_mouse_4.3 |
| 94 | Mouse | direct | pt | Dec. 10, 2009 | N/A | N/A | GK_mouse_4.3 |
| 95 | Mouse | direct | pt | Dec. 10, 2009 | N/A | N/A | GK_mouse_4.3 |
| 96 | Mouse | direct | pt | Dec. 10, 2009 | N/A | N/A | GK_mouse_4.3 |
| 97 | Mouse | direct | pt | Dec. 10, 2009 | N/A | N/A | GK_mouse_4.4 |
| 98 | Mouse | direct | pt | Dec. 10, 2009 | N/A | N/A | GK_mouse_4.4 |
| 102 | Mouse | direct | pt | Dec. 10, 2009 | N/A | N/A | GK_mouse_4.4 |
| 108 | Mouse | direct | pt | Dec. 10, 2009 | N/A | N/A | GK_mouse_4.5 |
| 110 | Mouse | direct | pt | Dec. 10, 2009 | N/A | N/A | GK_mouse_4.6 |
| 111 | Mouse | direct | pt | Dec. 10, 2009 | N/A | N/A | GK_mouse_4.6 |
| 112 | Mouse | direct | pt | Dec. 10, 2009 | N/A | N/A | GK_mouse_4.6 |
| 113 | Mouse | direct | pt | Dec. 10, 2009 | N/A | N/A | GK_mouse_4.6 |
| 114 | Mouse | direct | pt | Dec. 10, 2009 | N/A | N/A | GK_mouse_4.6 |
| 115 | Mouse | direct | pt | Dec. 10, 2009 | N/A | N/A | GK_mouse_4.7 |
| 116 | Mouse | direct | pt | Dec. 10, 2009 | N/A | N/A | GK_mouse_4.7 |
| 120 | Mouse | direct | pt | Dec. 10, 2009 | N/A | N/A | GK_mouse_4.7 |
| 126 | Mouse | direct | pt | Dec. 10, 2009 | N/A | N/A | GK_mouse_4.8 |
| 132 | Mouse | direct | pt | Dec. 10, 2009 | N/A | N/A | GK_mouse_4.9 |
| 144 | Mouse | cultured | pt | Dec. 10, 2009 | Dec. 10, 2009 | Dec. 17, 2009 | GK_mouse_4.11 |
| 150 | Mouse | cultured | pt | Dec. 10, 2009 | Dec. 10, 2009 | Dec. 17, 2009 | GK_mouse_4.13 |
| 156 | Mouse | cultured | pt | Dec. 10, 2009 | Dec. 10, 2009 | Dec. 17, 2009 | GK_mouse_4.15 |
| 162 | Mouse | cultured | pt | Dec. 10, 2009 | Dec. 10, 2009 | Dec. 17, 2009 | GK_mouse_4.16 |
| 168 | Mouse | cultured | pt | Dec. 10, 2009 | Dec. 10, 2009 | Dec. 17, 2009 | GK_mouse_4.17 |
| 174 | Mouse | cultured | pt | Dec. 10, 2009 | Dec. 10, 2009 | Dec. 17, 2009 | GK_mouse_4.18 |
| 180 | Mouse | cultured | pt | Dec. 10, 2009 | Dec. 10, 2009 | Dec. 17, 2009 | GK_mouse_4.19 |
| 186 | Mouse | cultured | pt | Dec. 10, 2009 | Dec. 10, 2009 | Dec. 17, 2009 | GK_mouse_4.20 |
| 187 | Mouse | direct | fc | Dec. 10, 2009 | N/A | N/A | GK_mouse_4.1 |
| 188 | Mouse | direct | fc | Dec. 10, 2009 | N/A | N/A | GK_mouse_4.2 |
| 189 | Mouse | direct | fc | Dec. 10, 2009 | N/A | N/A | GK_mouse_4.3 |
| 190 | Mouse | direct | fc | Dec. 10, 2009 | N/A | N/A | GK_mouse_4.4 |
| 191 | Mouse | direct | fc | Dec. 10, 2009 | N/A | N/A | GK_mouse_4.5 |
| 192 | Mouse | direct | fc | Dec. 10, 2009 | N/A | N/A | GK_mouse_4.6 |
| 193 | Mouse | direct | fc | Dec. 10, 2009 | N/A | N/A | GK_mouse_4.7 |
| 194 | Mouse | direct | fc | Dec. 10, 2009 | N/A | N/A | GK_mouse_4.8 |
| 195 | Mouse | direct | fc | Dec. 10, 2009 | N/A | N/A | GK_mouse_4.9 |
| 196 | Mouse | direct | fc | Dec. 10, 2009 | N/A | N/A | GK_mouse_4.10 |
| 197 | Mouse | cultured | fc | Dec. 10, 2009 | Dec. 10, 2009 | Dec. 17, 2009 | GK_mouse_4.11 |
| 198 | Mouse | cultured | fc | Dec. 10, 2009 | Dec. 10, 2009 | Dec. 17, 2009 | GK_mouse_4.13 |
| 199 | Mouse | cultured | fc | Dec. 10, 2009 | Dec. 10, 2009 | Dec. 17, 2009 | GK_mouse_4.15 |
| 200 | Mouse | cultured | fc | Dec. 10, 2009 | Dec. 10, 2009 | Dec. 17, 2009 | GK_mouse_4.16 |
| 201 | Mouse | cultured | fc | Dec. 10, 2009 | Dec. 10, 2009 | Dec. 17, 2009 | GK_mouse_4.17 |
| 202 | Mouse | cultured | fc | Dec. 10, 2009 | Dec. 10, 2009 | Dec. 17, 2009 | GK_mouse_4.18 |
| 203 | Mouse | cultured | fc | Dec. 10, 2009 | Dec. 10, 2009 | Dec. 17, 2009 | GK_mouse_4.19 |
| 204 | Mouse | cultured | fc | Dec. 10, 2009 | Dec. 10, 2009 | Dec. 17, 2009 | GK_mouse_4.20 |
| 205 | Mouse | direct | fc | Dec. 10, 2009 | N/A | N/A | GK_mouse_4.1 |
| 207 | Mouse | direct | fc | Dec. 10, 2009 | N/A | N/A | GK_mouse_4.3 |
| 208 | Mouse | direct | fc | Dec. 10, 2009 | N/A | N/A | GK_mouse_4.4 |
| 209 | Mouse | direct | fc | Dec. 10, 2009 | N/A | N/A | GK_mouse_4.5 |
| 210 | Mouse | direct | fc | Dec. 10, 2009 | N/A | N/A | GK_mouse_4.6 |
| 211 | Mouse | direct | fc | Dec. 10, 2009 | N/A | N/A | GK_mouse_4.7 |
| 212 | Mouse | direct | fc | Dec. 10, 2009 | N/A | N/A | GK_mouse_4.8 |
| 213 | Mouse | direct | fc | Dec. 10, 2009 | N/A | N/A | GK_mouse_4.9 |
| 214 | Mouse | direct | fc | Dec. 10, 2009 | N/A | N/A | GK_mouse_4.10 |
| 215 | Mouse | cultured | fc | Dec. 10, 2009 | Dec. 10, 2009 | Dec. 17, 2009 | GK_mouse_4.11 |
| 216 | Mouse | cultured | fc | Dec. 10, 2009 | Dec. 10, 2009 | Dec. 17, 2009 | GK_mouse_4.13 |
| 217 | Mouse | cultured | fc | Dec. 10, 2009 | Dec. 10, 2009 | Dec. 17, 2009 | GK_mouse_4.15 |
| 218 | Mouse | cultured | fc | Dec. 10, 2009 | Dec. 10, 2009 | Dec. 17, 2009 | GK_mouse_4.16 |
| 219 | Mouse | cultured | fc | Dec. 10, 2009 | Dec. 10, 2009 | Dec. 17, 2009 | GK_mouse_4.17 |
| 220 | Mouse | cultured | fc | Dec. 10, 2009 | Dec. 10, 2009 | Dec. 17, 2009 | GK_mouse_4.18 |
| 221 | Mouse | cultured | fc | Dec. 10, 2009 | Dec. 10, 2009 | Dec. 17, 2009 | GK_mouse_4.19 |
| 222 | Mouse | cultured | fc | Dec. 10, 2009 | Dec. 10, 2009 | Dec. 17, 2009 | GK_mouse_4.20 |
| 223 | Mouse | direct | fc | Dec. 10, 2009 | N/A | N/A | GK_mouse_4.1 |
| 224 | Mouse | direct | fc | Dec. 10, 2009 | N/A | N/A | GK_mouse_4.2 |
| 225 | Mouse | direct | fc | Dec. 10, 2009 | N/A | N/A | GK_mouse_4.3 |
| 226 | Mouse | direct | fc | Dec. 10, 2009 | N/A | N/A | GK_mouse_4.4 |
| 227 | Mouse | direct | fc | Dec. 10, 2009 | N/A | N/A | GK_mouse_4.5 |
| 228 | Mouse | direct | fc | Dec. 10, 2009 | N/A | N/A | GK_mouse_4.6 |
| 229 | Mouse | direct | fc | Dec. 10, 2009 | N/A | N/A | GK_mouse_4.7 |
| 230 | Mouse | direct | fc | Dec. 10, 2009 | N/A | N/A | GK_mouse_4.8 |
| 231 | Mouse | direct | fc | Dec. 10, 2009 | N/A | N/A | GK_mouse_4.9 |
| 232 | Mouse | direct | fc | Dec. 10, 2009 | N/A | N/A | GK_mouse_4.10 |
| 233 | Mouse | cultured | fc | Dec. 10, 2009 | Dec. 10, 2009 | Dec. 17, 2009 | GK_mouse_4.11 |
| 234 | Mouse | cultured | fc | Dec. 10, 2009 | Dec. 10, 2009 | Dec. 17, 2009 | GK_mouse_4.13 |
| 235 | Mouse | cultured | fc | Dec. 10, 2009 | Dec. 10, 2009 | Dec. 17, 2009 | GK_mouse_4.15 |
| 236 | Mouse | cultured | fc | Dec. 10, 2009 | Dec. 10, 2009 | Dec. 17, 2009 | GK_mouse_4.16 |
| 238 | Mouse | cultured | fc | Dec. 10, 2009 | Dec. 10, 2009 | Dec. 17, 2009 | GK_mouse_4.18 |
| 240 | Mouse | cultured | fc | Dec. 10, 2009 | Dec. 10, 2009 | Dec. 17, 2009 | GK_mouse_4.20 |
| 261 | Mouse | direct | fc | Dec. 10, 2009 | N/A | N/A | GK_mouse_4.3 |
| 262 | Mouse | direct | fc | Dec. 10, 2009 | N/A | N/A | GK_mouse_4.4 |
| 263 | Mouse | direct | fc | Dec. 10, 2009 | N/A | N/A | GK_mouse_4.5 |
| 265 | Mouse | direct | fc | Dec. 10, 2009 | N/A | N/A | GK_mouse_4.7 |
| 266 | Mouse | direct | fc | Dec. 10, 2009 | N/A | N/A | GK_mouse_4.8 |
| 267 | Mouse | direct | fc | Dec. 10, 2009 | N/A | N/A | GK_mouse_4.9 |
| 268 | Mouse | direct | fc | Dec. 10, 2009 | N/A | N/A | GK_mouse_4.10 |
| 269 | Mouse | cultured | fc | Dec. 10, 2009 | Dec. 10, 2009 | Dec. 17, 2009 | GK_mouse_4.11 |
| 270 | Mouse | cultured | fc | Dec. 10, 2009 | Dec. 10, 2009 | Dec. 17, 2009 | GK_mouse_4.13 |
| 271 | Mouse | cultured | fc | Dec. 10, 2009 | Dec. 10, 2009 | Dec. 17, 2009 | GK_mouse_4.15 |
| 272 | Mouse | cultured | fc | Dec. 10, 2009 | Dec. 10, 2009 | Dec. 17, 2009 | GK_mouse_4.16 |
| 273 | Mouse | cultured | fc | Dec. 10, 2009 | Dec. 10, 2009 | Dec. 17, 2009 | GK_mouse_4.17 |
| 274 | Mouse | cultured | fc | Dec. 10, 2009 | Dec. 10, 2009 | Dec. 17, 2009 | GK_mouse_4.18 |
| 275 | Mouse | cultured | fc | Dec. 10, 2009 | Dec. 10, 2009 | Dec. 17, 2009 | GK_mouse_4.19 |
| 276 | Mouse | cultured | fc | Dec. 10, 2009 | Dec. 10, 2009 | Dec. 17, 2009 | GK_mouse_4.20 |
| 277 | Mouse | direct | pt | Dec. 10, 2009 | N/A | N/A | GK_mouse_4.1 |
| 278 | Mouse | direct | pt | Dec. 10, 2009 | N/A | N/A | GK_mouse_4.2 |
| 279 | Mouse | direct | pt | Dec. 10, 2009 | N/A | N/A | GK_mouse_4.3 |
| 280 | Mouse | direct | pt | Dec. 10, 2009 | N/A | N/A | GK_mouse_4.4 |
| 281 | Mouse | direct | pt | Dec. 10, 2009 | N/A | N/A | GK_mouse_4.5 |
| 282 | Mouse | direct | pt | Dec. 10, 2009 | N/A | N/A | GK_mouse_4.6 |
| 284 | Mouse | direct | pt | Dec. 10, 2009 | N/A | N/A | GK_mouse_4.8 |
| 285 | Mouse | direct | pt | Dec. 10, 2009 | N/A | N/A | GK_mouse_4.9 |
| 287 | Mouse | cultured | pt | Dec. 10, 2009 | Dec. 10, 2009 | Dec. 17, 2009 | GK_mouse_4.11 |
| 288 | Mouse | cultured | pt | Dec. 10, 2009 | Dec. 10, 2009 | Dec. 17, 2009 | GK_mouse_4.13 |
| 289 | Mouse | cultured | pt | Dec. 10, 2009 | Dec. 10, 2009 | Dec. 17, 2009 | GK_mouse_4.15 |
| 290 | Mouse | cultured | pt | Dec. 10, 2009 | Dec. 10, 2009 | Dec. 17, 2009 | GK_mouse_4.16 |
| 291 | Mouse | cultured | pt | Dec. 10, 2009 | Dec. 10, 2009 | Dec. 17, 2009 | GK_mouse_4.17 |
| 292 | Mouse | cultured | pt | Dec. 10, 2009 | Dec. 10, 2009 | Dec. 17, 2009 | GK_mouse_4.18 |
| 293 | Mouse | cultured | pt | Dec. 10, 2009 | Dec. 10, 2009 | Dec. 17, 2009 | GK_mouse_4.19 |
| 294 | Mouse | cultured | pt | Dec. 10, 2009 | Dec. 10, 2009 | Dec. 17, 2009 | GK_mouse_4.20 |
| 295 | Mouse | direct | fc | Dec. 10, 2009 | N/A | N/A | GK_mouse_4.1 |
| 296 | Mouse | direct | fc | Dec. 10, 2009 | N/A | N/A | GK_mouse_4.2 |
| 297 | Mouse | direct | fc | Dec. 10, 2009 | N/A | N/A | GK_mouse_4.3 |
| 298 | Mouse | direct | fc | Dec. 10, 2009 | N/A | N/A | GK_mouse_4.4 |
| 299 | Mouse | direct | fc | Dec. 10, 2009 | N/A | N/A | GK_mouse_4.5 |
| 300 | Mouse | direct | fc | Dec. 10, 2009 | N/A | N/A | GK_mouse_4.6 |
| 301 | Mouse | direct | fc | Dec. 10, 2009 | N/A | N/A | GK_mouse_4.7 |
| 302 | Mouse | direct | fc | Dec. 10, 2009 | N/A | N/A | GK_mouse_4.8 |
| 303 | Mouse | direct | fc | Dec. 10, 2009 | N/A | N/A | GK_mouse_4.9 |
| 304 | Mouse | direct | fc | Dec. 10, 2009 | N/A | N/A | GK_mouse_4.10 |
| 305 | Mouse | cultured | fc | Dec. 10, 2009 | Dec. 10, 2009 | Dec. 17, 2009 | GK_mouse_4.11 |
| 306 | Mouse | cultured | fc | Dec. 10, 2009 | Dec. 10, 2009 | Dec. 17, 2009 | GK_mouse_4.13 |
| 307 | Mouse | cultured | fc | Dec. 10, 2009 | Dec. 10, 2009 | Dec. 17, 2009 | GK_mouse_4.15 |
| 308 | Mouse | cultured | fc | Dec. 10, 2009 | Dec. 10, 2009 | Dec. 17, 2009 | GK_mouse_4.16 |
| 309 | Mouse | cultured | fc | Dec. 10, 2009 | Dec. 10, 2009 | Dec. 17, 2009 | GK_mouse_4.17 |
| 310 | Mouse | cultured | fc | Dec. 10, 2009 | Dec. 10, 2009 | Dec. 17, 2009 | GK_mouse_4.18 |
| 311 | Mouse | cultured | fc | Dec. 10, 2009 | Dec. 10, 2009 | Dec. 17, 2009 | GK_mouse_4.19 |
| 312 | Mouse | cultured | fc | Dec. 10, 2009 | Dec. 10, 2009 | Dec. 17, 2009 | GK_mouse_4.20 |
| 313 | Mouse | direct | fc | Dec. 10, 2009 | N/A | N/A | GK_mouse_4.1 |
| 314 | Mouse | direct | fc | Dec. 10, 2009 | N/A | N/A | GK_mouse_4.2 |
| 315 | Mouse | direct | fc | Dec. 10, 2009 | N/A | N/A | GK_mouse_4.3 |
| 316 | Mouse | direct | fc | Dec. 10, 2009 | N/A | N/A | GK_mouse_4.4 |
| 317 | Mouse | direct | fc | Dec. 10, 2009 | N/A | N/A | GK_mouse_4.5 |
| 318 | Mouse | direct | fc | Dec. 10, 2009 | N/A | N/A | GK_mouse_4.6 |
| 319 | Mouse | direct | fc | Dec. 10, 2009 | N/A | N/A | GK_mouse_4.7 |
| 321 | Mouse | direct | fc | Dec. 10, 2009 | N/A | N/A | GK_mouse_4.9 |
| 323 | Mouse | cultured | fc | Dec. 10, 2009 | Dec. 10, 2009 | Dec. 17, 2009 | GK_mouse_4.11 |
| 324 | Mouse | cultured | fc | Dec. 10, 2009 | Dec. 10, 2009 | Dec. 17, 2009 | GK_mouse_4.13 |
| 325 | Mouse | cultured | fc | Dec. 10, 2009 | Dec. 10, 2009 | Dec. 17, 2009 | GK_mouse_4.15 |
| 326 | Mouse | cultured | fc | Dec. 10, 2009 | Dec. 10, 2009 | Dec. 17, 2009 | GK_mouse_4.16 |
| 328 | Mouse | cultured | fc | Dec. 10, 2009 | Dec. 10, 2009 | Dec. 17, 2009 | GK_mouse_4.18 |
| 329 | Mouse | cultured | fc | Dec. 10, 2009 | Dec. 10, 2009 | Dec. 17, 2009 | GK_mouse_4.19 |
| 330 | Mouse | cultured | fc | Dec. 10, 2009 | Dec. 10, 2009 | Dec. 17, 2009 | GK_mouse_4.20 |
| 332 | Mouse | direct | fc | Dec. 10, 2009 | N/A | N/A | GK_mouse_4.2 |
| 333 | Mouse | direct | fc | Dec. 10, 2009 | N/A | N/A | GK_mouse_4.3 |
| 334 | Mouse | direct | fc | Dec. 10, 2009 | N/A | N/A | GK_mouse_4.4 |
| 335 | Mouse | direct | fc | Dec. 10, 2009 | N/A | N/A | GK_mouse_4.5 |
| 338 | Mouse | direct | fc | Dec. 10, 2009 | N/A | N/A | GK_mouse_4.8 |
| 339 | Mouse | direct | fc | Dec. 10, 2009 | N/A | N/A | GK_mouse_4.9 |
| 340 | Mouse | direct | fc | Dec. 10, 2009 | N/A | N/A | GK_mouse_4.10 |
| 341 | Mouse | cultured | fc | Dec. 10, 2009 | Dec. 10, 2009 | Dec. 17, 2009 | GK_mouse_4.11 |
| 342 | Mouse | cultured | fc | Dec. 10, 2009 | Dec. 10, 2009 | Dec. 17, 2009 | GK_mouse_4.13 |
| 343 | Mouse | cultured | fc | Dec. 10, 2009 | Dec. 10, 2009 | Dec. 17, 2009 | GK_mouse_4.15 |
| 344 | Mouse | cultured | fc | Dec. 10, 2009 | Dec. 10, 2009 | Dec. 17, 2009 | GK_mouse_4.16 |
| 345 | Mouse | cultured | fc | Dec. 10, 2009 | Dec. 10, 2009 | Dec. 17, 2009 | GK_mouse_4.17 |
| 347 | Mouse | cultured | fc | Dec. 10, 2009 | Dec. 10, 2009 | Dec. 17, 2009 | GK_mouse_4.19 |
| 348 | Mouse | cultured | fc | Dec. 10, 2009 | Dec. 10, 2009 | Dec. 17, 2009 | GK_mouse_4.20 |
| 349 | Mouse | direct | pt | Dec. 10, 2009 | N/A | N/A | GK_mouse_4.1 |
| 350 | Mouse | direct | pt | Dec. 10, 2009 | N/A | N/A | GK_mouse_4.2 |
| 351 | Mouse | direct | pt | Dec. 10, 2009 | N/A | N/A | GK_mouse_4.3 |
| 352 | Mouse | direct | pt | Dec. 10, 2009 | N/A | N/A | GK_mouse_4.4 |
| 353 | Mouse | direct | pt | Dec. 10, 2009 | N/A | N/A | GK_mouse_4.5 |
| 354 | Mouse | direct | pt | Dec. 10, 2009 | N/A | N/A | GK_mouse_4.6 |
| 355 | Mouse | direct | pt | Dec. 10, 2009 | N/A | N/A | GK_mouse_4.7 |
| 356 | Mouse | direct | pt | Dec. 10, 2009 | N/A | N/A | GK_mouse_4.8 |
| 357 | Mouse | direct | pt | Dec. 10, 2009 | N/A | N/A | GK_mouse_4.9 |
| 358 | Mouse | direct | pt | Dec. 10, 2009 | N/A | N/A | GK_mouse_4.10 |
| 359 | Mouse | cultured | pt | Dec. 10, 2009 | Dec. 10, 2009 | Dec. 17, 2009 | GK_mouse_4.11 |
| 360 | Mouse | cultured | pt | Dec. 10, 2009 | Dec. 10, 2009 | Dec. 17, 2009 | GK_mouse_4.13 |
| 361 | Mouse | cultured | pt | Dec. 10, 2009 | Dec. 10, 2009 | Dec. 17, 2009 | GK_mouse_4.15 |
| 362 | Mouse | cultured | pt | Dec. 10, 2009 | Dec. 10, 2009 | Dec. 17, 2009 | GK_mouse_4.16 |
| 363 | Mouse | cultured | pt | Dec. 10, 2009 | Dec. 10, 2009 | Dec. 17, 2009 | GK_mouse_4.17 |
| 364 | Mouse | cultured | pt | Dec. 10, 2009 | Dec. 10, 2009 | Dec. 17, 2009 | GK_mouse_4.18 |
| 365 | Mouse | cultured | pt | Dec. 10, 2009 | Dec. 10, 2009 | Dec. 17, 2009 | GK_mouse_4.19 |
| 366 | Mouse | cultured | pt | Dec. 10, 2009 | Dec. 10, 2009 | Dec. 17, 2009 | GK_mouse_4.20 |
| 367 | Mouse | direct | fc | Dec. 10, 2009 | N/A | N/A | GK_mouse_4.1 |
| 369 | Mouse | direct | fc | Dec. 10, 2009 | N/A | N/A | GK_mouse_4.3 |
| 370 | Mouse | direct | fc | Dec. 10, 2009 | N/A | N/A | GK_mouse_4.4 |
| 371 | Mouse | direct | fc | Dec. 10, 2009 | N/A | N/A | GK_mouse_4.5 |
| 372 | Mouse | direct | fc | Dec. 10, 2009 | N/A | N/A | GK_mouse_4.6 |
| 373 | Mouse | direct | fc | Dec. 10, 2009 | N/A | N/A | GK_mouse_4.7 |
| 374 | Mouse | direct | fc | Dec. 10, 2009 | N/A | N/A | GK_mouse_4.8 |
| 375 | Mouse | direct | fc | Dec. 10, 2009 | N/A | N/A | GK_mouse_4.9 |
| 376 | Mouse | direct | fc | Dec. 10, 2009 | N/A | N/A | GK_mouse_4.10 |
| 377 | Mouse | cultured | fc | Dec. 10, 2009 | Dec. 10, 2009 | Dec. 17, 2009 | GK_mouse_4.11 |
| 378 | Mouse | cultured | fc | Dec. 10, 2009 | Dec. 10, 2009 | Dec. 17, 2009 | GK_mouse_4.13 |
| 379 | Mouse | cultured | fc | Dec. 10, 2009 | Dec. 10, 2009 | Dec. 17, 2009 | GK_mouse_4.15 |
| 380 | Mouse | cultured | fc | Dec. 10, 2009 | Dec. 10, 2009 | Dec. 17, 2009 | GK_mouse_4.16 |
| 381 | Mouse | cultured | fc | Dec. 10, 2009 | Dec. 10, 2009 | Dec. 17, 2009 | GK_mouse_4.17 |
| 382 | Mouse | cultured | fc | Dec. 10, 2009 | Dec. 10, 2009 | Dec. 17, 2009 | GK_mouse_4.18 |
| 383 | Mouse | cultured | fc | Dec. 10, 2009 | Dec. 10, 2009 | Dec. 17, 2009 | GK_mouse_4.19 |
| 384 | Mouse | cultured | fc | Dec. 10, 2009 | Dec. 10, 2009 | Dec. 17, 2009 | GK_mouse_4.20 |
| 385 | Mouse | direct | fc | Dec. 10, 2009 | N/A | N/A | GK_mouse_4.1 |
| 386 | Mouse | direct | fc | Dec. 10, 2009 | N/A | N/A | GK_mouse_4.2 |
| 387 | Mouse | direct | fc | Dec. 10, 2009 | N/A | N/A | GK_mouse_4.3 |
| 388 | Mouse | direct | fc | Dec. 10, 2009 | N/A | N/A | GK_mouse_4.4 |
| 389 | Mouse | direct | fc | Dec. 10, 2009 | N/A | N/A | GK_mouse_4.5 |
| 390 | Mouse | direct | fc | Dec. 10, 2009 | N/A | N/A | GK_mouse_4.6 |
| 391 | Mouse | direct | fc | Dec. 10, 2009 | N/A | N/A | GK_mouse_4.7 |
| 392 | Mouse | direct | fc | Dec. 10, 2009 | N/A | N/A | GK_mouse_4.8 |
| 393 | Mouse | direct | fc | Dec. 10, 2009 | N/A | N/A | GK_mouse_4.9 |
| 394 | Mouse | direct | fc | Dec. 10, 2009 | N/A | N/A | GK_mouse_4.10 |
| 395 | Mouse | cultured | fc | Dec. 10, 2009 | Dec. 10, 2009 | Dec. 17, 2009 | GK_mouse_4.11 |
| 396 | Mouse | cultured | fc | Dec. 10, 2009 | Dec. 10, 2009 | Dec. 17, 2009 | GK_mouse_4.13 |
| 397 | Mouse | cultured | fc | Dec. 10, 2009 | Dec. 10, 2009 | Dec. 17, 2009 | GK_mouse_4.15 |
| 398 | Mouse | cultured | fc | Dec. 10, 2009 | Dec. 10, 2009 | Dec. 17, 2009 | GK_mouse_4.16 |
| 399 | Mouse | cultured | fc | Dec. 10, 2009 | Dec. 10, 2009 | Dec. 17, 2009 | GK_mouse_4.17 |
| 400 | Mouse | cultured | fc | Dec. 10, 2009 | Dec. 10, 2009 | Dec. 17, 2009 | GK_mouse_4.18 |
| 401 | Mouse | cultured | fc | Dec. 10, 2009 | Dec. 10, 2009 | Dec. 17, 2009 | GK_mouse_4.19 |
| 402 | Mouse | cultured | fc | Dec. 10, 2009 | Dec. 10, 2009 | Dec. 17, 2009 | GK_mouse_4.20 |
| 403 | Mouse | direct | pt | Dec. 10, 2009 | N/A | N/A | GK_mouse_4.1 |
| 404 | Mouse | direct | pt | Dec. 10, 2009 | N/A | N/A | GK_mouse_4.2 |
| 405 | Mouse | direct | pt | Dec. 10, 2009 | N/A | N/A | GK_mouse_4.3 |
| 406 | Mouse | direct | pt | Dec. 10, 2009 | N/A | N/A | GK_mouse_4.4 |
| 407 | Mouse | direct | pt | Dec. 10, 2009 | N/A | N/A | GK_mouse_4.5 |
| 408 | Mouse | direct | pt | Dec. 10, 2009 | N/A | N/A | GK_mouse_4.6 |
| 409 | Mouse | direct | pt | Dec. 10, 2009 | N/A | N/A | GK_mouse_4.7 |
| 410 | Mouse | direct | pt | Dec. 10, 2009 | N/A | N/A | GK_mouse_4.8 |
| 411 | Mouse | direct | pt | Dec. 10, 2009 | N/A | N/A | GK_mouse_4.9 |
| 412 | Mouse | direct | pt | Dec. 10, 2009 | N/A | N/A | GK_mouse_4.10 |
| 413 | Mouse | cultured | pt | Dec. 10, 2009 | Dec. 10, 2009 | Dec. 17, 2009 | GK_mouse_4.11 |
| 414 | Mouse | cultured | pt | Dec. 10, 2009 | Dec. 10, 2009 | Dec. 17, 2009 | GK_mouse_4.13 |
| 416 | Mouse | cultured | pt | Dec. 10, 2009 | Dec. 10, 2009 | Dec. 17, 2009 | GK_mouse_4.16 |
| 417 | Mouse | cultured | pt | Dec. 10, 2009 | Dec. 10, 2009 | Dec. 17, 2009 | GK_mouse_4.17 |
| 418 | Mouse | cultured | pt | Dec. 10, 2009 | Dec. 10, 2009 | Dec. 17, 2009 | GK_mouse_4.18 |
| 419 | Mouse | cultured | pt | Dec. 10, 2009 | Dec. 10, 2009 | Dec. 17, 2009 | GK_mouse_4.19 |
| 420 | Mouse | cultured | pt | Dec. 10, 2009 | Dec. 10, 2009 | Dec. 17, 2009 | GK_mouse_4.20 |
| 421 | Mouse | direct | fc | Dec. 10, 2009 | N/A | N/A | GK_mouse_4.1 |
| 422 | Mouse | direct | fc | Dec. 10, 2009 | N/A | N/A | GK_mouse_4.2 |
| 423 | Mouse | direct | fc | Dec. 10, 2009 | N/A | N/A | GK_mouse_4.3 |
| 424 | Mouse | direct | fc | Dec. 10, 2009 | N/A | N/A | GK_mouse_4.4 |
| 425 | Mouse | direct | fc | Dec. 10, 2009 | N/A | N/A | GK_mouse_4.5 |
| 426 | Mouse | direct | fc | Dec. 10, 2009 | N/A | N/A | GK_mouse_4.6 |
| 427 | Mouse | direct | fc | Dec. 10, 2009 | N/A | N/A | GK_mouse_4.7 |
| 428 | Mouse | direct | fc | Dec. 10, 2009 | N/A | N/A | GK_mouse_4.8 |
| 429 | Mouse | direct | fc | Dec. 10, 2009 | N/A | N/A | GK_mouse_4.9 |
| 430 | Mouse | direct | fc | Dec. 10, 2009 | N/A | N/A | GK_mouse_4.10 |
| 431 | Mouse | cultured | fc | Dec. 10, 2009 | Dec. 10, 2009 | Dec. 17, 2009 | GK_mouse_4.11 |
| 432 | Mouse | cultured | fc | Dec. 10, 2009 | Dec. 10, 2009 | Dec. 17, 2009 | GK_mouse_4.13 |
| 433 | Mouse | cultured | fc | Dec. 10, 2009 | Dec. 10, 2009 | Dec. 17, 2009 | GK_mouse_4.15 |
| 434 | Mouse | cultured | fc | Dec. 10, 2009 | Dec. 10, 2009 | Dec. 17, 2009 | GK_mouse_4.16 |
| 435 | Mouse | cultured | fc | Dec. 10, 2009 | Dec. 10, 2009 | Dec. 17, 2009 | GK_mouse_4.17 |
| 436 | Mouse | cultured | fc | Dec. 10, 2009 | Dec. 10, 2009 | Dec. 17, 2009 | GK_mouse_4.18 |
| 437 | Mouse | cultured | fc | Dec. 10, 2009 | Dec. 10, 2009 | Dec. 17, 2009 | GK_mouse_4.19 |
| 438 | Mouse | cultured | fc | Dec. 10, 2009 | Dec. 10, 2009 | Dec. 17, 2009 | GK_mouse_4.20 |
| 439 | Mouse | direct | fc | Dec. 10, 2009 | N/A | N/A | GK_mouse_4.1 |
| 440 | Mouse | direct | fc | Dec. 10, 2009 | N/A | N/A | GK_mouse_4.2 |
| 441 | Mouse | direct | fc | Dec. 10, 2009 | N/A | N/A | GK_mouse_4.3 |
| 442 | Mouse | direct | fc | Dec. 10, 2009 | N/A | N/A | GK_mouse_4.4 |
| 443 | Mouse | direct | fc | Dec. 10, 2009 | N/A | N/A | GK_mouse_4.5 |
| 444 | Mouse | direct | fc | Dec. 10, 2009 | N/A | N/A | GK_mouse_4.6 |
| 445 | Mouse | direct | fc | Dec. 10, 2009 | N/A | N/A | GK_mouse_4.7 |
| 446 | Mouse | direct | fc | Dec. 10, 2009 | N/A | N/A | GK_mouse_4.8 |
| 447 | Mouse | direct | fc | Dec. 10, 2009 | N/A | N/A | GK_mouse_4.9 |
| 448 | Mouse | direct | fc | Dec. 10, 2009 | N/A | N/A | GK_mouse_4.10 |
| 449 | Mouse | cultured | fc | Dec. 10, 2009 | Dec. 10, 2009 | Dec. 17, 2009 | GK_mouse_4.11 |
| 450 | Mouse | cultured | fc | Dec. 10, 2009 | Dec. 10, 2009 | Dec. 17, 2009 | GK_mouse_4.13 |
| 451 | Mouse | cultured | fc | Dec. 10, 2009 | Dec. 10, 2009 | Dec. 17, 2009 | GK_mouse_4.15 |
| 452 | Mouse | cultured | fc | Dec. 10, 2009 | Dec. 10, 2009 | Dec. 17, 2009 | GK_mouse_4.16 |
| 453 | Mouse | cultured | fc | Dec. 10, 2009 | Dec. 10, 2009 | Dec. 17, 2009 | GK_mouse_4.17 |
| 454 | Mouse | cultured | fc | Dec. 10, 2009 | Dec. 10, 2009 | Dec. 17, 2009 | GK_mouse_4.18 |
| 455 | Mouse | cultured | fc | Dec. 10, 2009 | Dec. 10, 2009 | Dec. 17, 2009 | GK_mouse_4.19 |
| 456 | Mouse | cultured | fc | Dec. 10, 2009 | Dec. 10, 2009 | Dec. 17, 2009 | GK_mouse_4.20 |
| 457 | Mouse | direct | fc | Dec. 10, 2009 | N/A | N/A | GK_mouse_4.1 |
| 458 | Mouse | direct | fc | Dec. 10, 2009 | N/A | N/A | GK_mouse_4.2 |
| 459 | Mouse | direct | fc | Dec. 10, 2009 | N/A | N/A | GK_mouse_4.3 |
| 460 | Mouse | direct | fc | Dec. 10, 2009 | N/A | N/A | GK_mouse_4.4 |
| 461 | Mouse | direct | fc | Dec. 10, 2009 | N/A | N/A | GK_mouse_4.5 |
| 462 | Mouse | direct | fc | Dec. 10, 2009 | N/A | N/A | GK_mouse_4.6 |
| 463 | Mouse | direct | fc | Dec. 10, 2009 | N/A | N/A | GK_mouse_4.7 |
| 465 | Mouse | direct | fc | Dec. 10, 2009 | N/A | N/A | GK_mouse_4.9 |
| 466 | Mouse | direct | fc | Dec. 10, 2009 | N/A | N/A | GK_mouse_4.10 |
| 467 | Mouse | cultured | fc | Dec. 10, 2009 | Dec. 10, 2009 | Dec. 17, 2009 | GK_mouse_4.11 |
| 468 | Mouse | cultured | fc | Dec. 10, 2009 | Dec. 10, 2009 | Dec. 17, 2009 | GK_mouse_4.13 |
| 469 | Mouse | cultured | fc | Dec. 10, 2009 | Dec. 10, 2009 | Dec. 17, 2009 | GK_mouse_4.15 |
| 470 | Mouse | cultured | fc | Dec. 10, 2009 | Dec. 10, 2009 | Dec. 17, 2009 | GK_mouse_4.16 |
| 471 | Mouse | cultured | fc | Dec. 10, 2009 | Dec. 10, 2009 | Dec. 17, 2009 | GK_mouse_4.17 |
| 472 | Mouse | cultured | fc | Dec. 10, 2009 | Dec. 10, 2009 | Dec. 17, 2009 | GK_mouse_4.18 |
| 473 | Mouse | cultured | fc | Dec. 10, 2009 | Dec. 10, 2009 | Dec. 17, 2009 | GK_mouse_4.19 |
| 474 | Mouse | cultured | fc | Dec. 10, 2009 | Dec. 10, 2009 | Dec. 17, 2009 | GK_mouse_4.20 |
| 493 | Mouse | direct | Si2 | Dec. 10, 2009 | N/A | N/A | GK_mouse_4.1 |
| 494 | Mouse | direct | Si2 | Dec. 10, 2009 | N/A | N/A | GK_mouse_4.2 |
| 495 | Mouse | direct | Si2 | Dec. 10, 2009 | N/A | N/A | GK_mouse_4.3 |
| 496 | Mouse | direct | Si2 | Dec. 10, 2009 | N/A | N/A | GK_mouse_4.4 |
| 498 | Mouse | direct | Si2 | Dec. 10, 2009 | N/A | N/A | GK_mouse_4.6 |
| 499 | Mouse | direct | Si2 | Dec. 10, 2009 | N/A | N/A | GK_mouse_4.7 |
| 500 | Mouse | direct | Si2 | Dec. 10, 2009 | N/A | N/A | GK_mouse_4.8 |
| 501 | Mouse | direct | Si2 | Dec. 10, 2009 | N/A | N/A | GK_mouse_4.9 |
| 502 | Mouse | direct | Si2 | Dec. 10, 2009 | N/A | N/A | GK_mouse_4.10 |
| 503 | Mouse | cultured | Si2 | Dec. 10, 2009 | Dec. 10, 2009 | Dec. 17, 2009 | GK_mouse_4.11 |
| 505 | Mouse | cultured | Si2 | Dec. 10, 2009 | Dec. 10, 2009 | Dec. 17, 2009 | GK_mouse_4.15 |
| 506 | Mouse | cultured | Si2 | Dec. 10, 2009 | Dec. 10, 2009 | Dec. 17, 2009 | GK_mouse_4.16 |
| 507 | Mouse | cultured | Si2 | Dec. 10, 2009 | Dec. 10, 2009 | Dec. 17, 2009 | GK_mouse_4.17 |
| 509 | Mouse | cultured | Si2 | Dec. 10, 2009 | Dec. 10, 2009 | Dec. 17, 2009 | GK_mouse_4.19 |
| 510 | Mouse | cultured | Si2 | Dec. 10, 2009 | Dec. 10, 2009 | Dec. 17, 2009 | GK_mouse_4.20 |
| 511 | Mouse | direct | Si5 | Dec. 10, 2009 | N/A | N/A | GK_mouse_4.1 |
| 512 | Mouse | direct | Si5 | Dec. 10, 2009 | N/A | N/A | GK_mouse_4.2 |
| 513 | Mouse | direct | Si5 | Dec. 10, 2009 | N/A | N/A | GK_mouse_4.3 |
| 514 | Mouse | direct | Si5 | Dec. 10, 2009 | N/A | N/A | GK_mouse_4.4 |
| 515 | Mouse | direct | Si5 | Dec. 10, 2009 | N/A | N/A | GK_mouse_4.5 |
| 517 | Mouse | direct | Si5 | Dec. 10, 2009 | N/A | N/A | GK_mouse_4.7 |
| 518 | Mouse | direct | Si5 | Dec. 10, 2009 | N/A | N/A | GK_mouse_4.8 |
| 519 | Mouse | direct | Si5 | Dec. 10, 2009 | N/A | N/A | GK_mouse_4.9 |
| 520 | Mouse | direct | Si5 | Dec. 10, 2009 | N/A | N/A | GK_mouse_4.10 |
| 521 | Mouse | cultured | Si5 | Dec. 10, 2009 | Dec. 10, 2009 | Dec. 17, 2009 | GK_mouse_4.11 |
| 522 | Mouse | cultured | Si5 | Dec. 10, 2009 | Dec. 10, 2009 | Dec. 17, 2009 | GK_mouse_4.13 |
| 523 | Mouse | cultured | Si5 | Dec. 10, 2009 | Dec. 10, 2009 | Dec. 17, 2009 | GK_mouse_4.15 |
| 524 | Mouse | cultured | Si5 | Dec. 10, 2009 | Dec. 10, 2009 | Dec. 17, 2009 | GK_mouse_4.16 |
| 525 | Mouse | cultured | Si5 | Dec. 10, 2009 | Dec. 10, 2009 | Dec. 17, 2009 | GK_mouse_4.17 |
| 526 | Mouse | cultured | Si5 | Dec. 10, 2009 | Dec. 10, 2009 | Dec. 17, 2009 | GK_mouse_4.18 |
| 527 | Mouse | cultured | Si5 | Dec. 10, 2009 | Dec. 10, 2009 | Dec. 17, 2009 | GK_mouse_4.19 |
| 547 | Mouse | direct | Si13 | Dec. 10, 2009 | N/A | N/A | GK_mouse_4.1 |
| 548 | Mouse | direct | Si13 | Dec. 10, 2009 | N/A | N/A | GK_mouse_4.2 |
| 549 | Mouse | direct | Si13 | Dec. 10, 2009 | N/A | N/A | GK_mouse_4.3 |
| 550 | Mouse | direct | Si13 | Dec. 10, 2009 | N/A | N/A | GK_mouse_4.4 |
| 551 | Mouse | direct | Si13 | Dec. 10, 2009 | N/A | N/A | GK_mouse_4.5 |
| 552 | Mouse | direct | Si13 | Dec. 10, 2009 | N/A | N/A | GK_mouse_4.6 |
| 553 | Mouse | direct | Si13 | Dec. 10, 2009 | N/A | N/A | GK_mouse_4.7 |
| 554 | Mouse | direct | Si13 | Dec. 10, 2009 | N/A | N/A | GK_mouse_4.8 |
| 555 | Mouse | direct | Si13 | Dec. 10, 2009 | N/A | N/A | GK_mouse_4.9 |
| 557 | Mouse | cultured | Si13 | Dec. 10, 2009 | Dec. 10, 2009 | Dec. 17, 2009 | GK_mouse_4.11 |
| 558 | Mouse | cultured | Si13 | Dec. 10, 2009 | Dec. 10, 2009 | Dec. 17, 2009 | GK_mouse_4.13 |
| 559 | Mouse | cultured | Si13 | Dec. 10, 2009 | Dec. 10, 2009 | Dec. 17, 2009 | GK_mouse_4.15 |
| 560 | Mouse | cultured | Si13 | Dec. 10, 2009 | Dec. 10, 2009 | Dec. 17, 2009 | GK_mouse_4.16 |
| 561 | Mouse | cultured | Si13 | Dec. 10, 2009 | Dec. 10, 2009 | Dec. 17, 2009 | GK_mouse_4.17 |
| 562 | Mouse | cultured | Si13 | Dec. 10, 2009 | Dec. 10, 2009 | Dec. 17, 2009 | GK_mouse_4.18 |
| 563 | Mouse | cultured | Si13 | Dec. 10, 2009 | Dec. 10, 2009 | Dec. 17, 2009 | GK_mouse_4.19 |
| 583 | Mouse | direct | cec | Dec. 10, 2009 | N/A | N/A | GK_mouse_4.1 |
| 584 | Mouse | direct | cec | Dec. 10, 2009 | N/A | N/A | GK_mouse_4.2 |
| 587 | Mouse | direct | cec | Dec. 10, 2009 | N/A | N/A | GK_mouse_4.5 |
| 588 | Mouse | direct | cec | Dec. 10, 2009 | N/A | N/A | GK_mouse_4.6 |
| 589 | Mouse | direct | cec | Dec. 10, 2009 | N/A | N/A | GK_mouse_4.7 |
| 591 | Mouse | direct | cec | Dec. 10, 2009 | N/A | N/A | GK_mouse_4.9 |
| 592 | Mouse | direct | cec | Dec. 10, 2009 | N/A | N/A | GK_mouse_4.10 |
| 593 | Mouse | cultured | cec | Dec. 10, 2009 | Dec. 10, 2009 | Dec. 17, 2009 | GK_mouse_4.11 |
| 594 | Mouse | cultured | cec | Dec. 10, 2009 | Dec. 10, 2009 | Dec. 17, 2009 | GK_mouse_4.13 |
| 595 | Mouse | cultured | cec | Dec. 10, 2009 | Dec. 10, 2009 | Dec. 17, 2009 | GK_mouse_4.15 |
| 596 | Mouse | cultured | cec | Dec. 10, 2009 | Dec. 10, 2009 | Dec. 17, 2009 | GK_mouse_4.16 |
| 597 | Mouse | cultured | cec | Dec. 10, 2009 | Dec. 10, 2009 | Dec. 17, 2009 | GK_mouse_4.17 |
| 598 | Mouse | cultured | cec | Dec. 10, 2009 | Dec. 10, 2009 | Dec. 17, 2009 | GK_mouse_4.18 |
| 599 | Mouse | cultured | cec | Dec. 10, 2009 | Dec. 10, 2009 | Dec. 17, 2009 | GK_mouse_4.19 |
| 600 | Mouse | cultured | cec | Dec. 10, 2009 | Dec. 10, 2009 | Dec. 17, 2009 | GK_mouse_4.20 |
| 601 | Mouse | direct | col | Dec. 10, 2009 | N/A | N/A | GK_mouse_4.1 |
| 602 | Mouse | direct | col | Dec. 10, 2009 | N/A | N/A | GK_mouse_4.2 |
| 603 | Mouse | direct | col | Dec. 10, 2009 | N/A | N/A | GK_mouse_4.3 |
| 604 | Mouse | direct | col | Dec. 10, 2009 | N/A | N/A | GK_mouse_4.4 |
| 605 | Mouse | direct | col | Dec. 10, 2009 | N/A | N/A | GK_mouse_4.5 |
| 606 | Mouse | direct | col | Dec. 10, 2009 | N/A | N/A | GK_mouse_4.6 |
| 607 | Mouse | direct | col | Dec. 10, 2009 | N/A | N/A | GK_mouse_4.7 |
| 608 | Mouse | direct | col | Dec. 10, 2009 | N/A | N/A | GK_mouse_4.8 |
| 609 | Mouse | direct | col | Dec. 10, 2009 | N/A | N/A | GK_mouse_4.9 |
| 610 | Mouse | direct | col | Dec. 10, 2009 | N/A | N/A | GK_mouse_4.10 |
| 611 | Mouse | cultured | col | Dec. 10, 2009 | Dec. 10, 2009 | Dec. 17, 2009 | GK_mouse_4.11 |
| 612 | Mouse | cultured | col | Dec. 10, 2009 | Dec. 10, 2009 | Dec. 17, 2009 | GK_mouse_4.13 |
| 613 | Mouse | cultured | col | Dec. 10, 2009 | Dec. 10, 2009 | Dec. 17, 2009 | GK_mouse_4.15 |
| 614 | Mouse | cultured | col | Dec. 10, 2009 | Dec. 10, 2009 | Dec. 17, 2009 | GK_mouse_4.16 |
| 615 | Mouse | cultured | col | Dec. 10, 2009 | Dec. 10, 2009 | Dec. 17, 2009 | GK_mouse_4.17 |
| 616 | Mouse | cultured | col | Dec. 10, 2009 | Dec. 10, 2009 | Dec. 17, 2009 | GK_mouse_4.18 |
| 617 | Mouse | cultured | col | Dec. 10, 2009 | Dec. 10, 2009 | Dec. 17, 2009 | GK_mouse_4.19 |
| 618 | Mouse | cultured | col | Dec. 10, 2009 | Dec. 10, 2009 | Dec. 17, 2009 | GK_mouse_4.20 |
| 1001 | Donor | direct | fc | Jun. 24, 2009 | N/A | N/A | N/A |
| 1002 | Donor | direct | fc | Jun. 24, 2009 | N/A | N/A | N/A |
| 1003 | Donor | cultured | pt | Jun. 24, 2009 | Jun. 24, 2009 | Jun. 30, 2009 | N/A |
| 2000 | Donor | cultured | pt | Jul. 15, 2009 | Jul. 15, 2009 | Jul. 22, 2009 | N/A |
| 2001 | Donor | direct | fc | Jul. 15, 2009 | N/A | N/A | N/A |
| 2002 | Donor | direct | fc | Jul. 15, 2009 | N/A | N/A | N/A |
| 2003 | Donor | cultured | pt | Jul. 16, 2009 | Jul. 16, 2009 | Jul. 23, 2009 | N/A |
| 2004 | Donor | direct | fc | Jul. 16, 2009 | N/A | N/A | N/A |
| 2005 | Donor | direct | fc | Jul. 16, 2009 | N/A | N/A | N/A |
| 2006 | Donor | cultured | pt | Jul. 17, 2009 | Jul. 17, 2009 | Jul. 24, 2009 | N/A |
| 2007 | Donor | direct | fc | Jul. 17, 2009 | N/A | N/A | N/A |
| 2008 | Donor | direct | fc | Jul. 17, 2009 | N/A | N/A | N/A |
| 2009 | Donor | cultured | pt | Jun. 24, 2009 | Jun. 24, 2009 | Jun. 30, 2009 | N/A |
| 2010 | Donor | cultured | pt | Jul. 15, 2009 | Jul. 15, 2009 | Jul. 22, 2009 | N/A |
| 2011 | Donor | direct | fc | Jul. 15, 2009 | N/A | N/A | N/A |
| 2012 | Donor | direct | fc | Jul. 15, 2009 | N/A | N/A | N/A |
| 2013 | Donor | cultured | pt | Jul. 16, 2009 | Jul. 16, 2009 | Jul. 23, 2009 | N/A |
| 2014 | Donor | direct | fc | Jul. 16, 2009 | N/A | N/A | N/A |
| 2015 | Donor | direct | fc | Jul. 16, 2009 | N/A | N/A | N/A |
| 2016 | Donor | cultured | pt | Jul. 17, 2009 | Jul. 17, 2009 | Jul. 24, 2009 | N/A |
| 2017 | Donor | direct | fc | Jul. 17, 2009 | N/A | N/A | N/A |
| 2018 | Donor | direct | fc | Jul. 17, 2009 | N/A | N/A | N/A |
| 2019 | Donor | cultured | pt | Jun. 24, 2009 | Jun. 24, 2009 | Jun. 30, 2009 | N/A |
| 3001 | Donor | cultured | pt | Aug. 21, 2009 | Aug. 21, 2009 | Aug. 27, 2009 | N/A |
| 3002 | Donor | cultured | pt | Aug. 21, 2009 | Aug. 21, 2009 | Aug. 27, 2009 | N/A |
| 3003 | Donor | cultured | pt | Aug. 21, 2009 | Aug. 21, 2009 | Aug. 27, 2009 | N/A |
| 3004 | Donor | cultured | pt | Aug. 21, 2009 | Aug. 21, 2009 | Aug. 27, 2009 | N/A |
| 3005 | Donor | cultured | pt | Aug. 21, 2009 | Aug. 21, 2009 | Aug. 27, 2009 | N/A |
| 3006 | Donor | cultured | pt | Aug. 21, 2009 | Aug. 21, 2009 | Aug. 27, 2009 | N/A |
| 3007 | Donor | cultured | pt | Aug. 21, 2009 | Aug. 21, 2009 | Aug. 27, 2009 | N/A |
| 3008 | Donor | cultured | pt | Aug. 21, 2009 | Aug. 21, 2009 | Aug. 27, 2009 | N/A |
| 3009 | Donor | cultured | pt | Aug. 21, 2009 | Aug. 21, 2009 | Aug. 27, 2009 | N/A |
| 3010 | Donor | cultured | pt | Aug. 21, 2009 | Aug. 21, 2009 | Aug. 27, 2009 | N/A |
| 3011 | Donor | direct | fc | Aug. 21, 2009 | N/A | N/A | N/A |
| 3012 | Donor | direct | fc | Aug. 21, 2009 | N/A | N/A | N/A |
| 4000 | Donor | cultured | pt | May 25, 2010 | May 25, 2010 | Jun. 1, 2010 | N/A |
| 4001 | Donor | cultured | pt | May 25, 2010 | May 25, 2010 | Jun. 1, 2010 | N/A |
| 4002 | Donor | cultured | pt | May 25, 2010 | May 25, 2010 | Jun. 1, 2010 | N/A |
| 4003 | Mouse | cultured | fc | May 25, 2010 | May 25, 2010 | Jun. 1, 2010 | GK_mouse_5.1 |
| 4004 | Mouse | cultured | fc | May 25, 2010 | May 25, 2010 | Jun. 1, 2010 | GK_mouse_5.2 |
| 4005 | Mouse | cultured | fc | May 25, 2010 | May 25, 2010 | Jun. 1, 2010 | GK_mouse_5.3 |
| 4006 | Mouse | cultured | fc | May 25, 2010 | May 25, 2010 | Jun. 1, 2010 | GK_mouse_5.4 |
| 4007 | Mouse | cultured | fc | May 25, 2010 | May 25, 2010 | Jun. 1, 2010 | GK_mouse_5.5 |
| 4008 | Mouse | cultured | fc | May 25, 2010 | May 25, 2010 | Jun. 1, 2010 | GK_mouse_5.1 |
| 4009 | Mouse | cultured | fc | May 25, 2010 | May 25, 2010 | Jun. 1, 2010 | GK_mouse_5.2 |
| 4010 | Mouse | cultured | fc | May 25, 2010 | May 25, 2010 | Jun. 1, 2010 | GK_mouse_5.3 |
| 4011 | Mouse | cultured | fc | May 25, 2010 | May 25, 2010 | Jun. 1, 2010 | GK_mouse_5.4 |
| 4012 | Mouse | cultured | fc | May 25, 2010 | May 25, 2010 | Jun. 1, 2010 | GK_mouse_5.5 |
| 4013 | Mouse | cultured | fc | May 25, 2010 | May 25, 2010 | Jun. 1, 2010 | GK_mouse_5.1 |
| 4014 | Mouse | cultured | fc | May 25, 2010 | May 25, 2010 | Jun. 1, 2010 | GK_mouse_5.2 |
| 4015 | Mouse | cultured | fc | May 25, 2010 | May 25, 2010 | Jun. 1, 2010 | GK_mouse_5.3 |
| 4016 | Mouse | cultured | fc | May 25, 2010 | May 25, 2010 | Jun. 1, 2010 | GK_mouse_5.4 |
| 4017 | Mouse | cultured | fc | May 25, 2010 | May 25, 2010 | Jun. 1, 2010 | GK_mouse_5.5 |

| | | | | | | | Date |
|---|---|---|---|---|---|---|---|
| | | | | | Date mouse | Date | replating |
| Sample | | | | | sample | replating | from mouse |
| ID | Mouse DOB | Date gavage | Timepoint | Mouse Diet | collection | from mouse | collection |
| 2 | N/A | N/A | initial | N/A | N/A | N/A | N/A |
| 3 | N/A | N/A | initial | N/A | N/A | N/A | N/A |
| 5 | N/A | N/A | initial | N/A | N/A | N/A | N/A |
| 6 | N/A | N/A | initial | N/A | N/A | N/A | N/A |
| 7 | Oct. 5, 2009 | Dec. 10, 2009 | 4pg | LF/PP | Dec. 14, 2009 | N/A | N/A |
| 8 | Oct. 5, 2009 | Dec. 10, 2009 | 4pg | LF/PP | Dec. 14, 2009 | N/A | N/A |
| 9 | Oct. 5, 2009 | Dec. 10, 2009 | 4pg | LF/PP | Dec. 14, 2009 | N/A | N/A |
| 10 | Oct. 5, 2009 | Dec. 10, 2009 | 4pg | LF/PP | Dec. 14, 2009 | N/A | N/A |
| 11 | Oct. 5, 2009 | Dec. 10, 2009 | 4pg | LF/PP | Dec. 14, 2009 | N/A | N/A |
| 12 | Oct. 5, 2009 | Dec. 10, 2009 | 4pg | LF/PP | Dec. 14, 2009 | N/A | N/A |
| 14 | Oct. 5, 2009 | Dec. 10, 2009 | 4pg | LF/PP | Dec. 14, 2009 | N/A | N/A |
| 15 | Oct. 5, 2009 | Dec. 10, 2009 | 4pg | LF/PP | Dec. 14, 2009 | N/A | N/A |
| 16 | Oct. 5, 2009 | Dec. 10, 2009 | 4pg | LF/PP | Dec. 14, 2009 | N/A | N/A |
| 17 | Oct. 5, 2009 | Dec. 17, 2009 | 4pg | LF/PP | Dec. 21, 2009 | N/A | N/A |
| 18 | Oct. 5, 2009 | Dec. 17, 2009 | 4pg | LF/PP | Dec. 21, 2009 | N/A | N/A |
| 19 | Oct. 5, 2009 | Dec. 17, 2009 | 4pg | LF/PP | Dec. 21, 2009 | N/A | N/A |
| 20 | Oct. 5, 2009 | Dec. 17, 2009 | 4pg | LF/PP | Dec. 21, 2009 | N/A | N/A |
| 21 | Oct. 5, 2009 | Dec. 17, 2009 | 4pg | LF/PP | Dec. 21, 2009 | N/A | N/A |
| 22 | Oct. 5, 2009 | Dec. 17, 2009 | 4pg | LF/PP | Dec. 21, 2009 | N/A | N/A |
| 23 | Oct. 5, 2009 | Dec. 17, 2009 | 4pg | LF/PP | Dec. 21, 2009 | N/A | N/A |
| 24 | Oct. 5, 2009 | Dec. 17, 2009 | 4pg | LF/PP | Dec. 21, 2009 | N/A | N/A |
| 25 | Oct. 5, 2009 | Dec. 10, 2009 | 7pg | LF/PP | Dec. 17, 2009 | N/A | N/A |
| 26 | Oct. 5, 2009 | Dec. 10, 2009 | 7pg | LF/PP | Dec. 17, 2009 | N/A | N/A |
| 27 | Oct. 5, 2009 | Dec. 10, 2009 | 7pg | LF/PP | Dec. 17, 2009 | N/A | N/A |
| 28 | Oct. 5, 2009 | Dec. 10, 2009 | 7pg | LF/PP | Dec. 17, 2009 | N/A | N/A |
| 29 | Oct. 5, 2009 | Dec. 10, 2009 | 7pg | LF/PP | Dec. 17, 2009 | N/A | N/A |
| 30 | Oct. 5, 2009 | Dec. 10, 2009 | 7pg | LF/PP | Dec. 17, 2009 | N/A | N/A |
| 31 | Oct. 5, 2009 | Dec. 10, 2009 | 7pg | LF/PP | Dec. 17, 2009 | N/A | N/A |
| 32 | Oct. 5, 2009 | Dec. 10, 2009 | 7pg | LF/PP | Dec. 17, 2009 | N/A | N/A |
| 33 | Oct. 5, 2009 | Dec. 10, 2009 | 7pg | LF/PP | Dec. 17, 2009 | N/A | N/A |
| 34 | Oct. 5, 2009 | Dec. 10, 2009 | 7pg | LF/PP | Dec. 17, 2009 | N/A | N/A |
| 35 | Oct. 5, 2009 | Dec. 17, 2009 | 7pg | LF/PP | Dec. 24, 2009 | N/A | N/A |
| 36 | Oct. 5, 2009 | Dec. 17, 2009 | 7pg | LF/PP | Dec. 24, 2009 | N/A | N/A |
| 37 | Oct. 5, 2009 | Dec. 17, 2009 | 7pg | LF/PP | Dec. 24, 2009 | N/A | N/A |
| 38 | Oct. 5, 2009 | Dec. 17, 2009 | 7pg | LF/PP | Dec. 24, 2009 | N/A | N/A |
| 39 | Oct. 5, 2009 | Dec. 17, 2009 | 7pg | LF/PP | Dec. 24, 2009 | N/A | N/A |
| 40 | Oct. 5, 2009 | Dec. 17, 2009 | 7pg | LF/PP | Dec. 24, 2009 | N/A | N/A |
| 41 | Oct. 5, 2009 | Dec. 17, 2009 | 7pg | LF/PP | Dec. 24, 2009 | N/A | N/A |
| 42 | Oct. 5, 2009 | Dec. 17, 2009 | 7pg | LF/PP | Dec. 24, 2009 | N/A | N/A |
| 43 | Oct. 5, 2009 | Dec. 10, 2009 | 14pg | LF/PP | Dec. 24, 2009 | N/A | N/A |
| 44 | Oct. 5, 2009 | Dec. 10, 2009 | 14pg | LF/PP | Dec. 24, 2009 | N/A | N/A |
| 45 | Oct. 5, 2009 | Dec. 10, 2009 | 14pg | LF/PP | Dec. 24, 2009 | N/A | N/A |
| 46 | Oct. 5, 2009 | Dec. 10, 2009 | 14pg | LF/PP | Dec. 24, 2009 | N/A | N/A |
| 47 | Oct. 5, 2009 | Dec. 10, 2009 | 14pg | LF/PP | Dec. 24, 2009 | N/A | N/A |
| 48 | Oct. 5, 2009 | Dec. 10, 2009 | 14pg | LF/PP | Dec. 24, 2009 | N/A | N/A |
| 49 | Oct. 5, 2009 | Dec. 10, 2009 | 14pg | LF/PP | Dec. 24, 2009 | N/A | N/A |
| 50 | Oct. 5, 2009 | Dec. 10, 2009 | 14pg | LF/PP | Dec. 24, 2009 | N/A | N/A |
| 51 | Oct. 5, 2009 | Dec. 10, 2009 | 14pg | LF/PP | Dec. 24, 2009 | N/A | N/A |
| 52 | Oct. 5, 2009 | Dec. 10, 2009 | 14pg | LF/PP | Dec. 24, 2009 | N/A | N/A |
| 53 | Oct. 5, 2009 | Dec. 17, 2009 | 14pg | LF/PP | Dec. 31, 2009 | N/A | N/A |
| 54 | Oct. 5, 2009 | Dec. 17, 2009 | 14pg | LF/PP | Dec. 31, 2009 | N/A | N/A |
| 55 | Oct. 5, 2009 | Dec. 17, 2009 | 14pg | LF/PP | Dec. 31, 2009 | N/A | N/A |
| 56 | Oct. 5, 2009 | Dec. 17, 2009 | 14pg | LF/PP | Dec. 31, 2009 | N/A | N/A |
| 57 | Oct. 5, 2009 | Dec. 17, 2009 | 14pg | LF/PP | Dec. 31, 2009 | N/A | N/A |
| 58 | Oct. 5, 2009 | Dec. 17, 2009 | 14pg | LF/PP | Dec. 31, 2009 | N/A | N/A |
| 59 | Oct. 5, 2009 | Dec. 17, 2009 | 14pg | LF/PP | Dec. 31, 2009 | N/A | N/A |
| 60 | Oct. 5, 2009 | Dec. 17, 2009 | 14pg | LF/PP | Dec. 31, 2009 | N/A | N/A |
| 61 | Oct. 5, 2009 | Dec. 10, 2009 | 32pg | LF/PP | Jan. 11, 2010 | N/A | N/A |
| 62 | Oct. 5, 2009 | Dec. 10, 2009 | 32pg | LF/PP | Jan. 11, 2010 | N/A | N/A |
| 63 | Oct. 5, 2009 | Dec. 10, 2009 | 32pg | LF/PP | Jan. 11, 2010 | N/A | N/A |
| 64 | Oct. 5, 2009 | Dec. 10, 2009 | 32pg | LF/PP | Jan. 11, 2010 | N/A | N/A |
| 65 | Oct. 5, 2009 | Dec. 10, 2009 | 32pg | LF/PP | Jan. 11, 2010 | N/A | N/A |
| 66 | Oct. 5, 2009 | Dec. 10, 2009 | 32pg | LF/PP | Jan. 11, 2010 | N/A | N/A |
| 67 | Oct. 5, 2009 | Dec. 10, 2009 | 32pg | LF/PP | Jan. 11, 2010 | N/A | N/A |
| 68 | Oct. 5, 2009 | Dec. 10, 2009 | 32pg | LF/PP | Jan. 11, 2010 | N/A | N/A |
| 69 | Oct. 5, 2009 | Dec. 10, 2009 | 32pg | LF/PP | Jan. 11, 2010 | N/A | N/A |
| 70 | Oct. 5, 2009 | Dec. 10, 2009 | 32pg | LF/PP | Jan. 11, 2010 | N/A | N/A |
| 71 | Oct. 5, 2009 | Dec. 17, 2009 | 25pg | LF/PP | Jan. 11, 2010 | N/A | N/A |
| 72 | Oct. 5, 2009 | Dec. 17, 2009 | 25pg | LF/PP | Jan. 11, 2010 | N/A | N/A |
| 73 | Oct. 5, 2009 | Dec. 17, 2009 | 25pg | LF/PP | Jan. 11, 2010 | N/A | N/A |
| 74 | Oct. 5, 2009 | Dec. 17, 2009 | 25pg | LF/PP | Jan. 11, 2010 | N/A | N/A |
| 75 | Oct. 5, 2009 | Dec. 17, 2009 | 25pg | LF/PP | Jan. 11, 2010 | N/A | N/A |
| 76 | Oct. 5, 2009 | Dec. 17, 2009 | 25pg | LF/PP | Jan. 11, 2010 | N/A | N/A |
| 77 | Oct. 5, 2009 | Dec. 17, 2009 | 25pg | LF/PP | Jan. 11, 2010 | N/A | N/A |
| 78 | Oct. 5, 2009 | Dec. 17, 2009 | 25pg | LF/PP | Jan. 11, 2010 | N/A | N/A |
| 79 | Oct. 5, 2009 | Dec. 10, 2009 | 32pg | LF/PP | Jan. 11, 2010 | Jan. 11, 2010 | Jan. 18, 2010 |
| 80 | Oct. 5, 2009 | Dec. 10, 2009 | 32pg | LF/PP | Jan. 11, 2010 | Jan. 11, 2010 | Jan. 18, 2010 |
| 81 | Oct. 5, 2009 | Dec. 10, 2009 | 32pg | LF/PP | Jan. 11, 2010 | Jan. 11, 2010 | Jan. 18, 2010 |
| 82 | Oct. 5, 2009 | Dec. 10, 2009 | 32pg | LF/PP | Jan. 11, 2010 | Jan. 11, 2010 | Jan. 18, 2010 |
| 83 | Oct. 5, 2009 | Dec. 10, 2009 | 32pg | LF/PP | Jan. 11, 2010 | Jan. 11, 2010 | Jan. 18, 2010 |
| 84 | Oct. 5, 2009 | Dec. 10, 2009 | 32pg | LF/PP | Jan. 11, 2010 | Jan. 11, 2010 | Jan. 18, 2010 |
| 85 | Oct. 5, 2009 | Dec. 10, 2009 | 32pg | LF/PP | Jan. 11, 2010 | Jan. 11, 2010 | Jan. 18, 2010 |
| 86 | Oct. 5, 2009 | Dec. 10, 2009 | 32pg | LF/PP | Jan. 11, 2010 | Jan. 11, 2010 | Jan. 18, 2010 |
| 87 | Oct. 5, 2009 | Dec. 10, 2009 | 32pg | LF/PP | Jan. 11, 2010 | Jan. 11, 2010 | Jan. 18, 2010 |
| 88 | Oct. 5, 2009 | Dec. 10, 2009 | 32pg | LF/PP | Jan. 11, 2010 | Jan. 11, 2010 | Jan. 18, 2010 |
| 89 | Oct. 5, 2009 | Dec. 10, 2009 | 32pg | LF/PP | Jan. 11, 2010 | Jan. 11, 2010 | Jan. 18, 2010 |
| 90 | Oct. 5, 2009 | Dec. 10, 2009 | 32pg | LF/PP | Jan. 11, 2010 | Jan. 11, 2010 | Jan. 18, 2010 |
| 91 | Oct. 5, 2009 | Dec. 10, 2009 | 32pg | LF/PP | Jan. 11, 2010 | Jan. 11, 2010 | Jan. 18, 2010 |
| 92 | Oct. 5, 2009 | Dec. 10, 2009 | 32pg | LF/PP | Jan. 11, 2010 | Jan. 11, 2010 | Jan. 18, 2010 |
| 93 | Oct. 5, 2009 | Dec. 10, 2009 | 32pg | LF/PP | Jan. 11, 2010 | Jan. 11, 2010 | Jan. 18, 2010 |
| 94 | Oct. 5, 2009 | Dec. 10, 2009 | 32pg | LF/PP | Jan. 11, 2010 | Jan. 11, 2010 | Jan. 18, 2010 |
| 95 | Oct. 5, 2009 | Dec. 10, 2009 | 32pg | LF/PP | Jan. 11, 2010 | Jan. 11, 2010 | Jan. 18, 2010 |
| 96 | Oct. 5, 2009 | Dec. 10, 2009 | 32pg | LF/PP | Jan. 11, 2010 | Jan. 11, 2010 | Jan. 18, 2010 |
| 97 | Oct. 5, 2009 | Dec. 10, 2009 | 32pg | LF/PP | Jan. 11, 2010 | Jan. 11, 2010 | Jan. 18, 2010 |
| 98 | Oct. 5, 2009 | Dec. 10, 2009 | 32pg | LF/PP | Jan. 11, 2010 | Jan. 11, 2010 | Jan. 18, 2010 |
| 102 | Oct. 5, 2009 | Dec. 10, 2009 | 32pg | LF/PP | Jan. 11, 2010 | Jan. 11, 2010 | Jan. 18, 2010 |
| 108 | Oct. 5, 2009 | Dec. 10, 2009 | 32pg | LF/PP | Jan. 11, 2010 | Jan. 11, 2010 | Jan. 18, 2010 |
| 110 | Oct. 5, 2009 | Dec. 10, 2009 | 32pg | LF/PP | Jan. 11, 2010 | Jan. 11, 2010 | Jan. 18, 2010 |
| 111 | Oct. 5, 2009 | Dec. 10, 2009 | 32pg | LF/PP | Jan. 11, 2010 | Jan. 11, 2010 | Jan. 18, 2010 |
| 112 | Oct. 5, 2009 | Dec. 10, 2009 | 32pg | LF/PP | Jan. 11, 2010 | Jan. 11, 2010 | Jan. 18, 2010 |
| 113 | Oct. 5, 2009 | Dec. 10, 2009 | 32pg | LF/PP | Jan. 11, 2010 | Jan. 11, 2010 | Jan. 18, 2010 |
| 114 | Oct. 5, 2009 | Dec. 10, 2009 | 32pg | LF/PP | Jan. 11, 2010 | Jan. 11, 2010 | Jan. 18, 2010 |
| 115 | Oct. 5, 2009 | Dec. 10, 2009 | 32pg | LF/PP | Jan. 11, 2010 | Jan. 11, 2010 | Jan. 18, 2010 |
| 116 | Oct. 5, 2009 | Dec. 10, 2009 | 32pg | LF/PP | Jan. 11, 2010 | Jan. 11, 2010 | Jan. 18, 2010 |
| 120 | Oct. 5, 2009 | Dec. 10, 2009 | 32pg | LF/PP | Jan. 11, 2010 | Jan. 11, 2010 | Jan. 18, 2010 |
| 126 | Oct. 5, 2009 | Dec. 10, 2009 | 32pg | LF/PP | Jan. 11, 2010 | Jan. 11, 2010 | Jan. 18, 2010 |
| 132 | Oct. 5, 2009 | Dec. 10, 2009 | 32pg | LF/PP | Jan. 11, 2010 | Jan. 11, 2010 | Jan. 18, 2010 |
| 144 | Oct. 5, 2009 | Dec. 17, 2009 | 25pg | LF/PP | Jan. 11, 2010 | Jan. 11, 2010 | Jan. 18, 2010 |
| 150 | Oct. 5, 2009 | Dec. 17, 2009 | 25pg | LF/PP | Jan. 11, 2010 | Jan. 11, 2010 | Jan. 18, 2010 |
| 156 | Oct. 5, 2009 | Dec. 17, 2009 | 25pg | LF/PP | Jan. 11, 2010 | Jan. 11, 2010 | Jan. 18, 2010 |
| 162 | Oct. 5, 2009 | Dec. 17, 2009 | 25pg | LF/PP | Jan. 11, 2010 | Jan. 11, 2010 | Jan. 18, 2010 |
| 168 | Oct. 5, 2009 | Dec. 17, 2009 | 25pg | LF/PP | Jan. 11, 2010 | Jan. 11, 2010 | Jan. 18, 2010 |
| 174 | Oct. 5, 2009 | Dec. 17, 2009 | 25pg | LF/PP | Jan. 11, 2010 | Jan. 11, 2010 | Jan. 18, 2010 |
| 180 | Oct. 5, 2009 | Dec. 17, 2009 | 25pg | LF/PP | Jan. 11, 2010 | Jan. 11, 2010 | Jan. 18, 2010 |
| 186 | Oct. 5, 2009 | Dec. 17, 2009 | 25pg | LF/PP | Jan. 11, 2010 | Jan. 11, 2010 | Jan. 18, 2010 |
| 187 | Oct. 5, 2009 | Dec. 10, 2009 | 1pw | Western | Jan. 13, 2010 | N/A | N/A |
| 188 | Oct. 5, 2009 | Dec. 10, 2009 | 1pw | Western | Jan. 13, 2010 | N/A | N/A |
| 189 | Oct. 5, 2009 | Dec. 10, 2009 | 1pw | Western | Jan. 13, 2010 | N/A | N/A |
| 190 | Oct. 5, 2009 | Dec. 10, 2009 | 1pw | Western | Jan. 13, 2010 | N/A | N/A |
| 191 | Oct. 5, 2009 | Dec. 10, 2009 | 1pw | Western | Jan. 13, 2010 | N/A | N/A |
| 192 | Oct. 5, 2009 | Dec. 10, 2009 | 1pw | Western | Jan. 13, 2010 | N/A | N/A |
| 193 | Oct. 5, 2009 | Dec. 10, 2009 | 1pw | Western | Jan. 13, 2010 | N/A | N/A |
| 194 | Oct. 5, 2009 | Dec. 10, 2009 | 1pw | Western | Jan. 13, 2010 | N/A | N/A |
| 195 | Oct. 5, 2009 | Dec. 10, 2009 | 1pw | Western | Jan. 13, 2010 | N/A | N/A |
| 196 | Oct. 5, 2009 | Dec. 10, 2009 | 1pw | Western | Jan. 13, 2010 | N/A | N/A |
| 197 | Oct. 5, 2009 | Dec. 17, 2009 | 1pw | Western | Jan. 13, 2010 | N/A | N/A |
| 198 | Oct. 5, 2009 | Dec. 17, 2009 | 1pw | Western | Jan. 13, 2010 | N/A | N/A |
| 199 | Oct. 5, 2009 | Dec. 17, 2009 | 1pw | Western | Jan. 13, 2010 | N/A | N/A |
| 200 | Oct. 5, 2009 | Dec. 17, 2009 | 1pw | Western | Jan. 13, 2010 | N/A | N/A |
| 201 | Oct. 5, 2009 | Dec. 17, 2009 | 1pw | Western | Jan. 13, 2010 | N/A | N/A |
| 202 | Oct. 5, 2009 | Dec. 17, 2009 | 1pw | Western | Jan. 13, 2010 | N/A | N/A |
| 203 | Oct. 5, 2009 | Dec. 17, 2009 | 1pw | Western | Jan. 13, 2010 | N/A | N/A |
| 204 | Oct. 5, 2009 | Dec. 17, 2009 | 1pw | Western | Jan. 13, 2010 | N/A | N/A |
| 205 | Oct. 5, 2009 | Dec. 10, 2009 | 3pw | Western | Jan. 15, 2010 | N/A | N/A |
| 207 | Oct. 5, 2009 | Dec. 10, 2009 | 3pw | Western | Jan. 15, 2010 | N/A | N/A |
| 208 | Oct. 5, 2009 | Dec. 10, 2009 | 3pw | Western | Jan. 15, 2010 | N/A | N/A |
| 209 | Oct. 5, 2009 | Dec. 10, 2009 | 3pw | Western | Jan. 15, 2010 | N/A | N/A |
| 210 | Oct. 5, 2009 | Dec. 10, 2009 | 3pw | Western | Jan. 15, 2010 | N/A | N/A |
| 211 | Oct. 5, 2009 | Dec. 10, 2009 | 3pw | Western | Jan. 15, 2010 | N/A | N/A |
| 212 | Oct. 5, 2009 | Dec. 10, 2009 | 3pw | Western | Jan. 15, 2010 | N/A | N/A |
| 213 | Oct. 5, 2009 | Dec. 10, 2009 | 3pw | Western | Jan. 15, 2010 | N/A | N/A |
| 214 | Oct. 5, 2009 | Dec. 10, 2009 | 3pw | Western | Jan. 15, 2010 | N/A | N/A |
| 215 | Oct. 5, 2009 | Dec. 17, 2009 | 3pw | Western | Jan. 15, 2010 | N/A | N/A |
| 216 | Oct. 5, 2009 | Dec. 17, 2009 | 3pw | Western | Jan. 15, 2010 | N/A | N/A |
| 217 | Oct. 5, 2009 | Dec. 17, 2009 | 3pw | Western | Jan. 15, 2010 | N/A | N/A |
| 218 | Oct. 5, 2009 | Dec. 17, 2009 | 3pw | Western | Jan. 15, 2010 | N/A | N/A |
| 219 | Oct. 5, 2009 | Dec. 17, 2009 | 3pw | Western | Jan. 15, 2010 | N/A | N/A |
| 220 | Oct. 5, 2009 | Dec. 17, 2009 | 3pw | Western | Jan. 15, 2010 | N/A | N/A |
| 221 | Oct. 5, 2009 | Dec. 17, 2009 | 3pw | Western | Jan. 15, 2010 | N/A | N/A |
| 222 | Oct. 5, 2009 | Dec. 17, 2009 | 3pw | Western | Jan. 15, 2010 | N/A | N/A |
| 223 | Oct. 5, 2009 | Dec. 10, 2009 | 7pw | Western | Jan. 19, 2010 | N/A | N/A |
| 224 | Oct. 5, 2009 | Dec. 10, 2009 | 7pw | Western | Jan. 19, 2010 | N/A | N/A |
| 225 | Oct. 5, 2009 | Dec. 10, 2009 | 7pw | Western | Jan. 19, 2010 | N/A | N/A |
| 226 | Oct. 5, 2009 | Dec. 10, 2009 | 7pw | Western | Jan. 19, 2010 | N/A | N/A |
| 227 | Oct. 5, 2009 | Dec. 10, 2009 | 7pw | Western | Jan. 19, 2010 | N/A | N/A |
| 228 | Oct. 5, 2009 | Dec. 10, 2009 | 7pw | Western | Jan. 19, 2010 | N/A | N/A |
| 229 | Oct. 5, 2009 | Dec. 10, 2009 | 7pw | Western | Jan. 19, 2010 | N/A | N/A |
| 230 | Oct. 5, 2009 | Dec. 10, 2009 | 7pw | Western | Jan. 19, 2010 | N/A | N/A |
| 231 | Oct. 5, 2009 | Dec. 10, 2009 | 7pw | Western | Jan. 19, 2010 | N/A | N/A |
| 232 | Oct. 5, 2009 | Dec. 10, 2009 | 7pw | Western | Jan. 19, 2010 | N/A | N/A |
| 233 | Oct. 5, 2009 | Dec. 17, 2009 | 7pw | Western | Jan. 19, 2010 | N/A | N/A |
| 234 | Oct. 5, 2009 | Dec. 17, 2009 | 7pw | Western | Jan. 19, 2010 | N/A | N/A |
| 235 | Oct. 5, 2009 | Dec. 17, 2009 | 7pw | Western | Jan. 19, 2010 | N/A | N/A |
| 236 | Oct. 5, 2009 | Dec. 17, 2009 | 7pw | Western | Jan. 19, 2010 | N/A | N/A |
| 238 | Oct. 5, 2009 | Dec. 17, 2009 | 7pw | Western | Jan. 19, 2010 | N/A | N/A |
| 240 | Oct. 5, 2009 | Dec. 17, 2009 | 7pw | Western | Jan. 19, 2010 | N/A | N/A |
| 261 | Oct. 5, 2009 | Dec. 10, 2009 | 14pw | Western | Jan. 26, 2010 | N/A | N/A |
| 262 | Oct. 5, 2009 | Dec. 10, 2009 | 14pw | Western | Jan. 26, 2010 | N/A | N/A |
| 263 | Oct. 5, 2009 | Dec. 10, 2009 | 14pw | Western | Jan. 26, 2010 | N/A | N/A |
| 265 | Oct. 5, 2009 | Dec. 10, 2009 | 14pw | Western | Jan. 26, 2010 | N/A | N/A |
| 266 | Oct. 5, 2009 | Dec. 10, 2009 | 14pw | Western | Jan. 26, 2010 | N/A | N/A |
| 267 | Oct. 5, 2009 | Dec. 10, 2009 | 14pw | Western | Jan. 26, 2010 | N/A | N/A |
| 268 | Oct. 5, 2009 | Dec. 10, 2009 | 14pw | Western | Jan. 26, 2010 | N/A | N/A |
| 269 | Oct. 5, 2009 | Dec. 17, 2009 | 14pw | Western | Jan. 26, 2010 | N/A | N/A |
| 270 | Oct. 5, 2009 | Dec. 17, 2009 | 14pw | Western | Jan. 26, 2010 | N/A | N/A |
| 271 | Oct. 5, 2009 | Dec. 17, 2009 | 14pw | Western | Jan. 26, 2010 | N/A | N/A |
| 272 | Oct. 5, 2009 | Dec. 17, 2009 | 14pw | Western | Jan. 26, 2010 | N/A | N/A |
| 273 | Oct. 5, 2009 | Dec. 17, 2009 | 14pw | Western | Jan. 26, 2010 | N/A | N/A |
| 274 | Oct. 5, 2009 | Dec. 17, 2009 | 14pw | Western | Jan. 26, 2010 | N/A | N/A |
| 275 | Oct. 5, 2009 | Dec. 17, 2009 | 14pw | Western | Jan. 26, 2010 | N/A | N/A |
| 276 | Oct. 5, 2009 | Dec. 17, 2009 | 14pw | Western | Jan. 26, 2010 | N/A | N/A |
| 277 | Oct. 5, 2009 | Dec. 10, 2009 | 14pw | Western | Jan. 26, 2010 | Jan. 26, 2010 | Feb. 2, 2010 |
| 278 | Oct. 5, 2009 | Dec. 10, 2009 | 14pw | Western | Jan. 26, 2010 | Jan. 26, 2010 | Feb. 2, 2010 |
| 279 | Oct. 5, 2009 | Dec. 10, 2009 | 14pw | Western | Jan. 26, 2010 | Jan. 26, 2010 | Feb. 2, 2010 |
| 280 | Oct. 5, 2009 | Dec. 10, 2009 | 14pw | Western | Jan. 26, 2010 | Jan. 26, 2010 | Feb. 2, 2010 |
| 281 | Oct. 5, 2009 | Dec. 10, 2009 | 14pw | Western | Jan. 26, 2010 | Jan. 26, 2010 | Feb. 2, 2010 |
| 282 | Oct. 5, 2009 | Dec. 10, 2009 | 14pw | Western | Jan. 26, 2010 | Jan. 26, 2010 | Feb. 2, 2010 |
| 284 | Oct. 5, 2009 | Dec. 10, 2009 | 14pw | Western | Jan. 26, 2010 | Jan. 26, 2010 | Feb. 2, 2010 |
| 285 | Oct. 5, 2009 | Dec. 10, 2009 | 14pw | Western | Jan. 26, 2010 | Jan. 26, 2010 | Feb. 2, 2010 |
| 287 | Oct. 5, 2009 | Dec. 17, 2009 | 14pw | Western | Jan. 26, 2010 | Jan. 26, 2010 | Feb. 2, 2010 |
| 288 | Oct. 5, 2009 | Dec. 17, 2009 | 14pw | Western | Jan. 26, 2010 | Jan. 26, 2010 | Feb. 2, 2010 |
| 289 | Oct. 5, 2009 | Dec. 17, 2009 | 14pw | Western | Jan. 26, 2010 | Jan. 26, 2010 | Feb. 2, 2010 |
| 290 | Oct. 5, 2009 | Dec. 17, 2009 | 14pw | Western | Jan. 26, 2010 | Jan. 26, 2010 | Feb. 2, 2010 |
| 291 | Oct. 5, 2009 | Dec. 17, 2009 | 14pw | Western | Jan. 26, 2010 | Jan. 26, 2010 | Feb. 2, 2010 |
| 292 | Oct. 5, 2009 | Dec. 17, 2009 | 14pw | Western | Jan. 26, 2010 | Jan. 26, 2010 | Feb. 2, 2010 |
| 293 | Oct. 5, 2009 | Dec. 17, 2009 | 14pw | Western | Jan. 26, 2010 | Jan. 26, 2010 | Feb. 2, 2010 |
| 294 | Oct. 5, 2009 | Dec. 17, 2009 | 14pw | Western | Jan. 26, 2010 | Jan. 26, 2010 | Feb. 2, 2010 |
| 295 | Oct. 5, 2009 | Dec. 10, 2009 | 1pb | LF/PP | Jan. 27, 2010 | N/A | N/A |
| 296 | Oct. 5, 2009 | Dec. 10, 2009 | 1pb | LF/PP | Jan. 27, 2010 | N/A | N/A |
| 297 | Oct. 5, 2009 | Dec. 10, 2009 | 1pb | LF/PP | Jan. 27, 2010 | N/A | N/A |
| 298 | Oct. 5, 2009 | Dec. 10, 2009 | 1pb | LF/PP | Jan. 27, 2010 | N/A | N/A |
| 299 | Oct. 5, 2009 | Dec. 10, 2009 | 1pb | LF/PP | Jan. 27, 2010 | N/A | N/A |
| 300 | Oct. 5, 2009 | Dec. 10, 2009 | 1pb | LF/PP | Jan. 27, 2010 | N/A | N/A |
| 301 | Oct. 5, 2009 | Dec. 10, 2009 | 1pb | LF/PP | Jan. 27, 2010 | N/A | N/A |
| 302 | Oct. 5, 2009 | Dec. 10, 2009 | 1pb | LF/PP | Jan. 27, 2010 | N/A | N/A |
| 303 | Oct. 5, 2009 | Dec. 10, 2009 | 1pb | LF/PP | Jan. 27, 2010 | N/A | N/A |
| 304 | Oct. 5, 2009 | Dec. 10, 2009 | 1pb | LF/PP | Jan. 27, 2010 | N/A | N/A |
| 305 | Oct. 5, 2009 | Dec. 17, 2009 | 1pb | LF/PP | Jan. 27, 2010 | N/A | N/A |
| 306 | Oct. 5, 2009 | Dec. 17, 2009 | 1pb | LF/PP | Jan. 27, 2010 | N/A | N/A |
| 307 | Oct. 5, 2009 | Dec. 17, 2009 | 1pb | LF/PP | Jan. 27, 2010 | N/A | N/A |
| 308 | Oct. 5, 2009 | Dec. 17, 2009 | 1pb | LF/PP | Jan. 27, 2010 | N/A | N/A |
| 309 | Oct. 5, 2009 | Dec. 17, 2009 | 1pb | LF/PP | Jan. 27, 2010 | N/A | N/A |
| 310 | Oct. 5, 2009 | Dec. 17, 2009 | 1pb | LF/PP | Jan. 27, 2010 | N/A | N/A |
| 311 | Oct. 5, 2009 | Dec. 17, 2009 | 1pb | LF/PP | Jan. 27, 2010 | N/A | N/A |
| 312 | Oct. 5, 2009 | Dec. 17, 2009 | 1pb | LF/PP | Jan. 27, 2010 | N/A | N/A |
| 313 | Oct. 5, 2009 | Dec. 10, 2009 | 3pb | LF/PP | Jan. 29, 2010 | N/A | N/A |
| 314 | Oct. 5, 2009 | Dec. 10, 2009 | 3pb | LF/PP | Jan. 29, 2010 | N/A | N/A |
| 315 | Oct. 5, 2009 | Dec. 10, 2009 | 3pb | LF/PP | Jan. 29, 2010 | N/A | N/A |
| 316 | Oct. 5, 2009 | Dec. 10, 2009 | 3pb | LF/PP | Jan. 29, 2010 | N/A | N/A |
| 317 | Oct. 5, 2009 | Dec. 10, 2009 | 3pb | LF/PP | Jan. 29, 2010 | N/A | N/A |
| 318 | Oct. 5, 2009 | Dec. 10, 2009 | 3pb | LF/PP | Jan. 29, 2010 | N/A | N/A |
| 319 | Oct. 5, 2009 | Dec. 10, 2009 | 3pb | LF/PP | Jan. 29, 2010 | N/A | N/A |
| 321 | Oct. 5, 2009 | Dec. 10, 2009 | 3pb | LF/PP | Jan. 29, 2010 | N/A | N/A |
| 323 | Oct. 5, 2009 | Dec. 17, 2009 | 3pb | LF/PP | Jan. 29, 2010 | N/A | N/A |
| 324 | Oct. 5, 2009 | Dec. 17, 2009 | 3pb | LF/PP | Jan. 29, 2010 | N/A | N/A |
| 325 | Oct. 5, 2009 | Dec. 17, 2009 | 3pb | LF/PP | Jan. 29, 2010 | N/A | N/A |
| 326 | Oct. 5, 2009 | Dec. 17, 2009 | 3pb | LF/PP | Jan. 29, 2010 | N/A | N/A |
| 328 | Oct. 5, 2009 | Dec. 17, 2009 | 3pb | LF/PP | Jan. 29, 2010 | N/A | N/A |
| 329 | Oct. 5, 2009 | Dec. 17, 2009 | 3pb | LF/PP | Jan. 29, 2010 | N/A | N/A |
| 330 | Oct. 5, 2009 | Dec. 17, 2009 | 3pb | LF/PP | Jan. 29, 2010 | N/A | N/A |
| 332 | Oct. 5, 2009 | Dec. 10, 2009 | 8pb | LF/PP | Feb. 3, 2010 | N/A | N/A |
| 333 | Oct. 5, 2009 | Dec. 10, 2009 | 8pb | LF/PP | Feb. 3, 2010 | N/A | N/A |
| 334 | Oct. 5, 2009 | Dec. 10, 2009 | 8pb | LF/PP | Feb. 3, 2010 | N/A | N/A |
| 335 | Oct. 5, 2009 | Dec. 10, 2009 | 8pb | LF/PP | Feb. 3, 2010 | N/A | N/A |
| 338 | Oct. 5, 2009 | Dec. 10, 2009 | 8pb | LF/PP | Feb. 3, 2010 | N/A | N/A |
| 339 | Oct. 5, 2009 | Dec. 10, 2009 | 8pb | LF/PP | Feb. 3, 2010 | N/A | N/A |
| 340 | Oct. 5, 2009 | Dec. 10, 2009 | 8pb | LF/PP | Feb. 3, 2010 | N/A | N/A |
| 341 | Oct. 5, 2009 | Dec. 17, 2009 | 8pb | LF/PP | Feb. 3, 2010 | N/A | N/A |
| 342 | Oct. 5, 2009 | Dec. 17, 2009 | 8pb | LF/PP | Feb. 3, 2010 | N/A | N/A |
| 343 | Oct. 5, 2009 | Dec. 17, 2009 | 8pb | LF/PP | Feb. 3, 2010 | N/A | N/A |
| 344 | Oct. 5, 2009 | Dec. 17, 2009 | 8pb | LF/PP | Feb. 3, 2010 | N/A | N/A |
| 345 | Oct. 5, 2009 | Dec. 17, 2009 | 8pb | LF/PP | Feb. 3, 2010 | N/A | N/A |
| 347 | Oct. 5, 2009 | Dec. 17, 2009 | 8pb | LF/PP | Feb. 3, 2010 | N/A | N/A |
| 348 | Oct. 5, 2009 | Dec. 17, 2009 | 8pb | LF/PP | Feb. 3, 2010 | N/A | N/A |
| 349 | Oct. 5, 2009 | Dec. 10, 2009 | 8pb | LF/PP | Feb. 3, 2010 | Feb. 3, 2010 | Feb. 10, 2010 |
| 350 | Oct. 5, 2009 | Dec. 10, 2009 | 8pb | LF/PP | Feb. 3, 2010 | Feb. 3, 2010 | Feb. 10, 2010 |
| 351 | Oct. 5, 2009 | Dec. 10, 2009 | 8pb | LF/PP | Feb. 3, 2010 | Feb. 3, 2010 | Feb. 10, 2010 |
| 352 | Oct. 5, 2009 | Dec. 10, 2009 | 8pb | LF/PP | Feb. 3, 2010 | Feb. 3, 2010 | Feb. 10, 2010 |
| 353 | Oct. 5, 2009 | Dec. 10, 2009 | 8pb | LF/PP | Feb. 3, 2010 | Feb. 3, 2010 | Feb. 10, 2010 |
| 354 | Oct. 5, 2009 | Dec. 10, 2009 | 8pb | LF/PP | Feb. 3, 2010 | Feb. 3, 2010 | Feb. 10, 2010 |
| 355 | Oct. 5, 2009 | Dec. 10, 2009 | 8pb | LF/PP | Feb. 3, 2010 | Feb. 3, 2010 | Feb. 10, 2010 |
| 356 | Oct. 5, 2009 | Dec. 10, 2009 | 8pb | LF/PP | Feb. 3, 2010 | Feb. 3, 2010 | Feb. 10, 2010 |
| 357 | Oct. 5, 2009 | Dec. 10, 2009 | 8pb | LF/PP | Feb. 3, 2010 | Feb. 3, 2010 | Feb. 10, 2010 |
| 358 | Oct. 5, 2009 | Dec. 10, 2009 | 8pb | LF/PP | Feb. 3, 2010 | Feb. 3, 2010 | Feb. 10, 2010 |
| 359 | Oct. 5, 2009 | Dec. 17, 2009 | 8pb | LF/PP | Feb. 3, 2010 | Feb. 3, 2010 | Feb. 10, 2010 |
| 360 | Oct. 5, 2009 | Dec. 17, 2009 | 8pb | LF/PP | Feb. 3, 2010 | Feb. 3, 2010 | Feb. 10, 2010 |
| 361 | Oct. 5, 2009 | Dec. 17, 2009 | 8pb | LF/PP | Feb. 3, 2010 | Feb. 3, 2010 | Feb. 10, 2010 |
| 362 | Oct. 5, 2009 | Dec. 17, 2009 | 8pb | LF/PP | Feb. 3, 2010 | Feb. 3, 2010 | Feb. 10, 2010 |
| 363 | Oct. 5, 2009 | Dec. 17, 2009 | 8pb | LF/PP | Feb. 3, 2010 | Feb. 3, 2010 | Feb. 10, 2010 |
| 364 | Oct. 5, 2009 | Dec. 17, 2009 | 8pb | LF/PP | Feb. 3, 2010 | Feb. 3, 2010 | Feb. 10, 2010 |
| 365 | Oct. 5, 2009 | Dec. 17, 2009 | 8pb | LF/PP | Feb. 3, 2010 | Feb. 3, 2010 | Feb. 10, 2010 |
| 366 | Oct. 5, 2009 | Dec. 17, 2009 | 8pb | LF/PP | Feb. 3, 2010 | Feb. 3, 2010 | Feb. 10, 2010 |
| 367 | Oct. 5, 2009 | Dec. 10, 2009 | 15pb | LF/PP | Feb. 10, 2010 | N/A | N/A |
| 369 | Oct. 5, 2009 | Dec. 10, 2009 | 15pb | LF/PP | Feb. 10, 2010 | N/A | N/A |
| 370 | Oct. 5, 2009 | Dec. 10, 2009 | 15pb | LF/PP | Feb. 10, 2010 | N/A | N/A |
| 371 | Oct. 5, 2009 | Dec. 10, 2009 | 15pb | LF/PP | Feb. 10, 2010 | N/A | N/A |
| 372 | Oct. 5, 2009 | Dec. 10, 2009 | 15pb | LF/PP | Feb. 10, 2010 | N/A | N/A |
| 373 | Oct. 5, 2009 | Dec. 10, 2009 | 15pb | LF/PP | Feb. 10, 2010 | N/A | N/A |
| 374 | Oct. 5, 2009 | Dec. 10, 2009 | 15pb | LF/PP | Feb. 10, 2010 | N/A | N/A |
| 375 | Oct. 5, 2009 | Dec. 10, 2009 | 15pb | LF/PP | Feb. 10, 2010 | N/A | N/A |
| 376 | Oct. 5, 2009 | Dec. 10, 2009 | 15pb | LF/PP | Feb. 10, 2010 | N/A | N/A |
| 377 | Oct. 5, 2009 | Dec. 17, 2009 | 15pb | LF/PP | Feb. 10, 2010 | N/A | N/A |
| 378 | Oct. 5, 2009 | Dec. 17, 2009 | 15pb | LF/PP | Feb. 10, 2010 | N/A | N/A |
| 379 | Oct. 5, 2009 | Dec. 17, 2009 | 15pb | LF/PP | Feb. 10, 2010 | N/A | N/A |
| 380 | Oct. 5, 2009 | Dec. 17, 2009 | 15pb | LF/PP | Feb. 10, 2010 | N/A | N/A |
| 381 | Oct. 5, 2009 | Dec. 17, 2009 | 15pb | LF/PP | Feb. 10, 2010 | N/A | N/A |
| 382 | Oct. 5, 2009 | Dec. 17, 2009 | 15pb | LF/PP | Feb. 10, 2010 | N/A | N/A |
| 383 | Oct. 5, 2009 | Dec. 17, 2009 | 15pb | LF/PP | Feb. 10, 2010 | N/A | N/A |
| 384 | Oct. 5, 2009 | Dec. 17, 2009 | 15pb | LF/PP | Feb. 10, 2010 | N/A | N/A |
| 385 | Oct. 5, 2009 | Dec. 10, 2009 | 1pf | Fasting | Feb. 11, 2010 | N/A | N/A |
| 386 | Oct. 5, 2009 | Dec. 10, 2009 | 1pf | Fasting | Feb. 11, 2010 | N/A | N/A |
| 387 | Oct. 5, 2009 | Dec. 10, 2009 | 1pf | Fasting | Feb. 11, 2010 | N/A | N/A |
| 388 | Oct. 5, 2009 | Dec. 10, 2009 | 1pf | Fasting | Feb. 11, 2010 | N/A | N/A |
| 389 | Oct. 5, 2009 | Dec. 10, 2009 | 1pf | Fasting | Feb. 11, 2010 | N/A | N/A |
| 390 | Oct. 5, 2009 | Dec. 10, 2009 | 1pf | Fasting | Feb. 11, 2010 | N/A | N/A |
| 391 | Oct. 5, 2009 | Dec. 10, 2009 | 1pf | Fasting | Feb. 11, 2010 | N/A | N/A |
| 392 | Oct. 5, 2009 | Dec. 10, 2009 | 1pf | Fasting | Feb. 11, 2010 | N/A | N/A |
| 393 | Oct. 5, 2009 | Dec. 10, 2009 | 1pf | Fasting | Feb. 11, 2010 | N/A | N/A |
| 394 | Oct. 5, 2009 | Dec. 10, 2009 | 1pf | Fasting | Feb. 11, 2010 | N/A | N/A |
| 395 | Oct. 5, 2009 | Dec. 17, 2009 | 1pf | Fasting | Feb. 11, 2010 | N/A | N/A |
| 396 | Oct. 5, 2009 | Dec. 17, 2009 | 1pf | Fasting | Feb. 11, 2010 | N/A | N/A |
| 397 | Oct. 5, 2009 | Dec. 17, 2009 | 1pf | Fasting | Feb. 11, 2010 | N/A | N/A |
| 398 | Oct. 5, 2009 | Dec. 17, 2009 | 1pf | Fasting | Feb. 11, 2010 | N/A | N/A |
| 399 | Oct. 5, 2009 | Dec. 17, 2009 | 1pf | Fasting | Feb. 11, 2010 | N/A | N/A |
| 400 | Oct. 5, 2009 | Dec. 17, 2009 | 1pf | Fasting | Feb. 11, 2010 | N/A | N/A |
| 401 | Oct. 5, 2009 | Dec. 17, 2009 | 1pf | Fasting | Feb. 11, 2010 | N/A | N/A |
| 402 | Oct. 5, 2009 | Dec. 17, 2009 | 1pf | Fasting | Feb. 11, 2010 | N/A | N/A |
| 403 | Oct. 5, 2009 | Dec. 10, 2009 | 1pf | Fasting | Feb. 11, 2010 | Feb. 11, 2010 | Feb. 18, 2010 |
| 404 | Oct. 5, 2009 | Dec. 10, 2009 | 1pf | Fasting | Feb. 11, 2010 | Feb. 11, 2010 | Feb. 18, 2010 |
| 405 | Oct. 5, 2009 | Dec. 10, 2009 | 1pf | Fasting | Feb. 11, 2010 | Feb. 11, 2010 | Feb. 18, 2010 |
| 406 | Oct. 5, 2009 | Dec. 10, 2009 | 1pf | Fasting | Feb. 11, 2010 | Feb. 11, 2010 | Feb. 18, 2010 |
| 407 | Oct. 5, 2009 | Dec. 10, 2009 | 1pf | Fasting | Feb. 11, 2010 | Feb. 11, 2010 | Feb. 18, 2010 |
| 408 | Oct. 5, 2009 | Dec. 10, 2009 | 1pf | Fasting | Feb. 11, 2010 | Feb. 11, 2010 | Feb. 18, 2010 |
| 409 | Oct. 5, 2009 | Dec. 10, 2009 | 1pf | Fasting | Feb. 11, 2010 | Feb. 11, 2010 | Feb. 18, 2010 |
| 410 | Oct. 5, 2009 | Dec. 10, 2009 | 1pf | Fasting | Feb. 11, 2010 | Feb. 11, 2010 | Feb. 18, 2010 |
| 411 | Oct. 5, 2009 | Dec. 10, 2009 | 1pf | Fasting | Feb. 11, 2010 | Feb. 11, 2010 | Feb. 18, 2010 |
| 412 | Oct. 5, 2009 | Dec. 10, 2009 | 1pf | Fasting | Feb. 11, 2010 | Feb. 11, 2010 | Feb. 18, 2010 |
| 413 | Oct. 5, 2009 | Dec. 17, 2009 | 1pf | Fasting | Feb. 11, 2010 | Feb. 11, 2010 | Feb. 18, 2010 |
| 414 | Oct. 5, 2009 | Dec. 17, 2009 | 1pf | Fasting | Feb. 11, 2010 | Feb. 11, 2010 | Feb. 18, 2010 |
| 416 | Oct. 5, 2009 | Dec. 17, 2009 | 1pf | Fasting | Feb. 11, 2010 | Feb. 11, 2010 | Feb. 18, 2010 |
| 417 | Oct. 5, 2009 | Dec. 17, 2009 | 1pf | Fasting | Feb. 11, 2010 | Feb. 11, 2010 | Feb. 18, 2010 |
| 418 | Oct. 5, 2009 | Dec. 17, 2009 | 1pf | Fasting | Feb. 11, 2010 | Feb. 11, 2010 | Feb. 18, 2010 |
| 419 | Oct. 5, 2009 | Dec. 17, 2009 | 1pf | Fasting | Feb. 11, 2010 | Feb. 11, 2010 | Feb. 18, 2010 |
| 420 | Oct. 5, 2009 | Dec. 17, 2009 | 1pf | Fasting | Feb. 11, 2010 | Feb. 11, 2010 | Feb. 18, 2010 |
| 421 | Oct. 5, 2009 | Dec. 10, 2009 | 2pf | LF/PP | Feb. 12, 2010 | N/A | N/A |
| 422 | Oct. 5, 2009 | Dec. 10, 2009 | 2pf | LF/PP | Feb. 12, 2010 | N/A | N/A |
| 423 | Oct. 5, 2009 | Dec. 10, 2009 | 2pf | LF/PP | Feb. 12, 2010 | N/A | N/A |
| 424 | Oct. 5, 2009 | Dec. 10, 2009 | 2pf | LF/PP | Feb. 12, 2010 | N/A | N/A |
| 425 | Oct. 5, 2009 | Dec. 10, 2009 | 2pf | LF/PP | Feb. 12, 2010 | N/A | N/A |
| 426 | Oct. 5, 2009 | Dec. 10, 2009 | 2pf | LF/PP | Feb. 12, 2010 | N/A | N/A |
| 427 | Oct. 5, 2009 | Dec. 10, 2009 | 2pf | LF/PP | Feb. 12, 2010 | N/A | N/A |
| 428 | Oct. 5, 2009 | Dec. 10, 2009 | 2pf | LF/PP | Feb. 12, 2010 | N/A | N/A |
| 429 | Oct. 5, 2009 | Dec. 10, 2009 | 2pf | LF/PP | Feb. 12, 2010 | N/A | N/A |
| 430 | Oct. 5, 2009 | Dec. 10, 2009 | 2pf | LF/PP | Feb. 12, 2010 | N/A | N/A |
| 431 | Oct. 5, 2009 | Dec. 17, 2009 | 2pf | LF/PP | Feb. 12, 2010 | N/A | N/A |
| 432 | Oct. 5, 2009 | Dec. 17, 2009 | 2pf | LF/PP | Feb. 12, 2010 | N/A | N/A |
| 433 | Oct. 5, 2009 | Dec. 17, 2009 | 2pf | LF/PP | Feb. 12, 2010 | N/A | N/A |
| 434 | Oct. 5, 2009 | Dec. 17, 2009 | 2pf | LF/PP | Feb. 12, 2010 | N/A | N/A |
| 435 | Oct. 5, 2009 | Dec. 17, 2009 | 2pf | LF/PP | Feb. 12, 2010 | N/A | N/A |
| 436 | Oct. 5, 2009 | Dec. 17, 2009 | 2pf | LF/PP | Feb. 12, 2010 | N/A | N/A |
| 437 | Oct. 5, 2009 | Dec. 17, 2009 | 2pf | LF/PP | Feb. 12, 2010 | N/A | N/A |
| 438 | Oct. 5, 2009 | Dec. 17, 2009 | 2pf | LF/PP | Feb. 12, 2010 | N/A | N/A |
| 439 | Oct. 5, 2009 | Dec. 10, 2009 | 5pf | LF/PP | Feb. 15, 2010 | N/A | N/A |
| 440 | Oct. 5, 2009 | Dec. 10, 2009 | 5pf | LF/PP | Feb. 15, 2010 | N/A | N/A |
| 441 | Oct. 5, 2009 | Dec. 10, 2009 | 5pf | LF/PP | Feb. 15, 2010 | N/A | N/A |
| 442 | Oct. 5, 2009 | Dec. 10, 2009 | 5pf | LF/PP | Feb. 15, 2010 | N/A | N/A |
| 443 | Oct. 5, 2009 | Dec. 10, 2009 | 5pf | LF/PP | Feb. 15, 2010 | N/A | N/A |
| 444 | Oct. 5, 2009 | Dec. 10, 2009 | 5pf | LF/PP | Feb. 15, 2010 | N/A | N/A |
| 445 | Oct. 5, 2009 | Dec. 10, 2009 | 5pf | LF/PP | Feb. 15, 2010 | N/A | N/A |
| 446 | Oct. 5, 2009 | Dec. 10, 2009 | 5pf | LF/PP | Feb. 15, 2010 | N/A | N/A |
| 447 | Oct. 5, 2009 | Dec. 10, 2009 | 5pf | LF/PP | Feb. 15, 2010 | N/A | N/A |
| 448 | Oct. 5, 2009 | Dec. 10, 2009 | 5pf | LF/PP | Feb. 15, 2010 | N/A | N/A |
| 449 | Oct. 5, 2009 | Dec. 17, 2009 | 5pf | LF/PP | Feb. 15, 2010 | N/A | N/A |
| 450 | Oct. 5, 2009 | Dec. 17, 2009 | 5pf | LF/PP | Feb. 15, 2010 | N/A | N/A |
| 451 | Oct. 5, 2009 | Dec. 17, 2009 | 5pf | LF/PP | Feb. 15, 2010 | N/A | N/A |
| 452 | Oct. 5, 2009 | Dec. 17, 2009 | 5pf | LF/PP | Feb. 15, 2010 | N/A | N/A |
| 453 | Oct. 5, 2009 | Dec. 17, 2009 | 5pf | LF/PP | Feb. 15, 2010 | N/A | N/A |
| 454 | Oct. 5, 2009 | Dec. 17, 2009 | 5pf | LF/PP | Feb. 15, 2010 | N/A | N/A |
| 455 | Oct. 5, 2009 | Dec. 17, 2009 | 5pf | LF/PP | Feb. 15, 2010 | N/A | N/A |
| 456 | Oct. 5, 2009 | Dec. 17, 2009 | 5pf | LF/PP | Feb. 15, 2010 | N/A | N/A |
| 457 | Oct. 5, 2009 | Dec. 10, 2009 | 9pf | LF/PP | Feb. 19, 2010 | N/A | N/A |
| 458 | Oct. 5, 2009 | Dec. 10, 2009 | 9pf | LF/PP | Feb. 19, 2010 | N/A | N/A |
| 459 | Oct. 5, 2009 | Dec. 10, 2009 | 9pf | LF/PP | Feb. 19, 2010 | N/A | N/A |
| 460 | Oct. 5, 2009 | Dec. 10, 2009 | 9pf | LF/PP | Feb. 19, 2010 | N/A | N/A |
| 461 | Oct. 5, 2009 | Dec. 10, 2009 | 9pf | LF/PP | Feb. 19, 2010 | N/A | N/A |
| 462 | Oct. 5, 2009 | Dec. 10, 2009 | 9pf | LF/PP | Feb. 19, 2010 | N/A | N/A |
| 463 | Oct. 5, 2009 | Dec. 10, 2009 | 9pf | LF/PP | Feb. 19, 2010 | N/A | N/A |
| 465 | Oct. 5, 2009 | Dec. 10, 2009 | 9pf | LF/PP | Feb. 19, 2010 | N/A | N/A |
| 466 | Oct. 5, 2009 | Dec. 10, 2009 | 9pf | LF/PP | Feb. 19, 2010 | N/A | N/A |
| 467 | Oct. 5, 2009 | Dec. 17, 2009 | 9pf | LF/PP | Feb. 19, 2010 | N/A | N/A |
| 468 | Oct. 5, 2009 | Dec. 17, 2009 | 9pf | LF/PP | Feb. 19, 2010 | N/A | N/A |
| 469 | Oct. 5, 2009 | Dec. 17, 2009 | 9pf | LF/PP | Feb. 19, 2010 | N/A | N/A |
| 470 | Oct. 5, 2009 | Dec. 17, 2009 | 9pf | LF/PP | Feb. 19, 2010 | N/A | N/A |
| 471 | Oct. 5, 2009 | Dec. 17, 2009 | 9pf | LF/PP | Feb. 19, 2010 | N/A | N/A |
| 472 | Oct. 5, 2009 | Dec. 17, 2009 | 9pf | LF/PP | Feb. 19, 2010 | N/A | N/A |
| 473 | Oct. 5, 2009 | Dec. 17, 2009 | 9pf | LF/PP | Feb. 19, 2010 | N/A | N/A |
| 474 | Oct. 5, 2009 | Dec. 17, 2009 | 9pf | LF/PP | Feb. 19, 2010 | N/A | N/A |
| 493 | Oct. 5, 2009 | Dec. 10, 2009 | sac | LF/PP | Feb. 19, 2010 | N/A | N/A |
| 494 | Oct. 5, 2009 | Dec. 10, 2009 | sac | LF/PP | Feb. 19, 2010 | N/A | N/A |
| 495 | Oct. 5, 2009 | Dec. 10, 2009 | sac | LF/PP | Feb. 19, 2010 | N/A | N/A |
| 496 | Oct. 5, 2009 | Dec. 10, 2009 | sac | LF/PP | Feb. 19, 2010 | N/A | N/A |
| 498 | Oct. 5, 2009 | Dec. 10, 2009 | sac | LF/PP | Feb. 19, 2010 | N/A | N/A |
| 499 | Oct. 5, 2009 | Dec. 10, 2009 | sac | LF/PP | Feb. 19, 2010 | N/A | N/A |
| 500 | Oct. 5, 2009 | Dec. 10, 2009 | sac | LF/PP | Feb. 19, 2010 | N/A | N/A |
| 501 | Oct. 5, 2009 | Dec. 10, 2009 | sac | LF/PP | Feb. 19, 2010 | N/A | N/A |
| 502 | Oct. 5, 2009 | Dec. 10, 2009 | sac | LF/PP | Feb. 19, 2010 | N/A | N/A |
| 503 | Oct. 5, 2009 | Dec. 17, 2009 | sac | LF/PP | Feb. 19, 2010 | N/A | N/A |
| 505 | Oct. 5, 2009 | Dec. 17, 2009 | sac | LF/PP | Feb. 19, 2010 | N/A | N/A |
| 506 | Oct. 5, 2009 | Dec. 17, 2009 | sac | LF/PP | Feb. 19, 2010 | N/A | N/A |
| 507 | Oct. 5, 2009 | Dec. 17, 2009 | sac | LF/PP | Feb. 19, 2010 | N/A | N/A |
| 509 | Oct. 5, 2009 | Dec. 17, 2009 | sac | LF/PP | Feb. 19, 2010 | N/A | N/A |
| 510 | Oct. 5, 2009 | Dec. 17, 2009 | sac | LF/PP | Feb. 19, 2010 | N/A | N/A |
| 511 | Oct. 5, 2009 | Dec. 10, 2009 | sac | LF/PP | Feb. 19, 2010 | N/A | N/A |
| 512 | Oct. 5, 2009 | Dec. 10, 2009 | sac | LF/PP | Feb. 19, 2010 | N/A | N/A |
| 513 | Oct. 5, 2009 | Dec. 10, 2009 | sac | LF/PP | Feb. 19, 2010 | N/A | N/A |
| 514 | Oct. 5, 2009 | Dec. 10, 2009 | sac | LF/PP | Feb. 19, 2010 | N/A | N/A |
| 515 | Oct. 5, 2009 | Dec. 10, 2009 | sac | LF/PP | Feb. 19, 2010 | N/A | N/A |
| 517 | Oct. 5, 2009 | Dec. 10, 2009 | sac | LF/PP | Feb. 19, 2010 | N/A | N/A |
| 518 | Oct. 5, 2009 | Dec. 10, 2009 | sac | LF/PP | Feb. 19, 2010 | N/A | N/A |
| 519 | Oct. 5, 2009 | Dec. 10, 2009 | sac | LF/PP | Feb. 19, 2010 | N/A | N/A |
| 520 | Oct. 5, 2009 | Dec. 10, 2009 | sac | LF/PP | Feb. 19, 2010 | N/A | N/A |
| 521 | Oct. 5, 2009 | Dec. 17, 2009 | sac | LF/PP | Feb. 19, 2010 | N/A | N/A |
| 522 | Oct. 5, 2009 | Dec. 17, 2009 | sac | LF/PP | Feb. 19, 2010 | N/A | N/A |
| 523 | Oct. 5, 2009 | Dec. 17, 2009 | sac | LF/PP | Feb. 19, 2010 | N/A | N/A |
| 524 | Oct. 5, 2009 | Dec. 17, 2009 | sac | LF/PP | Feb. 19, 2010 | N/A | N/A |
| 525 | Oct. 5, 2009 | Dec. 17, 2009 | sac | LF/PP | Feb. 19, 2010 | N/A | N/A |
| 526 | Oct. 5, 2009 | Dec. 17, 2009 | sac | LF/PP | Feb. 19, 2010 | N/A | N/A |
| 527 | Oct. 5, 2009 | Dec. 17, 2009 | sac | LF/PP | Feb. 19, 2010 | N/A | N/A |
| 547 | Oct. 5, 2009 | Dec. 10, 2009 | sac | LF/PP | Feb. 19, 2010 | N/A | N/A |
| 548 | Oct. 5, 2009 | Dec. 10, 2009 | sac | LF/PP | Feb. 19, 2010 | N/A | N/A |
| 549 | Oct. 5, 2009 | Dec. 10, 2009 | sac | LF/PP | Feb. 19, 2010 | N/A | N/A |
| 550 | Oct. 5, 2009 | Dec. 10, 2009 | sac | LF/PP | Feb. 19, 2010 | N/A | N/A |
| 551 | Oct. 5, 2009 | Dec. 10, 2009 | sac | LF/PP | Feb. 19, 2010 | N/A | N/A |
| 552 | Oct. 5, 2009 | Dec. 10, 2009 | sac | LF/PP | Feb. 19, 2010 | N/A | N/A |
| 553 | Oct. 5, 2009 | Dec. 10, 2009 | sac | LF/PP | Feb. 19, 2010 | N/A | N/A |
| 554 | Oct. 5, 2009 | Dec. 10, 2009 | sac | LF/PP | Feb. 19, 2010 | N/A | N/A |
| 555 | Oct. 5, 2009 | Dec. 10, 2009 | sac | LF/PP | Feb. 19, 2010 | N/A | N/A |
| 557 | Oct. 5, 2009 | Dec. 17, 2009 | sac | LF/PP | Feb. 19, 2010 | N/A | N/A |
| 558 | Oct. 5, 2009 | Dec. 17, 2009 | sac | LF/PP | Feb. 19, 2010 | N/A | N/A |
| 559 | Oct. 5, 2009 | Dec. 17, 2009 | sac | LF/PP | Feb. 19, 2010 | N/A | N/A |
| 560 | Oct. 5, 2009 | Dec. 17, 2009 | sac | LF/PP | Feb. 19, 2010 | N/A | N/A |
| 561 | Oct. 5, 2009 | Dec. 17, 2009 | sac | LF/PP | Feb. 19, 2010 | N/A | N/A |
| 562 | Oct. 5, 2009 | Dec. 17, 2009 | sac | LF/PP | Feb. 19, 2010 | N/A | N/A |
| 563 | Oct. 5, 2009 | Dec. 17, 2009 | sac | LF/PP | Feb. 19, 2010 | N/A | N/A |
| 583 | Oct. 5, 2009 | Dec. 10, 2009 | sac | LF/PP | Feb. 19, 2010 | N/A | N/A |
| 584 | Oct. 5, 2009 | Dec. 10, 2009 | sac | LF/PP | Feb. 19, 2010 | N/A | N/A |
| 587 | Oct. 5, 2009 | Dec. 10, 2009 | sac | LF/PP | Feb. 19, 2010 | N/A | N/A |
| 588 | Oct. 5, 2009 | Dec. 10, 2009 | sac | LF/PP | Feb. 19, 2010 | N/A | N/A |
| 589 | Oct. 5, 2009 | Dec. 10, 2009 | sac | LF/PP | Feb. 19, 2010 | N/A | N/A |
| 591 | Oct. 5, 2009 | Dec. 10, 2009 | sac | LF/PP | Feb. 19, 2010 | N/A | N/A |
| 592 | Oct. 5, 2009 | Dec. 10, 2009 | sac | LF/PP | Feb. 19, 2010 | N/A | N/A |
| 593 | Oct. 5, 2009 | Dec. 17, 2009 | sac | LF/PP | Feb. 19, 2010 | N/A | N/A |
| 594 | Oct. 5, 2009 | Dec. 17, 2009 | sac | LF/PP | Feb. 19, 2010 | N/A | N/A |
| 595 | Oct. 5, 2009 | Dec. 17, 2009 | sac | LF/PP | Feb. 19, 2010 | N/A | N/A |
| 596 | Oct. 5, 2009 | Dec. 17, 2009 | sac | LF/PP | Feb. 19, 2010 | N/A | N/A |
| 597 | Oct. 5, 2009 | Dec. 17, 2009 | sac | LF/PP | Feb. 19, 2010 | N/A | N/A |
| 598 | Oct. 5, 2009 | Dec. 17, 2009 | sac | LF/PP | Feb. 19, 2010 | N/A | N/A |
| 599 | Oct. 5, 2009 | Dec. 17, 2009 | sac | LF/PP | Feb. 19, 2010 | N/A | N/A |
| 600 | Oct. 5, 2009 | Dec. 17, 2009 | sac | LF/PP | Feb. 19, 2010 | N/A | N/A |
| 601 | Oct. 5, 2009 | Dec. 10, 2009 | sac | LF/PP | Feb. 19, 2010 | N/A | N/A |
| 602 | Oct. 5, 2009 | Dec. 10, 2009 | sac | LF/PP | Feb. 19, 2010 | N/A | N/A |
| 603 | Oct. 5, 2009 | Dec. 10, 2009 | sac | LF/PP | Feb. 19, 2010 | N/A | N/A |
| 604 | Oct. 5, 2009 | Dec. 10, 2009 | sac | LF/PP | Feb. 19, 2010 | N/A | N/A |
| 605 | Oct. 5, 2009 | Dec. 10, 2009 | sac | LF/PP | Feb. 19, 2010 | N/A | N/A |
| 606 | Oct. 5, 2009 | Dec. 10, 2009 | sac | LF/PP | Feb. 19, 2010 | N/A | N/A |
| 607 | Oct. 5, 2009 | Dec. 10, 2009 | sac | LF/PP | Feb. 19, 2010 | N/A | N/A |
| 608 | Oct. 5, 2009 | Dec. 10, 2009 | sac | LF/PP | Feb. 19, 2010 | N/A | N/A |
| 609 | Oct. 5, 2009 | Dec. 10, 2009 | sac | LF/PP | Feb. 19, 2010 | N/A | N/A |
| 610 | Oct. 5, 2009 | Dec. 10, 2009 | sac | LF/PP | Feb. 19, 2010 | N/A | N/A |
| 611 | Oct. 5, 2009 | Dec. 17, 2009 | sac | LF/PP | Feb. 19, 2010 | N/A | N/A |
| 612 | Oct. 5, 2009 | Dec. 17, 2009 | sac | LF/PP | Feb. 19, 2010 | N/A | N/A |
| 613 | Oct. 5, 2009 | Dec. 17, 2009 | sac | LF/PP | Feb. 19, 2010 | N/A | N/A |
| 614 | Oct. 5, 2009 | Dec. 17, 2009 | sac | LF/PP | Feb. 19, 2010 | N/A | N/A |
| 615 | Oct. 5, 2009 | Dec. 17, 2009 | sac | LF/PP | Feb. 19, 2010 | N/A | N/A |
| 616 | Oct. 5, 2009 | Dec. 17, 2009 | sac | LF/PP | Feb. 19, 2010 | N/A | N/A |
| 617 | Oct. 5, 2009 | Dec. 17, 2009 | sac | LF/PP | Feb. 19, 2010 | N/A | N/A |
| 618 | Oct. 5, 2009 | Dec. 17, 2009 | sac | LF/PP | Feb. 19, 2010 | N/A | N/A |
| 1001 | N/A | N/A | N/A | N/A | N/A | N/A | N/A |
| 1002 | N/A | N/A | N/A | N/A | N/A | N/A | N/A |
| 1003 | N/A | N/A | N/A | N/A | N/A | N/A | N/A |
| 2000 | N/A | N/A | N/A | N/A | N/A | N/A | N/A |
| 2001 | N/A | N/A | N/A | N/A | N/A | N/A | N/A |
| 2002 | N/A | N/A | N/A | N/A | N/A | N/A | N/A |
| 2003 | N/A | N/A | N/A | N/A | N/A | N/A | N/A |
| 2004 | N/A | N/A | N/A | N/A | N/A | N/A | N/A |
| 2005 | N/A | N/A | N/A | N/A | N/A | N/A | N/A |
| 2006 | N/A | N/A | N/A | N/A | N/A | N/A | N/A |
| 2007 | N/A | N/A | N/A | N/A | N/A | N/A | N/A |
| 2008 | N/A | N/A | N/A | N/A | N/A | N/A | N/A |
| 2009 | N/A | N/A | N/A | N/A | N/A | N/A | N/A |
| 2010 | N/A | N/A | N/A | N/A | N/A | N/A | N/A |
| 2011 | N/A | N/A | N/A | N/A | N/A | N/A | N/A |
| 2012 | N/A | N/A | N/A | N/A | N/A | N/A | N/A |
| 2013 | N/A | N/A | N/A | N/A | N/A | N/A | N/A |
| 2014 | N/A | N/A | N/A | N/A | N/A | N/A | N/A |
| 2015 | N/A | N/A | N/A | N/A | N/A | N/A | N/A |
| 2016 | N/A | N/A | N/A | N/A | N/A | N/A | N/A |
| 2017 | N/A | N/A | N/A | N/A | N/A | N/A | N/A |
| 2018 | N/A | N/A | N/A | N/A | N/A | N/A | N/A |
| 2019 | N/A | N/A | N/A | N/A | N/A | N/A | N/A |
| 3001 | N/A | N/A | N/A | N/A | N/A | N/A | N/A |
| 3002 | N/A | N/A | N/A | N/A | N/A | N/A | N/A |
| 3003 | N/A | N/A | N/A | N/A | N/A | N/A | N/A |
| 3004 | N/A | N/A | N/A | N/A | N/A | N/A | N/A |
| 3005 | N/A | N/A | N/A | N/A | N/A | N/A | N/A |
| 3006 | N/A | N/A | N/A | N/A | N/A | N/A | N/A |
| 3007 | N/A | N/A | N/A | N/A | N/A | N/A | N/A |
| 3008 | N/A | N/A | N/A | N/A | N/A | N/A | N/A |
| 3009 | N/A | N/A | N/A | N/A | N/A | N/A | N/A |
| 3010 | N/A | N/A | N/A | N/A | N/A | N/A | N/A |
| 3011 | N/A | N/A | N/A | N/A | N/A | N/A | N/A |
| 3012 | N/A | N/A | N/A | N/A | N/A | N/A | N/A |
| 4000 | N/A | N/A | N/A | N/A | N/A | N/A | N/A |
| 4001 | N/A | N/A | N/A | N/A | N/A | N/A | N/A |
| 4002 | N/A | N/A | N/A | N/A | N/A | N/A | N/A |
| 4003 | Jan. 10, 2010 | Jun. 1, 2010 | 3pg | LF/PP | Jun. 4, 2010 | N/A | N/A |
| 4004 | Jan. 10, 2010 | Jun. 1, 2010 | 3pg | LF/PP | Jun. 4, 2010 | N/A | N/A |
| 4005 | Jan. 10, 2010 | Jun. 1, 2010 | 3pg | LF/PP | Jun. 4, 2010 | N/A | N/A |
| 4006 | Jan. 10, 2010 | Jun. 1, 2010 | 3pg | LF/PP | Jun. 4, 2010 | N/A | N/A |
| 4007 | Jan. 10, 2010 | Jun. 1, 2010 | 3pg | LF/PP | Jun. 4, 2010 | N/A | N/A |
| 4008 | Jan. 10, 2010 | Jun. 1, 2010 | 7pg | LF/PP | Jun. 8, 2010 | N/A | N/A |
| 4009 | Jan. 10, 2010 | Jun. 1, 2010 | 7pg | LF/PP | Jun. 8, 2010 | N/A | N/A |
| 4010 | Jan. 10, 2010 | Jun. 1, 2010 | 7pg | LF/PP | Jun. 8, 2010 | N/A | N/A |
| 4011 | Jan. 10, 2010 | Jun. 1, 2010 | 7pg | LF/PP | Jun. 8, 2010 | N/A | N/A |
| 4012 | Jan. 10, 2010 | Jun. 1, 2010 | 7pg | LF/PP | Jun. 8, 2010 | N/A | N/A |
| 4013 | Jan. 10, 2010 | Jun. 1, 2010 | 14pg | LF/PP | Jun. 15, 2010 | N/A | N/A |
| 4014 | Jan. 10, 2010 | Jun. 1, 2010 | 14pg | LF/PP | Jun. 15, 2010 | N/A | N/A |
| 4015 | Jan. 10, 2010 | Jun. 1, 2010 | 14pg | LF/PP | Jun. 15, 2010 | N/A | N/A |
| 4016 | Jan. 10, 2010 | Jun. 1, 2010 | 14pg | LF/PP | Jun. 15, 2010 | N/A | N/A |
| 4017 | Jan. 10, 2010 | Jun. 1, 2010 | 14pg | LF/PP | Jun. 15, 2010 | N/A | N/A |

What is claimed is:

1. A composition, the composition comprising (i) an in vitro cultured collection of a gut microbial community or (ii) a clonally arrayed culture collection of a gut microbial community.

2. The composition of claim 1, wherein the gut microbial community is from a human or a germfree mouse colonized with a gut microbial community or an arrayed culture collection of a gut microbial community.

3. The composition of claim 1, wherein the cultured microbial community was cultured on gut microbiota medium.

4. The composition of claim 1, wherein the cultured gut microbial community has (i) at least 60%, at least 70%, at least 80% or at least 90% of the order-level phylotopic composition of the original gut microbial community; or (ii) at least 60%, at least 70%, at least 80% or at least 90% of the metagenome, transcriptome, or proteome composition of the original gut microbial community; or (iii) at least 60%, at least 70%, at least 80% or at least 90% of the order-level phylotopic composition of the original gut microbial community and at least 60%, at least 70%, at least 80% or at least 90% of the metagenome, transcriptome, or proteome composition of the original gut microbial community.

5. The composition of claim 1, wherein the cultured gut microbial community has (i) at least 98.0% of the order-level phylotopic composition of the original gut microbial community; or (ii) at least 98.0% of the metagenome, transcriptome, or proteome composition of the original gut microbial community; or (iii) at least 98.0% of the order-level phylotopic composition of the original gut microbial community and at least 98.0% of the metagenome, transcriptome, or proteome composition of the original gut microbial community.

6. The composition of claim 4, wherein each member of the collection is assigned a barcode.

7. The composition of claim 1, wherein the clonally arrayed culture collection was prepared (i) without colony picking; or (ii) using a most probable number (MPN) technique.

8. A method of determining the effect of a perturbation on a gut microbial community, the method comprising applying the perturbation to a cultured collection of a gut microbial community and determining the difference in the community before and after the application of the perturbation, wherein the difference in the cultured collection represents the effect of the perturbation on the original gut microbial community.

9. The method of claim 8, wherein the perturbation is a diet related perturbation, an environmental perturbation, a genetic perturbation or a pharmaceutical perturbation.

10. A method of specifically manipulating the abundance of a member of a gut microbiome of a host to a target level by changing the diet of the host, the method comprising
(a) determining the linear coefficient for a particular gut microbiome member in relation to protein, fat, polysaccharide, and simple sugar;
(b) determining the amount of protein, fat, polysaccharide and sugar in a diet necessary to achieve the target level of the gut microbiome member based on the linear coefficients from (a); and
(c) feeding a diet to the host that contains the amount of protein, fat, polysaccharide and sugar determined in (b).

11. The method of claim 10, wherein the linear coefficient for a particular gut microbiome member for a particular food ingredient is determined using a gnotobiotic mouse model of a human gut microbiome community.

12. The method of claim 10, wherein the abundance of a member of a gut microbiome may be calculated with the equation
yᵢ=β₀+βₚᵣₒₜₑᵢₙXₚᵣₒₜₑᵢₙ+β_{polysaccharide}X_{polysaccharide}+β_{sucrose}X_{sucrose}+β_{fat}X_{fat}
where yᵢ is the abundance of the member of the gut microbiome, β₀ is the calculated parameter for the intercept, X is the amount in g/(kg of total diet) of the diet ingredient, and βₚᵣₒₜₑᵢₙ, β_{polysaccharide}, β_{sucrose}, and Nat are the linear coefficients for a particular gut microbiome member for each of the diet components.

13. The method of claim 12, wherein β₀ for a particular gut microbiome member for a particular food ingredient is determined using a gnotobiotic mouse model of a human gut microbiome community.

14. The method of claim 10, wherein the host is a rodent, a human, a livestock animal, a companion animal, or a zoological animal.

15. The method of claim 14, wherein the livestock animal is a pig, cow, horse, goat, sheep, llama, alpaca or swine.
